(19) United States
(12) Patent Application Publication
(10) Pub. No.: US 2004/0137423 A1
(43) Pub. Date: Jul. 15, 2004

(54) Compositions and methods for modulating HDL cholesterol and triglyceride levels

(76) Inventors: Michael R. Hayden, Vancouver (CA); Angela R. Brooks-Wilson, Richmond (CA); Simon N. Pimstone, Vancouver (CA); Susanne M. Clee, Vancouver (CA)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 10/745,377

(22) Filed: Dec. 23, 2003

Related U.S. Application Data
(62) Division of application No. 09/654,323, filed on Sep. 1, 2000.
(63) Continuation-in-part of application No. 09/526,193, filed on Mar. 15, 2000, now Pat. No. 6,617,122.
(60) Provisional application No. 60/213,958, filed on Jun. 23, 2000.
(60) Provisional application No. 60/124,702, filed on Mar. 15, 1999.
(60) Provisional application No. 60/138,048, filed on Jun. 8, 1999.
(60) Provisional application No. 60/139,600, filed on Jun. 17, 1999.
(60) Provisional application No. 60/151,977, filed on Sep. 1, 1999.

(51) Int. Cl. C12Q 1/00; C12Q 1/68

(52) U.S. Cl. 435/4; 435/6

(57) ABSTRACT

The invention provides methods for identifying agents that modulate HDL-levels in animals by evaluating the ability of LXR-modulating agents to increase ABCA1-gene expression in a cell as well as using such agents to treat conditions involving lower than normal HDL-levels, higher than normal triglyceride levels and the like.

This application is a divisional of U.S. application Ser. No. 09/654,323, filed Sep. 1, 2000, and claims priority of Provisional Application No. 60/213,958 filed Jun. 23, 2000, and U.S. Utility application Ser. No. 09/526,193, filed Mar. 15, 2000, which claims priority from U.S. Provisional Application No. 60/124,702, filed Mar. 15, 1999; U.S. Provisional Application No. 60/138,048, filed Jun. 8, 1999; U.S. Provisional Application No. 60/139,600, filed Jun. 17, 1999; and U.S. Provisional Application No. 60/151,977, filed Sep. 1, 1999.

### BACKGROUND OF THE INVENTION

Low HDL cholesterol (HDL-C), or hypoalphalipoproteinemia, is a blood lipid abnormality which correlates with a high risk of cardiovascular disease (CVD), in particular coronary artery disease (CAD), but also cerebrovascular disease, coronary restenosis, and peripheral vascular disease. HDL-C levels are influenced by both environmental and genetic factors.

Epidemiological studies have consistently demonstrated that plasma HDL-C concentration is inversely related to the incidence of CAD. HDL-C levels are a strong graded and independent cardiovascular risk factor. Protective effects of an elevated HDL-C persist until 80 years of age. A low HDL-C is associated with an increased CAD risk even with normal (<5.2 mmol/l) total plasma cholesterol levels. Coronary disease risk is increased by 2% in men and 3% in women for every 1 mg/dL (0.026 mmol/l) reduction in HDL-C and in the majority of studies this relationship is statistically significant even after adjustment for other lipid and non-lipid risk factors. Decreased HDL-C levels are the most common lipoprotein abnormality seen in patients with premature CAD. Four percent of patients with premature CAD have an isolated form of decreased HDL-C levels with no other lipoprotein abnormalities while 25% have low HDL-C levels with accompanying hypertriglyceridemia.

Even in the face of other dyslipidemias or secondary factors, HDL-C levels are important predictors of CAD. In a cohort of diabetics, those with isolated low HDL-C had a 65% increased death rate compared to diabetics with normal HDL-C levels (>0.9 mmol/l). Furthermore, it has been shown that even within high risk populations, such as those with familial hypercholesterolemia, HDL-C level is an important predictor of CAD. Low HDL-C levels thus constitute a major, independent, risk for CAD.

These findings have led to increased attention to HDL-C levels as a focus for treatment, following the recommendations of the National Cholesterol Education Program. These guidelines suggest that HDL-C values below 0.9 mmol/l confer a significant risk for men and women. As such, nearly half of patients with CAD would have low HDL-C. It is therefore crucial that we obtain a better understanding of factors which contribute to this phenotype. In view of the fact that pharmacological intervention of low HDL-C levels has so far proven unsatisfactory, it is also important to understand the factors that regulate these levels in the circulation as this understanding may reveal new therapeutic targets.

Absolute levels of HDL-C may not always predict risk of CAD. In the case of CETP deficiency, individuals display an increased risk of developing CAD, despite increased HDL-C levels. What seems to be important in this case is the functional activity of the reverse cholesterol transport pathway, the process by which intracellular cholesterol is trafficked out of the cell to acceptor proteins such as ApoAI or HDL. Other important genetic determinants of HDL-C levels, and its inverse relation with CAD, may reside in the processes leading to HDL formation and intracellular cholesterol trafficking and efflux. To date, this process is poorly understood, however, and clearly not all of the components of this pathway have been identified. Thus, defects preventing proper HDL-mediated cholesterol efflux may be important predictors of CAD. Therefore it is critical to identify and understand novel genes involved in the intracellular cholesterol trafficking and efflux pathways.

HDL particles are central to the process of reverse cholesterol transport and thus to the maintenance of tissue cholesterol homeostasis. This process has multiple steps which include the binding of HDL to cell surface components, the acquisition of cholesterol by passive absorption, the esterification of this cholesterol by LCAT and the subsequent transfer of esterified cholesterol by CETP, to VLDL and chylomicron remnants for liver uptake. Each of these steps is known to impact the plasma concentration of HDL.

Changes in genes for ApoAI-CIII, lipoprotein lipase, CETP, hepatic lipase, and LCAT all contribute to determination of HDL-C levels in humans. One rare form of genetic HDL deficiency is Tangier disease (TD), diagnosed in approximately 40 patients world-wide, and associated with almost complete absence of HDL-C levels (listed in OMIM as an autosomal recessive trait (OMIM 205400)). These patients have very low HDL-C and ApoAI levels, which have been ascribed to impairment of lipid transport and hypercatabolism of nascent HDL and ApoAI, due to a delayed acquisition of lipid and resulting failure of conversion to mature HDL. TD patients accumulate cholesterol esters in several tissues, resulting in characteristic features, such as enlarged yellow tonsils, corneal opacity, hepatosplenomegaly, peripheral neuropathy, and cholesterol ester deposition in the rectal mucosa. Defective removal of cellular cholesterol and phospholipids by ApoAI as well as a marked deficiency in HDL mediated efflux of intracellular cholesterol has been demonstrated in TD fibroblasts. Even though this is a rare disorder, defining its molecular basis could identify pathways relevant for cholesterol regulation in the general population. The decreased availability of free cholesterol for efflux in the surface membranes of cells in Tangier Disease patients appears to be due to a defect in cellular lipid metabolism or trafficking. Approximately 45% of Tangier patients have signs of premature CAD, suggesting a strong link between decreased cholesterol efflux, low HDL-C and CAD. increased As cholesterol is observed in the rectal mucosa of persons with TD, the molecular mechanism responsible for TD may also regulate cholesterol adsorption from the gastrointestinal (GI) tract.

A more common form of genetic HDL deficiency occurs in patients who have low plasma HDL-C usually below the 5th percentile for age and sex (OMIM 10768), but an absence of clinical manifestations specific to Tangier disease (Marcil et al., Arterioscler. Thromb. Vasc. Biol. 19:159-169, 1999; Marcil et al., Arterioscler. Thromb. Vasc. Biol. 15:1015-1024, 1995). These patients have no obvious environmental factors associated with this lipid phenotype, and do not have severe hypertriglyceridemia nor have known causes of severe HDL deficiency (mutations in ApoAI, LCAT, or LPL deficiency) and are not diabetic. The pattern of inheritance of this condition is most consistent with a Mendelian dominant trait (OMIM 10768).

The development of drugs that regulate cholesterol metabolism has so far progressed slowly. Thus, there is a need for a better understanding of the genetic components of the cholesterol efflux pathway. Newly-discovered components can then serve as targets for drugs.

### SUMMARY OF THE INVENTION

In a first aspect, the invention features a method for treating a patient diagnosed as having a lower than normal HDL-cholesterol level or a higher than normal triglyceride level. The method includes administering to the patient a compound that modulates LXR-mediated transcriptional activity. Preferably, the compound is administered to the patient with a pharmaceutically acceptable carrier. The compound may be selected, for example, from the group consisting of 24-(S),25-epoxycholesterol; 24(S)-hydroxycholesterol; 22-(R)-hydroxycholesterol; 24(R),25-epoxycholesterol; 22(R)-hydroxy-24(S),25-epoxycholesterol; 22(S)-hydroxy-24(R),25-epoxycholesterol; 24-(S),25-iminocholesterol; methyl-38-hydroxycholonate; N,N-dimethyl-3β-hydroxycholonamide; 24(R)-hydroxycholesterol; 22(S)-hydroxycholesterol; 22(R),24(S)-dihydroxycholesterol; 25-hydroxycholesterol; 22(R)-hydroxycholesterol; 22(S)-hydroxycholesterol; 24(S),25-dihydroxycholesterol; 24(R),25-dihydroxycholesterol; 24,25-dehydrocholesterol; 25-epoxy-22(R)-hydroxycholesterol; 20(S)-hydroxycholesterol; (20R,22R)-cholest-5-ene-3β,20,22-triol; 4,4-dimethyl-5-α-cholesta-8,14,24-trien-3-β-ol; 7α-hydroxy-24(S),25-epoxycholesterol; 7β-hydroxy-24(S),25-epoxycholesterol; 7-oxo-24(S),25-expoxycholesterol; 7α-hydroxycholesterol; 7-oxocholesterol;

and desmosterol. In one preferred embodiment, the compound is an oxysterol.

In a second aspect, the invention features another method for treating a patient diagnosed as having a lower than normal HDL-cholesterol level or a higher than normal triglyceride level. This method includes administering to the patient a compound that modulates RXR-mediated transcriptional activity. RXR-modulating compounds include hetero ethylene derivatives; tricyclic retinoids; trienoic retinoids; benzocycloalkenyl-alka:di- or trienoic acid derivatives; bicyclic-aromatic compounds and their derivatives; bicyclylmethyl-aryl acid derivatives; phenyl-methyl heterocyclic compounds; tetrahydro-napthyl compounds; arylthio-tetrahydro-naphthalene derivatives and heterocyclic analogues; 2,4-pentadienoic acid derivatives; tetralin-based compounds; nonatetraenoic acid derivatives; SR11237; dexamethasone; hydroxy, epoxy, and carboxy derivatives of methoprene; bicyclic benzyl, pyridinyl, thiophene, furanyl, and pyrrole derivatives; benzofuran-acrylic acid derivatives; aryl-substituted and aryl and (3-oxo-1-propenly)-substituted benzopyran, benzothiopyran, 1,2-dihydroquinoline, and 5,6-dihydronaphthalene derivatives; vitamin D3 (1,25-dihydroxyvitamin D3) and analogs; 24-hydroxylase inhibitor; mono-or polyenic carboxylic acid derivatives; tetrahdroquinolin-2-one-6 or 7-yl and related derivatives; tetrahydronaphthalene; oxyiminoalkanoic acid derivatives; LG 100268; and LGD 1069.

In a third aspect, the invention features a method for determining whether a candidate compound modulates ABC1 expression by performing the steps of: (a) providing a nucleic acid molecule that includes an ABC1 regulatory region or promoter operably linked to a reporter gene; (b) contacting the nucleic acid molecule with the candidate compound; and (c) measuring expression of the reporter gene, wherein altered reporter gene expression, relative to a control not contacted with the compound, indicates that the candidate compound modulates ABC1 expression. In various preferred embodiments, the regulatory region includes 50 or more consecutive amino acids selected from nucleotides 5854 to 6694, 7756 to 8318, 10479 to 10825, 15214 to 16068, 21636 to 22111, 27898 to 28721, 32951 to 33743, 36065 to 36847, 39730 to 40577, 4543 to 5287, or 45081 to 55639 of SEQ ID NO: 1. In other preferred embodiments, the regulatory region 50 or more consecutive amino acids selected from nucleotides 1 to 28,707 or 29,011 to 53,228 of SEQ ID NO: 1. Preferably, the regulatory region includes a binding site for a transcription factor selected from a group consisting of LXRs, RXRs, RORs, SREBPs, and PPARs.

In a fourth aspect, the invention features a method for determining whether a person has an altered risk for developing cardiovascular disease. This method includes examining the person's ABC1 gene for polymorphisms or mutations. The presence of a polymorphism or mutation associated with cardiovascular disease indicates the person has an altered risk for developing cardiovascular disease.

In a related aspect, the invention features a method for predicting a person's response to a drug by determining whether the person has a polymorphism in an ABC1 gene that alters the person's response to the drug. Preferred polymorphisms are depicted in FIG. 4. In preferred embodiments of the fifth and sixth aspects, the polymorphism is in the 5′ regulatory region of ABC1.

In a sixth aspect, the invention features a substantially purified LXR response element comprising the nucleotide sequence AGATCANNNNAGGTCA, wherein each N is, independently, C, T, G, or A (SEQ ID NO: 231). Preferably, the LXR response element has the sequence AGATCACTTGAGGTCA (SEQ ID NO: 232). Even more preferably, the LXR response element consists essentially of the nucleotide; sequence AGATCANNNNAGGTCA, wherein each N is, independently, C, T, G, or A (SEQ ID NO: 231).

In a seventh aspect, the invention features a substantially pure nucleic acid molecule that consists essentially of a region that is substantially identical to at least 50, 100, 150, 300, 500, 750, 1000, 2000, 3000, 4000, 5000 or all of the consecutive nucleotides selected from nucleotides 5854 to 6694, 7756 to 8318, 10479 to 10825, 15214 to 16068, 21636 to 22111, 27898 to 28721, 32951 to 33743, 36065 to 36847, 39730 to 40577, 45081 to 55639, 4543 to 5287, 59188 to 60306, 60689 to 63548, 63574 to 65110, 65030 to 68312, 68605 to 73375, 73395 to 74692, 75586 to 77103, 74774 to 74920, 77519 to 87679, 87651 to 94160, 96916 to 97634, 94408 to 96595, 97807 to 98989, 100369 to 107171, 107179 to 107983, 108039 to 108998, 109222 to 118212, 118612 to 123911, 124586 to 138185, 137773 to 138393, 147497 to 148051, 158490 to 159118, 123718 to 125077, 137773 to 138912, 139304 to 139699, 139351 to 146359, 146867 to 147637, 147733 to 149404, 149858 to 152699, 153064 to 153916, 153978 to 158516, 158719 to 160272, 160375 to 164458, 165279 to 169814, 164215 to 164592, 164786 to 165133, 165125 to 165429, 169882 to 170189, 170067 to 174018, 176845 to 178875, 179113 to 180606, and 181723 to 183284 of SEQ ID NO: 1. In a related aspect, the invention features a substantially pure nucleic acid molecule that has a region that is substantially identical to nucleotides 5854 to 6694, 7756 to 8318, 10479 to 10825, 15214 to 16068, 21636 to 22111, 27898 to 28721, 32951 to 33743, 36065 to 36847, 39730 to 40577, 45081 to 55639, 4543 to 5287, 59188 to 60306, 60689 to 63548, 63574 to 65110, 65030 to 68312, 68605 to 73375, 73395 to 74692, 75586 to 77103, 74774 to 74920, 77519 to 87679, 87651 to 94160, 96916 to 97634, 94408 to 96595, 97807 to 98989, 100369 to 107171, 107179 to 107983, 108039 to 108998, 109222 to 118212, 118612 to 123911, 124586 to 138185, 137773 to 138393, 147497 to 148051, 158490 to 159118, 123718 to 125077, 137773 to 138912, 139304 to 139699, 139351 to 146359, 146867 to 147637, 147733 to 149404, 149858 to 152699, 153064 to 153916, 153978 to 158516, 158719 to 160272, 160375 to 164458, 165279 to 169814, 164215 to 164592, 164786 to 165133, 165125 to 165429, 169882 to 170189, 170067 to 174018, 176845 to 178875, 179113 to 180606, or 181723 to 183284 of SEQ ID NO: 1. Preferred nucleic acid molecules have a region that is substantially identical or identical to nucleotides 1 to 28,707 of SEQ ID NO: 1 or nucleotides 29,011 to 53,228 of SEQ ID NO: 1.

In an eighth aspect, the invention features a method of treating a human having a lower than normal HDL-cholesterol level, a higher than normal triglyceride level, or a cardiovascular disease, including administering to the human an ABC1 polypeptide, or a cholesterol- or triglyceride-regulating fragment thereof, or a nucleic acid molecule encoding an ABC1 polypeptide, or a cholesterol- or triglyceride-regulating fragment thereof. In a preferred embodiment, the human has a low cholesterol or high triglyceride level relative to normal. Preferably, the ABC1 polypeptide is wild-type ABC1, or has a mutation that increases its stability or its biological activity. Preferably, the nucleic acid molecule is operably linked to a promoter and contained in an expression vector. Preferred mutations include the R_K mutation at position 219 and the V_A mutation at position 399 of ABC1. A preferred biological activity is improved regulation of cholesterol transport.

In a ninth aspect, the invention features a method of treating or preventing a lower than normal HDL-cholesterol level, a higher than normal triglyceride level, or a cardiovascular disease, including administering to an animal (e.g., a human) a compound that mimes the activity of wild-type ABC1, R219K ABC1, or V399A ABC1 or modulates the biological activity of ABC1. One preferred cardiovascular disease that can be treated using the methods of the invention is coronary artery disease. Others include cerebrovascular disease and peripheral vascular disease.

The discovery that the ABC1 gene and protein are involved in cholesterol transport that affects serum HDL levels allows the ABC1 protein and gene to be used in a variety of diagnostic tests and assays for identification of HDL-increasing, triglyceride-lowering, or CVD-inhibiting drugs. In one family of such assays, the ability of domains of the ABC1 protein to bind ATP is utilized; compounds that enhance this binding are potential HDL-increasing or triglyceride-lowering drugs. Similarly, the anion transport capabilities and membrane pore-forming functions in cell membranes can be used for drug screening.

In a tenth aspect, ABC1 expression can also serve as a diagnostic tool for a lower than normal HDL-cholesterol level, a higher than normal triglyceride level, or CVD; determination of the genetic subtyping of the ABC1 gene sequence can be used to subtype individuals or families with lower than normal HDL levels or higher than normal triglyceride levels to determine whether the lower than normal HDL or higher than normal triglyceride phenotype is related to ABC1 function. This diagnostic process can lead to the tailoring of drug treatments according to patient genotype (referred to as pharmacogenomics), including prediction of the patient's response (e.g., increased or decreased efficacy or undesired side effects upon administration of a compound or drug).

Antibodies to an ABC1 polypeptide can be used both as therapeutics and diagnostics. Antibodies are produced by immunologically challenging a B-cell-containing biological system, e.g., an animal such as a mouse, with an ABC1 polypeptide to stimulate production of anti-ABC1 protein by the B-cells, followed by isolation of the antibody from the biological system. Such antibodies can be used to measure ABC1 polypeptide in a biological sample such as serum, by contacting the sample with the antibody and then measuring immune complexes as a measure of the ABC1 polypeptide in the sample. Antibodies to ABC1 can also be used as therapeutics for the modulation of ABC1 biological activity.

Thus, in an eleventh aspect, the invention features a purified antibody that specifically binds to ABC1. In one preferred embodiment, the antibody modulates cholesterol or triglyceride levels when administered to a mammal.

In a twelfth aspect, the invention features a method for determining whether candidate compound is useful for modulating cholesterol or triglyceride levels, the method including the steps of: (a) providing an ABC1 polypeptide; (b) contacting the polypeptide with the candidate compound; and (c) measuring binding of the ABC1 polypeptide, wherein binding of the ABC1 polypeptide indicates that the candidate compound is useful for modulating cholesterol or triglyceride levels.

In a thirteenth aspect, the invention features a method for determining whether a candidate compound is useful for the treatment of a lower than normal HDL-cholesterol level, a higher than normal triglyceride level, or a cardiovascular disease. The method includes (a) providing an ABC transporter (e.g., ABC1); (b) contacting the transporter with the candidate compound; and (c) measuring ABC transporter biological activity, wherein increased ABC transporter biological activity, relative to a transporter not contacted with the compound, indicates that the candidate compound is useful for the treatment of a lower than normal HDL-cholesterol level, a higher than normal triglyceride level, or a cardiovascular disease. Preferably the ABC transporter is in a cell or a cell free assay system.

In a fourteenth aspect, the invention features a method for determining whether candidate compound is useful for modulating cholesterol or triglyceride levels. The method includes (a) providing a nucleic acid molecule comprising an ABC transporter promoter operably linked to a reporter gene; (b) contacting the nucleic acid molecule with the candidate compound; and (c) measuring expression of the reporter gene, wherein increased expression of the reporter gene, relative to a nucleic acid molecule not contacted with the compound, indicates that the candidate compound is useful for modulating cholesterol or triglyceride levels.

In a fifteenth aspect, the invention features a non-human mammal having a transgene comprising a nucleic acid molecule encoding a mutated ABC1 polypeptide. In one embodiment, the mutation is a dominant-negative mutation, such as the M_T mutation at position 1091 of ABC1.

In a sixteenth aspect, the invention features an expression vector, a cell, or a non-human mammal that includes an ABC1 nucleic acid molecule of the present invention.

In a related aspect, the invention features a cell from a non-human mammal having a transgene that includes a nucleic acid molecule of the present invention.

In an eighteenth aspect, the invention features a method for determining whether a candidate compound decreases the inhibition of a dominant-negative ABC1 polypeptide. The method includes (a) providing a cell expressing a dominant-negative ABC1 polypeptide; (b) contacting the cell with the candidate compound; and (c) measuring ABC1 biological activity of the cell, wherein an increase in the ABC1 biological activity, relative to a cell not contacted with the compound, indicates that the candidate compound decreases the inhibition of a dominant-negative ABC1 polypeptide. A preferred dominant-negative ABC1 polypeptide is M1091T ABC 1.

In a nineteenth aspect, the invention features a method of determining in a subject a propensity for a disease or condition selected from the group consisting of a lower than normal HDL level, a higher than normal triglyceride level, and a cardiovascular disease. This method involves determining the presence or absence of at least one ABC1 polymorphism in the polynucleotide sequence of an ABC1 regulatory region, promoter, or coding sequence or in the amino acid sequence of an ABC1 protein in a sample obtained from the subject, wherein the presence or absence of the ABC1 polymorphism is indicative of a risk for the disease or condition. Preferably, the method also includes analyzing at least five ABC1 polymorphic sites in the polynucleotide sequence or the amino acid sequence.

In a twentieth aspect, the invention features a method for determining whether an ABC1 polymorphism is indicative of a risk in a subject for a disease or condition selected from the group consisting of a lower than normal HDL level, a higher than normal triglyceride level, and a cardiovascular disease. The method includes (a) determining whether the prevalence of the disease or condition in a first subject or set of subjects differs from the prevalence of the disease or condition in a second subject or set of subjects; (b) analyzing the polynucleotide sequence of an ABC1 regulatory region, promoter, or coding sequence or the amino acid sequence of an ABC1 protein in a sample obtained from the first subject or set of subjects and the second subject or set of subjects; and

(c) determining whether at least one ABC1 polymorphism differs between the first subject or set of subjects and the second subject or set of subjects, wherein the presence or absence of the ABC1 polymorphism is correlated with the prevalence of the disease or condition, thereby determining whether the ABC1 polymorphism is indicative of the risk. Preferably, the method further includes analyzing at least five ABC1 polymorphic sites in the polynucleotide sequence of an ABC1 regulatory region, promoter, or coding sequence or in the amino acid sequence of ABC1.

In a twenty-first aspect, the invention provides an electronic database having a plurality of sequence records of ABC1 polymorphisms correlated to records of predisposition to or prevalence of a disease or condition selected from the group consisting of a lower than normal HDL cholesterol level, a higher than normal triglyceride level, and a cardiovascular disease.

In a twenty-second aspect, the invention features a method for selecting a preferred therapy for modulating ABC1 activity or expression in a subject. This method includes (a) determining the presence or absence of at least one ABC1 polymorphism in the polynucleotide sequence of an ABC1 regulatory region, promoter, or coding sequence or in the amino acid sequence of an ABC1 protein in a sample obtained from the subject, wherein the presence or absence of the ABC1 polymorphism is indicative of the safety or efficacy of at least one therapy for modulating ABC1 expression or activity; and (b) determining a preferred therapy for modulating ABC1 expression or activity in the subject. Preferably, the method further includes analyzing at least five ABC1 polymorphic sites in the polynucleotide sequence of an ABC1 regulatory region, promoter, or coding sequence or the amino acids sequence of ABC1.

In a twenty-third aspect, the invention provides a method for determining whether a candidate compound is useful for the treatment of a disease or condition selected from the group consisting of a lower than normal HDL cholesterol level, a higher than normal triglyceride level, and a cardiovascular disease. This method includes (a) providing an assay system having a measurable ABC1 biological activity; (b) contacting the assay system with the candidate compound; and (c) measuring ABC1 biological activity or ABC1 phosphorylation. Modulation of ABC1 biological activity or ABC1 phosphorylation in this assay system, relative to the ABC1 biological activity or ABC1 phosphorylation in a corresponding control assay system not contacted with the candidate compound, indicates that the candidate compound is useful for the treatment of the disease or condition. In preferred embodiments, the assay system is a cell based system or a cell free system. Preferably, the candidate compound modulates both ABC1 protein phosphorylation and ABC1 activity.

In a twenty-fourth aspect, the invention provides a method for identifying a compound to be tested for an ability to ameliorate a disease or condition selected from the group consisting of a lower than normal HDL cholesterol level, a higher than normal triglyceride level, and a cardiovascular disease. This method includes

(a) contacting a subject or cell with a candidate compound; (b) measuring ABC1 expression, activity, or protein phosphorylation in the subject or cell. Altered ABC1 expression, activity, or protein phosphorylation in this subject or cell; relative to the ABC1 expression, activity, or protein phosphorylation in a corresponding control subject or cell not contacted with the candidate compound; identifies the candidate compound as a compound to be tested for an ability to ameliorate the disease or condition. Preferably, the candidate compound modulates both ABC1 protein phosphorylation and the ABC1 activity.

In a twenty-fifth aspect, the invention provides a method for determining whether a candidate compound is useful for modulating a disease or condition selected from the group consisting of a lower than normal HDL cholesterol level, a higher than normal triglyceride level, and a cardiovascular disease. The method includes (a) providing a cell expressing an ABC1 gene or a fragment thereof;

(b) contacting the cell with the candidate compound; and (c) measuring ABC1 activity of the cell. Altered ABC1 activity in this cell, relative to the ABC1 activity in a corresponding control cell not contacted with the compound, indicates that the candidate compound is useful for modulating the disease or condition.

In a twenty-six aspect, the invention provides a method for determining whether a candidate compound is useful for modulating a disease or condition selected from the group consisting of a lower than normal HDL cholesterol level, a higher than normal triglyceride level, and a cardiovascular disease. This method includes (a) contacting a cell expressing an ABC1 protein with the candidate compound; and (b) measuring the phosphorylation of the ABC1 protein. Altered ABC1 protein phosphorylation in this cell, relative to the ABC1 protein phosphorylation in a corresponding control cell not contacted with the candidate compound, indicates that the is useful for modulating the disease or condition.

In a twenty-seventh aspect, the invention provides a compound useful for the treatment of a disease or condition selected from the group consisting of a lower than normal HDL cholesterol level, a higher than normal triglyceride level, and a cardiovascular disease. The compound modulates ABC1 biological activity, and is identified by the steps of (a) providing an assay system having a measurable ABC1 biological activity; (b) contacting the assay system with the compound; and (c) measuring ABC1 biological activity, wherein modulation of ABC1 biological activity, relative to the ABC1 biological activity in a corresponding control assay system not contacted with the compound, indicates that the compound is useful for the treatment of the disease or condition.

In a twenty-eighth aspect, the invention provides a compound useful for the treatment of a disease or condition selected from the group consisting of a lower than normal HDL cholesterol level, a higher than normal triglyceride level, and a cardiovascular disease. The compound induces a change in ABC1 biological activity that mimics the change in ABC1 biological activity induced by the R219K ABC1 mutation.

In a twenty-ninth aspect, the invention provides a compound useful for the treatment of a disease or condition selected from the group consisting of a lower than normal HDL cholesterol level, a higher than normal triglyceride level, and a cardiovascular disease. The compound binds or interacts with residue R219 of ABC1, thereby mimicking the change in ABC1 activity induced by the R219K ABC1 mutation.

In a thirtieth aspect, the invention provides a compound useful for the treatment of a disease or condition selected from the group consisting of a lower than normal HDL cholesterol level, a higher than normal triglyceride level, and a cardiovascular disease. The compound induces a change in ABC1 biological activity that mimics the change in ABC1 biological activity induced by the V339A ABC1 mutation.

In a thirty-first aspect, the invention provides a compound useful for the treatment of a disease or condition selected from the group consisting of a lower than normal HDL cholesterol level, a higher than normal triglyceride level, and a cardiovascular disease. The compound binds or interacts with residue V399 of ABC1, thereby mimicking the change in ABC1 activity induced by the V399A ABC1 mutation.

In a thirty-second aspect, the invention provides a compound that modulates ABC1 activity and binds or interacts with an amino acid of ABC1, wherein the amino acid is a residue selected from amino acids 119 to 319 of ABC1 (SEQ ID NO: 5) or amino acids 299 to 499 of ABC1 (SEQ ID NO: 5).

In a thirty-second aspect, the invention provides a method for determining whether a candidate compound is useful for the treatment a disease or condition selected from the group consisting of a lower than normal HDL cholesterol level, a higher than normal triglyceride level, and a cardiovascular disease. This method involves (a) providing an assay system having a measurable LXR biological activity; (b) contacting the assay system with the candidate compound; and

(c) measuring LXR biological activity, wherein modulation of LXR biological activity, relative to the LXR biological activity in a corresponding control assay system not contacted with the candidate compound, indicates that the candidate compound is useful for the treatment of the disease or condition.

In a thirty-third aspect, the invention provides method for determining whether a candidate compound is useful for modulating ABC1 biological activity. This method involves (a) providing an assay system having a measurable LXR biological activity; (b) contacting the assay system with the candidate compound; and (c) measuring LXR biological activity, wherein modulation of LXR biological activity, relative to the LXR biological activity in a corresponding control assay system not contacted with the candidate compound, indicates that the candidate compound is useful for modulating ABC1 biological activity. Preferably, the LXR biological activity is modulation of ABC1 expression.

In a thirty-fourth aspect, the invention provides method for identifying a compound to be tested for an ability to modulate ABC1 biological activity. This method involves (a) contacting a subject or cell with a candidate compound;

(b) assaying the activity of the LXR gene product in the subject or cell; wherein modulation of the activity, relative to the activity in a corresponding control subject or cell not contacted with the candidate compound, identifies the candidate compound as a compound to be tested for an ability to modulate the biological activity of ABC1.

In a thirty-fifth aspect, the invention provides the use of an LXR gene product in an assay to identify compounds useful for the treatment of a disease or condition selected from the group consisting of a lower than normal HDL cholesterol level, a higher than normal triglyceride level, and a cardiovascular disease.

In a thirty-sixth aspect, the invention features the use of a compound that modulates the activity or expression of an LXR gene product for the treatment of a disease or condition selected from the group consisting of a lower than normal HDL cholesterol level, a higher than normal triglyceride level, and a cardiovascular disease.

In a thirty-seventh aspect, the invention provides a method for identifying a compound to be tested for an ability to treat a disease or condition selected from the group consisting of a lower than normal HDL cholesterol level, a higher than normal triglyceride level, and a cardiovascular disease. This method involves

(a) providing an assay system having a measurable LXR biological activity;

(b) contacting the assay system with the candidate compound; and (c) measuring LXR biological activity, wherein modulation of the LXR biological activity, relative to the LXR biological activity in a corresponding control assay system not contacted with the candidate compound, identifies the candidate compound as a compound to be tested for an ability to treat the disease or condition.

In a thirty-eight aspect, the invention provides a method for screening a candidate LXR agonist for the ability to treat a disease or condition selected from the group consisting of a lower than normal HDL cholesterol level, a higher than normal triglyceride level, and a cardiovascular disease. This method involves

(a) contacting the cell with the candidate LXR agonist; and (b) measuring cholesterol efflux activity of the cell, wherein an increase in the cholesterol efflux activity in the cell, relative to the cholesterol efflux in a corresponding control cell not contacted with the candidate LXR agonist, indicates that the candidate LXR agonist is us ful for treating the diseas or condition.

In a thirty-ninth aspect, the invention provides a method for screening a candidate LXR modulating compound for the ability to treat a disease or condition selected from the group consisting of a lower than normal HDL cholesterol level, a higher than normal triglyceride level, and a cardiovascular disease. This method involves (a) contacting a cell with the candidate LXR modulating compound; and

(b) measuring ABC1 biological activity of the cell; wherein an increase in ABC1 biological activity in the cell, relative to the ABC1 biological activity in a corresponding control cell not contacted with the LXR modulating compound, indicates that the LXR modulating compound is useful for treating the disease or condition.

In another aspect, the invention provides a method for determining whether a candidate compound is useful for modulating triglyceride levels. The method involves (a) providing a cell comprising an ABC1 polypeptide comprising amino acids 1 to 60 of SEQ ID NO: 5; (b) contacting the cell with the candidate compound; and (c) measuring the half-life of the ABC1 polypeptide,

wherein an increase in said half-life, relative to the half-life in a corresponding control cell not contacted with the compound, indicates that the candidate compound is useful for modulating triglyceride levels.

In a related aspect, the invention features method for determining whether a candidate compound mimics ABC1 biological activity. The method includes (a) providing a cell that is not expressing an ABC1 polypeptide; (b) contacting the cell with the candidate compound; and (c) measuring ABC1 biological activity of the cell, wherein altered ABC1 biological activity, relative to a corresponding control cell not contacted with the compound, indicates that the candidate compound modulates ABC1 biological activity. Preferably, the cell has an ABC1 null mutation. In one preferred embodiment, the cell is in a mouse or a chicken (e.g., a WHAM chicken) in which its ABC1 gene has been mutated.

In a preferred embodiment of the screening methods of the present invention, the cell is in an animal. The preferred biological activity is transport of cholesterol (e.g., HDL cholesterol or LDL cholesterol) or interleukin-1, or is binding or hydrolysis of ATP by the ABC1 polypeptide. Preferably, the ABC1 polypeptide used in the screening methods includes amino acids 1-60 of SEQ ID NO: 5. Alternatively, the ABC1 polypeptide can include a region encoded by a nucleotide sequence that hybridizes under high stringency conditions to nucleotides 75 to 254 of SEQ ID NO: 6. Preferably, the subject is a human. Preferably, the cell or assay system has an exogenously supplied copy of an LXRE selected from the group consisting of SEQ ID NO: 94, SEQ ID NO: 92, and the LXRE consensus motif at nucleotide −7670 of the 3′ end of intron 1. For various methods of the invention, a preferred LXR biological activity is modulation of ABC1 expression. A preferred LXR gene product is an ABC1 nucleic acid molecule or protein.

It is also contemplated that additional sequence of the ABC1 regulatory regions may be determined by sequencing the rest of the 4I8, 31J20, 47O19, or 179G21 Research Genetics RPCI-11 BACs using the methods described herein. Substantially pure nucleic acids containing regions substantially identical to at least 50, 100, 150, 300, 500, 750, 1000, 2000, 3000, 4000, 5000 consecutive nucleotides of these regions may be used in the methods of the present invention.

By “polypeptide” is meant any chain of more than two amino acids, regardless of post-translational modification such as glycosylation or phosphorylation.

By “reporter gene” is meant any gene which encodes a product whose expression is detectable and/or quantifiable by physical, immunological, chemical, biochemical, or biological assays. A reporter gene product may, for example, have one of the following attributes, without restriction: a specific nucleic acid/chip hybridization pattern, fluorescence (e.g., green fluorescent protein), enzymatic activity (e.g., lacZ/β-galactosidase, luciferase, chloramphenicol acetyltransferase), toxicity (e.g., ricin A), or an ability to be specifically bound by a second molecule (e.g., biotin or a detectably labeled antibody). It is understood that any engineered variants of reporter genes, which are readily available to one skilled in the art, are also included, without restriction, in the foregoing definition.

By “operably linked” is meant that a gene and a regulatory sequence are connected in such a way as to permit expression of the gene product under the control of the regulatory sequence. A promoter may also be operably linked to a gene such that expression of the gene product is under control of the promoter.

By “regulatory region” is meant a region that, when operably linked to a promoter and a gene (e.g., a reporter gene), is capable of modulating the expression of the gene from the promoter. Regulatory regions include, for example, nuclear hormone transcription factor binding sites such as those described herein and may be found in intronic sequence.

By “promoter” is meant a minimal sequence sufficient to direct transcription of an operably-linked gene.

By “substantially identical” is meant a polypeptide or nucleic acid exhibiting at least 50%, preferably 85%, more preferably 90%, and most preferably 95% identity to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides. One sequence may include additions or deletions (i.e., gaps) of 20% or less when compared to the second sequence. Optimal alignment of sequences may be conducted, for example, by the methods of Gish and States (Nature Genet. 3:266-272, 1993), Altshul et al. (J. Mol. Biol. 215:403-410, 1990), Madden et al. (Meth. Enzymol. 266:131-141, 1996), Althsul et al (Nucleic Acids Res. 25:3389-3402, 1997), or Zhang et al (Genome Res. 7:649-656, 1997).

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

By “substantially pure nucleic acid” is meant nucleic acid that is free of the genes which, in the naturally-occurring genome of the organism from which the nucleic acid of the invention is derived, flank the nucleic acid. The term therefore includes, for example, a recombinant nucleic acid that is incorporated into a vector; into an autonomously replicating plasmid or virus; into the genomic nucleic acid of a prokaryote or a eukaryote cell; or that exists as a separate molecule (e.g., a cDNA or a genomic or cDNA fragment produced by PCR or restriction endonuclease digestion) independent of other sequences. It also includes a recombinant nucleic acid that is part of a hybrid gene encoding additional polypeptide sequence.

By “high stringency conditions” is meant hybridization in 2×SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “modulates” is meant increase or decrease. Preferably, a compound that modulates LXR-mediated transcription, RXR-mediated transcription, ABC1 gene expression, HDL-C levels, or triglyceride levels does so by at least 5%, more preferably by at least 10%, and most preferably by at least 25% or even at least 50%.

By “purified antibody” is meant antibody which is at least 60%, by weight, free from proteins and naturally occurring organic molecules with which it is naturally associated. Preferably, the preparation is at least 75%, more preferably 90%, and most preferably at least 99%, by weight, antibody. A purified antibody may be obtained, for example, by affinity chromatography using recombinantly-produced protein or conserved motif peptides and standard techniques.

By “specifically binds” is meant an antibody that recognizes and binds to, for example, a human ABC1 polypeptide but does not substantially recognize and bind to other non-ABC1 molecules in a sample, e.g., a biological sample, that naturally includes protein. A preferred antibody binds to the ABC1 polypeptide sequence of FIG. 2A (SEQ ID NO: 5).

By “polymorphism” is meant that a nucleotide or nucleotide region is characterized as occurring in several different forms. A “mutation” is a form of a polymorphism in which the expression level, stability, function, or biological activity of the encoded protein is substantially altered.

By “LXR” is meant nuclear receptors LXRα and LXRβ. Preferred LXRs include human LXRα (GenBank accession no. Q13133) and human LXRβ (GenBank accession no. P55055)(see Apfel et al., Mol. Cell. Biol. 14:7025-7035, 1994; Willy et al., Genes Dev. 9:1033-1045, 1995; and Song et al., Proc. Natl. Acad. Sci. USA 91:10809-10813, 1995, each of which is hereby incorporated by reference).

By “RXR” is meant nuclear receptors RXRα, RXRβ, and RXRγ. Preferred RXRs include human RXRα (GenBank accession no. Q13133), human RXRβ (GenBank accession no. S37781), and human RXRγ (GenBank accession no. Q13133).

By “ABC transporter” or “ABC polypeptide” is meant any transporter that hydrolyzes ATP and transports a substance across a membrane. Preferably, an ABC transporter polypeptide includes an ATP Binding Cassette and a transmembrane region. Examples of ABC transporters include, but are not limited to, ABC1, ABC2, ABCR, and ABC8.

By “ABC1 polypeptide” is meant a polypeptide having substantial identity to an ABC1 polypeptide having the amino acid sequence of SEQ ID NO: 5.

By “ABC biological activity” or “ABC1 biological activity” is meant hydrolysis or binding of ATP, transport of a compound (e.g., cholesterol, interleukin-1) or ion across a membrane, or regulation of cholesterol or phospholipid levels (e.g., either by increasing or decreasing HDL-cholesterol or LDL-cholesterol levels).

The invention provides methods for treating patients having low HDL-C and/or higher than normal triglyceride levels by administering compounds that modulate ABC1 biological activity or expression. For example, the compounds may modulate the transcriptional activity of LXR/RXR heterodimers. Many compounds that modulate LXR transcriptional activity or RXR transcriptional activity are known in the art. Preferred compounds of the invention are oxysterols; additional compounds are described herein.

The invention also provides screening procedures for identifying therapeutic compounds (cholesterol-modulating, triglyceride-modulating, or anti-CVD pharmaceuticals) which can be used in human patients. Compounds that modulate ABC1 biological activity or expression are consid red useful in the invention, as are compounds that modulate ABC concentration, protein stability, regulated catabolism, or its ability to bind other proteins or factors. In general, the screening methods of the invention involve screening any number of compounds for therapeutically active agents by employing any number of in vitro or in vivo experimental systems. Exemplary methods useful for the identification of such compounds are detailed below.

The methods of the invention simplify the evaluation, identification and development of active agents for the treatment and prevention of low HDL, higher than normal triglyceride levels, and CVD. In general, the screening methods provide a facile means for selecting natural product extracts or compounds of interest from a large population which are further evaluated and condensed to a few active and selective materials. Constituents of this pool are then purified and evaluated in the methods of the invention to determine their HDL-raising, triglyceride-lowering, anti-CVD activities, or a combination thereof.

Other features and advantages of the invention will be apparent from the following description of the preferred embodiments thereof.

### BRIEF DESCRIPTION OF THE DRAWINGS

FIG. 1 shows the genomic sequence of human ABC1, including exons 1-50 (SEQ ID NO: 1). Capital letters denote exonic sequence, lower case letters denote 5′ regulatory sequence or intronic sequence. “Z” denotes any nucleotide or other, including no nucleotide. The numbering used herein for the nucleotides in SEQ ID No:1 assumes that no nucleotide is present at the positions denoted “z;” however, it would be readily apparent to one skilled in the art that the numbering of the nucleotides in these sequences would change if a nucleotide is present at some or all of the positions denoted “z.” “K” denotes nucleotides G or T; “Y” denotes nucleotides C or T; “R” denotes nucleotides A or G; “M” denotes nucleotides A or C; “S” denotes nucleotides C or G; “H” denotes nucleotides A, C, or T; “B” denotes nucleotides C, G, or T. Because of the identification of an ABC1 exon upstream of exon 0 that we previously disclosed, the numbering used herein to refer to ABC1 exons has been increased by one compared to our previously used numbering (U.S. Ser. No. 09/526,193; U.S. Ser. No. 60/124,702; U.S. Ser. No. 60/138,048; U.S. Ser. No. 60/139,600; U.S. Ser. No. 60/151,977). For example, the previously described exon 0 is referred to herein as exon 1.

FIG. 2A is the amino acid sequence of the human ABC1 protein (SEQ ID NO: 5). FIG. 2B is the nucleotide sequence of the human ABC1 cDNA (SEQ ID NO: 6).

FIG. 3 is a summary of locations of consensus transcription factor binding sites in the human ABC1 5′ regulatory sequence. The abbreviations are as follows: PPRE=peroxisome proliferator-activated receptor. SREBP=steroid response element-binding protein site. ROR=RAR-related orphan receptor. The numbering used herein for the locations of the transcription factor binding sites assumes that no nucleotide is present at the positions denoted “z” in SEQ ID NO: 1. For the polymorphisms in the promoter region, the numbering is based on the first base of the promoter as nucleotide number −1. For exon 1, the numbering is based on the first base of exon 1 as nucleotide number +1. For the 5′ end intron 1, the numbering is based on the first position in intron 1 as +1. For the 3′ end of intron 1, the numbering is based on the first base ′5 to the start of exon 2 as nucleotide number −1.

FIG. 4 is a table summarizing polymorphisms in the genomic ABC1 sequences.

FIGS. 5A and 5B are bar graphs showing the percent of heterozygotes or unaffected family members with HDL (FIG. 5A) and triglycerides (TG) (FIG. 5B) within a given range of percentiles for age and sex, based on the LRC criteria (Heiss et al., Circulation 62:IV-116-IV-136, 1980). A broad distribution of HDL levels was seen in the heterozygotes, extending up to the 31^{st} percentile for age and sex. Although there is overlap in the distribution of triglycerides between heterozygotes and unaffected family members, a larger portion of heterozygotes have triglyceride levels greater than the 80^{th} percentile for age and sex.

FIG. 6 is a table characterizing TD patients, ABC1 heterozygotes, and unaffected family members.

FIG. 7 is a table summarizing the incidence of CAD in ABC1 heterozygotes.

FIG. 8 is a graph showing the average HDL levels in heterozygotes for each mutation versus the efflux levels measured in a heterozygous carrier of each mutation. The HDL levels are expressed as the percentage of the mean HDL level in the unaffected members of that family. The efflux levels are highly correlated with the levels of HDL cholesterol and are associated with 82% of the variation in HDL cholesterol levels.

FIG. 9 is a table summarizing the HDL levels and presence or absence of CAD in ABC1 heterozygotes. In the R2144X and R909X ABC1 mutations, the codon encoding Arg2133 or Arg909 is mutated to a STOP codon resulting in truncation of the encoded protein. In the “Del E,D 1893,94” mutation, the codons encoding Glu1893 and Asp1894 are deleted. The “invs25+1G→C” mutation converts the first nucleotide of intron 25 from a “G” to a “C,” removing a splice site. For the “del C6825→2145X” mutation, the deletion of C6825 in the nucleotide sequence is a frame-shift mutation that results in a STOP codon at the codon corresponding to amino acid 2145 of the encoded protein. For the “CTC6952-4TT→2203X” mutation, “CTC” is replaced by “TT” in the nucleotide sequence, resulting in the conversion of the codon encoding amino acid 2203 to a stop codon.

FIG. 10 is a table comparing the mean lipid levels in unaffected family members and ABC1 heterozygotes with either missense or sever mutations.

FIG. 11 is a schematic diagram of the ABC1 protein, illustrating the location of the mutations and the presence or absence of CAD in carriers of the mutations. The number (n) of heterozygotes who are 40 years or older and may have developed CAD are listed.

FIGS. 12A and 12B are pedigrees of two FHA kindreds, FHA3 and FHA1, respectively (Marcil et al., Lancet 354:1341-1346, 1999). Males are denoted by square symbols, females by circles. Individuals heterozygous for mutations are given half-shaded symbols, with the probands indicated by arrows. A diagonal line indicates a deceased individual. The youngest individuals have HDL cholesterol at higher percentile ranges than those in the older generations.

FIG. 13 is a bar graph showing the percentage of individuals less than 30 years of age and from 30 to less than 70 years of age with HDL cholesterol levels in a given percentile range. Younger individuals have a far broader distribution of HDL cholesterol levels, clearly indicating that the impact of ABC1 on HDL levels is influenced by age.

FIG. 14 is a table summarizing HDL and TG levels in different age groups for ABC1 heterozygotes and unaffected family members.

FIGS. 15A and 15B are graphs showing the mean HDL level in heterozygous males (FIG. 15A) and females (FIG. 15B) in 10 year age groups (plotted at the half-way point) compared to the 10^{th} percentile distribution in the LRC population (Heiss et al., supra). Error bars represent the standard deviation of each mean. The number of individuals in each group is shown under each data point. Beyond the age of 30, mean HDL levels in heterozygotes fall much lower than the 10^{th} percentile distribution; in contrast, mean HDL cholesterol levels in the heterozygotes less than 30 y ars old more closely approximate the 10^{th} percentile distribution.

FIGS. 16A and 16B are graphs showing the mean HDL (FIG. 16A) and triglyceride levels (FIG. 16B) in heterozygotes and unaffected family members falling within each tertile of BMI. The tertiles of BMI correspond to the following values: (1) BMI<21.4; (2) 21.4<BMI<25.1; (3) BMI>25.1.

FIG. 17 is a table showing the oligonucleotides and reaction conditions used for RFLP screening of ABC1 polymorphisms.

FIG. 18 is a picture of a gel showing RFLP genotyping of the R219K variant. The 177 base pair PCR product is not digested for the A allele, whereas the B allele is digested producing fragments of 107 and 70 base pairs.

FIG. 19 is a table showing the allele frequencies of polymorphisms in the ABC1 gene.

FIG. 20 is a table comparing MSD, MOD, and frequency of coronary events in R219K ABC1 variant carriers compared to controls.

FIG. 21 is a graph showing the event-free survival curves for carriers (AB+BB) and non-carriers (AA) of the R219K ABC1 variant. Carriers of the variant have a 29% increased event-free survival over the two years of the trial, compared with non-carriers.

FIG. 22 is a table showing the baseline demographics and lipid levels in the Regression Growth Evaluation Statin Study (REGRESS) cohort by R219K ABC1 genotype.

FIG. 23 is a table showing the lipid levels and CAD above and below the median age in R219K ABC1 carriers and controls.

FIG. 24 is a bar graph showing the percent difference in HDL cholesterol levels between those greater and less than the median age (56.7 years) for each R219K genotype.

FIGS. 25A and 25B are graphs showing the correlations of HDL cholesterol (FIG. 25A) and efflux (FIG. 25B) with age, by R219K genotype.

FIGS. 26A and 26B are graphs showing the change in MSD (FIG. 26A) and MOD (FIG. 26B) by median age in carriers (AB+BB) and non-carriers (AA) of the R219K ABC1 variant.

FIG. 27 is a table showing the ethnic distribution of the R219K ABC1 variant.

### DETAILED DESCRIPTION OF THE INVENTION

We have previously discovered that the human ABC1 (also known as ABCA1) genomic region contains consensus binding sites for transcription factors such as LXRs, RXRs, PPARs, SREBPs, and RORs. In the present invention, we report the sequence of additional regions of the ABC1 regulatory region which also contain consensus binding sites for transcription factors. We also discovered that heterozygotes for ABC1 mutations have age-modulated decreases in HDL, increases in triglyceride levels, and significantly increased risk for CAD. Furthermore, this phenotype was highly correlated with efflux, clearly demonstrating that impairment of reverse cholesterol transport is associated with decreased plasma HDL cholesterol, increased triglyceride levels, and increased atherogenesis. Accordingly, the present invention features screening methods to identify therapies that increase ABC1 function, resulting in increased plasma HDL cholesterol, decreased triglyceride levels, protection against atherosclerosis, or a combination of these effects.

Genes play a significant role influencing HDL levels. Tangier disease (TD) was the first reported genetic HDL deficiency. Until recently, the molecular basis for TD was unknown, but now mutations in ABC1 have been identified in TD patients (described below). For example, we have identified two additional probands and their families, and confirmed linkage and refined the locus to a limited genomic region. Mutations in the ABC1 gene accounting for all four alleles in these two families were detected. A more frequent cause of low HDL levels is a distinct disorder, familial HDL deficiency (FHA). On the basis of independent linkage, meiotic recombinants and disease associated haplotypes, FHA was localized to a small genomic region encompassing the ABC1 gene. A mutation in a conserved residue in ABC1 segregated with FHA. Antisense reduction of the ABC1 transcript in fibroblasts was associated with a significant decrease in cholesterol efflux.

Cholesterol is normally assembled with intracellular lipids and secreted, but in TD the process is diverted and cholesterol is degraded in lysosomes. This disturbance in intracellular trafficking of cholesterol results in an increase in intracellular cholesterol ester accumulation associated with morphological changes of lysosomes and the Golgi apparatus and cholesteryl ester storage in histiocytes, Schwann cells, smooth muscle cells, mast cells and fibroblasts.

The clinical and biochemical heterogeneity in patients with TD has led to the possibility that genetic heterogeneity may also underlie this disorder. Considering this, we initially performed linkage analysis on these two families of different ancestries (TD-1 is Dutch, TD-2 is British; Frohlich et al., Clin. Invest. Med. 10:377-382, 1987) and confirmed that the genetic mutations underlying TD in these families were localized to the same 9q31 region, to which a large family with TD had been assigned (Rust et al., Nature Genetics 20:96-98, 1998). Detailed haplotype analysis, together with the construction of a physical map, refined the localization of this gene. Mutations in the ABC1 gene were found in TD.

FHA is much more common than TD, although its precise frequency is not known. While TD has been described to date in only 40 families, we have identified more than 40 FHA families in the Netherlands and Quebec alone. After initial suggestions of linkage to 9q31, thirteen polymorphic markers spanning approximately 10 cM in this region were typed and demonstrated the highest LOD score at D9S277. Analysis of the homozygosity of markers in the TD-2 proband, who was expected to be homozygous for markers close to TD due to his parents' consanguinity, placed the TD gene distal to D95127. Combined genetic data from TD and FHA families pointed to the same genomic segment spanning approximately 1,000 kb between D9S127 and D9S1866. The ABC1 transporter gene was contained within the minimal genomic region. RT-PCR analysis in one family demonstrated a deletion of leucine at residue 693 (693) in the first transmembrane domain of ABC1, which segregated with the phenotype of HDL deficiency in this family.

ABC1 is part of the ATP-binding cassette (ABC transporter) superfamily, which is involved in energy-dependent transport of a wide variety of substrates across membranes (Dean et al., Curr. Opin. Gen. Dev. 5:779-785, 1995). These proteins have characteristic motifs conserved throughout evolution which distinguish this class of proteins from other ATP binding proteins. In humans these genes essentially encode two ATP binding segments and two transmembrane domains (Dean et al., Curr. Opin. Gen. Dev. 5:779-785, 1995). We have now shown that the ABC1 transporter is crucial for intracellular cholesterol transport.

We have demonstrated that reduction of the ABC1 transcript using oligonucleotide antisense approaches results in decreased efflux, clearly demonstrating the link between alterations in this gene and its functional effects. TD and FHA now join the growing list of genetic diseases due to defects in the ABC group of proteins including cystic fibrosis (Zielenski, et al., Annu. Rev. Genet. 29:777-807, 1995), adrenoleukodystrophy (Mosser et al., Nature 361: 726-730, 1993), Zellweger syndrome (Gärtner et al., Nat. Genet. 1:23, 1992), progressive familial intrahepatic cholestatis (Bull et al., Nat. Genet. 18:219-224, 1998), and different eye disorders including Stargardt disease (Allikmets et al., Nat. Genet.15:236-246, 1997), autosomal recessive retinitis pigmentosa (Allikmets et al., Science 277:1805-1807, 1997), and cone-rod dystrophy (Cremers et al., Hum. Mol. Genet. 7:355-362, 1998).

Patients with TD have been distinguished from patients with FHA on the basis that Tangier disease was an autosomal recessive disorder (OMIM 20540) while FHA is inherited as an autosomal dominant trait (OMIM 10768). Furthermore, patients with TD have obvious evidence for intracellular cholesterol accumulation which is not seen in FHA patients. It is now evident that heterozygotes for TD do have reduced HDL levels and that the same mechanisms underlie the HDL deficiency and cholesterol efflux defects seen in heterozygotes for TD as well as FHA. Furthermore, the more severe phenotype in TD represents loss of function from both alleles of the ABC1 gene.

ABC1 is activated by protein kinases, presumably via phosphorylation, which also provides one explanation for the essential role of activation of protein kinase C in promoting cholesterol efflux (Drobnick et al., Arterioscler. Thromb. Vasc. Biol. 15: 1369-1377, 1995). Brefeldin, which inhibits trafficking between the endoplasmic reticulum and the Golgi, significantly inhibits cholesterol efflux, essentially reproducing the effect of mutations in ABC1, presumably through the inhibition of ABC1 biological activity. This finding has significance for the understanding of mechanisms leading to premature atherosclerosis. TD homozygotes develop premature coronary artery disease, as seen in the proband of TD-1 (III-01) who had evidence for coronary artery disease at 38 years. This is particular noteworthy as TD patients, in addition to exhibiting significantly reduced HDL, also have low LDL cholesterol, and yet they develop atherosclerosis despite this. This highlights the importance of HDL intracellular transport as an important mechanism in atherogenesis. There is significant evidence that heterozygotes for TD are also at increased risk for premature vascular disease (Schaefer et al., Ann. Int. Med. 93:261-266, 1980; Serfaty-Lacrosniere et al., Atherosclerosis 107:85-98, 1994). There is also preliminary evidence for premature atherosclerosis in some probands with FHA (e.g., the proband in FHA-2 (III-01) had a coronary artery bypass graft at 46 years while the proband in FHA-3 had evidence for CAD around 50 years of age. The TD-1 proband had more severe efflux deficiency than the TD-2 proband. Interestingly, the TD-2 proband had no evidence for CAD by 62 when he died of unrelated causes, providing preliminary evidence for a relationship between the degree of cholesterol efflux (mediated in part by the nature of the mutation) and the likelihood of atherosclerosis.

The ABC1 gene plays a crucial role in cholesterol transport and, in particular, intracellular cholesterol trafficking in monocytes and fibroblasts. It also appears to play a significant role in other tissues such as the nervous system, GI tract, and the cornea. Completely defective intracellular cholesterol transport results in peripheral neuropathy, corneal opacities, and deposition of cholesterol esters in the rectal mucosa.

HDL deficiency is heterogeneous in nature. The delineation of the genetic basis of TD and FHA underlies the importance of this particular pathway in intracellular cholesterol transport, and its role in the pathogenesis of atherosclerosis. Unraveling of the molecular basis for TD and FHA defines a key step in a poorly defined pathway of cholesterol efflux from cells and could lead to new approaches to treatment of patients with HDL deficiency in the general population.

HDL has been implicated in numerous other biological processes, including but not limited to: prevention of lipoprotein oxidation; absorption of endotoxins; protection against Trypanosoma brucei infection; modulation of endothelial cells; and prevention of platelet aggregation (see Genest et al., J. Invest. Med. 47: 3142, 1999, hereby incorporated by reference). Any compound that modulates HDL levels may be useful in modulating one or more of the foregoing processes. Our previous discovery that ABC1 functions to regulate HDL I vels links, for the first time, ABC1 with the foregoing processes.

With the identification of the ABC1 protein as a key initiator of the efflux pathway, it has now been possible to directly examine the relationship between efflux, HDL, triglyceride level, and CAD. We have characterized the phenotypes of heterozygotes for several mutations in the ABC1 gene in a large cohort where diagnosis has been made by mutation identification. Furthermore, the phenotype of the heterozygotes was compared to that of unaffected family members, enabling the results to be controlled, at least in part, for other genetic and environmental influences. In contrast, prior studies in obligate heterozygotes have been limited to small numbers, often within a single family, and thus restricted in the ability to analyze the phenotypic expression with multiple mutations over a range of ages.

A cohort of 77 individuals heterozygous for multiple mutations in the ABC1 gene were identified, enabling the characterization of 13 ABC1 mutations in 11 families (5 TD, 6 FHA). The ABC1 heterozygotes have an approximate 50% decrease in HDL cholesterol and apoAI, and a mild but significant decrease in apoAII. In addition, ABC1 heterozygotes have increased triglycerides, but in contrast to TD patients, have no significant change in total or LDL cholesterol. The changes in HDL, apoAI, and triglycerides were gene-dose dependent, suggesting that they are directly related to ABC1 function. Furthermore, heterozygotes have an over three-fold increased risk of developing CAD, and younger average age-of-onset compared to unaffected individuals. Further, the heterozygotes with the most severe deficiency in efflux had a higher frequency and greater severity of CAD. Interestingly, the severity of the phenotype observed in the heterozygotes appeared to be mutation-dependent, but there was no obvious relationship between the site of mutation and the phenotype. There was a trend toward lower HDL in carriers of severe mutations that caused truncations or null all les than in carriers of missense mutations. One notable exception is the M1091T missense mutation which had the most severe phenotype, with marked reductions in HDL cholesterol and efflux in affected family members, suggesting that this mutation may act in a dominant-negative fashion, down-regulating the function of the wild-type allele. Another interesting finding is the small cluster of mutations at the very C-terminal region of the protein, which suggests that this region is critical for ABC1 function.

The severe HDL deficiency in ABC1 heterozygotes suggests that residual cholesterol efflux is the major determinant of HDL cholesterol levels. Here we demonstrated a strong correlation between cholesterol efflux and HDL cholesterol levels. From the regression equation of mean HDL on efflux, each 8% increase in relative efflux is predicted to be associated with a 0.1 mmol/L increase in HDL cholesterol levels. For example, to effect a 30% increase in HDL cholesterol in a 40 year old male, it would require a 50% increase in ABC1 mediated cholesterol efflux. Although these numbers may not directly extrapolate to what is observed in a general population where other genetic and environmental factors have not been controlled for, these data nonetheless suggest that relatively small changes in ABC1 function may have a significant impact on plasma HDL cholesterol levels. Furthermore, the data presented here suggest that variations in efflux due to variations in ABC1 function directly reflect not only plasma HDL cholesterol levels but also triglyceride levels and CAD susceptibility, thus providing direct validation of the reverse cholesterol transport hypothesis and validation of ABC1 as a therapeutic target to raise HDL cholesterol, lower triglyceride levels, and protect against atherosclerosis.

The phenotype in ABC1 heterozygotes is also age-modulated. From 20 years of age in members of the control cohort, there is a small but definite increase in HDL with advancing age that is obviously absent in the heterozygotes. One explanation for this finding is that there is normally an age-related increase in ABC1 function, which is not seen in heterozygotes, perhaps because the remaining functioning allele has already been maximally up-regulated secondary to an increase in intracellular cholesterol. This lack of age-related increase in ABC1 function in heterozygotes would exaggerate the difference in HDL levels between heterozygotes and control individuals in older age groups. There is some evidence for an age-modulated increase in expression of ABC transporters (Gupta, Drugs Aging 7:19-29, 1995). Further, evidence of a potential age-related increase in ABC1 function comes from the observation that the percentage of apoAI found in the preβ₁ subfraction of HDL, the predominant cholesterol acceptors, decreases with age, suggesting increased formation of mature α-migrating HDL with age.

In a Regression Growth Evaluation Statin Study, carriers of the R219K ABC1 mutation were found to have significantly lower triglyceride levels that individuals without this mutation. This result suggests that compounds that bind near Arg219 in wild-type ABC1 or otherwise mimic the function provided by Lys219 in the R219K ABC1 variant may lower triglyceride levels, and thus decrease risk of CAD. In addition, carriers of the V399A ABC1 variant had higher HDL levels and fewer coronary events than individuals without this variant. Thus, compounds that bind near Val399 in wild-type ABC1 or mimic the function provided by Ala399 in the V399A ABC1 variant may increase cholesterol levels and decrease risk of CAD. Determining the presence or absence of the R219K or V399A ABC1 variants in individuals may be useful in selecting therapies (such as HDL-lowering, triglyceride-raising, or anti-CAD therapies) for these subjects.

The following examples are to illustrate the invention. They are not meant to limit the invention in any way.

### Analysis of TD Families

Studies of Cholesterol Efflux

Both probands had evidence of marked deficiency of cholesterol efflux similar to that previously demonstrated in TD patients. TD-1 is of Dutch descent while TD-2 is of British descent.

Linkage Analysis and Establishment of a Physical Map

Multiple DNA markers were genotyped in the region of 9q31 to which linkage to TD had been described (Rust et al., Nat. Genet. 20, 96-98, 1998). Two point linkage analysis gave a maximal peak LOD score of 6.49 at D9S1832 with significant evidence of linkage to all markers in a ˜10 cM interval. Recombination with the most proximal marker, D9S1690 was seen in II-09 in Family TD-1, providing a centromeric boundary for the disease gene. Multipoint linkage analysis of these data did not increase the precision of the positioning of the disease trait locus.

A physical map spanning approximately 10 cM in this region was established with the development of a YAC contig. In addition, 22 other polymorphic multi-allelic markers which spanned this particular region were mapped to the contig, and a subset of these were used in construction of a haplotype for further analysis.

While the family of Dutch decent did not demonstrate any consanguinity, the proband in TD-2 was the offspring of a first-cousin consanguineous marriage. We postulated, therefore, that it was most likely that this proband would be homozygous for the mutation while the proband in the Dutch family was likely to be a compound heterozygote. The Dutch proband shows completely different mutation bearing haplotypes, supporting this hypothesis.

The TD-2 proband was homozygous for all markers tested distal to D9S127 but was heterozygous at D9S127 and DNA markers centromeric to it. This suggested that the gene for TD was likely located to the genomic region telomeric of D9S127 and encompassed by the markers demonstrating homozygosity.

Mutation Detection

Based on the defect in intracellular cholesterol transport in patients with TD, we reviewed the EST database for genes in this region which might be relevant to playing a role in this process. The ABC1 transporter gene had previously been mapped to 9q31, but its precise physical location had not been determined (Luciani et al., Genomics 21:150-159, 1994). The ABC1 gene is a member of the ATP binding cassette transporters which represents a super family of highly conserved proteins involved in membrane transport of diverse substrates including amino acids, peptides, vitamins and steroid hormones (Luciani et al., Genomics 21:150-159, 1994; Dean et al., Curr. Opin. Gen. Dev. 5:779-785, 1995). Primers to the 3′ UTR of this gene mapped to YACs spanning D9S306 (887-B2 and 930-D3) compatible with it being a strong candidate for TD. We initiated large scale genomic sequencing of BACs spanning approximately 800 kb around marker D9S306 (Research Genetics RPCI-11 BACs: 4I8, 31J20, 47O19, and 179G21, which are publicly available from Research Genetics which is located at 2130 Memorial Parkway, Huntsville, Ala. 35801) BACs 4I8, 31J20, 47O19, and 179G21 are identical to previously described BACs 269, 274, 279 and 291, respectively (U.S. Ser. No. 09/526,193; U.S. Ser. No. 60/124,702; U.S. Ser. No. 60/138,048; U.S. Ser. No. 60/139,600; U.S. Ser. No. 60/151,977; Brooks-Wilson et al., Nat. Genet. 22:336-345, 1999). The ABC1 gene was revealed encompassing 49 exons and a minimum of 75 Kb of genomic sequence. In view of the potential function of a gene in this family as a cholesterol transporter, its expression in fibroblasts and localization to the minimal genomic segment underlying TD, we formally assessed ABC1 as a candidate.

Patient and control total fibroblast RNA was used in Northern blot analysis and RT-PCR and sequence analyses. RT-PCR and sequence analysis of TD-1 revealed a heterozygous T to C substitution in the TD-1 proband, which would result in a substitution of arginine for cysteine at a conserved residue between mouse and man. This mutation, confirmed by sequencing exon 31 of the ABC1 gene, exhibited complete segregation with the phenotype on one side of this family. This substitution creates a Hgal site, allowing for RFLP analysis of amplified genomic DNA and confirmation of the mutation. The point mutation in exon 31 was not seen on over 200 normal chromosomes from unaffected persons of Dutch decent, and 250 chromosomes of Western European decent, indicating it is unlikely to be a polymorphism. Northern blot analysis of fibroblast RNA from this patient, using a cDNA encompassing exons 2 to 50 of the gene, revealed a normal sized ˜8 Kb transcript and a truncated mutant transcript which was not visible in control RNA or in RNA from other patients with HDL deficiency. Additionally, Northern blot analysis using clones encompassing discrete regions of the cDNA revealed that the mutant transcript was detected with a cDNA compassing exons 2 to 50, 2 to 42, 2 to 23, much more faintly with a probe spanning exon 24 to 30, and not seen with probes encompassing exons 31 to 43 or a probe spanning exons 31 to 50. This was repeated on multiple filters with control RNA, RNA from other patients with HDL deficiency and the other TD proband, and only in TD-1 was the truncated transcript observed. Sequence analysis of the coding region did not reveal an alteration in sequence that could account for this finding. Furthermore, DNA analysis by Southern blot did not reveal any major rearrangements. Completion of exon sequencing in genomic DNA showed that this mutation was a G to C transversion at position (+1) of intron 24, affecting a splice donor site and causing aberrant splicing.

RT-PCR analysis of fibroblast RNA encoding the ABC1 gene from the proband in TD-2 revealed a homozygous nucleotide change of A to G at nucleotide 1864. in exon 14, resulting in a substitution of arginine for glutamine at residue 597, occurring just proximal to the first predicted transmembrane domain of ABC1 at a residue conserved in mouse and as well as a C. elegans homolog. This mutation creates a second AciI site within exon 14. Segregation analysis of the mutation in this family revealed complete concordance between the mutation and the low HDL phenotype as predicted. The proband in TD-2 is homozygous for this mutation, consistent with our expectation of a disease causing mutation in this consanguineous family.

### Analysis of FHA Families

Linkage Analysis and Refinement of the Minimal Genomic Region Containing the Gene for FHA

Data from microsatellite typing of individual family members from the four pedigrees of French Canadian origin were analyzed. A maximum LOD score of 9.67 at a recombination fraction of 0.0 was detected at D9S277 on chromosome 9q31. Thereafter, 22 markers were typed in a region spanning 10 cM around this locus in these families. The frequency for these markers were estimated from a sample of unrelated and unaffected subjects of French ancestry.

TD and FHA have thus far been deemed distinct with separate clinical and biochemical characteristics. Even though the genes for these disorders mapped to the same region, it was uncertain whether FHA and TD were due to mutations in the same gene or, alternatively, due to mutations in genes in a similar region. Refinement of the region containing the gene for FHA was possible by examining haplotype sharing and identification of critical recombination events. Seven separate meiotic recombination events were seen in these families, clearly indicating that the minimal genomic region containing the potential disease gene was a region of approximately 4.4 cM genomic DNA spanned by marker D9S1690 and D9S1866. This region is consistent with the results of two point linkage analysis which revealed maximal LOD scores with markers D9S277 and D9S306 and essentially excluded the region centromeric to D9S1690 or telomeric to D9S1866. An 8^{th} meiotic recombination event further refined the FHA region to distal to D9S277.

As described herein, the ABC1 gene mapped within this interval. The overlapping genetic data strongly suggested that FHA may in fact be allelic to TD. Utilization of sets of genetic data from FHA and TD provided a telomeric boundary at D9S1866 (meiotic recombinant) and a centromeric marker at D9S127 based on the homozygosity data of TD-2. This refined the locus to approximately 1 mb between D9S127 and D9S1866. The ABC1 gene mapped within this minimal region.

Mutation Detection in FHA

Mutation assessment of the ABC1 gene was undertaken in FHA-1. Using primers that spanned overlapping segments of the mRNA we performed RT-PCR analysis and subjected these fragments to mutational analysis. A deletion of three nucleotides is evident in the RT-PCR sequence of FHA-1 III.01, resulting in a loss of nucleotides 2151-2153 and deletion of a leucine (ΔL693) at amino acid position 693. This leucine is conserved in mouse and C. elegans. The alteration was detected in the RT-PCR products as well as in genomic sequence from exon 15 specific amplification. This mutation results in a loss of an EarI restriction site. Analysis of genomic DNA from the family indicated that the mutation segregated completely with the phenotype of HDL deficiency. The loss of the EarI site results in a larger fragment being remaining in persons heterozygous for this mutation. This mutation maps to the first putative transmembrane domain of ABC1 and was not seen in 130 chromosomes from persons of French Canadian descent nor seen in over 400 chromosomes from persons of other Western European ancestry.

A mutation has also been found in patient genomic DNA in pedigree FHA-3 from Quebec. The alteration, a 6 bp deletion of nucleotides 5752-5757 within exon 42, results in a deletion of amino acids 1893 (Glu) and 1894 (Asp). The deletion was detected as a double superimposed sequence starting from the point of the deletion, and was detected in sequence reads in both directions. The deletion can be detected on 3% agarose or 10% polyacrylamide gels, and segregates with disease in FHA-3. It was not seen in 128 normal chromosomes of French-Canadian origin or in 434 other control chromosomes. Amino acids 1893 and 1894 are in a region of the ABC1 protein that is conserved between human, mouse, and C. elegans, implying that it is of functional importance.

An additional mutation has been found in patient genomic DNA in pedigree FHA-2 from Quebec. The alteration, a C to T transition at position 6504, converts an arginine at position 2144 to a STOP codon, causing truncation of the last 118 amino acids of the ABC1 protein. This alteration segregates with disease in family FHA-2.

Functional Relationship Between Changes in ABC1 Transcript Levels and Cholesterol Efflux

Antisense approaches were undertaken to decrease the ABC1 transcript and assess the effect of alteration of the transcript on intracellular cholesterol transport. The use of antisense primers to the 5′ end of ABC1 clearly resulted in a decrease to approximately 50% of normal RNA levels. This would be expected to mimic in part the loss of function due to mutations on one allele, similar to that seen in heterozygotes for TD and patients with FHA. Importantly, reduction in the mRNA for the ABC1 gene resulted in a significant reduction in cellular cholesterol efflux, further establishing the role of this protein in reverse cholesterol transport and providing evidence that the mutations detected are likely to constitute loss of function mutations. Furthermore, these data support the functional importance of the first 60 amino acids of the protein. Antisense oligonucleotide AN-6 is directed to the novel start codon 5′ to the one indicated in AJ012376.1; this antisense oligonucleotide effectively suppresses efflux.

Polymorphisms in ABC1 5′ Regulatory Region and 5′ UTR

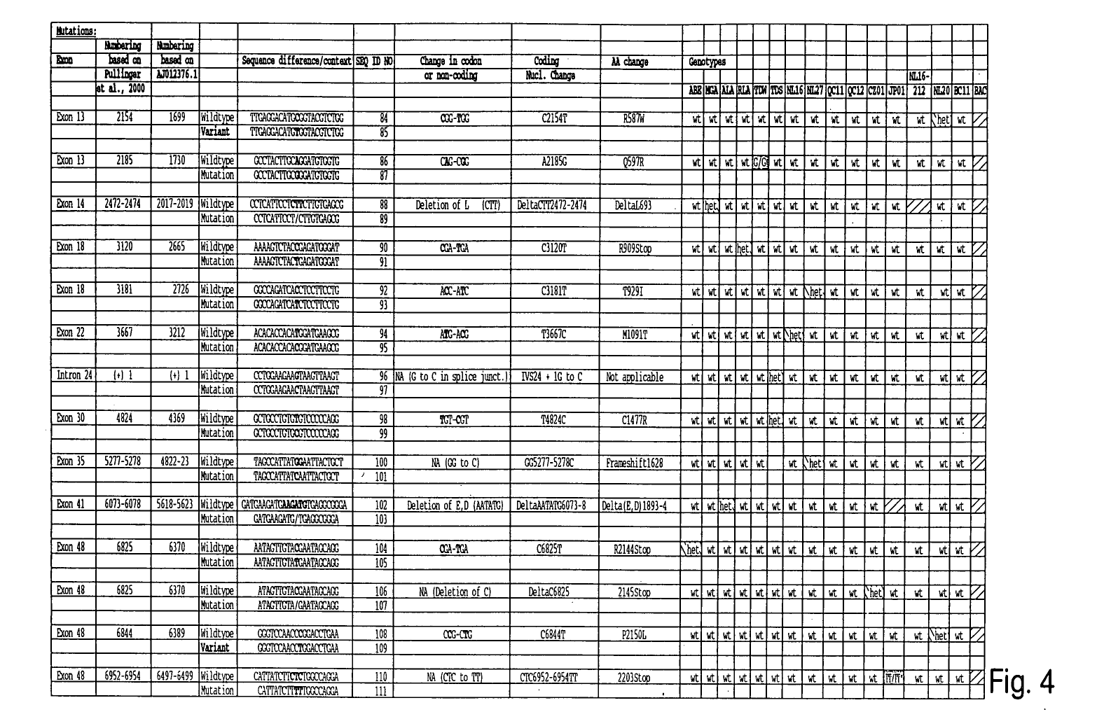

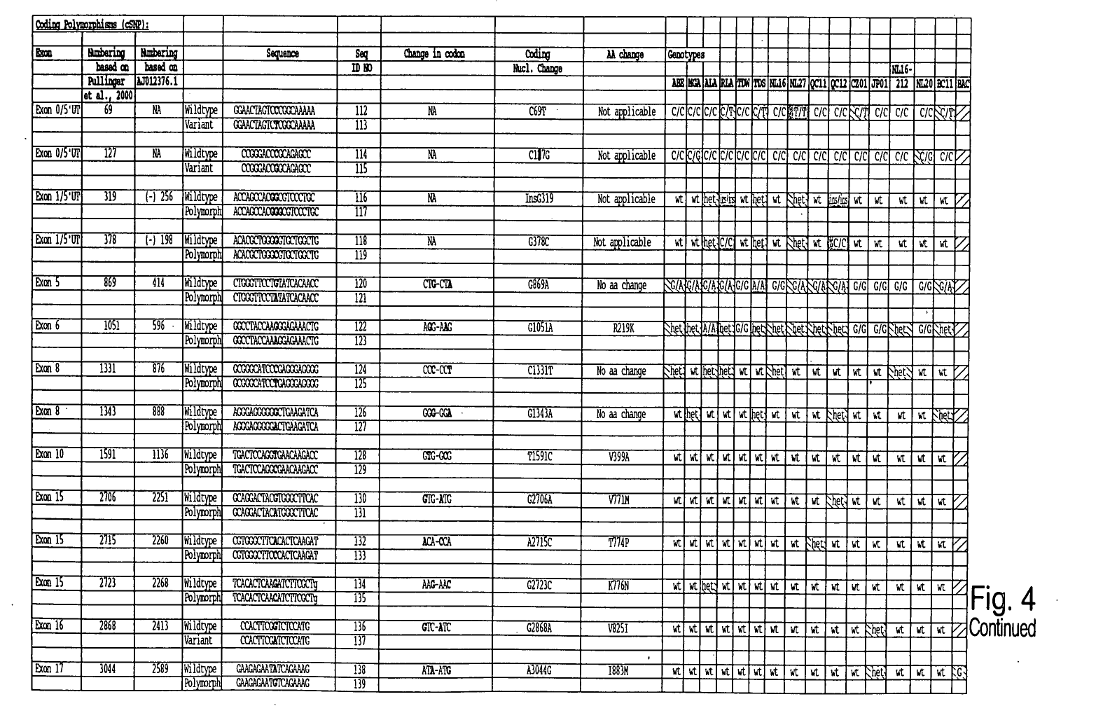

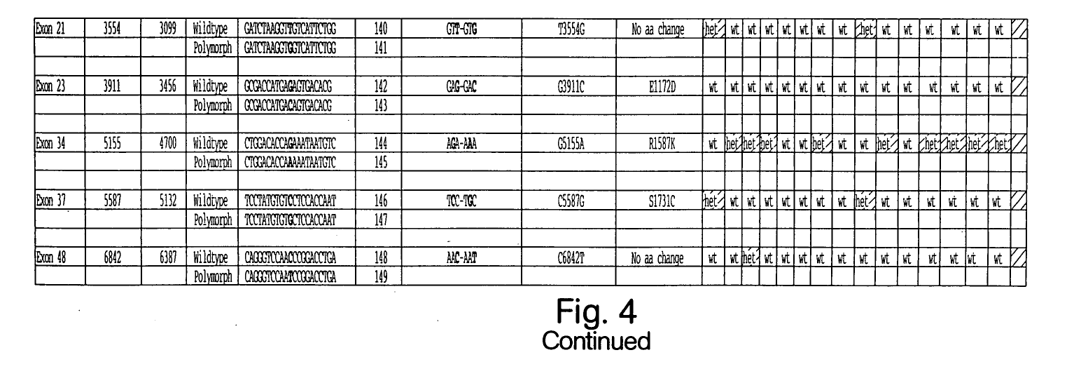

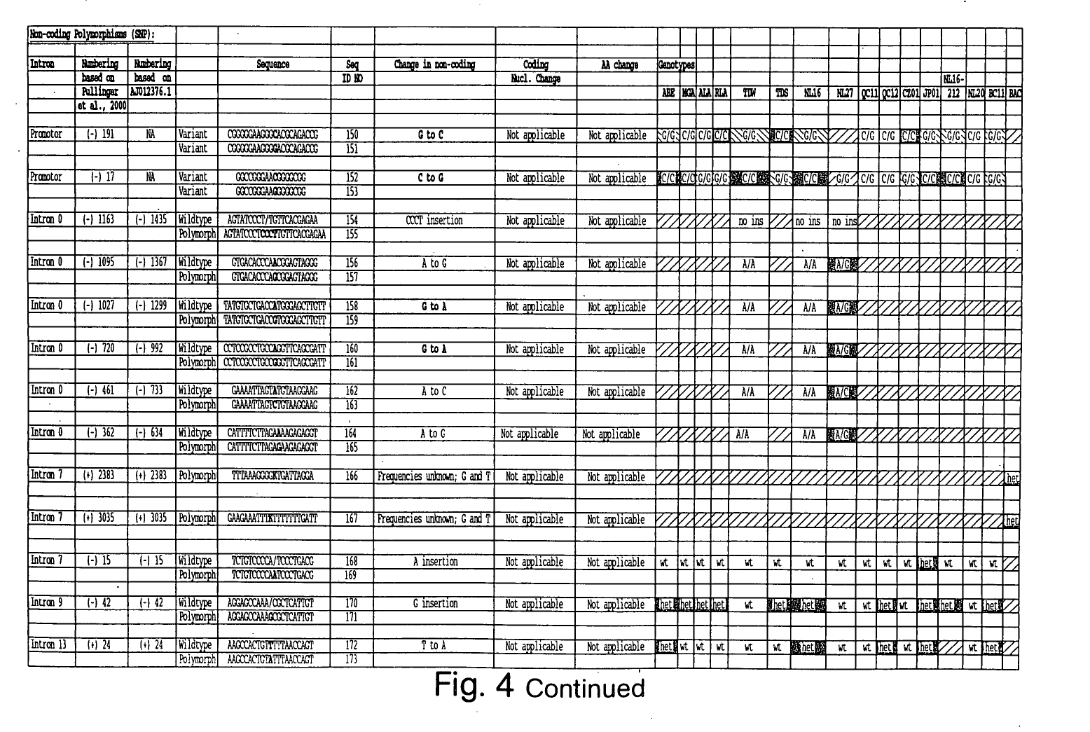

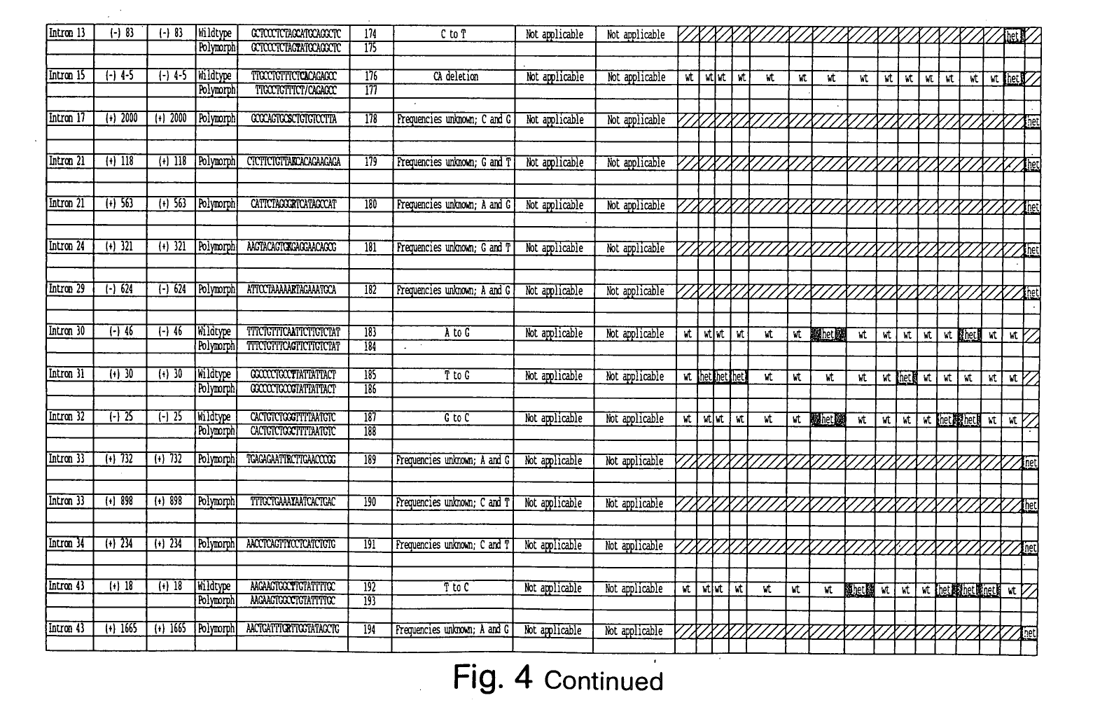

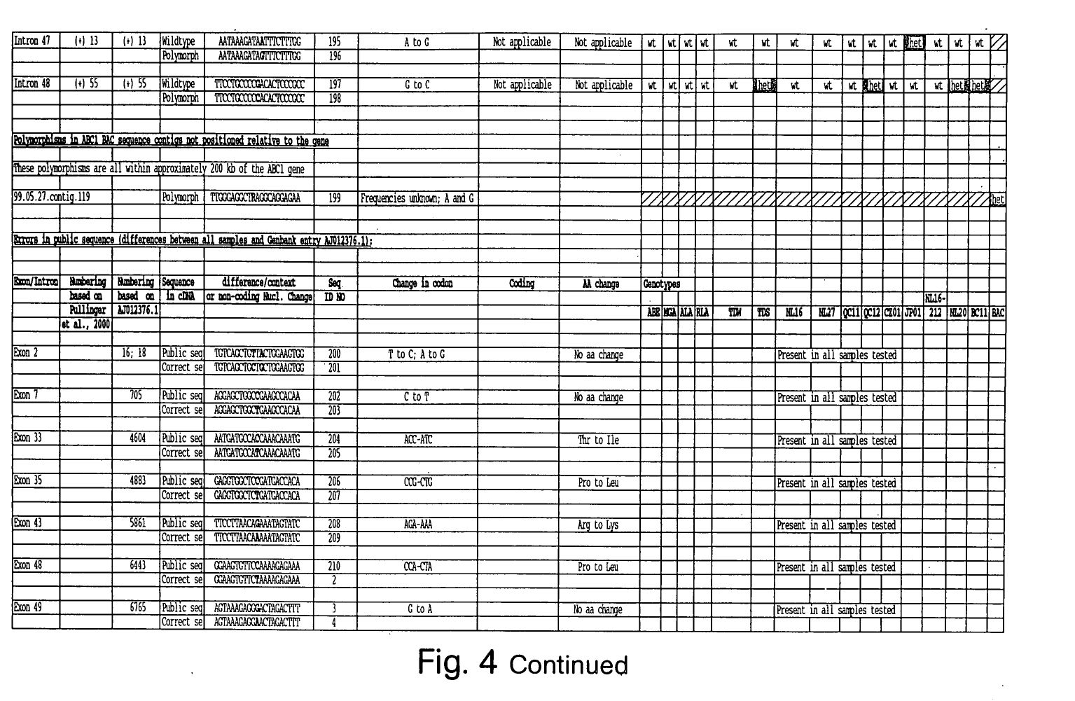

Several polymorphisms in the 5′ regulatory region of human ABC1 (SEQ ID NO: 1) have been identified (FIG. 4). Because of their location, it is likely that ABC1 gene expression will differ among humans having different promoter polymorphisms, and these individuals may also respond differently to the same drug treatment. Thus, using these newly-identified polymorphisms, one can tailor drug treatment depending on which polymorphism(s) is/are present in a patient. The presence or absence of particular ABC1 polymorphisms may also be used in determining an individual's predisposition to developing CVD.

The methods of the invention may be performed using the following materials and methods.

Biochemical Studies

Blood is withdrawn in EDTA-containing tubes for plasma lipid, lipoprotein cholesterol, ApoAI, and triglyceride analyses, as well as storage at −80° C. Leukocytes arere isolated from the buffy coat for DNA extraction.

Lipoprotein measurement is performed on fresh plasma as described elsewhere (Rogler et al., Arterioscler. Thromb. Vasc. Biol. 15:683-690, 1995). Lipids, cholesterol and triglyceride levels are determined in total plasma and plasma at density d<1.006 g/mL (obtained after preparative ultracentrifugation) before and after precipitation with dextran manganese. Apolipoprotein measurement is performed by nephelometry for ApoB and ApoAI.

Genomic Clone Assembly and Physical Map Construction of the 9q31 Region

Using the Whitehead Institute/MIT Center for Genome Research map as a reference, the genetic markers of interest at 9q31 were identified within YAC contigs. Additional markers that mapped to the approximate 9q31 interval from public databases and the literature were then assayed against the YAC clones by PCR and hybridization analysis. The order of markers was based on their presence or absence in the anchored YAC contigs and later in the BAC contig. Based on the haplotype analysis, the region between D9S277 and D9S306 was targeted for higher resolution physical mapping studies using bacterial artificial chromosomes (BACs). BACs within the region of interest were isolated by hybridization of DNA marker probes and whole YACs to high-density filters containing clones from the RPCI-11 human BAC library.

Sequence Retrieval and Alignment

The human ABC1 mRNA sequence was retrieved from GenBank using the Entrez nucleotide query (Baxevanis et al., A Practical Guide to the Analysis of Genes and Proteins, eds. Baxevanis, A. D. & Ouellette, B. F. F. 98:120, 1998) as GenBank accession number AJ012376.1. The version of the protein sequence we used as wild-type (normal) was CAA10005.1.

We identified an additional 60 amino acids in-frame with the previously-believed start methionine. Bioinformatic analysis of the additional amino acids indicates the presence of a short stretch of basic amino acid residues, followed by a hydrophobic stretch, then several polar residues. This may represent a leader sequence, or another transmembrane or membrane-associated region of the ABC1 protein. In order to differentiate among the foregoing possibilities, antibodies directed to the region of amino acids 1-60 are raised against and used to determine the physical relationship of amino acids 1-60 in relation to the cell membrane. Other standard methods can also be employed, including, for example, expression of fusion proteins and cell fractionation.

The mouse ABC1 sequence used has accession number X75926. It is very likely that this mouse sequence is incomplete, as it lacks the additional 60 amino acids described herein for human ABC1.

Version 1.7 of ClustalW was used for multiple sequence alignments with BOXSHADE for graphical enhancement (http://www.isrec.isb-sib.ch:8080/software/BOX_form.html) with the default parameter. A Caenorhabditis elegans ABC1 orthologue was identified with BLAST (version 2.08) using CAA1005.1 (see above) as a query, with the default parameter except for doing an organism filter for C. elegans. The selected protein sequence has accession version number AAC69223.1 with a score of 375, and an E value of 103.

Genomic DNA Sequencing

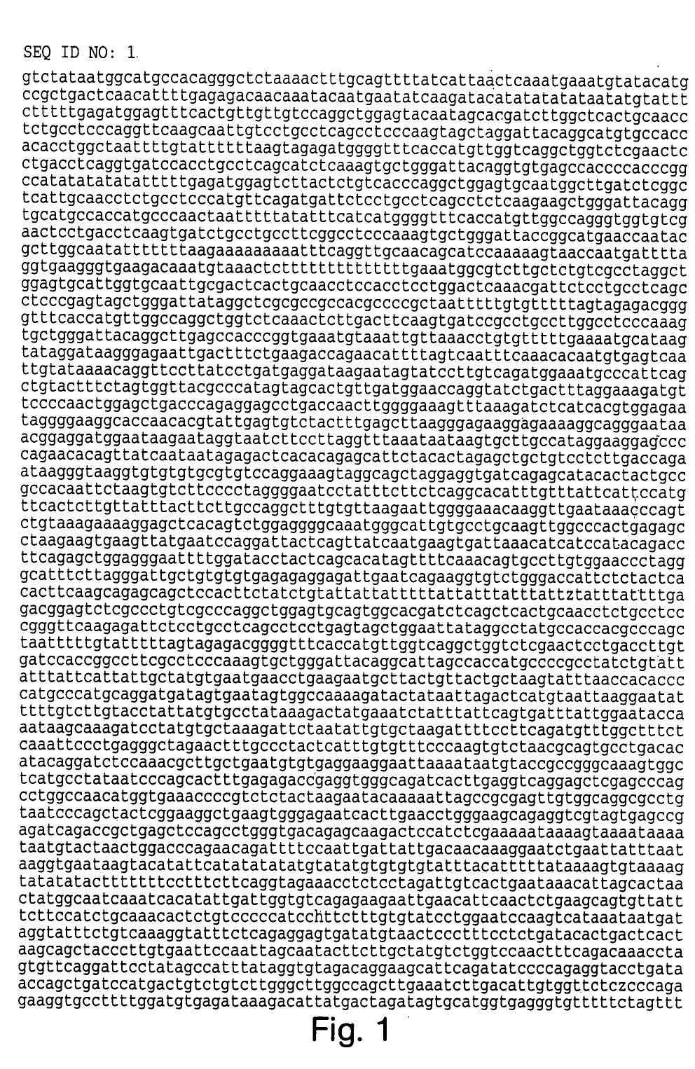

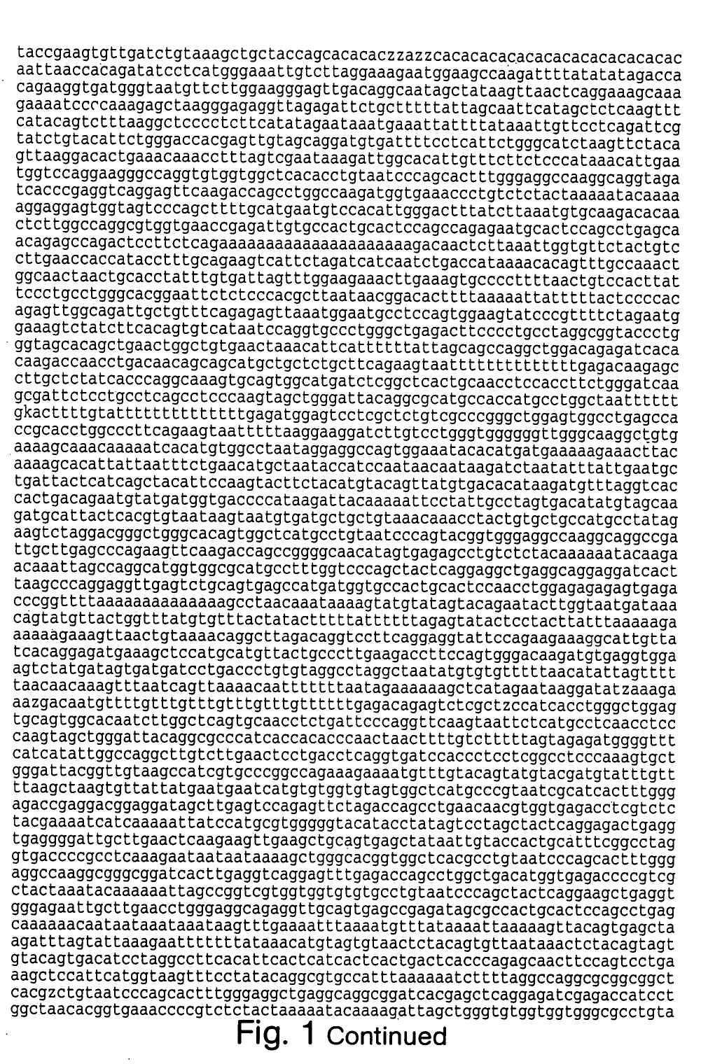

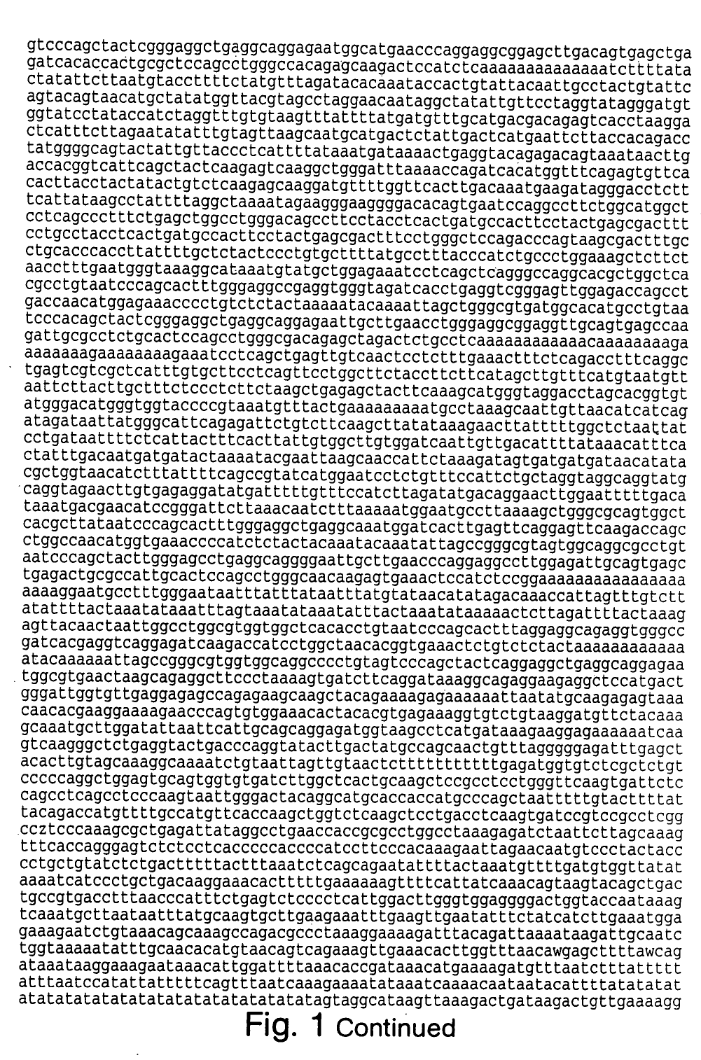

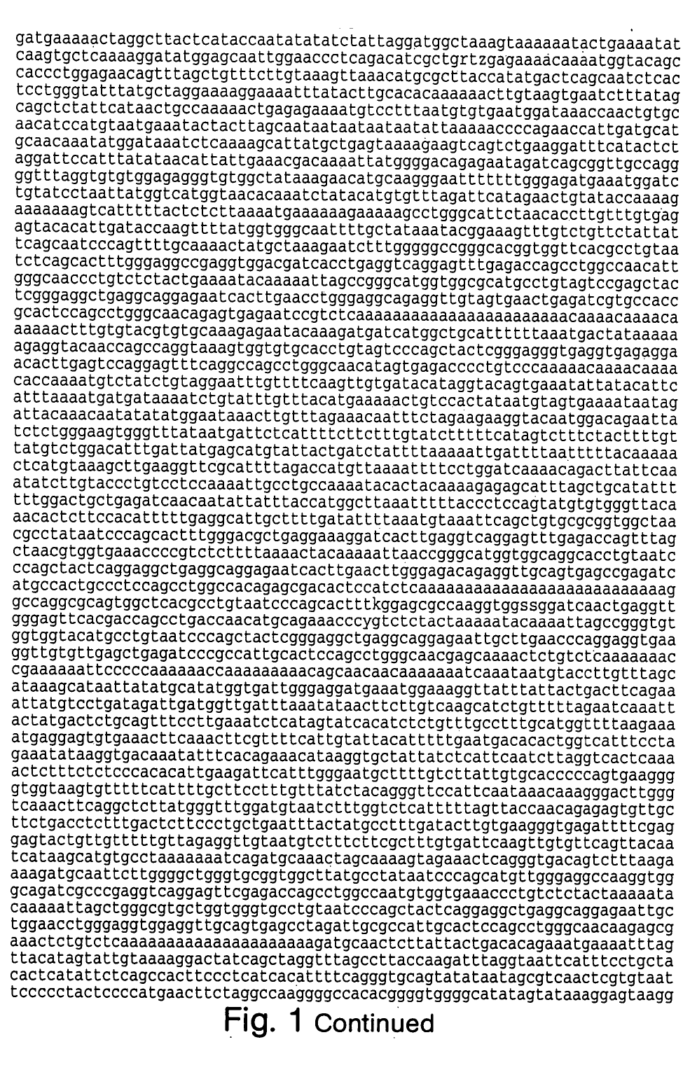

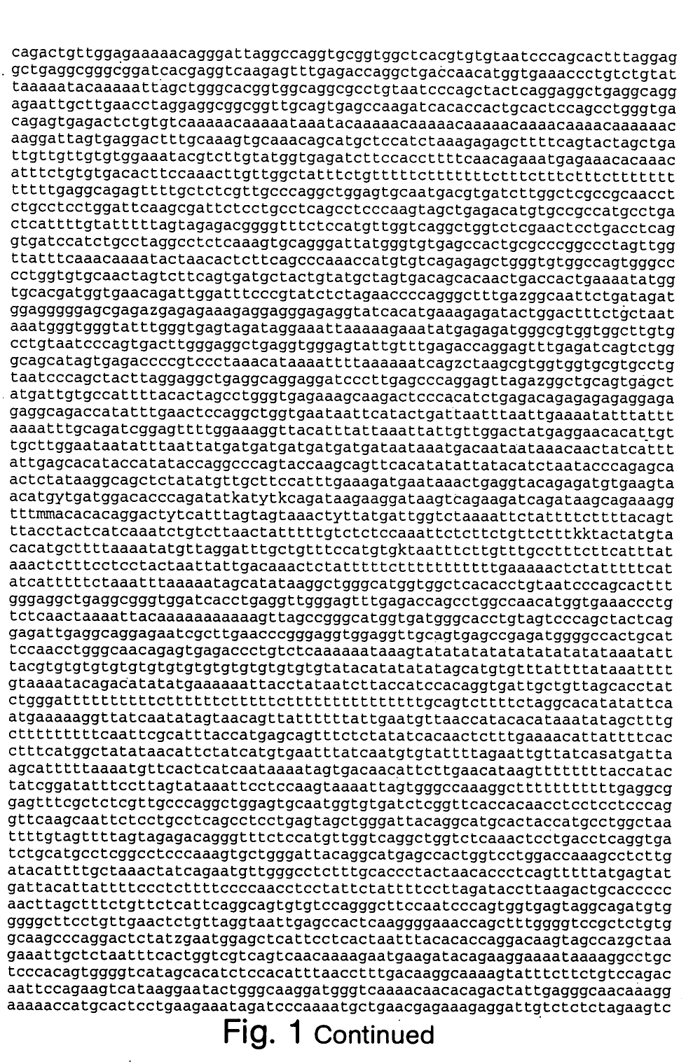

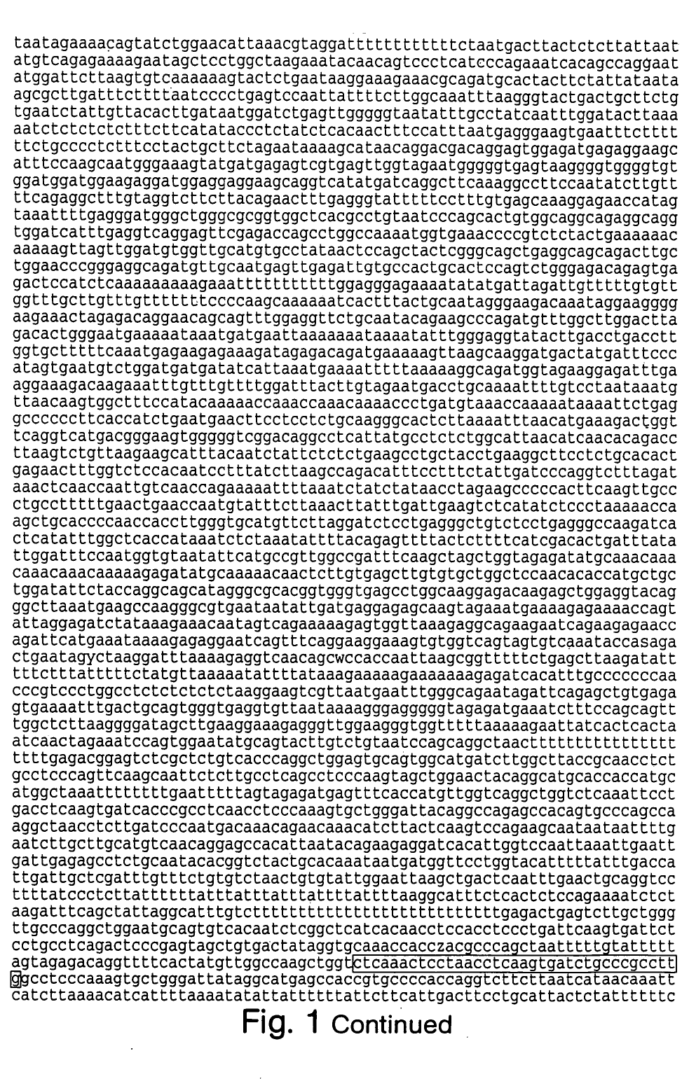

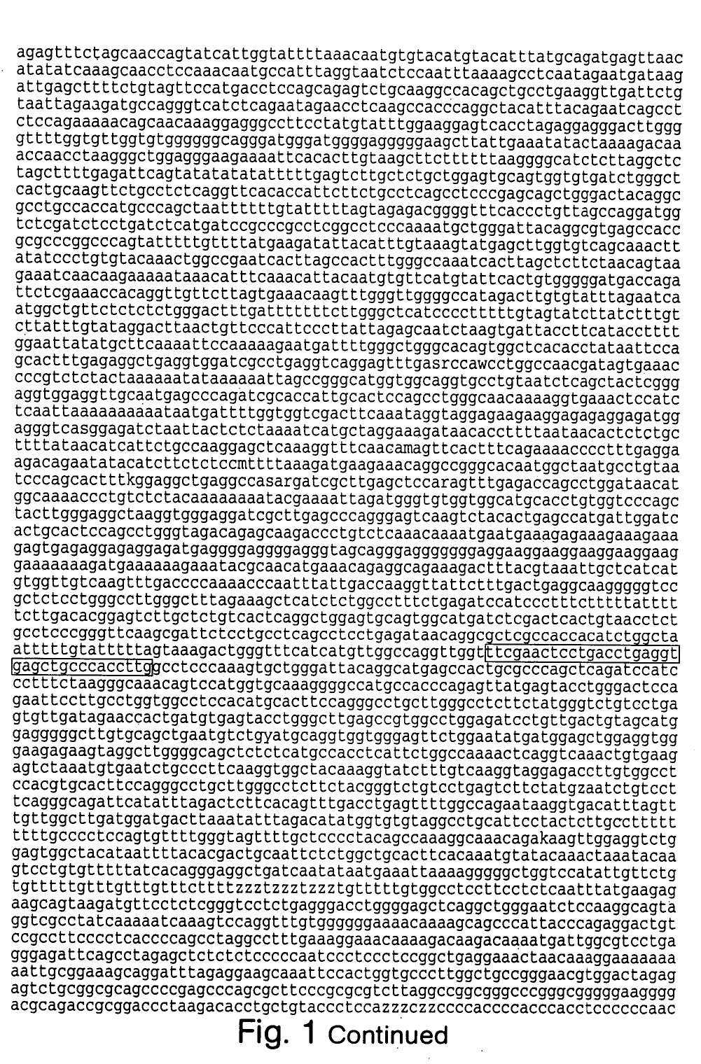

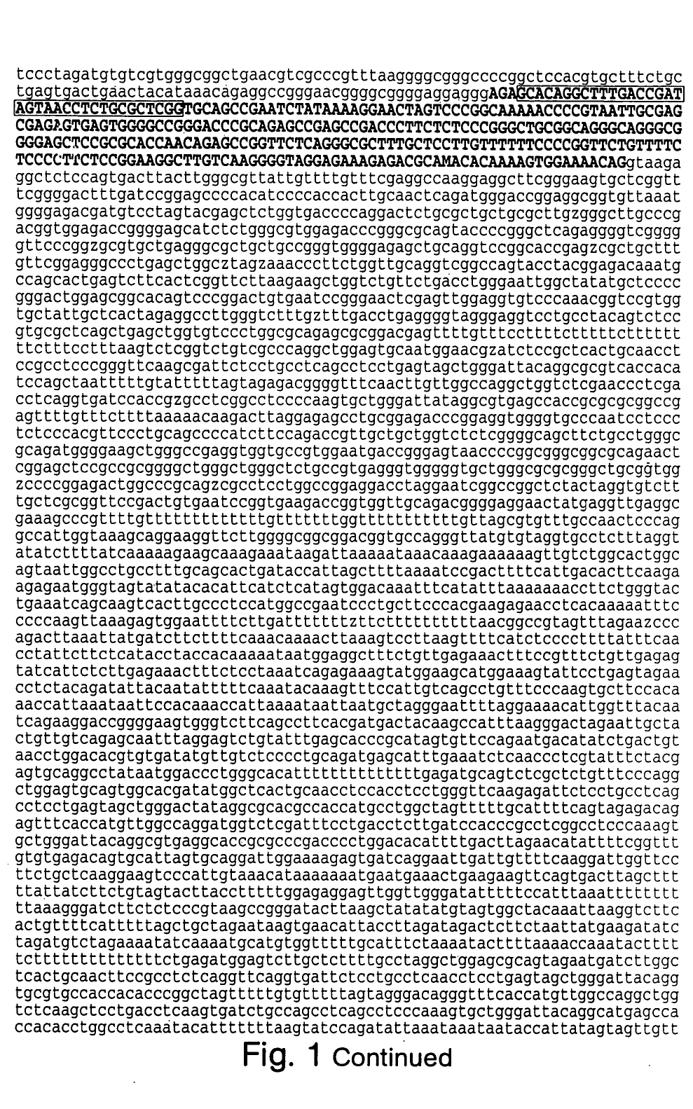

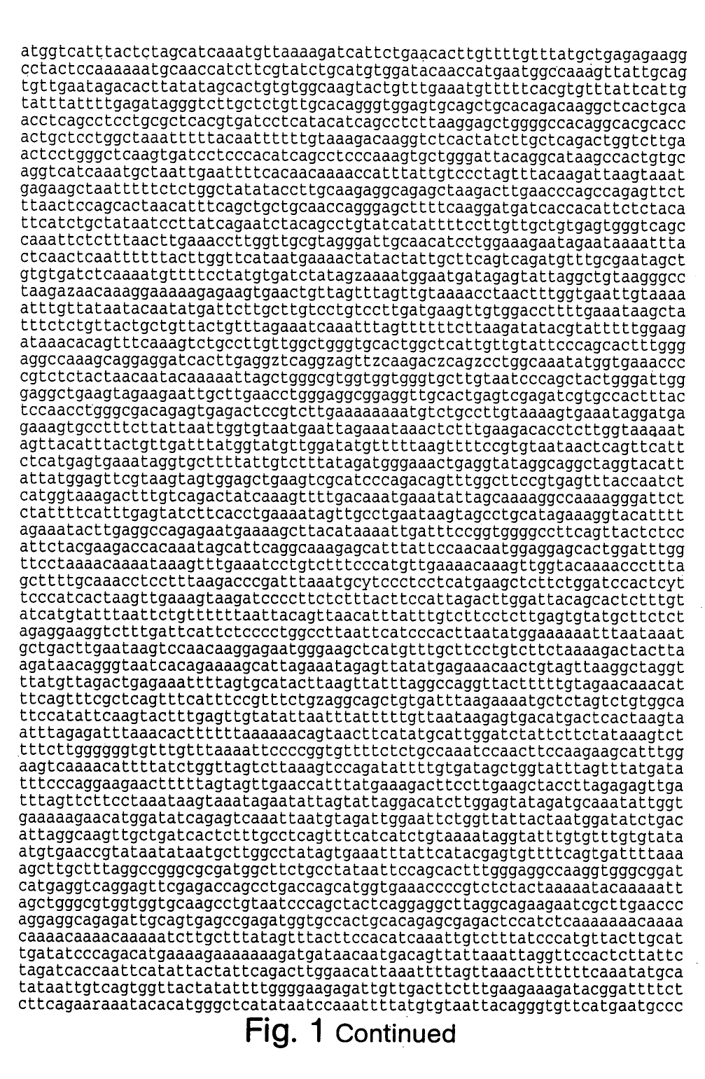

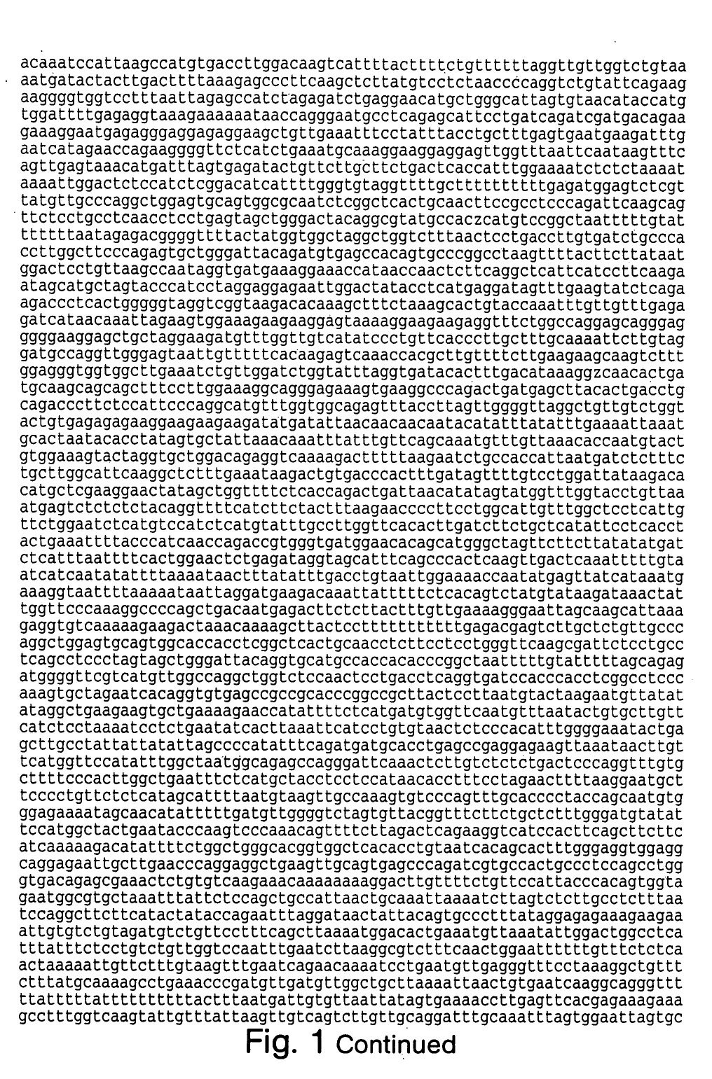

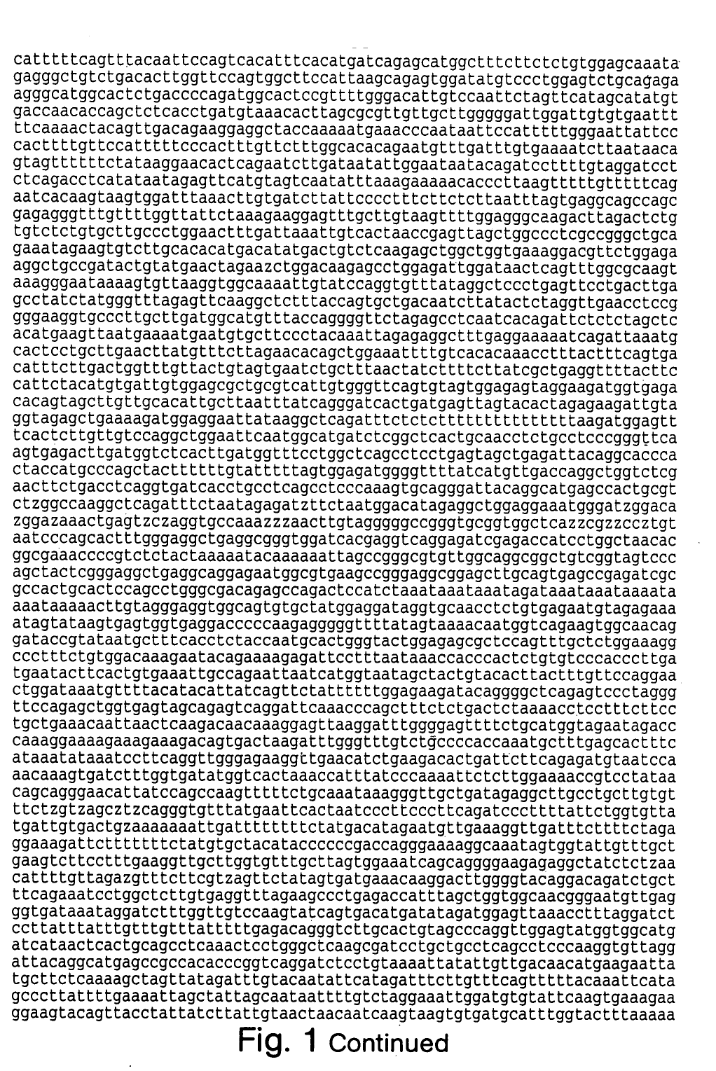

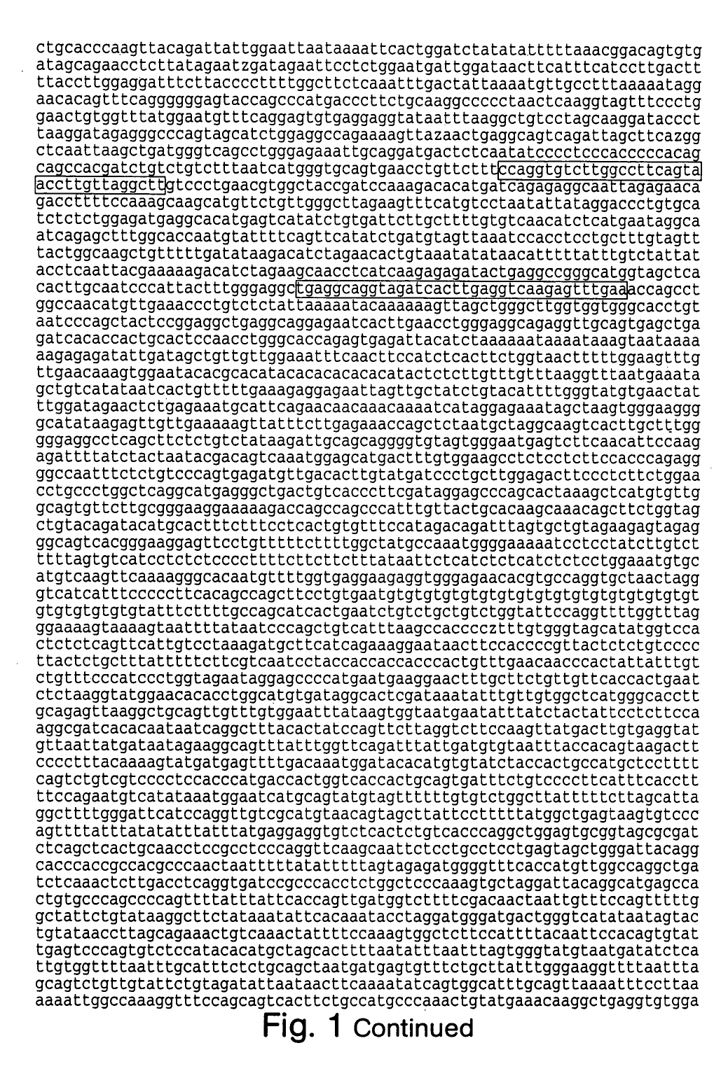

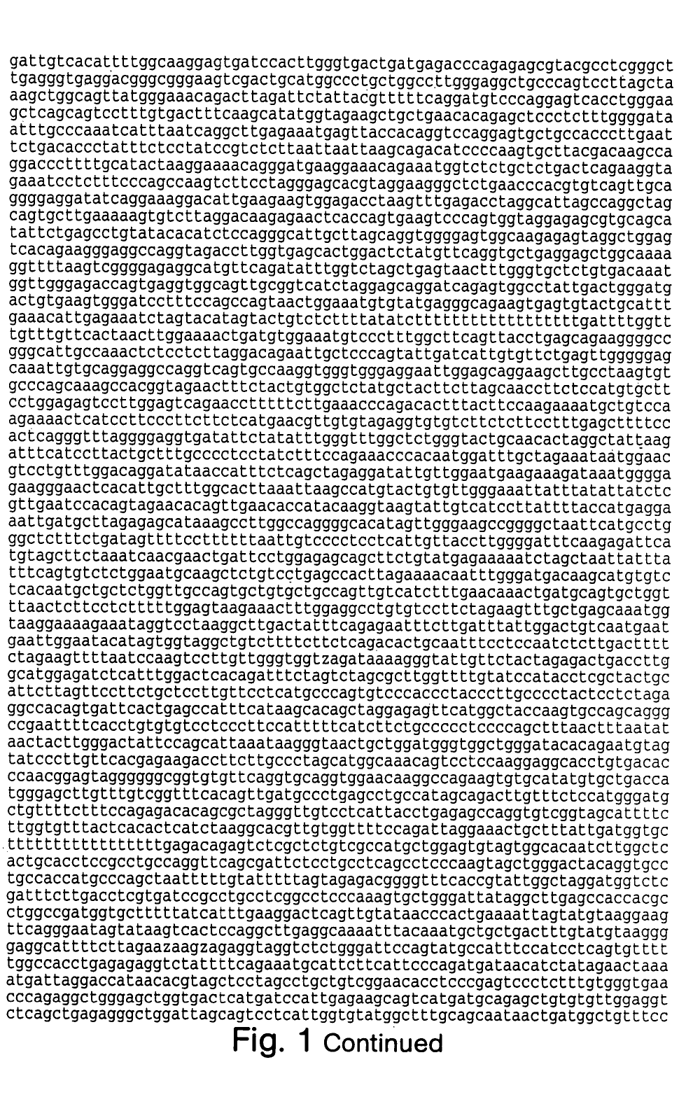

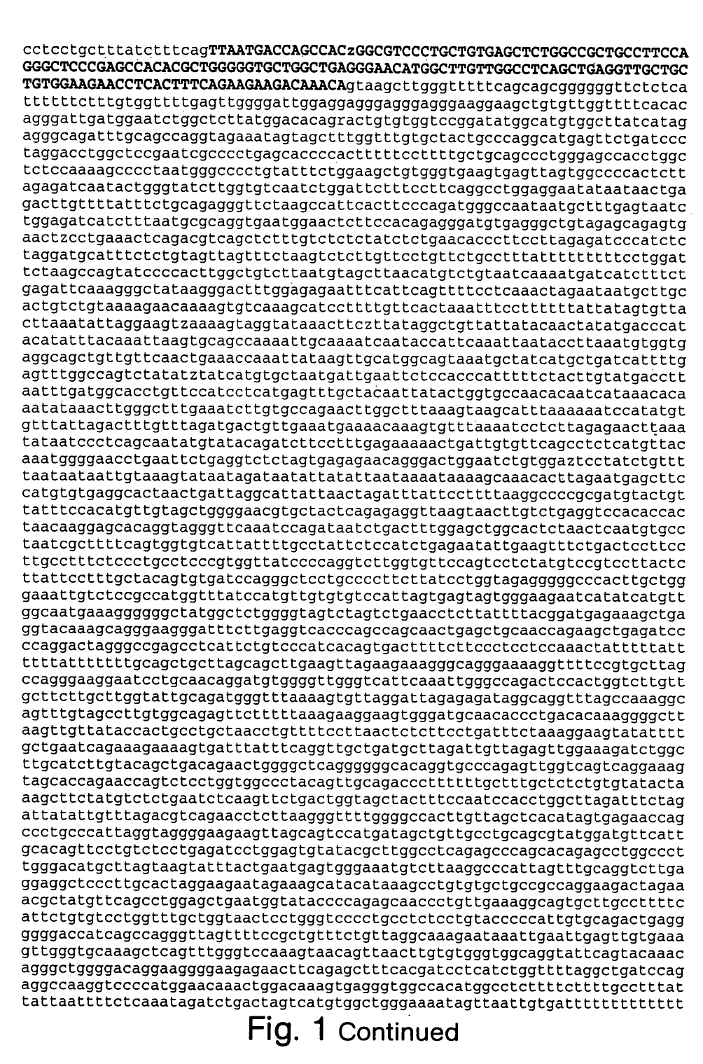

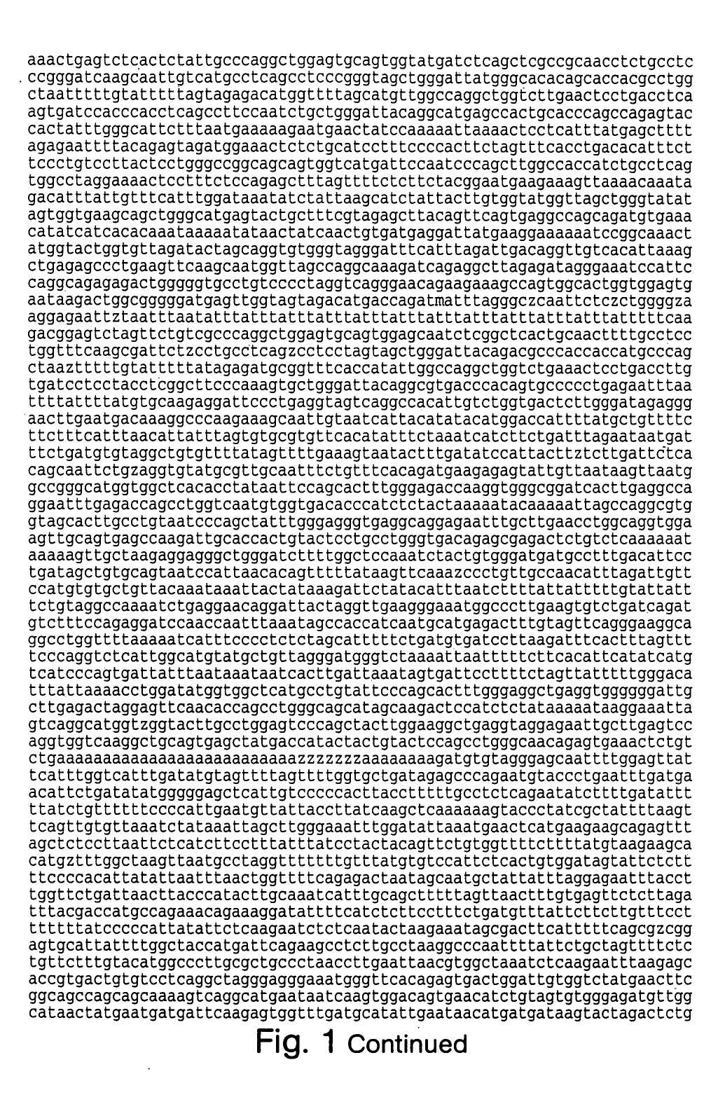

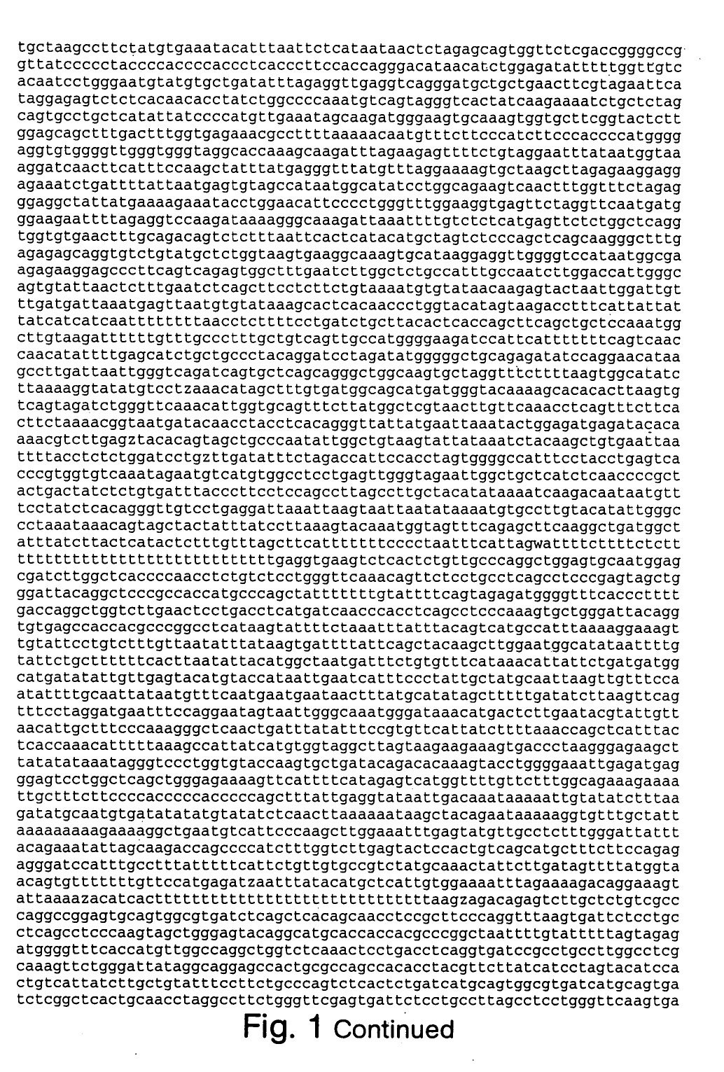

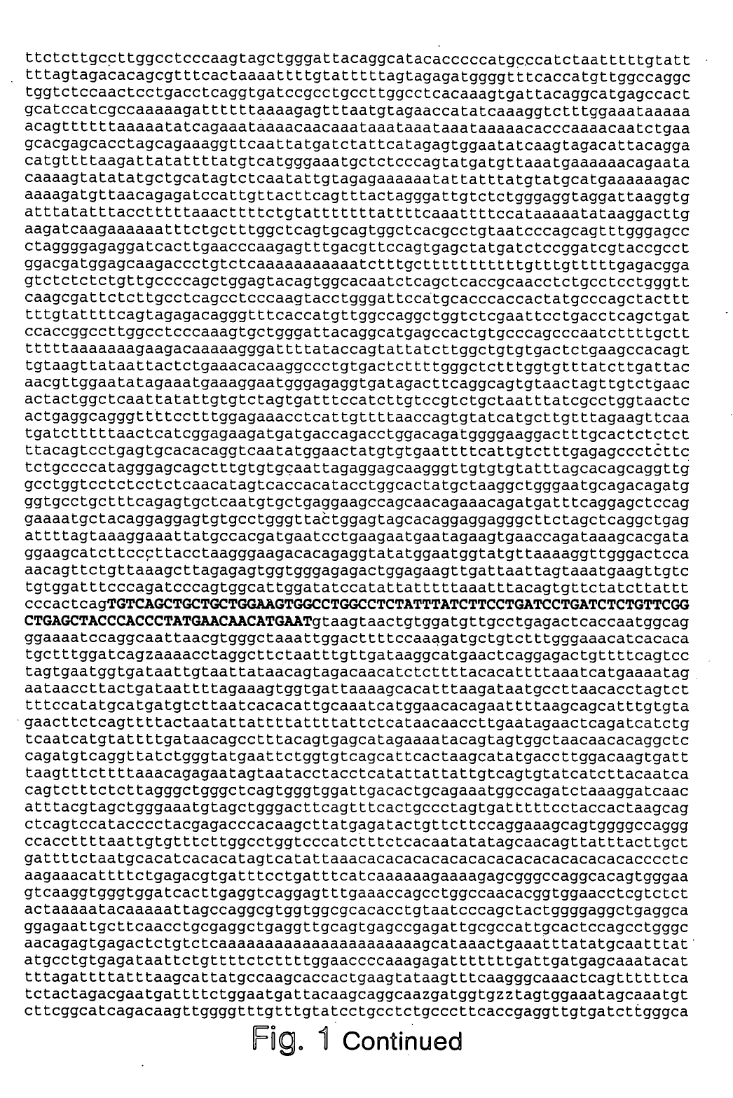

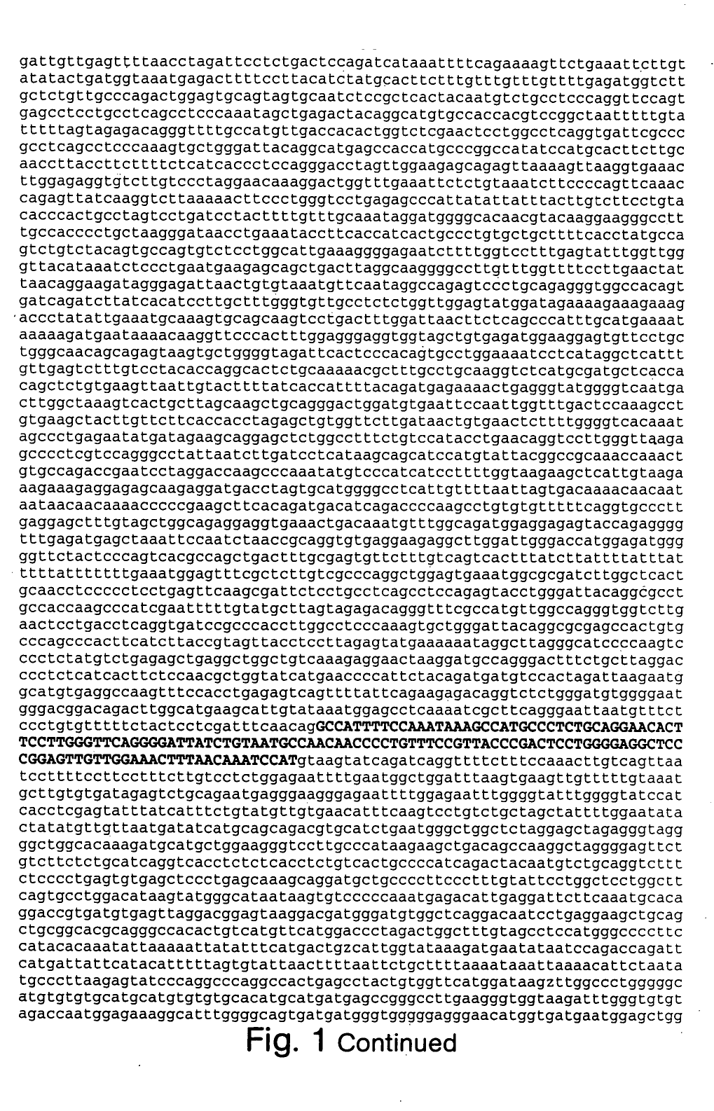

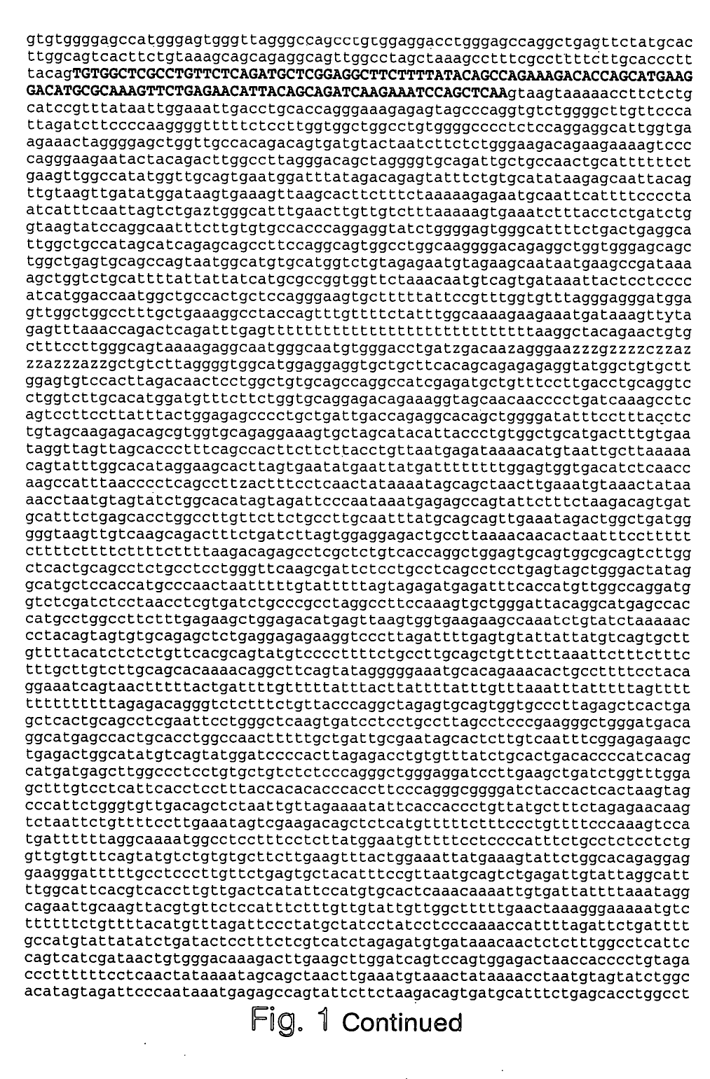

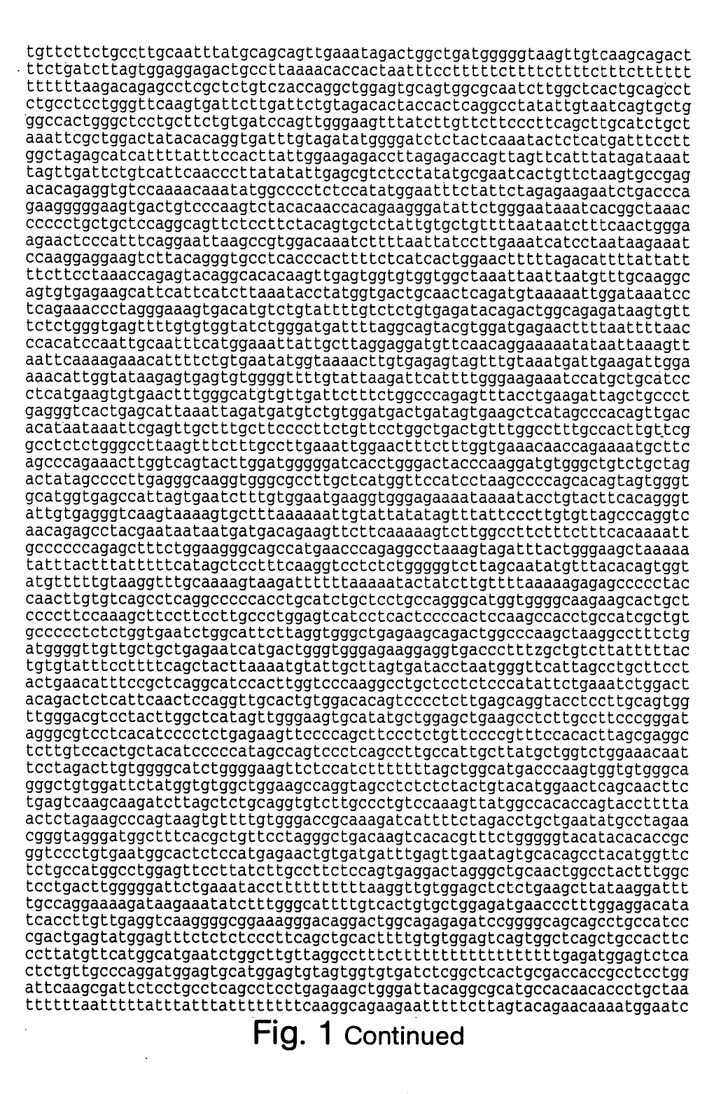

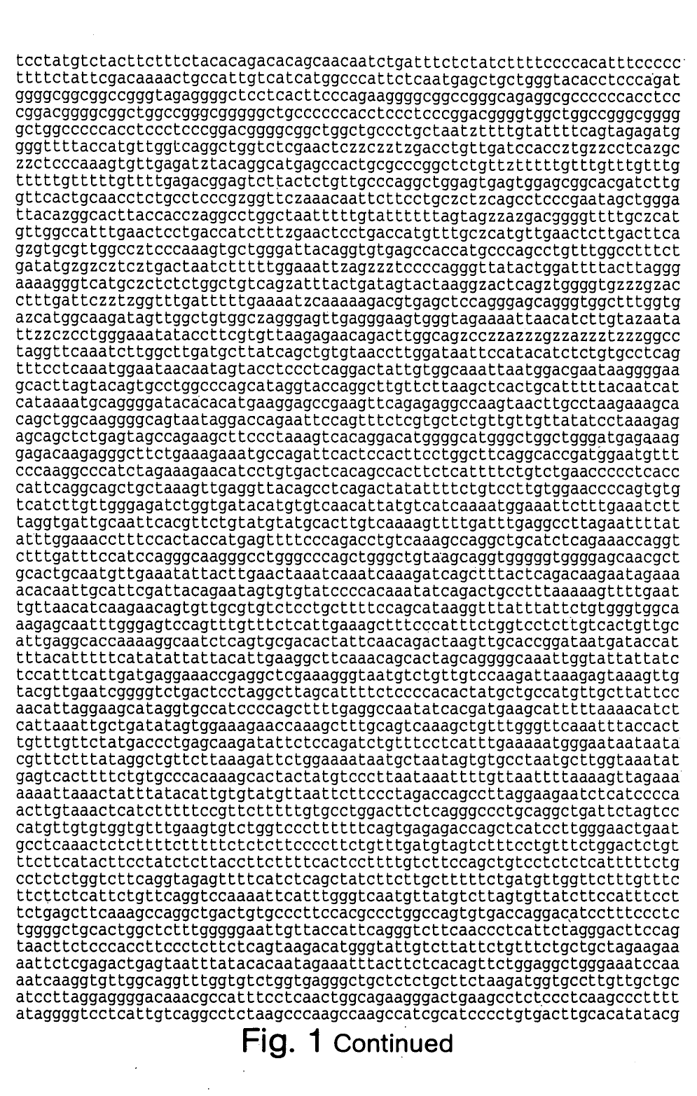

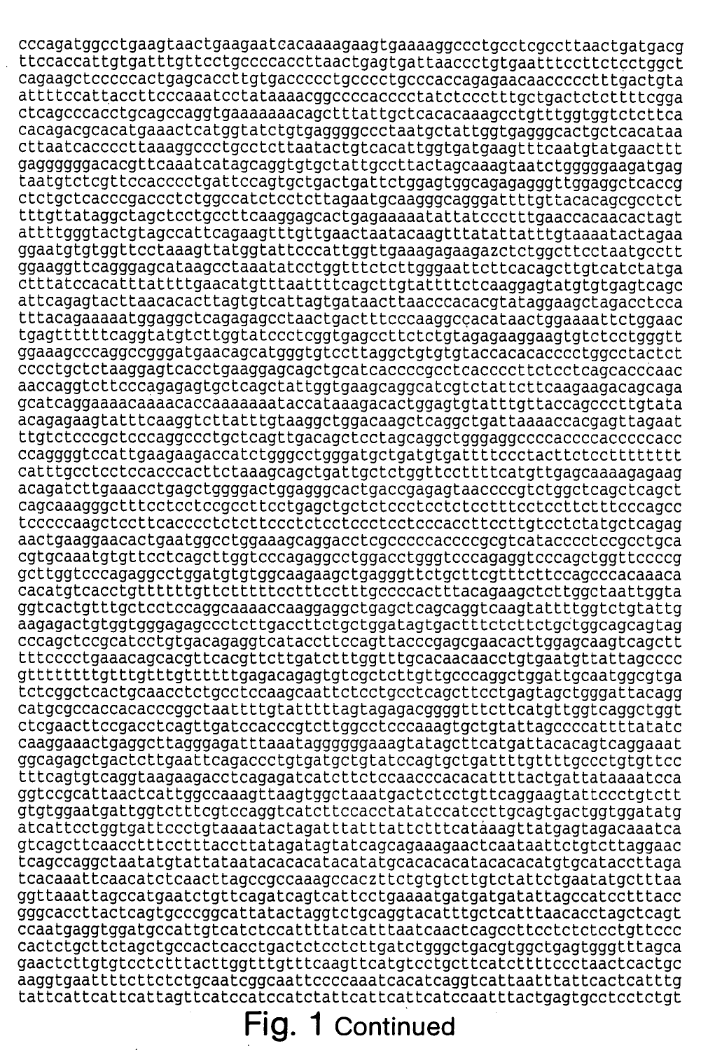

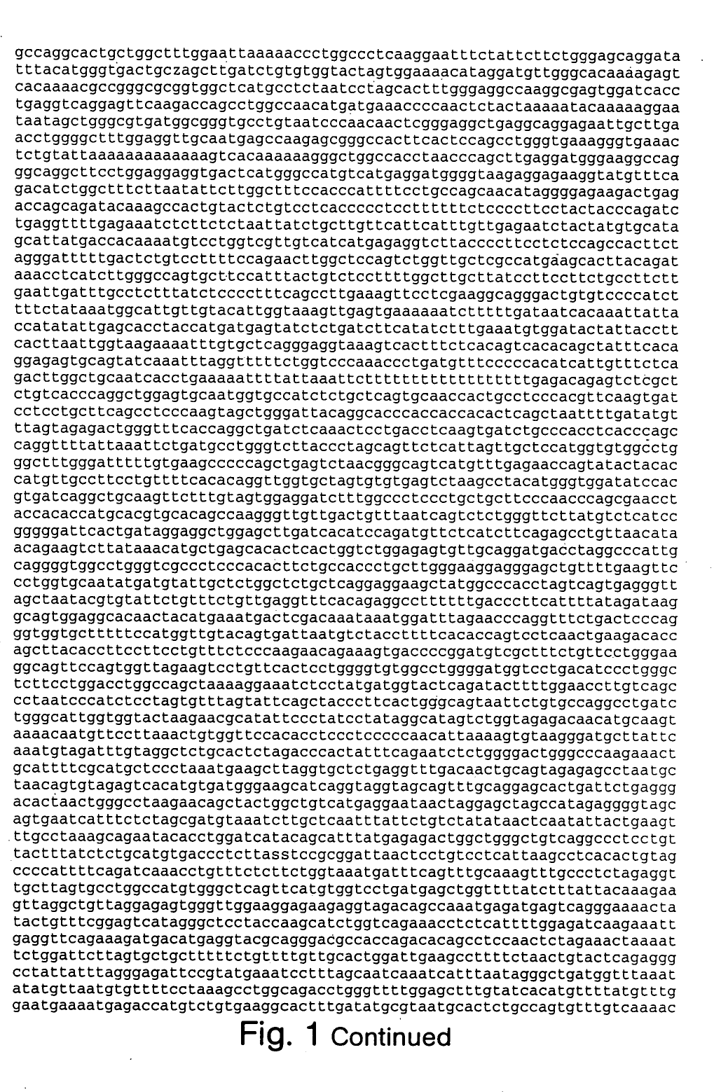

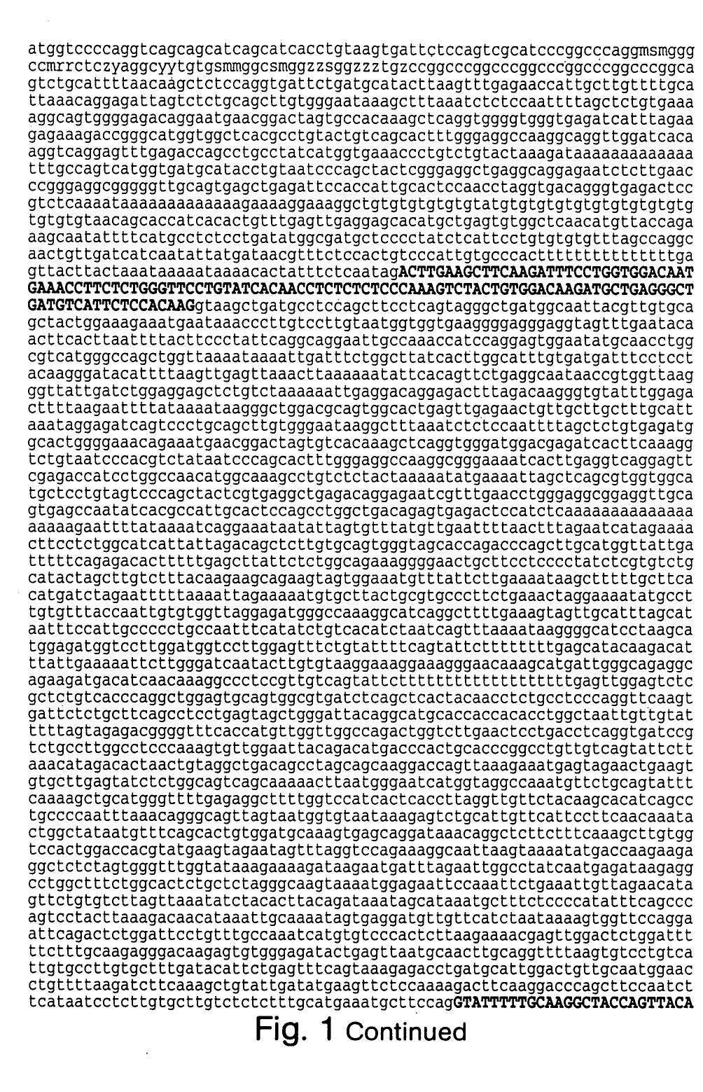

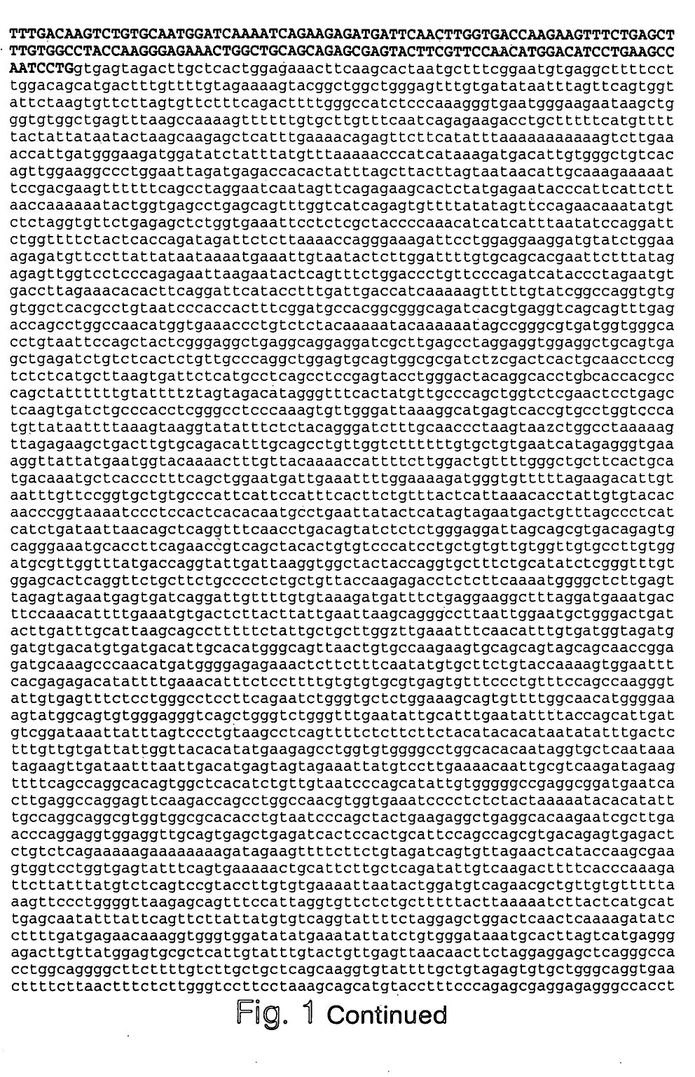

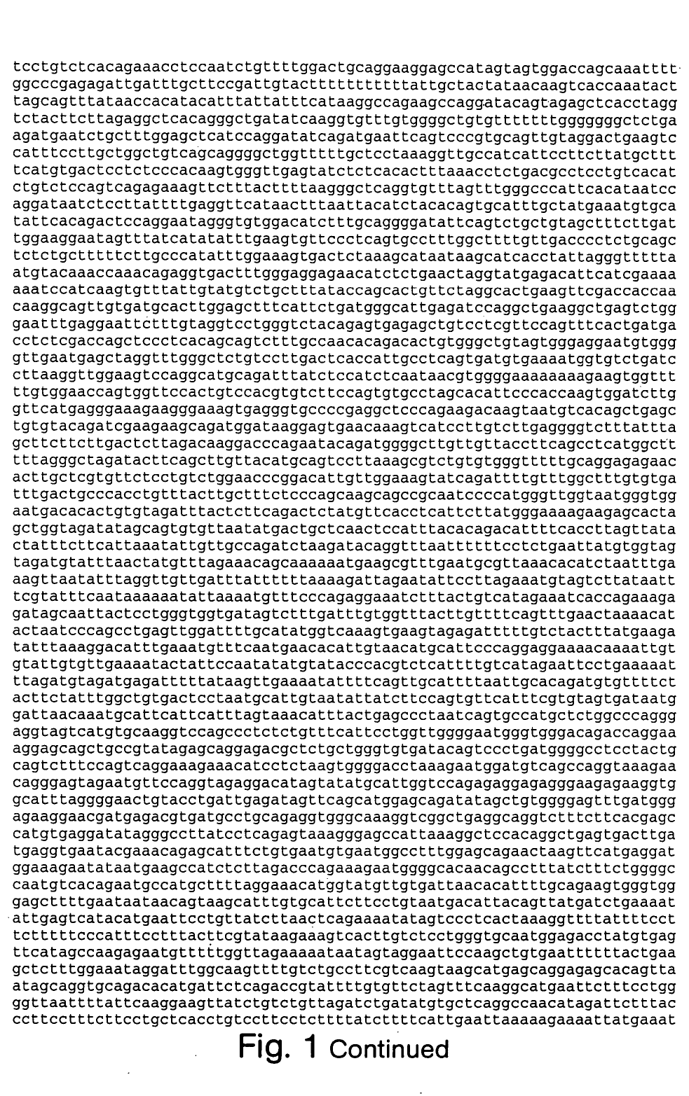

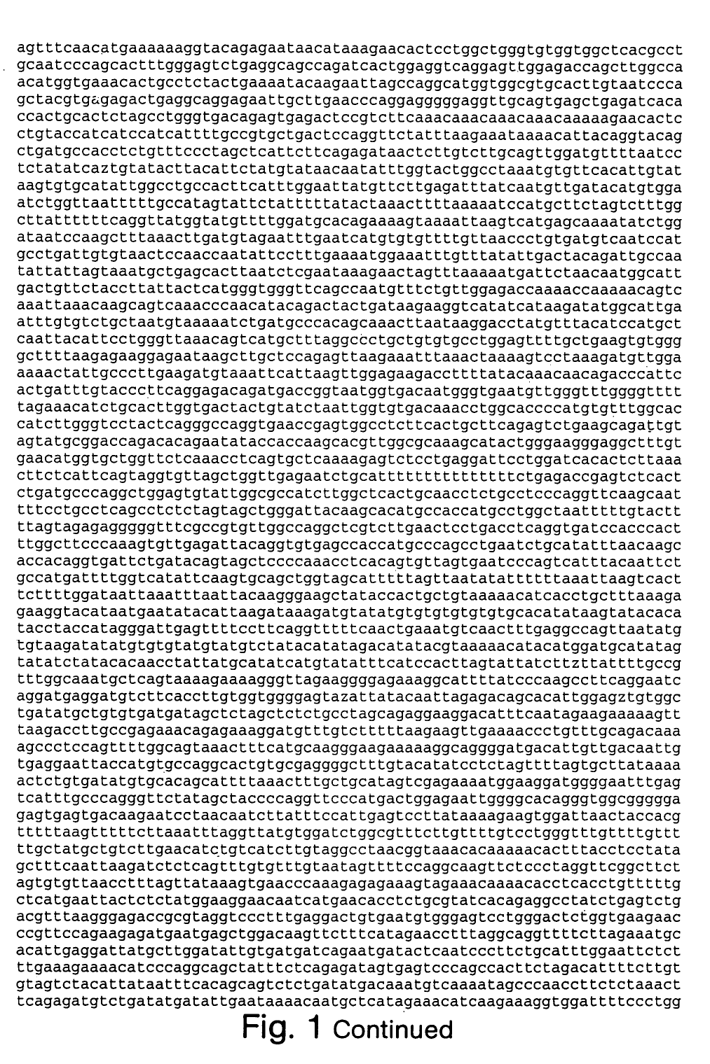

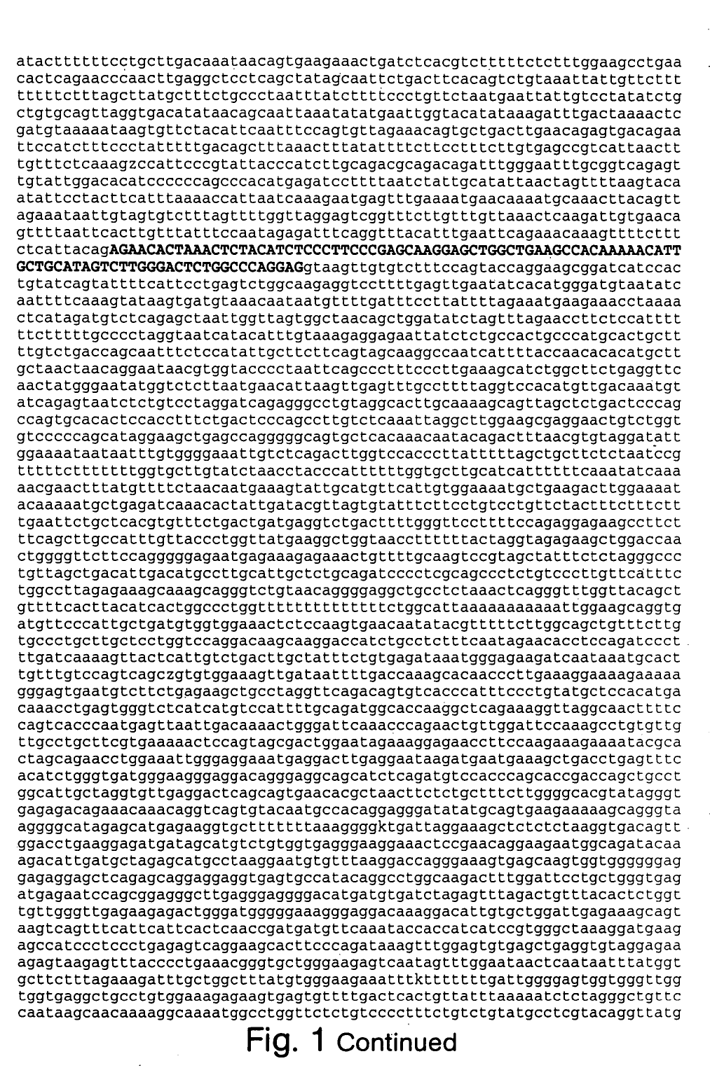

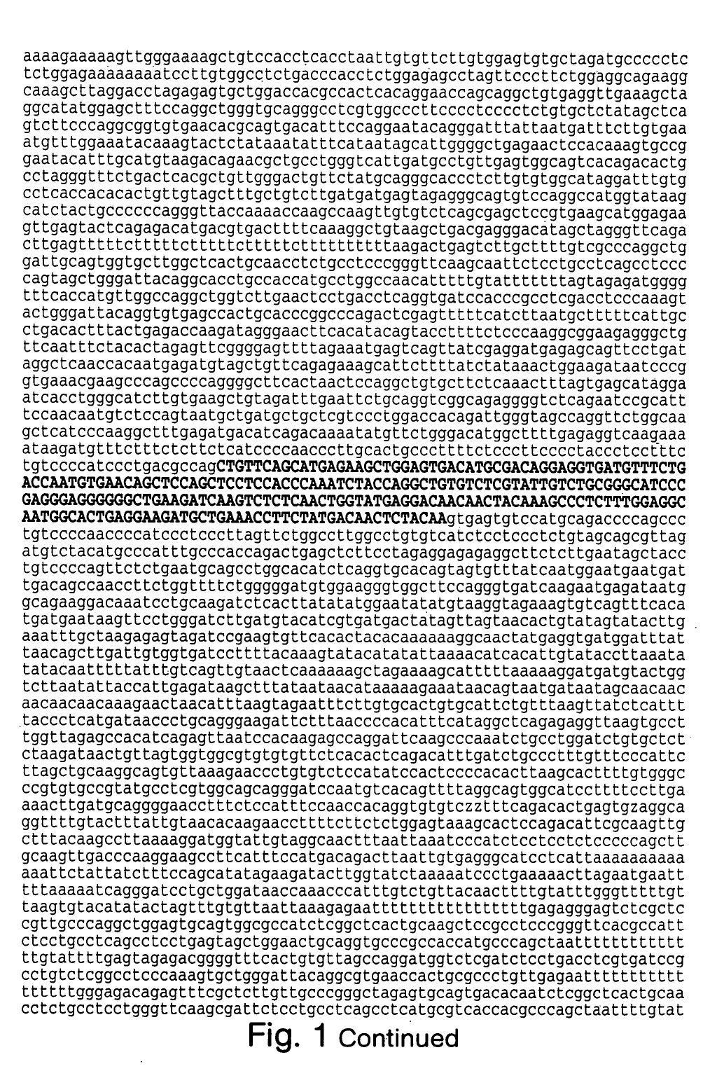

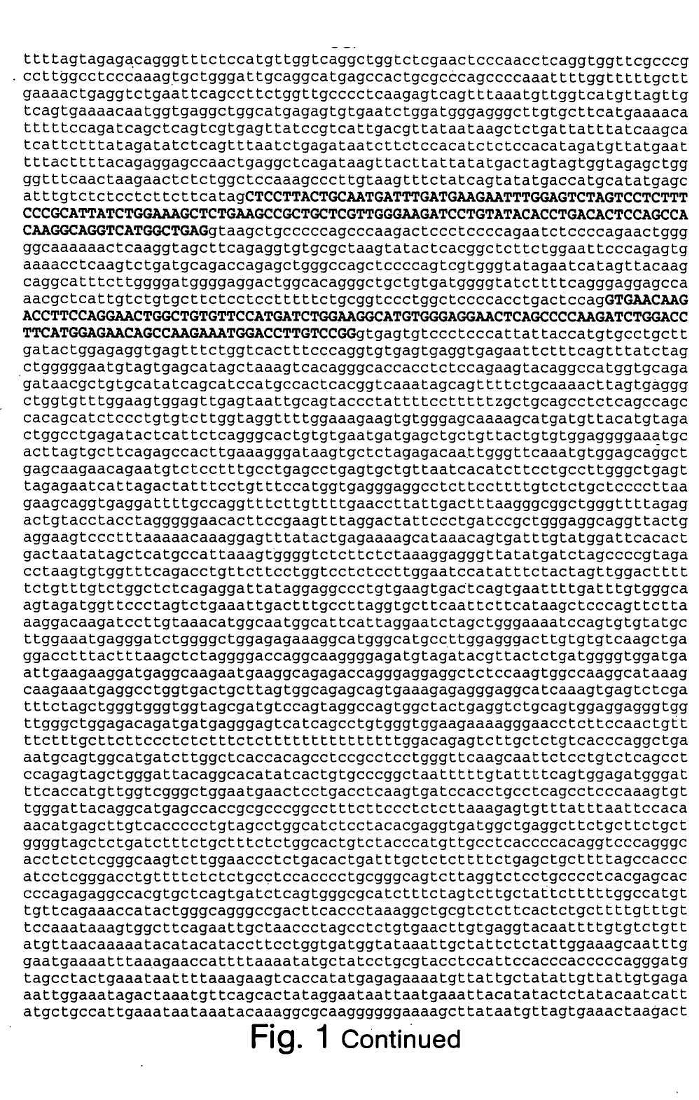

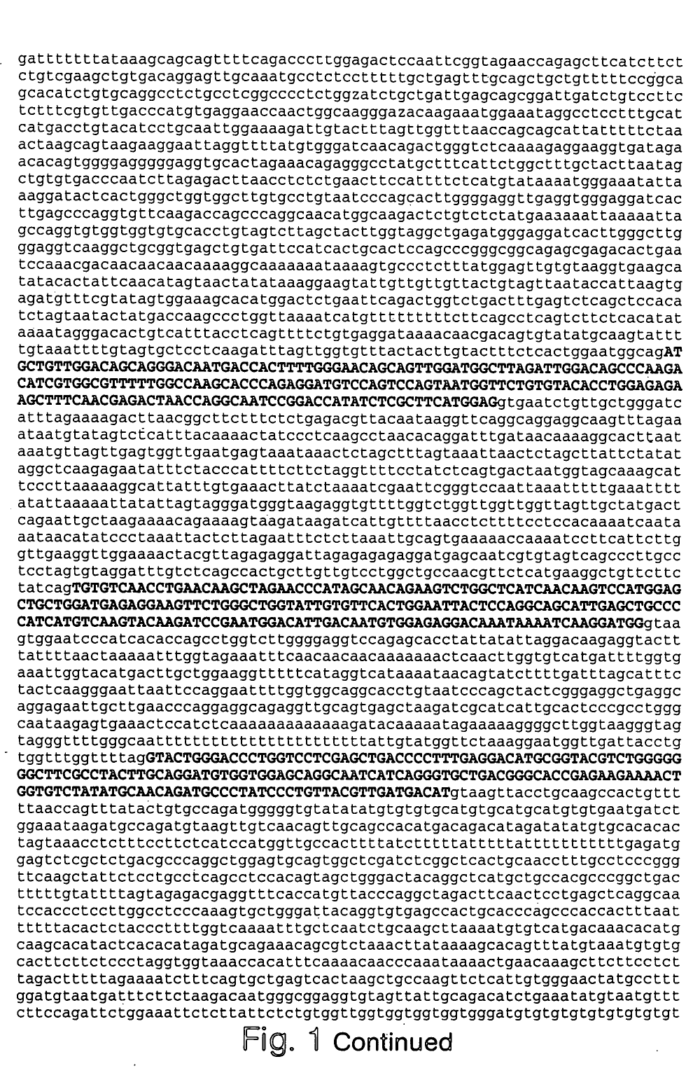

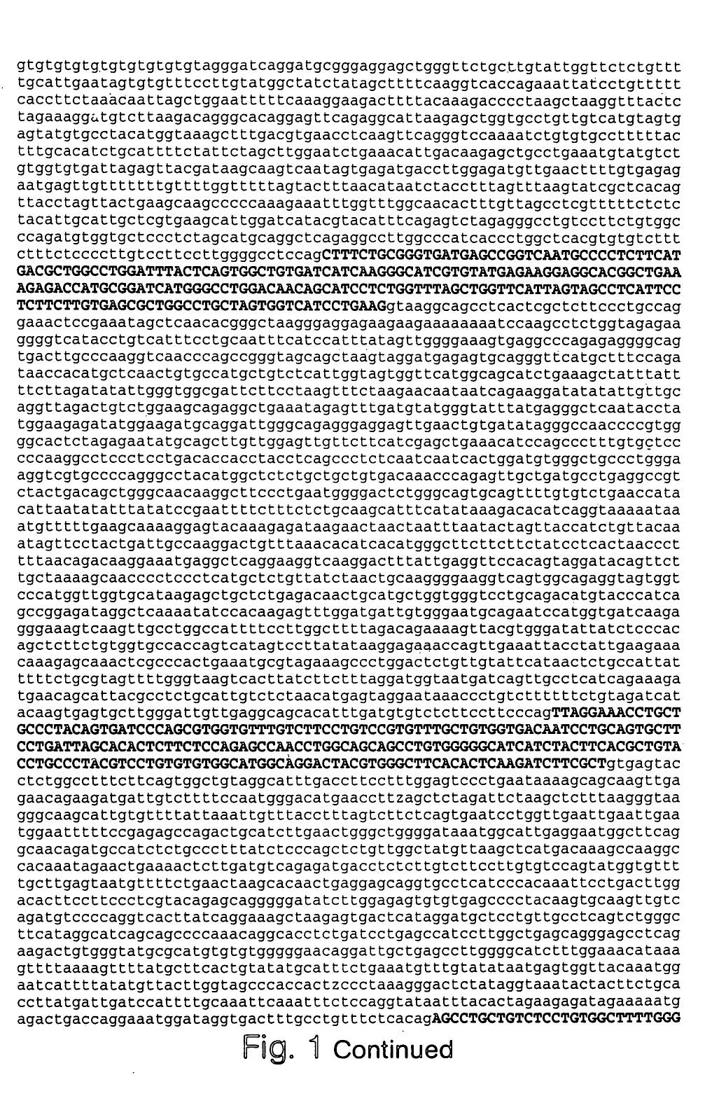

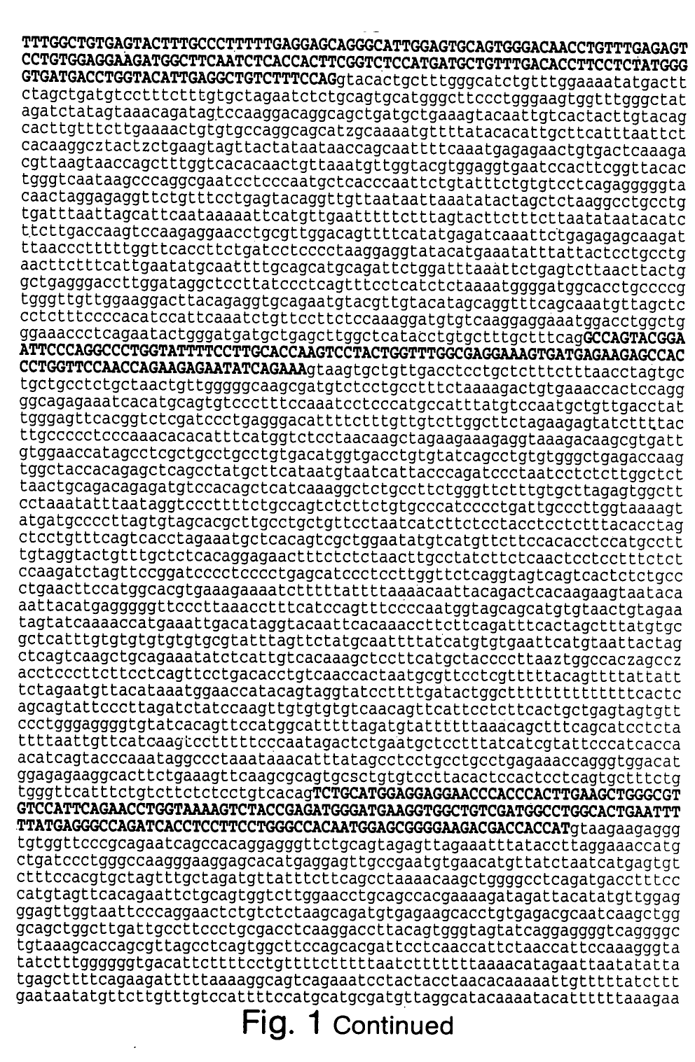

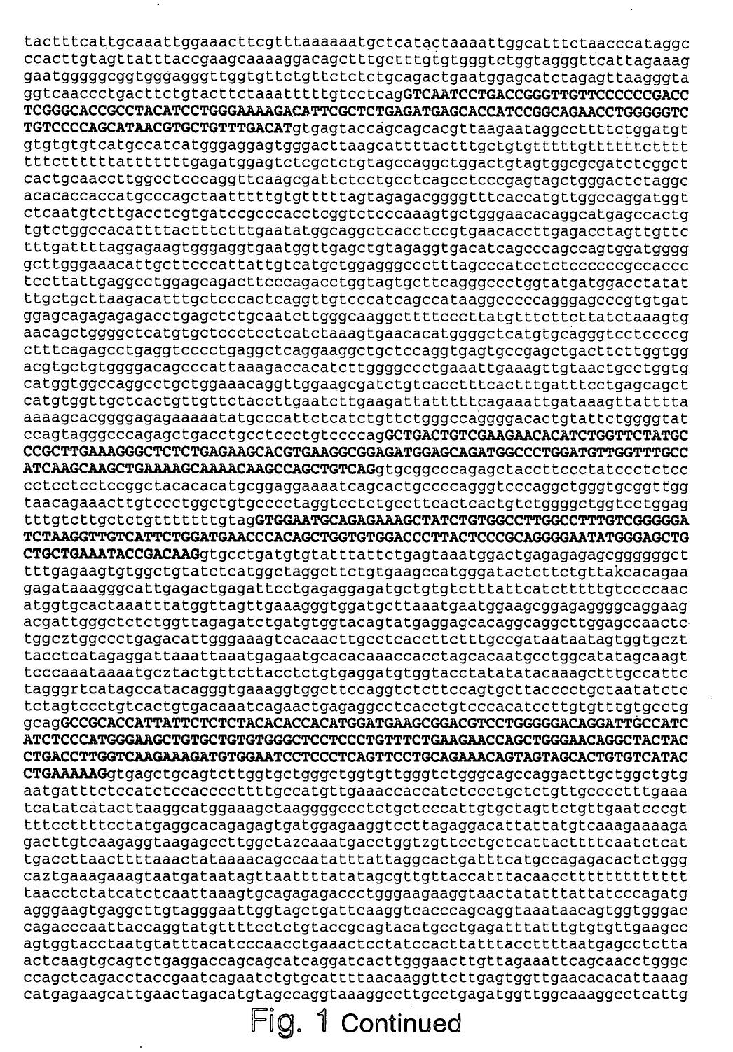

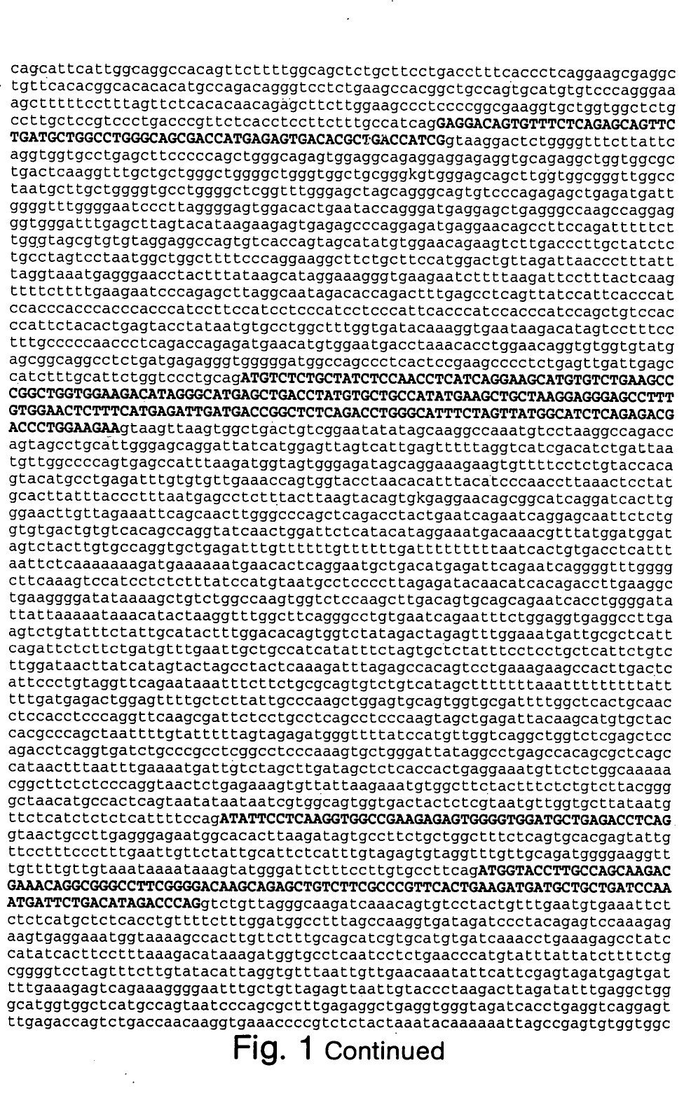

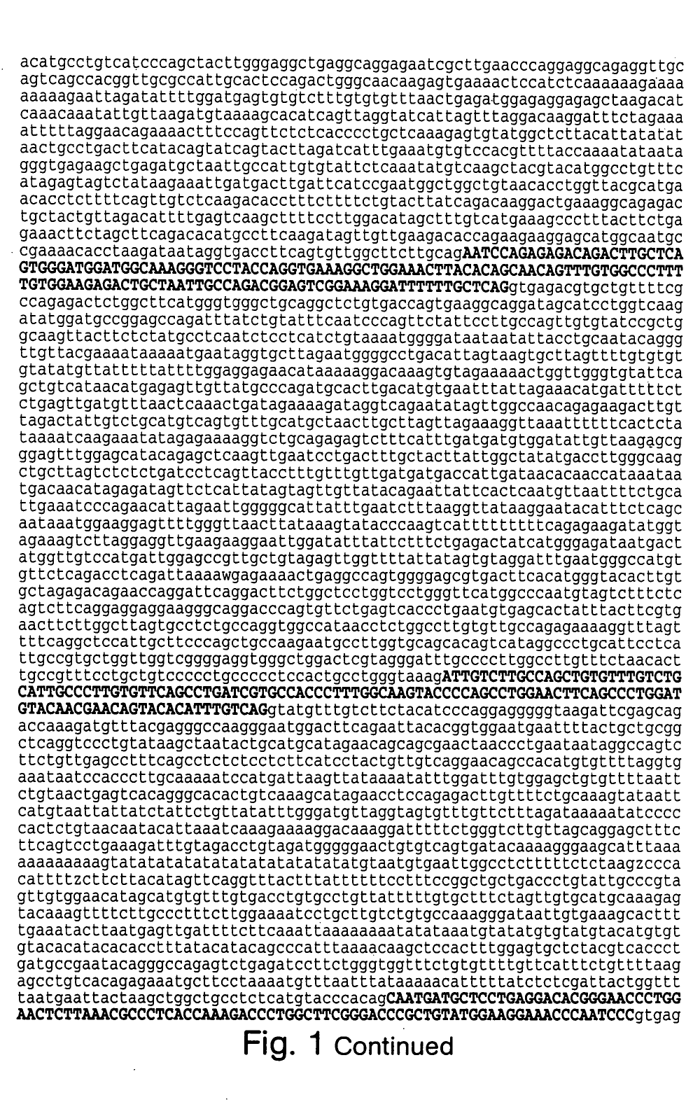

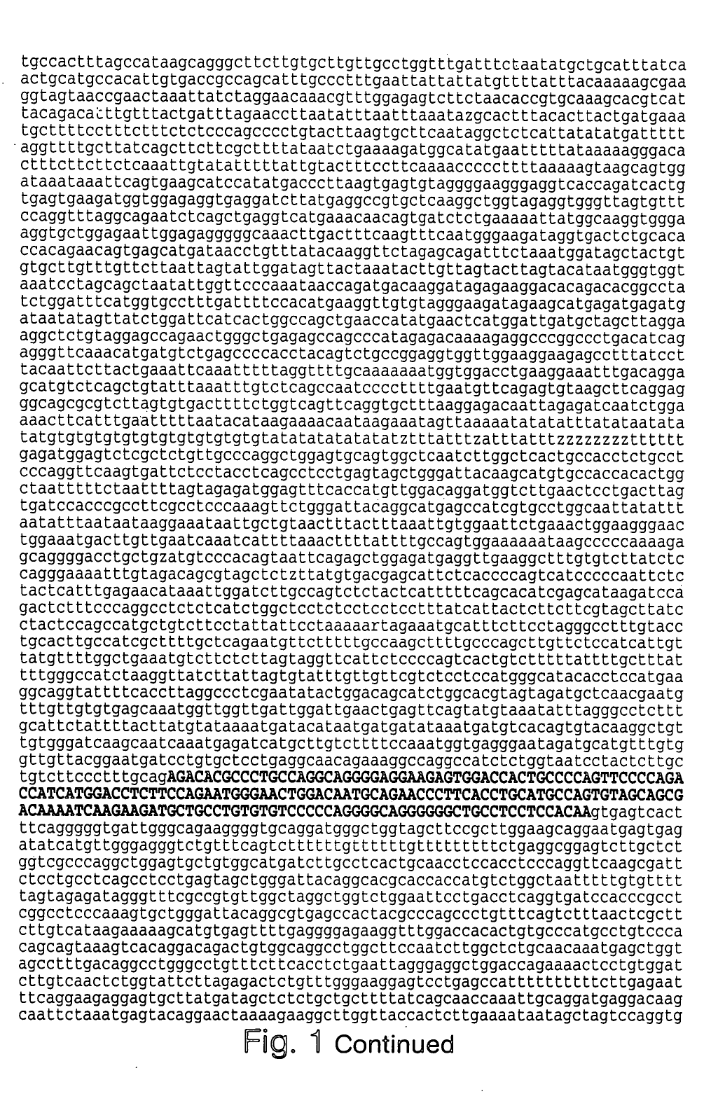

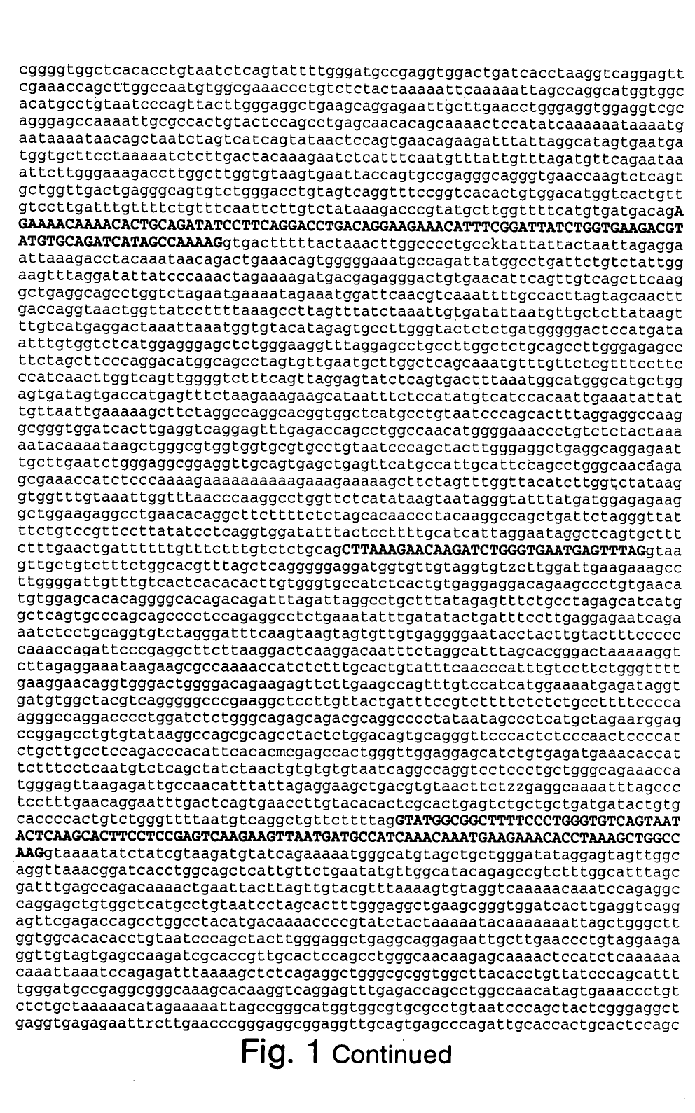

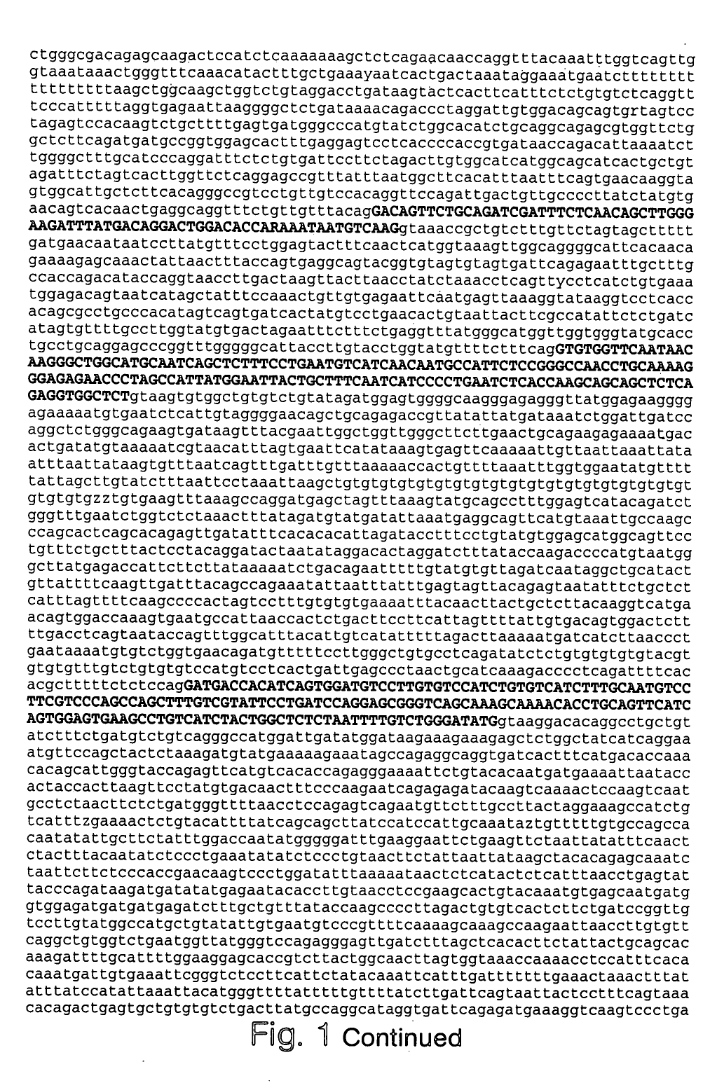

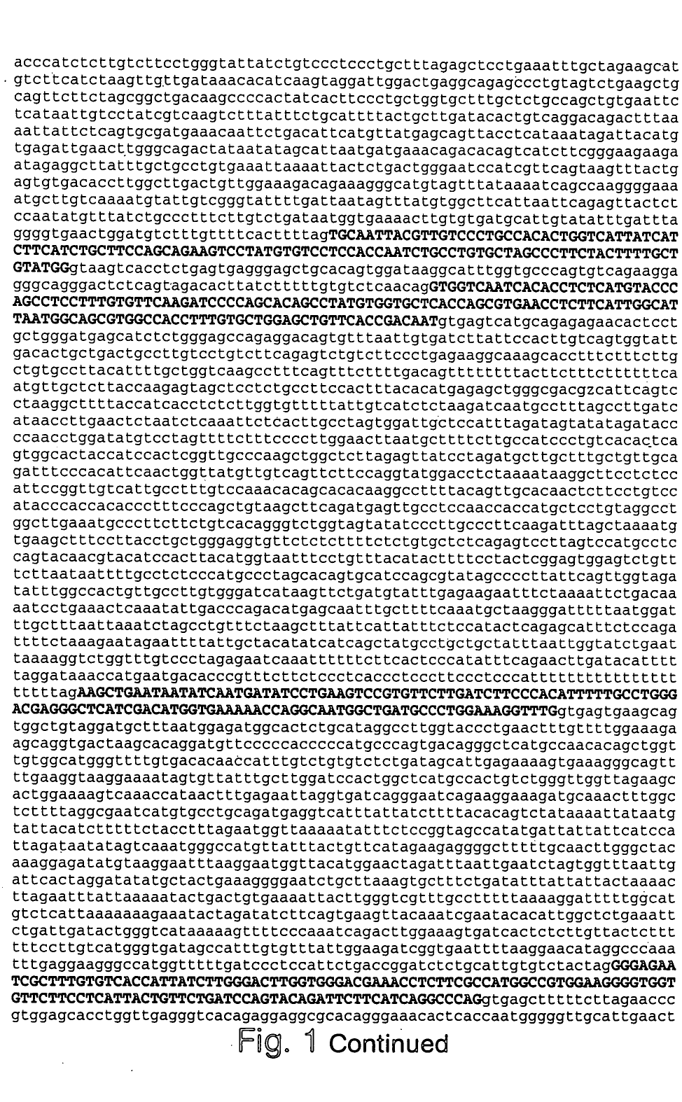

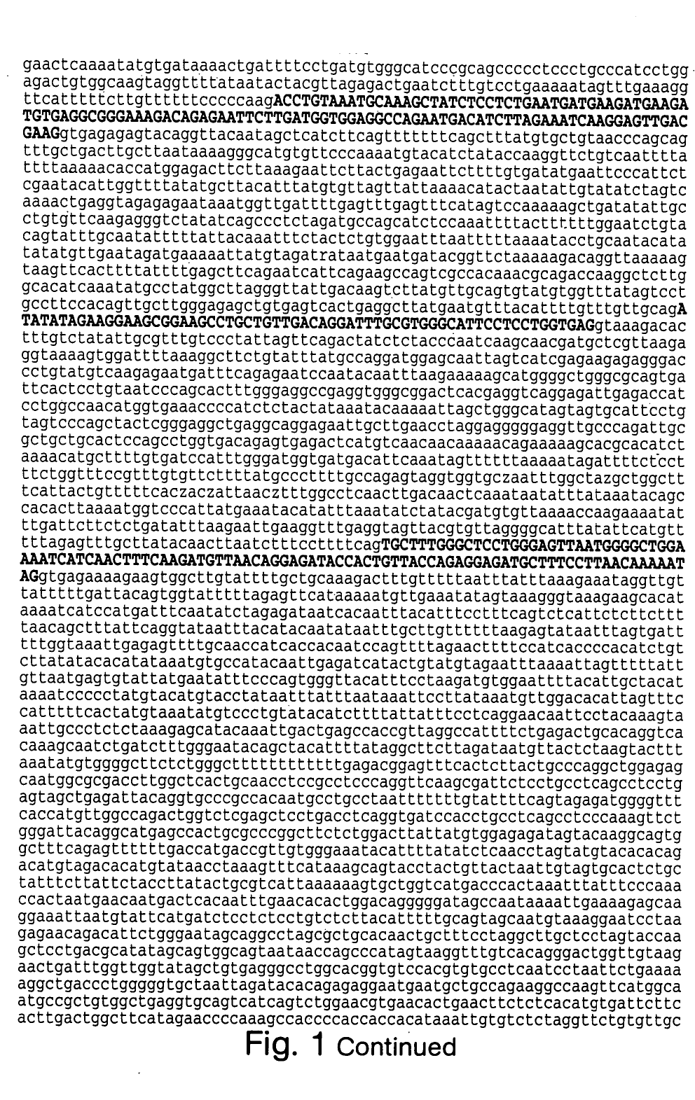

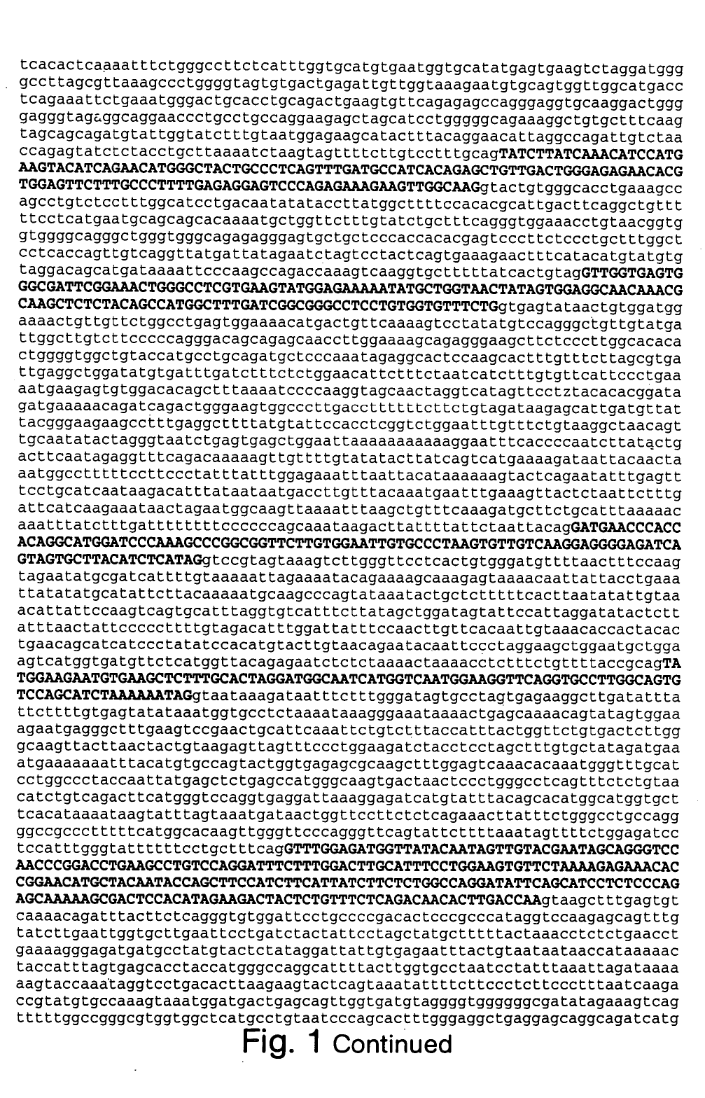

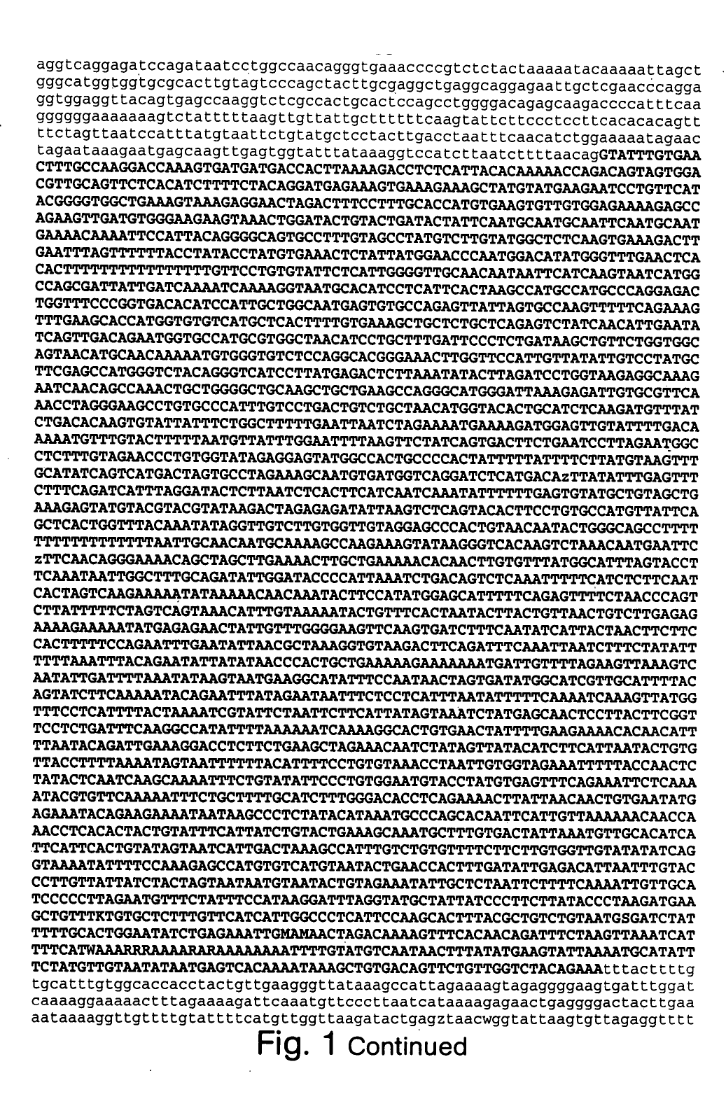

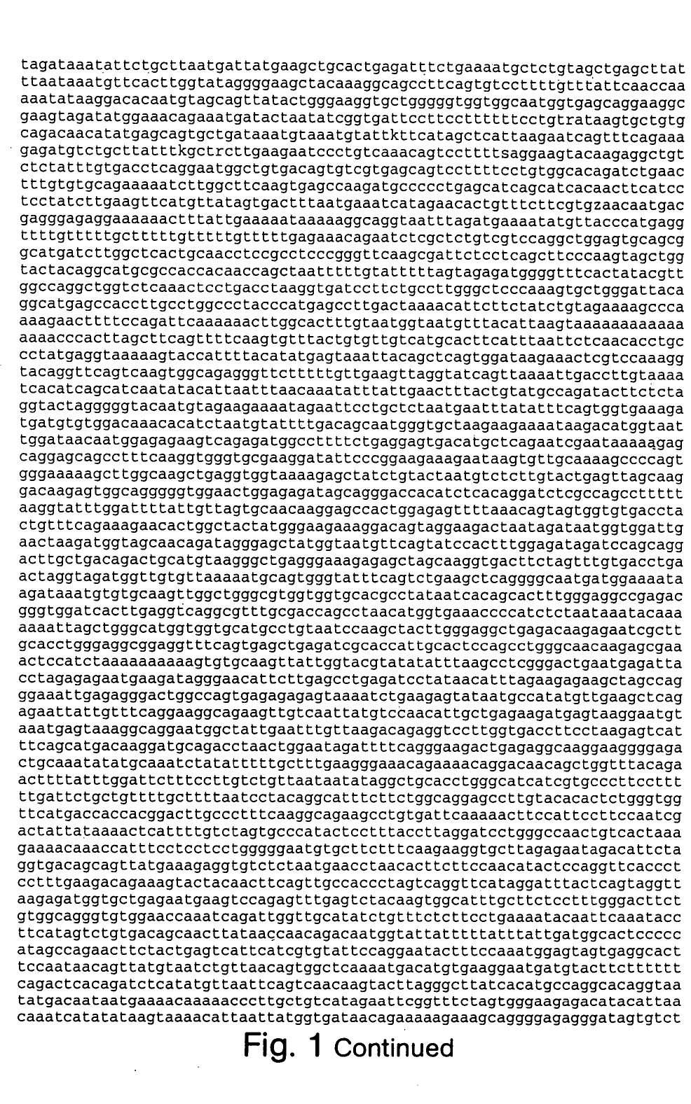

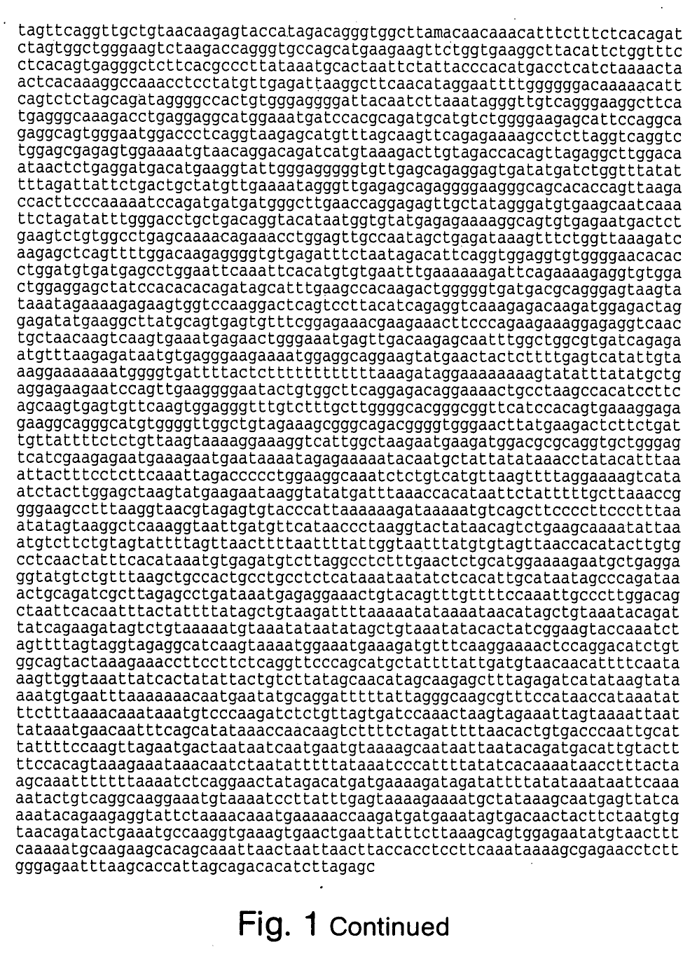

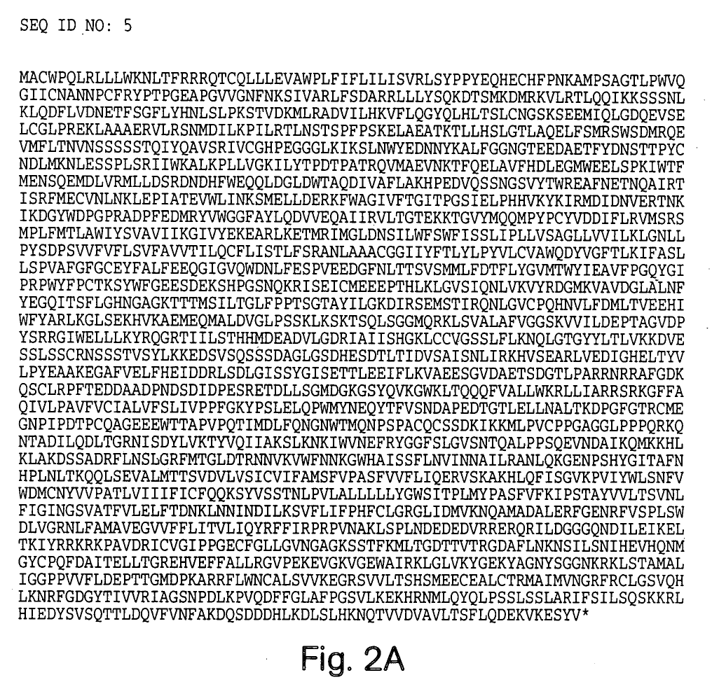

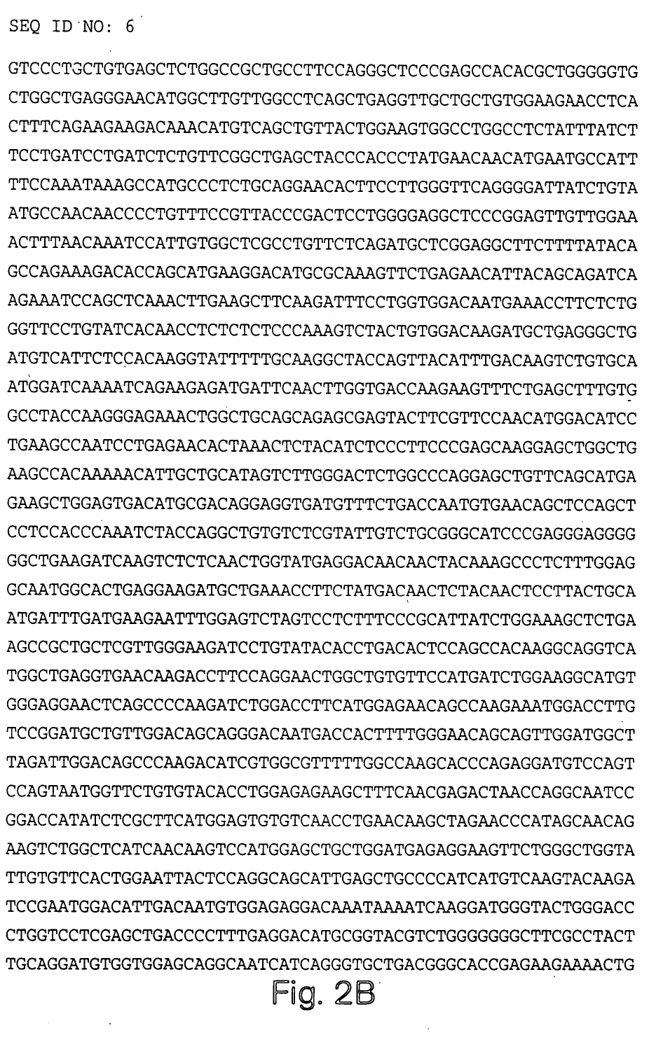

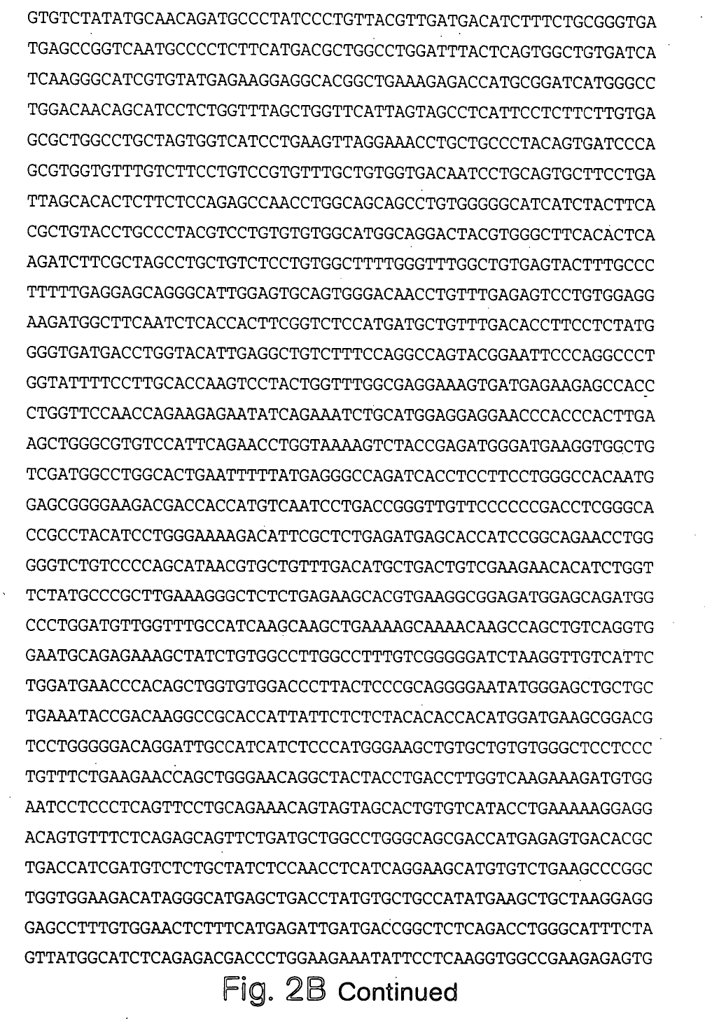

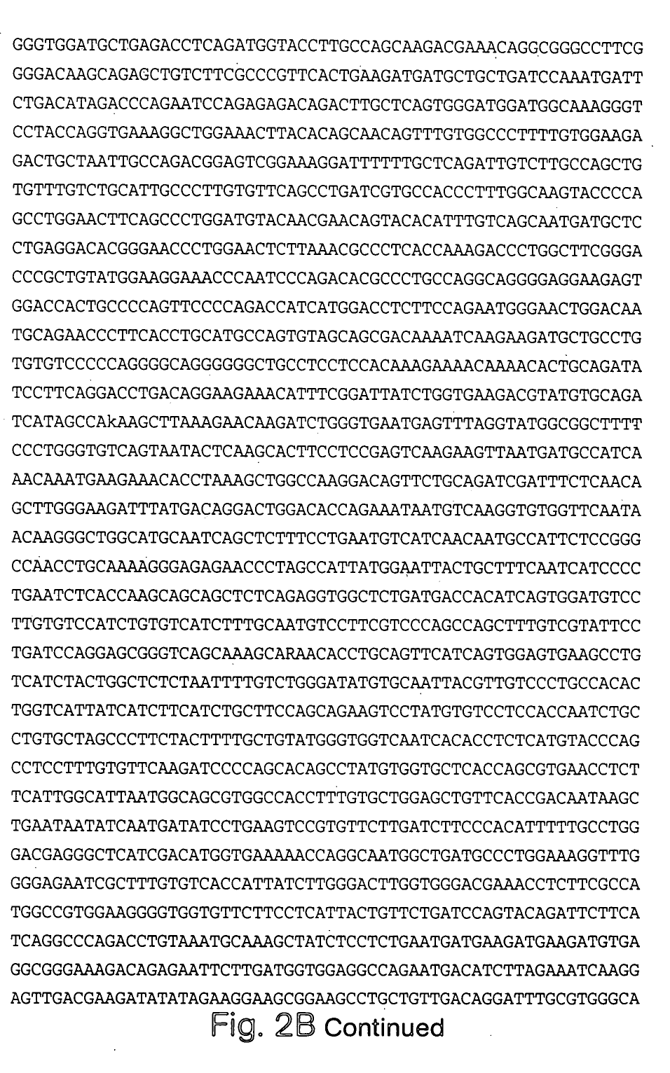

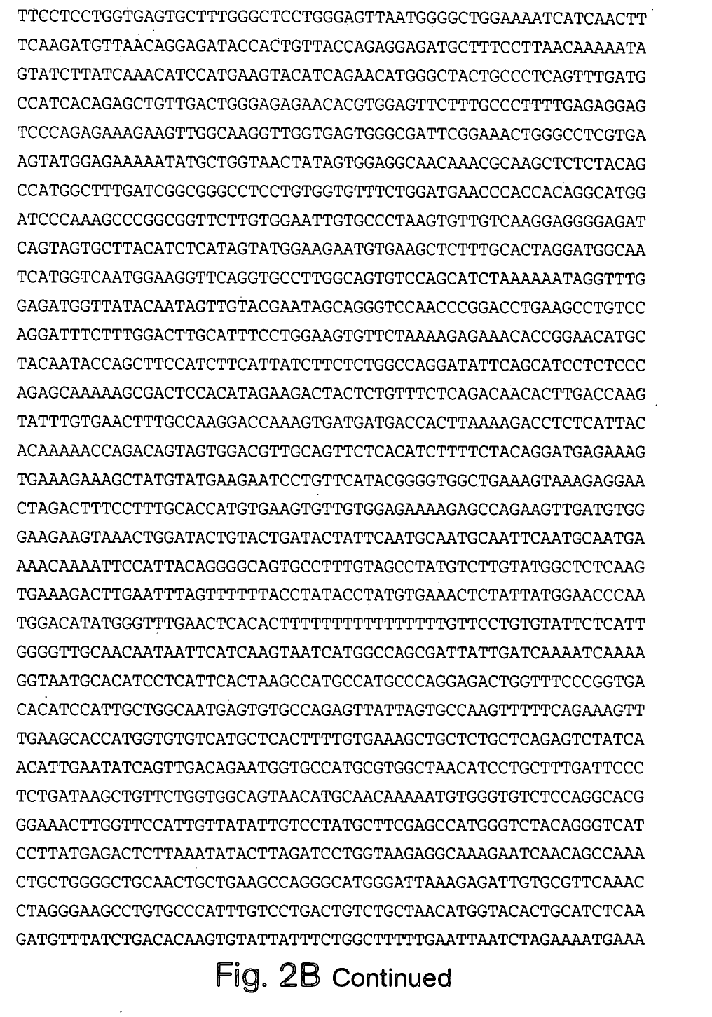

BAC DNA was extracted from bacterial cultures using NucleoBond Plasmid Maxi Kits (Clontech, Palo Alto, Calif.). For DNA sequencing, a sublibrary was first constructed from each of the BAC DNAs (Rowen et al., Automated DNA Sequencing and Analysis, eds. Adams, M. D., Fields, C. & Venter, J. C., 1994). In brief, the BAC DNA was isolated and randomly sheared by nebulization. The sheared DNA was then size fractionated by agarose gel electrophoresis and fragments above 2 kb were collected, treated with Mung Bean nuclease followed by T4 DNA polymerase and klenow enzyme to ensure blunt-ends, and cloned into Smal-cut M13mp19. Random clones were sequenced with an ABI373 or 377 sequencer and fluorescently labeled primers (Applied BioSystems, Foster City, Calif.). DNAStar software was used for gel trace analysis and contig assembly. All DNA sequences were examined against available public databases primarily using BLASTn with RepeatMasker (University of Washington). The sequence of each of the assembled contigs is shown in FIGS. 1A-D.

Reverse Transcription (RT)-PCR Amplification and Sequence Analysis

Total RNA was isolated from the cultured fibroblasts of TD and FHA patients, and reverse transcribed With a CDS primer containing oligo d(T)18 using 250 units of SuperScript II reverse transcriptase (Life Technologies, Inc., Rockville, Md.) as described (Zhang et al., J. Biol. Chem. 27:1776-1783, 1996).

cDNA was amplified with Taq DNA polymerase using primers derived from the published human ABC1 cDNA sequence (Luciani et al., Genomics 21:150-159, 1994). Six sets of primer pairs were designed to amplify each cDNA sample, generating six DNA fragments which are sequentially overlapped covering 135 to 7014 bp of the full-length human ABC1 cDNA. The nucleotides are numbered according to the order of the published human cDNA sequence (AJ012376.1). Primer pairs (1): 135-158 (f) and 1183-1199 (r); (2): 1080-1107 (f) and 2247-2273 (r); (3): 2171-2197 (f) and 3376-3404 (r); (4): 3323-3353 (f) and 4587-4617 (r); (5) 4515-4539 (f) and 5782-5811 (r); (6): 5742-5769 (f) and 6985-7014 (r). RT-PCR products were purified by Qiagen spin columns. Sequencing was carried out in a Model 373A Automated DNA sequencer (Applied Biosystems) using Taq di-deoxy terminator cycle sequencing and Big Dye Kits according to the manufacturer's protocol.

Northem Blot Analysis

Northern transfer and hybridizations were performed essentially as described (Zhang et al., J. Biol. Chem. 27:1776-1783, 1996). Briefly, 20 □g of total fibroblast RNA samples were resolved by electrophoresis in a denaturing agarose (1.2%; w/v) gel in the presence of 7% formaldehyde, and transferred to nylon membranes. The filters were probed with ³²P-labeled human ABC1 cDNA as indicated. Pre-hybridization and hybridizations were carried out in an ExpressHyb solution (ClonTech) at 68° C. according to the manufacturer's protocol.

Cell Culture

Skin fibroblast cultures are established from 3.0 mm punch biopsies of the forearm of FHD patients and healthy control subjects as described (Marcil et al., Arterioscler. Thromb. Vasc. Biol. 19:159-169,1999).

Cellular Cholesterol Labeling and Loading

The protocol for cellular cholesterol efflux experiments has been described in detail elsewhere (Marcil et al., Arterioscler. Thromb. Vasc. Biol. 19:159-169, 1999). The cells are ³H-cholesterol labeled during growth and free cholesterol loaded in growth arrest.

Cholesterol Efflux Studies

Efflux studies are carried out from 0 to 24 hours in the presence of purified ApoAI (10 μg protein/mL medium). Efflux is determined as a percent of free cholesterol in the medium after the cells were incubated for specified periods of time. All experiments are preferably performed in triplicate, in the presence of cells from one control subject and the cells from the study subjects to be examined.

Determination of Genomic Structure of the ABC1 Gene

Most splice junction sequences were determined from genomic sequence generated from BAC clones spanning the ABC1 gene. More than 160 kb of genomic sequence were generated. Genomic sequences were aligned with cDNA sequences to identify intron/exon boundaries. In some cases, long distance PCR between adjacent exons was used to amplify intron/exon boundary sequences using amplification primers designed according to the cDNA sequence. The genomic sequence of human ABC1 is shown in FIGS. 1A-D.

### Analysis of ABC1 Heterozygotes

Identification of Subjects

Subjects heterozygous for mutations in the ABC1 gene were individuals identified from the seven TD and FHA families previously described (Brooks-Wilson et al., supra; Marcil et al., supra). In addition, heterozygous individuals from three new Tangier disease families (TD3-5) and one new FHA kindred (FHA6) were included. The second mutation has not been identified in one of the TD kindreds (TD4); however, a marker immediately adjacent to ABC1 cosegregates with the low HDL phenotype. Individuals bearing the affected haplotype were considered heterozygotes. The presence or absence of mutations identified by genomic sequencing of probands from each family was subsequently confirmed by restriction fragment length polymorphism (RFLP) assays, to define heterozygous and unaffected individuals, respectively.

The control cohort consisted of unaffected members of the 11 families. These individuals share a genetic background with the heterozygotes, and environmental factors are expected. to be similar amongst family members. Thus, many additional factors that influence HDL are controlled for, and the phenotypic differences between heterozygotes and unaffected individuals can be largely attributed to variation in ABC1 gene activity.

All subjects gave informed consent to their participation in this study, and the genetic analysis protocol was approved by the Ethics committees of the University of British Columbia, the Academic Medical Centre in Amsterdam and the Clinical Research Institute of Montreal (IRCM).

Lipid and Cholesterol Efflux Measurements

Lipid levels in ABCA1 heterozygotes were measured as previously described (Brooks-Wilson et al., supra; Marcil et al., supra), at standardized lipid clinics in Vancouver, Montreal and Amsterdam. LDL was calculated by the method of Friedewald et al. (Clin. Chem. 18:499-502, 1972), modified to account for lipid measurements in mmol/L.

Cellular cholesterol efflux from fibroblast cultures was measured as previously described (Brooks-Wilson et al., supra; Marcil et al., supra). Each experiment was performed in triplicate wells and averaged. Measurements are reported as the percentage efflux in each subject relative to an average of at least two healthy controls included within the same experiment. Individual experiments were repeated at least twice, and the average relative efflux over all experiments was used.

Statistics

In analysis of the heterozygotes, differences in mean baseline demographics and lipid levels between groups were compared by Student's t-test. Comparisons of frequency either between the male to female ratio or of distributions across various percentile ranges were made using the chi-square test. Analyses of potential interactions between affected status and either sex or BMI were performed using a general linear model. Statistical analysis was performed using Prism (version 3.00, Graphpad Software) or Systat (version 8.0, SPSS Inc.). All values are reported as mean A standard deviation.

Decreased HDL Cholesterol and an Increased Risk for CAD in ABC1 Heterozygotes

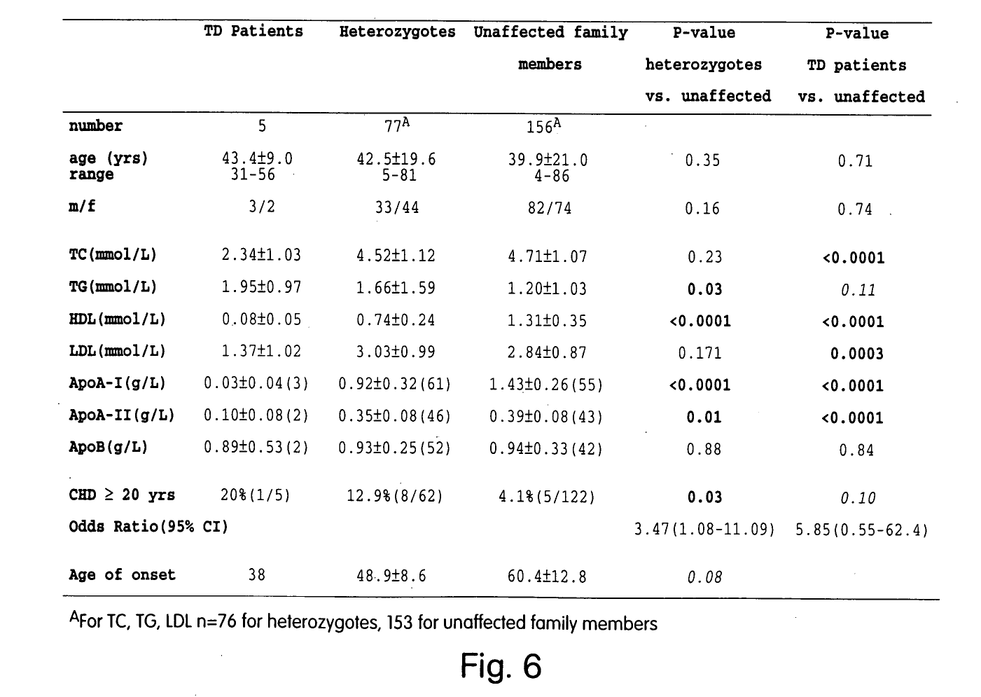

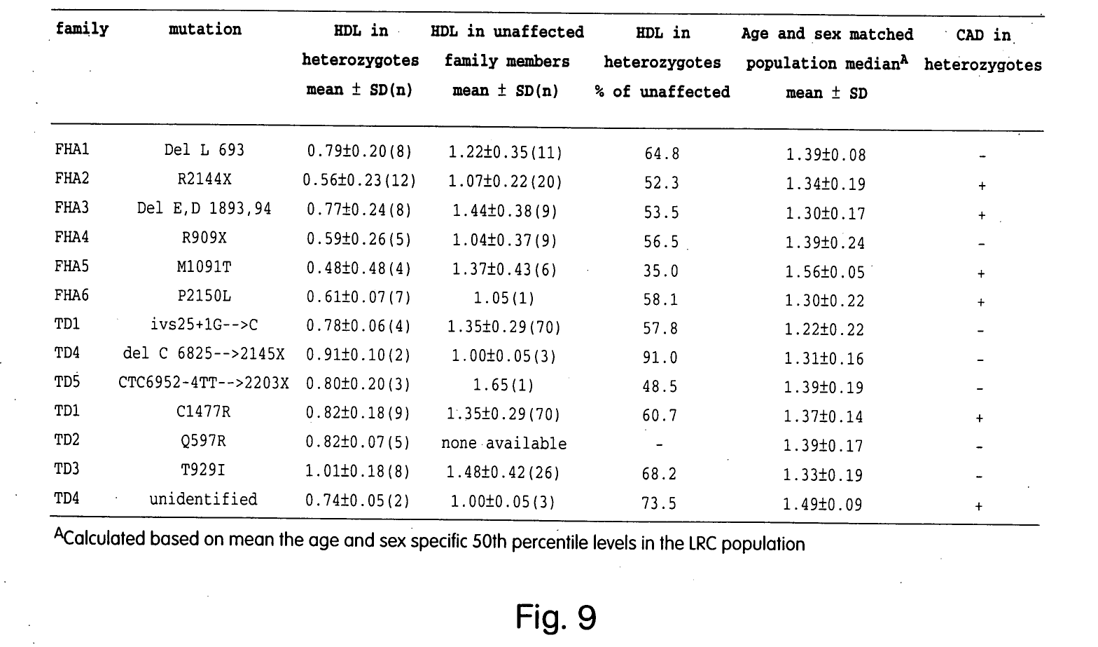

The analyzed cohort comprised 77 individuals from 11 families identified as heterozygous for mutations in the ABC1 gene. A comparison of mean lipid levels in heterozygotes with mean levels in all available unaffected family members (n=156) is presented in FIG. 6. Heterozygotes have an approximately 40-45% decrease in HDL and apoA-I and a mild (approximately 10%) decrease in apoA-II compared to unaffected family members. Mean triglycerides (TG) were increased by approximately 40% in heterozygotes compared to unaffected family members, and were further increased in TD patients. Unlike TD patients, there is no significant decrease in either total cholesterol (TC) or LDL cholesterol in heterozygotes, and apoB levels were not different in heterozygotes from controls. Mean HDL levels in carriers of each of the mutations were similarly reduced by approximately 40-50% compared to unaffected family members (FIG. 9).

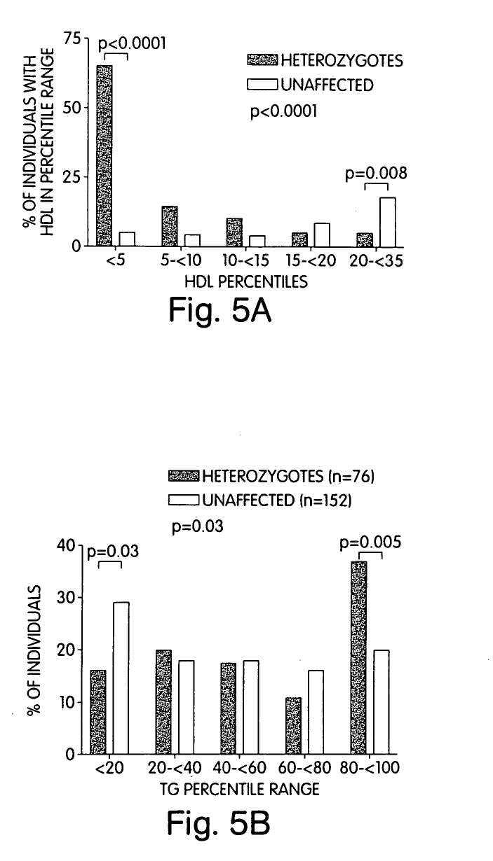

The heterozygote phenotype was further examined by calculating the percentage of individuals falling within a given range of age and sex specific percentiles (based on LRC criteria (Heiss et al., supra; Heiss et al., Circulation 61:302-315, 1980). Much variability in the heterozygote phenotype was evident. As illustrated in FIG. 5A, although a significantly higher percentage of heterozygotes had HDL cholesterol less than the 5^{th} percentile for age and sex compared to unaffected controls (65% vs. 5%, p<0.0001), 5% of the heterozygotes had HDL greater than the 20^{th} percentile, with HDL ranging up to the 31^{st} percentile for age and sex. Thus, in some individuals clearly the low HDL phenotype is less severe. A broad distribution of triglyceride (TG) levels was also evident (FIG. 5B). A significantly lower percentage of heterozygous individuals had TG below the 20^{th} percentile for age and sex (p=0.03), and a significantly larger percentage had TG>80^{th} percentile (p=0.005) compared to unaffected family members, but substantial overlap between the two distributions was seen.

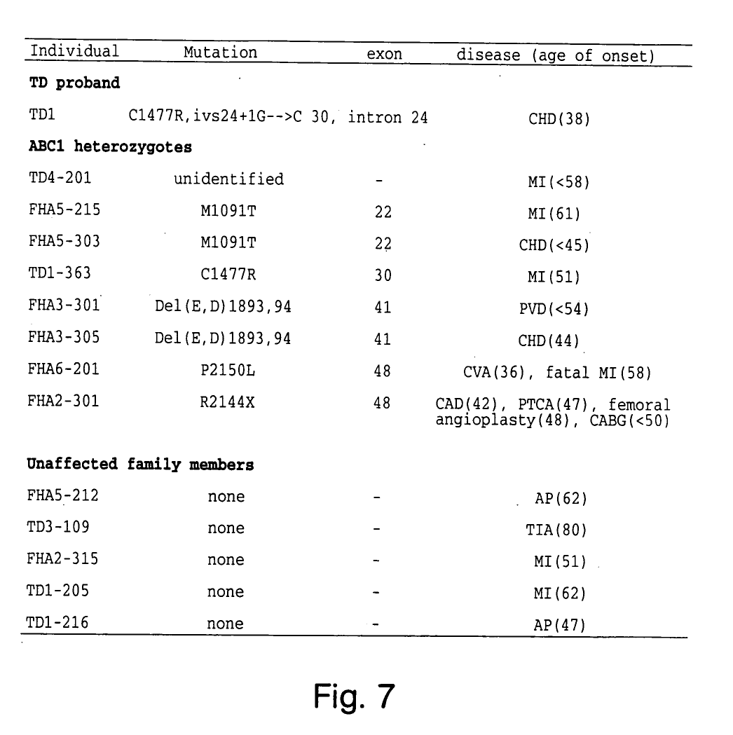

Another important question is whether individuals heterozygous for ABC1 mutations are at an increased risk of developing coronary artery disease (CAD). In our large cohort, symptomatic vascular disease was over three times as frequent in the adult heterozygotes as in unaffected family members (FIG. 6). The forms of vascular disease were generally more severe in the heterozygotes than in their unaffected family members (FIG. 7). Heterozygotes had myocardial infarctions (five, one fatal) and severe vascular disease requiring multiple interventions, whereas in unaffected individuals, CAD was manifest as angina in two cases and as a transient ischemic attack at the age of 80 in another. Furthermore, the mean age-of-onset was on average a decade younger in heterozygotes compared to unaffected controls (FIG. 6)

Cholesterol Efflux, HDL Level, and CAD in ABC1 Heterozygotes

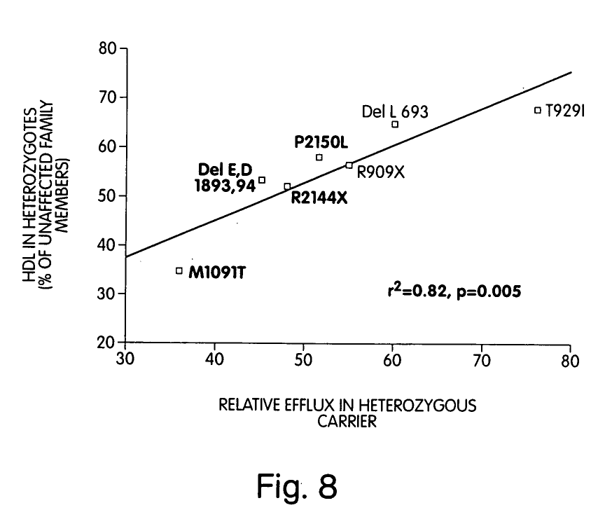

We have previously shown that individuals heterozygous for ABC1 mutations have decreased cholesterol efflux. In the present study, the relationship between cholesterol efflux levels, HDL cholesterol levels, and CAD was further assessed. Relative cholesterol efflux in individuals heterozygous for an ABC1 mutation was plotted against the mean HDL cholesterol levels observed in the carriers of that mutation, expressed as a percentage of the unaffected members within that family (FIG. 8). Cholesterol efflux levels associated with each mutation strongly predict the corresponding HDL cholesterol levels in the families, accounting for 82% of the variation in HDL cholesterol (r²=0.82, p=0.005). Furthermore, in one large family (FHA2), where efflux has been measured in three independent heterozygotes, an r² value of 0.81 was obtained when individual plasma HDL levels were plotted against individual efflux measurements. Using the regression equation of mean HDL levels in the heterozygotes on the efflux level of the heterozygous carrier (p=0.02), the relationship between expected changes in ABC1 efflux activity and HDL levels was estimated. Based on this analysis, we predict that ach 8% change in efflux levels would be associated with a 0.1 mmol/L change in HDL cholesterol.

Relative cholesterol efflux levels are also related to CAD within the family. Families with clearest evidence for premature CAD had individuals with the lowest cholesterol efflux (bold on FIG. 8 and FIG. 9). These data suggest that the level of residual ABC1 function is a critical determinant of both HDL cholesterol levels and susceptibility to CAD.

Comparison of Mutation Type and Location to the Severity of Phenotype in Individuals Heterozygous for ABC1 Mutations

We have previously noted that the phenotypic presentation of our FHA heterozygotes was more severe than that of our TD heterozygotes. Furthermore, we initially noted more deletions and premature truncations of the protein in our FHA families than our TD families. Thus, as residual ABC1 activity is an important predictor of severity of the phenotype, the influence of the nature of the mutation on the phenotypic expression of mutations in the ABC1 gene was examined. Severe mutations which would be expected to result in a non-functional allele were defined as deletions or mutations that caused premature truncation of the protein (frameshifts and nonsense mutations) or that disrupted natural splicing of the protein. Missense mutations, on the other hand, result in the change of only a single amino acid and may result in a protein product that still retains partial activity.

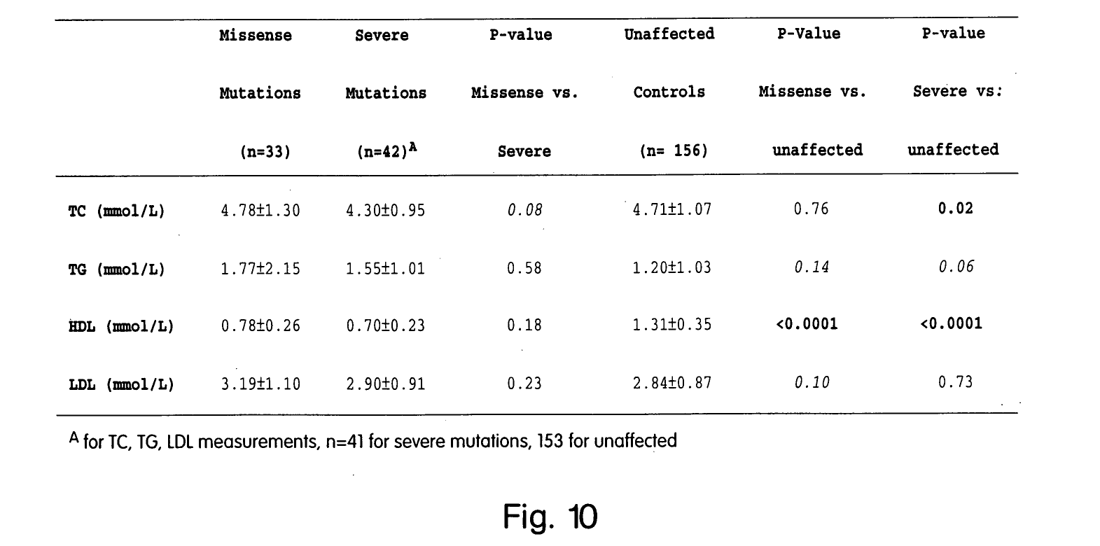

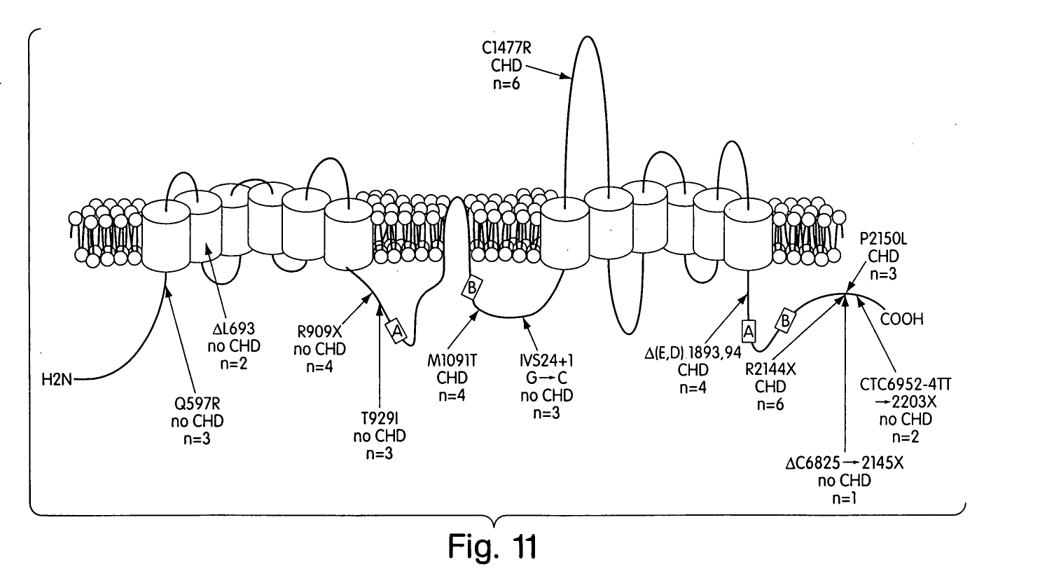

Lipid levels were compared in heterozygous carriers of severe and missense mutations. While there was a trend to decreased HDL levels in carriers of severe compared to missense mutations, this trend did not reach significance (FIG. 10). A range of HDL levels in individual missense and severe mutations were observed (FIG. 9). The M1091T missense mutation is the most severe mutation in terms of effects on efflux and HDL levels, with a more severe phenotype than even early truncations of the protein (e.g. R909X). The site of mutation (e.g. N-terminal or C-terminal) within the ABC1 protein did not influence the phenotype (FIG. 11). The presence of CAD is seen in carriers of mutations in several domains of the protein. Patients with mutations on both alleles manifest with splenomegaly alone or in association with CAD (TD1). Thus the phenotype appears to be mutation specific, and most likely dependent on remaining ABC1 function of the wild-type allele and residual function of the mutant allele, similar to what has been shown for mutations in ABCR, a close homologue of ABC1 (van Driel et al., Ophthalmic Genet. 19:117-122, 1998).

Relationship Between Phenotype of Mutations in the ABC1 Gene and Age

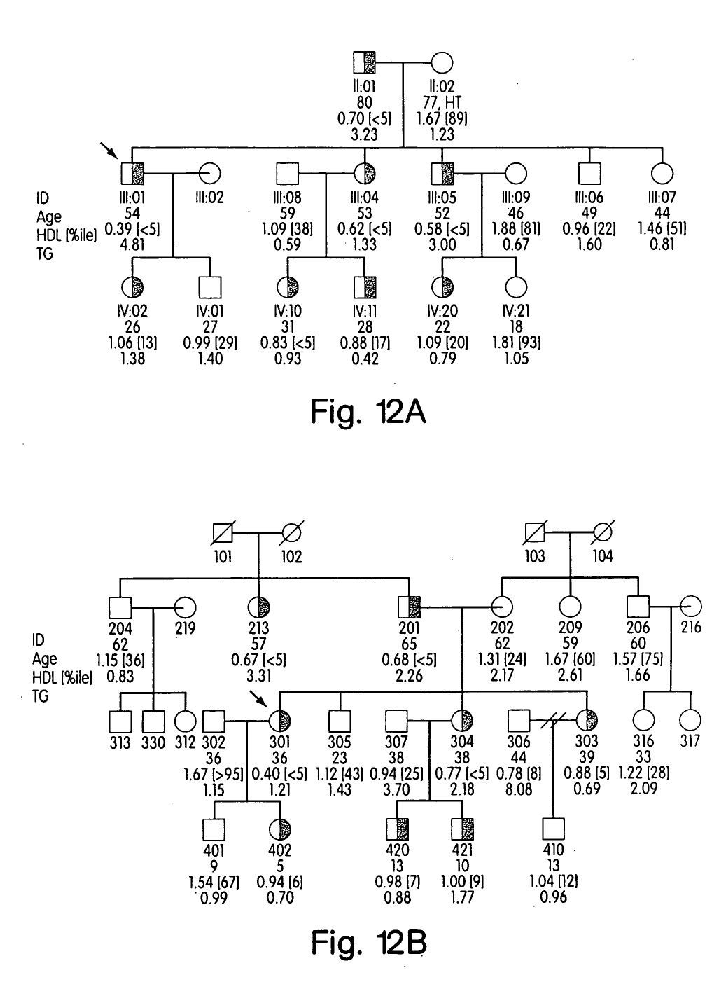

One factor influencing phenotypic expression that became apparent in the families was age. We first characterized the effect of age on the HDL levels of individuals in two previously reported families (Marcil et al., supra) (FIGS. 12A and 12B). In family FHA3, while heterozygous individuals in generations II and III all had HDL cholesterol levels less than the 5^{th} percentile for age and sex, those in generation IV had a much more variable phenotype, with HDL cholesterol ranging up to the 20^{th} percentile. In family FHA1, the same pattern was observed.

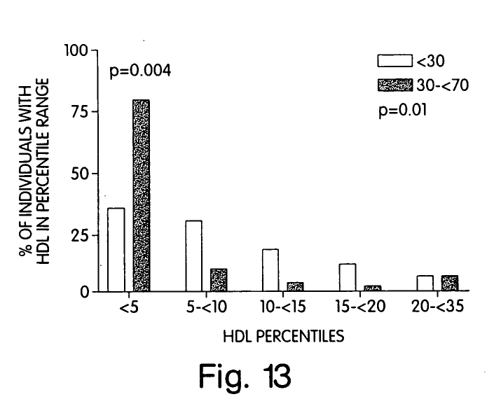

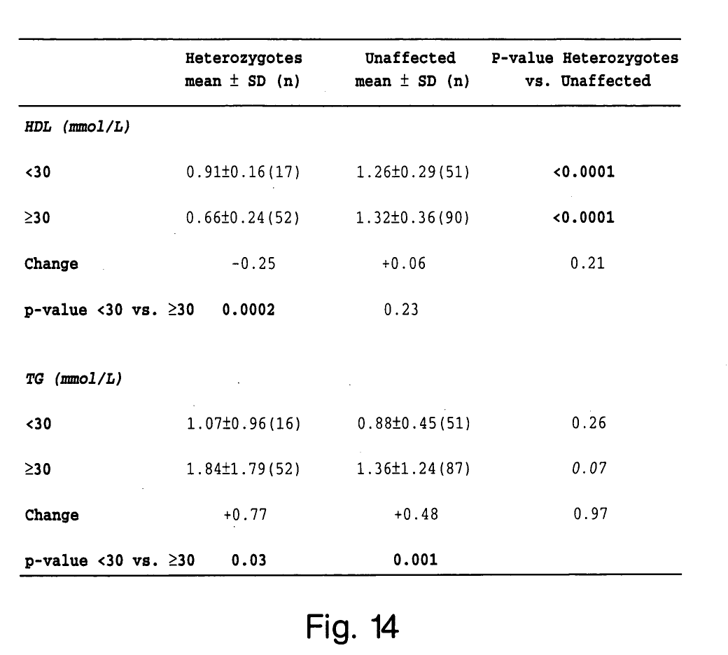

The distribution of iridividuals less than 30 years old across HDL percentile ranges was compared to the corresponding distribution of individuals 30-70 years old across HDL percentile ranges (FIG. 13). A significantly larger percentage of individuals 30-70 years old had HDL cholesterol less than the 5^{th} percentile compared to the percentage of individuals less than 30 years old with HDL cholesterol less than the 5^{th} percentile. Mean HDL decreases in heterozygotes greater than 30 years of age compared to those less than 30 years of age; in contrast, there is no significant change in unaffected controls (FIG. 14). Similar results are seen in males and females separately and in both pre- and post-menopausal ages in women (FIGS. 15A and 15B). Triglyceride levels increase with age in both heterozygotes and unaffected family members.

Assessment of the Influences of Gender and BMI on the Phenotypic Expression of ABC1 Mutations

Females are known to have elevated HDL and decreased triglyceride levels compared to males. Thus, the phenotype of ABC1 heterozygotes was analyzed to determine whether the phenotype was influenced by gender. HDL cholesterol is significantly lower than unaffected controls in both heterozygous males and females (0.70Å0.24 versus 1.21+0.29, p<0.0001; 0.76Å0.25 versus 1.41Å0.38, p<0.0001, respectively). This was reflected in decreased apoAI (0.92Å0.27 versus 1.36Å0.22, p<0.0001; 0.92Å0.36 versus 1.49Å0.28, p<0.0001 in males and females, respectively), and a trend towards a mild decrease in apoAII in both males and females compared to unaffected family members (0.35Å0.08 versus 0.40Å0.09, p=0.08; 0.35Å0.09 versus 0.39Å0.07, p=0.06, respectively). Triglycerides are higher in both male (2.07Å2.16 versus 1.30Å1.30, p=0.02) and female (1.34Å0.86 versus 1.09Å0.63, p=0.08) heterozygotes compared to unaffected family members. The difference in HDL between males and females was reduced in heterozygotes compared to controls (p=0.11), while the difference in triglycerides was increased compared to controls (p=0.13).

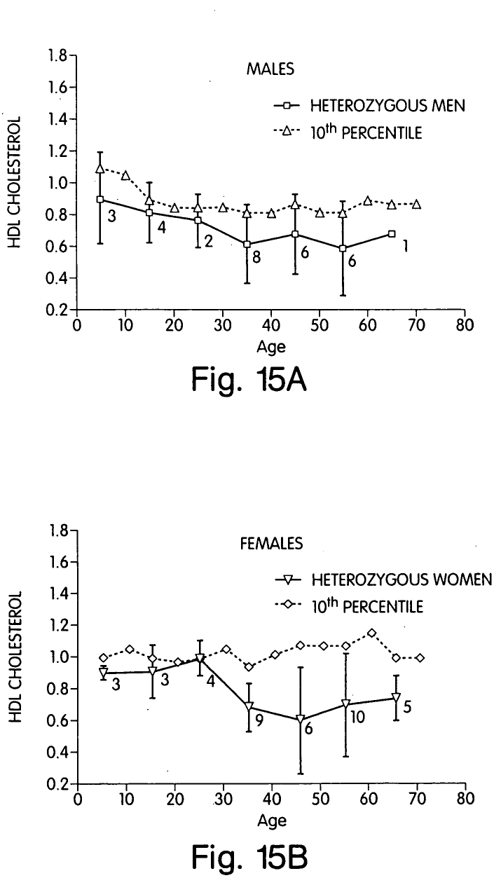

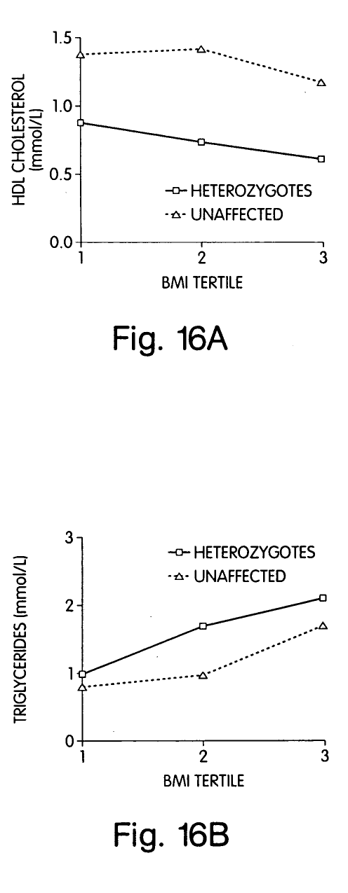

Another factor known to influence HDL and triglyceride levels is BMI. The entire cohort was divided into tertiles of BMI, and the mean HDL and triglyceride levels of heterozygotes and unaffected individuals by BMI tertile are shown in FIGS. 16A and 16B. BMI had a significant effect on both HDL and triglycerides in both heterozygotes and controls (p<0.0001). The effect of BMI on HDL-C and triglyceride levels was more severe in heterozygotes for ABC1 than in controls, being evident at lower BMIs (mid-tertile) in heterozygotes. A raised BMI was more obviously associated with changes in HDL and triglyceride levels in heterozygotes compared to controls. However, neither effect reached statistical significance. HDL was reduced in heterozygotes compared to controls in all BMI tertiles (p<0.0001 in each tertile). While triglyceride levels were increased in all BMI tertiles in heterozygotes compared to unaffected family members, this difference was only significant in the middle BMI tertile (p=0.009).

### Analysis of ABC1 SNPs from REGRESS Study

Identification of SNPs

SNPs in the ABC1 gene were identified during the complete genomic sequencing of 14 unrelated probands with low HDL-C (Brooks-Wilson et al., supra 1999; Marcil et al., supra 1999). Variants that were identified within the low HDL families that did not co-segregate with the low HDL phenotype or that were observed in unaffected individuals were assumed to be SNPs. Based on the sequencing of BAC clones spanning the entire ABC1 region (described above), sites identified as heterozygous or different from that found in sequenced individuals were also identified as polymporphisms. Sequence data was available from at least one control individual at all variant coding sites. The SNPs are numbered from the nucleotide described as position 1 (Pullinger et al., Biochemical and Biophysical Research Communications 271:451-455, 2000), naming the first exon number 1. As a standardized nomenclature for all variants, the “wild-type” allele (more frequent in the REGRESS population) is designated A, while the variant (less frequent) allele is designated B.

Subjects

To assess the effects of these SNPs on lipid levels and CAD, w studied a cohort of 804 men with proven coronary artery disease who participated in the Regression Growth Evaluation Statin Study (REGRESS), which has previously been described in detail (Jukema et al., Circulation 91:2528-2540, 1995). Briefly, study participants were required to have at least one coronary artery with a stenosis of more than 50% as assessed by coronary angiography, a plasma total cholesterol concentration of 4 to 8 mmol/L (155 to 310 mg/dL), and a plasma triglyceride concentration below 4 mmol/L (350 mg/dL). Phenotypic effects of the cSNPs were examined in relationship to baseline lipid parameters.

Patients were randomly assigned to treatment with pravastatin (Pravachol, Bristol-Myers Squibb, Princeton, N.J.) or placebo for a period of two years. Computer-assisted quantitative coronary angiography was carried out at the start and at the end of the study as previously described (Jukema et al., supra (1995)). The baseline values and changes in the average mean segment diameter (MSD), which is a measurement of the average unobstructed diameter along the vessel, and in the minimal obstructive diameter (MOD), which is a measurement of the smallest unobstructed segment, were used as the primary measures of CAD. The MSD reflects diffuse changes of atherosclerosis, and the MOD reflects focal atherosclerotic changes. Larger MSD and MOD measurements reflect less occlusion of the vessel, and a decrease in these parameters reflects progression of coronary atherosclerosis. In addition, the prevalence of coronary events; defined as death, myocardial infarction, unscheduled coronary angioplasty or bypass surgery (PTCA, CABG), or stroke/transient ischemic attack; was examined.

Blood was collected from each patient at baseline, and DNA was extracted according to standard procedures. Several subsequent genetic studies have been performed on this cohort (Reymer et al., Nature Genetics 10:28-34, 1995; Jukema et al., Circulation 94:1913-1918, 1996; Kluijtmans et al., Circulation 96:2573-2577, 1997; Kastelein et al., Clinical Genetics 53:27-33, 1998; Kuivenhoven et al., New England Journal of Medicine 338:86-93, 1998). The REGRESS and its DNA substudies were approved by all seven institutional review boards of the participating centers and by their. medical ethics committees.

Additional Dutch subjects with low HDL and premature coronary artery disease were obtained from previously described populations (Kuivenhoven et al., Arteriosclerosis, Thrombosis and Vascular Biology 17:560-568, 1997; Verhoff et al., Atherosclerosis 141:161-166 1998; Franco et al., British Journal of Hematology 102:1172-1175, 1998; Wittekoek et al., Artherosclerosis 146:271-279, 1999; Franko et al., British Journal of Hematology 104:50-54, 1999). Dutch control subjects were taken from a large population based study designed to assess the effects of various risk factors on CAD (Seidell et al., International Journals of Obesity 19:924-927 1995; Kuivenhoven et al. Arteriosclerosis, Thrombosis and Vascular Biology 17:595-599, 1997). French Canadian subjects were a random sample of individuals. The South African Black and Cantonese cohorts have previously been described (Ehrenborg et al., Arteriosclerosis, Thrombosis and Vascular Biology 17:2672-2678, 1997). All subjects gave informed consent.

cSNP Screening

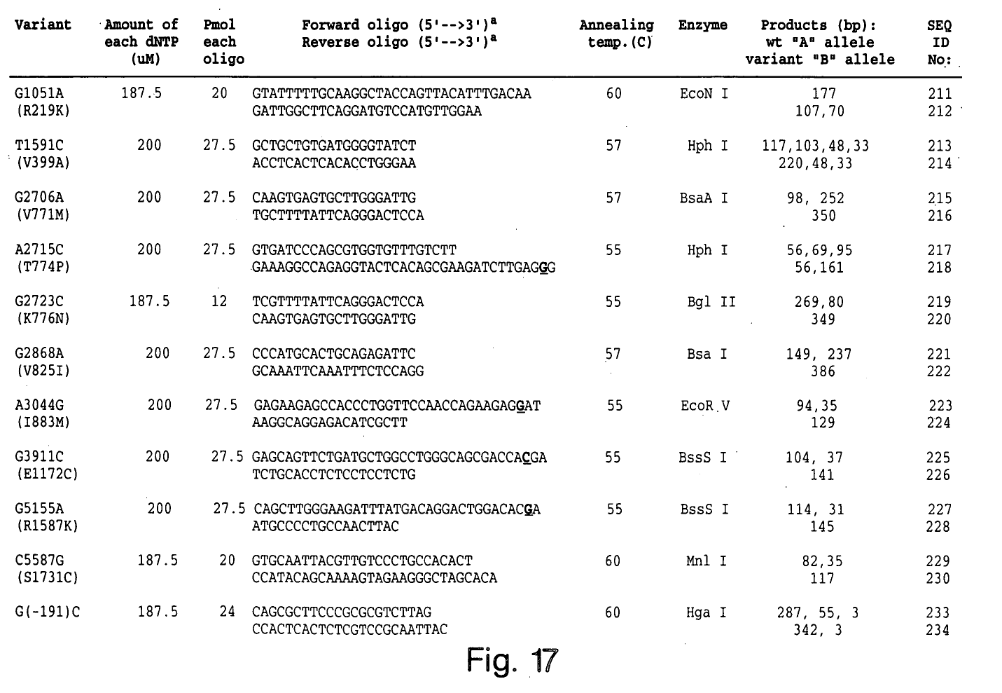

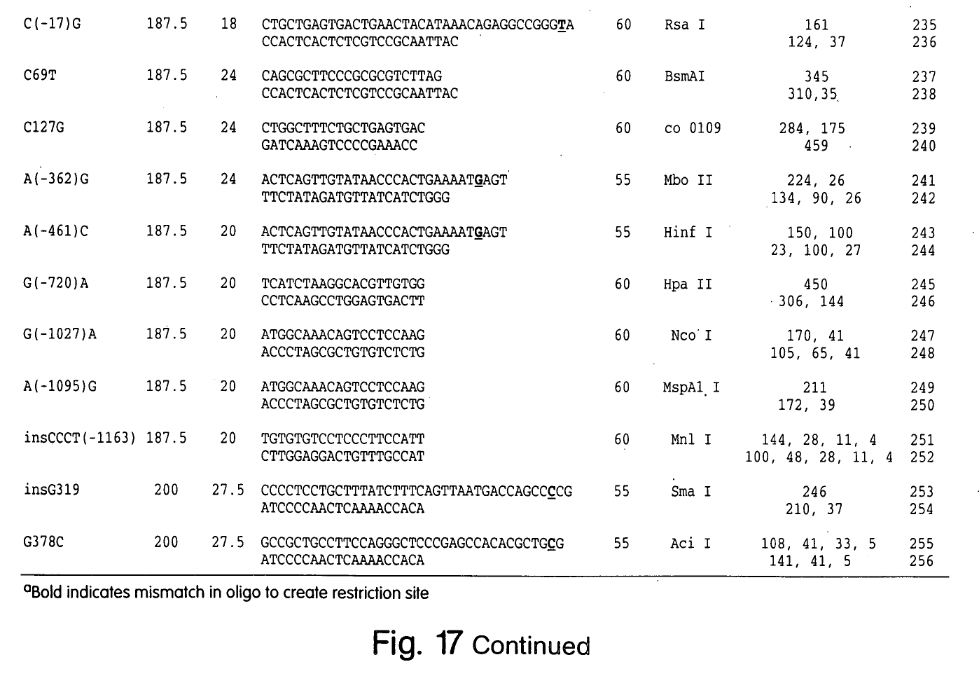

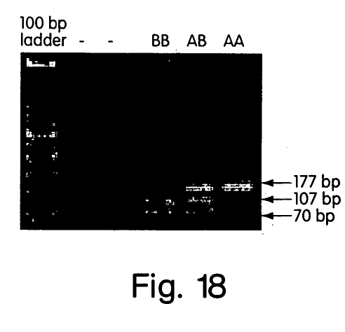

For each variant, a restriction enzyme whose cleavage pattern was altered by the variant was identified for development of an RFLP assay. If no suitable enzyme was found, a mismatch strategy was employed, whereby a single nucleotide mismatch was incorporated into the PCR primer, creating a restriction site in combination with either the wild-type or variant allele. The specific conditions of all assays are described in FIG. 17. All PCR reactions were carried out in 50 μL volumes, in the presence of 1×PCR buffer and 1.5 μM MgCl₂ (Life Technologies). Thermocycling parameters for all assays were as follows: 95° C. for 3 min; 35 cycles of denaturation at 95° C. for 10 seconds, annealing for 30 seconds at the temperature specified in FIG. 17, and elongation for 30 seconds at 72° C.; and a final elongation at 72° C. for 10 min. All digestions (15-20 μL PCR product) were carried out in manufacturer's buffer (New England Biolabs) for 2 hours at the temperature specified by the manufacturer. As an example, the digest results for the R219K are shown in FIG. 18. A 177 base pair fragment with the A allele is not cut by EcoNI, whereas the B allele is digested to produce fragments of 107 and 70 base pairs. Heterozygous individuals thus display all three bands (177, 107, and 70 base pairs).

Genotyping with the TaqMan(r) Assay

To facilitate the mass screening of some variants, TaqMan based polymerase chain reaction (PCR) assays (Holland et al., Proc.Natl.Acad.Sci. 88 (16):7276-7280, 1991; Lee et al., Nucleic Acids Research 21 (16):3761-3766, 1993) were developed for the detection of polymporphisms in the ABC1 gene. In this one-tube assay, two fluorogenic hybridization probes (one for each allele) are labeled with different fluorescent reporter dyes (FAM or TET) at their 5′ terminus and a common quencher dye (TAMRA) at their 3′ terminus. These probes are cleaved by the 5′ nuclease activity of Taq enzyme during PCR amplification. This cleavage separates the reporter from the quencher dye and generates an increase in reporter fluorescence. By using two different reporter dyes, cleavage of allele-specific probes can be detected in a single PCR. The difference in the measured fluorescence intensity between the two TaqMan probes allows for accurate allele calling.

PCR amplifications with flanking sets of primers (300 nM) in the presence of two TaqMan probes (25 nM each) and 4.5 mM MgCl₂ were performed using the following thermocycling protocol: initial denaturation at 96° C. for 10 min, followed by 39 cycles of 96° C. for 30 sec, 63° C. for 1 min and 72° C. for 15 sec, followed by a final extension at 72° C. for 10 min. Each plate included controls (no DNA template) as well as standards of each known genotype. Fluorescence quantification and genotype determination were performed on a Perkin Elmer LS50B or ABI Prism 7700 Sequence Detector. The fluorescence from each reaction was normalized to the signal from the no-template controls.

Cellular Cholesterol Efflux Studies

Cellular cholesterol efflux from fibroblast cultures was measured as described above in the “Analysis of ABC1 Heterozygotes” section.

Statistics

Within the REGRESS population, the baseline characteristics of the patients in the three genotypes (AA, AB, BB) was compared using one way analysis of variance and the chi-square test, where appropriate. In cases where the BB genotype was rare, the M group was compared to the combined group AB+BB.

The cumulative coronary event incidence in these genotypic groups was compared using the logrank test. The relation between age and HDL level was investigated using a linear regression model, and the difference between genotypes with respect to the slopes of this regression line was tested using covariance analysis. In addition to this regression analysis, the R219K genotypes were compared among age-defined subgroups. Randomization to placebo and pravastatin was assessed by chi square analysis and was equivalent in all genotypic groups for all variants except the R1587K variant. For this variant, a lower proportion of carriers was randomized to pravastatin treatment. In addition, change in MOD, MSD, and prevalence of coronary events (the three variables measured during the trial and thus following randomization) were analyzed for the placebo and pravastatin subgroups separately. Similar genotypic effects for each of the variants were observed in the treatment subgroups (i.e. the pravastatin and placebo control groups).

All lipid levels are reported in mmol/L. All values are reported as mean Å standard deviation.

Association of R219K Polymorphism with Reduced Triglyceride Levels and a Decreased Risk of CAD

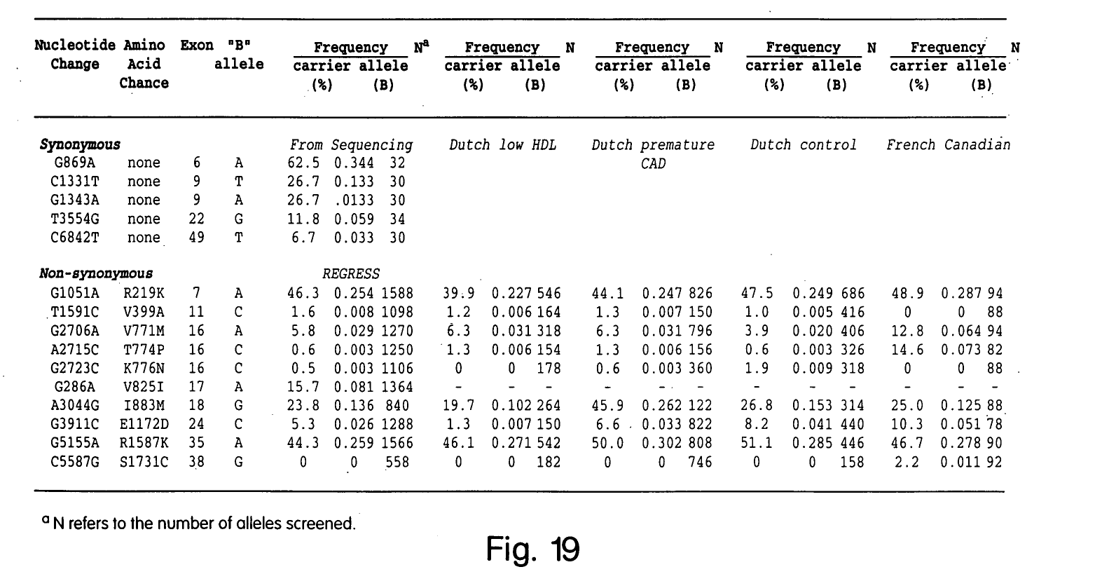

The common R219K polymorphism results in the substitution of a lysine for an arginine at amino acid 219 of the ABC1 protein. The allele frequency of the variant, or “B”, allele was 0.25, and its carrier frequency was 46.3%, as shown in FIG. 19.

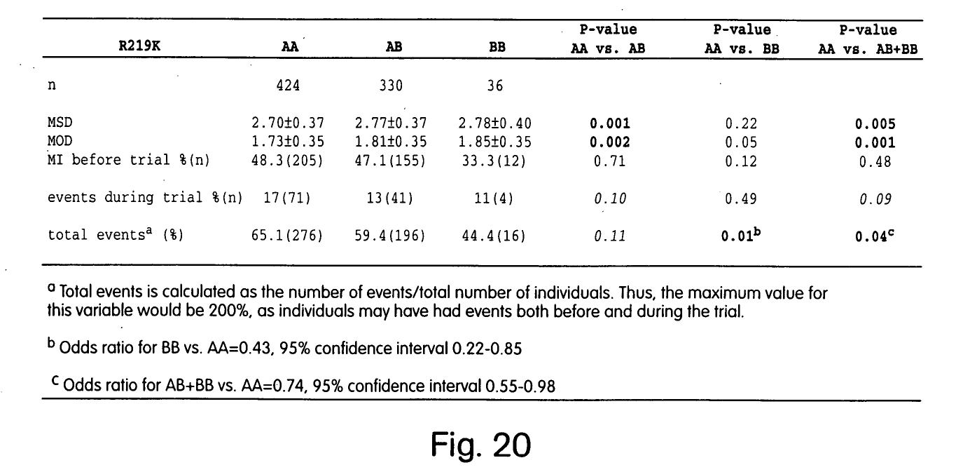

The relationship between this polymorphism and CAD was examined. The B allele of the R219K polymorphism was associated with decreased baseline CAD. Both the MSD and the MOD, were significantly increased in an allele-dose dependent fashion from AA to AB to BB (FIG. 20).

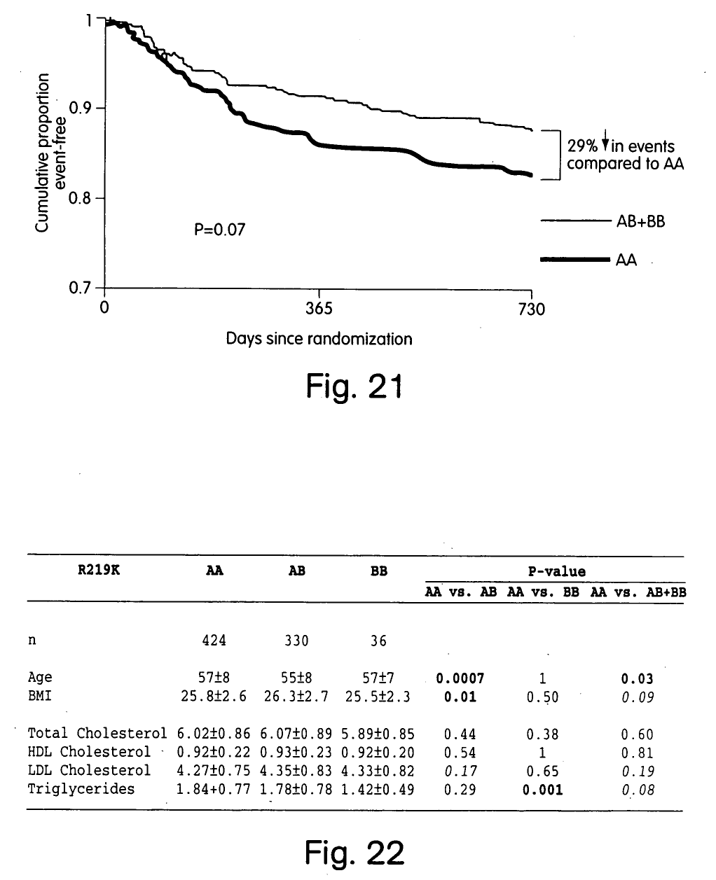

The angiographic data was paralleled by differences in clinical coronary events rates. A smaller percentage of individuals homozygous for the rarer B allele (BB) had had a myocardial infarction (MI) prior to the initiation of the trial. Carriers of the B allele exhibited a strong trend towards an increased prevalence of no events over the course of the study (FIG. 21, p=0.07).

The association of the R219K polymorphism with reduced CAD was further supported by the reduced frequency of this allele in the REGRESS cohort which was selected for CAD. In fact, the genotype frequencies observed for this variant are not consistent with Hardy-Weinberg equilibrium (p=0.004). There are fewer BB individuals and more AB individuals than would be expected (424 AA, 330 AB, and 36 BB individuals were observed compared to the expected 439 AA, 300 AB, and 51 BB individuals). As the REGRESS cohort was selected for men with CAD, the lack of Hardy-Weinberg equilibrium suggests that there was a preferential selection against BB individuals, consistent with the reduced CAD observed in this group.

There were no obvious differences in mean HDL-C levels between the genotypes in the groups as a whole (FIG. 22); however, triglycerides were significantly lower in the carriers of the B allele. This suggests that ABC1 function may also directly influence plasma triglyceride levels, and that this variant may be associated with a gain of ABC1 function. These findings are also consistent with the decreased CAD observed in carriers, suggesting that ABC1 modulation of triglyceride levels may be another mechanism whereby ABC1 activity influences risk of CAD.

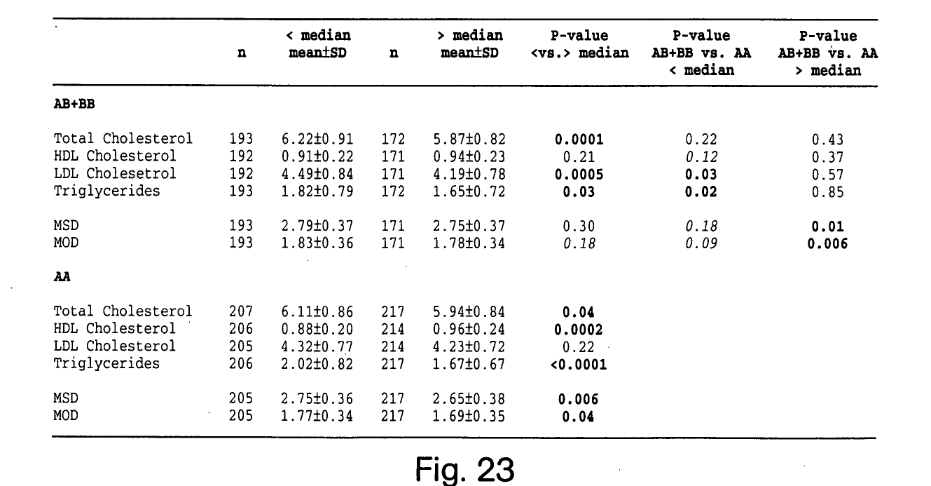

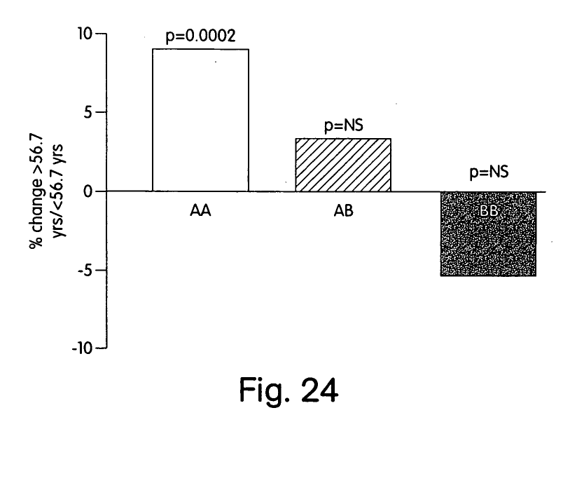

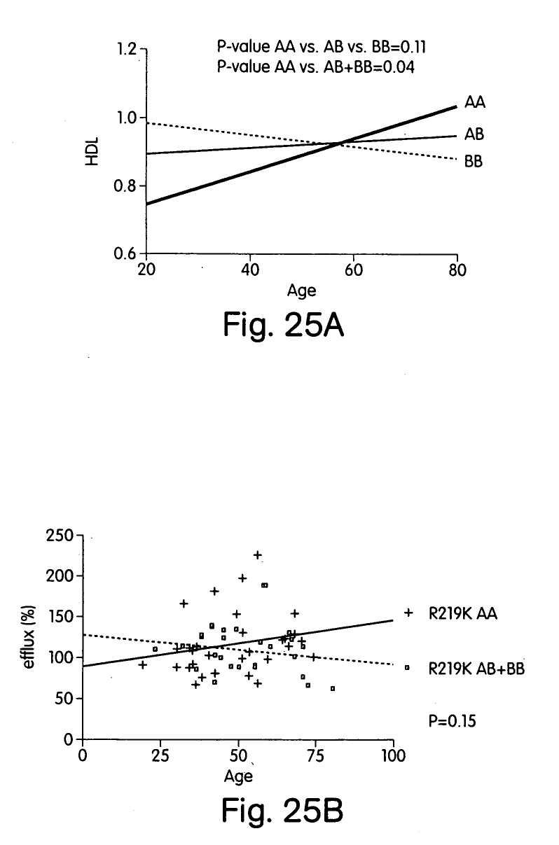

To further explore the apparent lack of effect of this variant on HDL-C, the relationship between R219K genotype and HDL at various ages was examined. In younger individuals, the carriers of the B allele had increased HDL-C compared to non-carriers (FIG. 23) Furthermore, while HDL-C increased with age in the AA individuals, heterozygous carriers showed a much milder increase, and HDL cholesterol decreased with age in homozygous carriers (FIG. 24). In the AA individuals, HDL cholesterol is positively correlated with age (p<0.001). In contrast, this relation was not apparent in individuals heterozygous for this variant, and HDL cholesterol was negatively correlated with age in the BB homozygotes, although neither the correlation for AB nor the correlation for BB individuals was statistically different from zero (FIG. 25A). The age-related increase in HDL-C in AA individuals is not maintained in carriers of the B allele (p value comparing slopes=0.04). Thus, the decreased CAD in carriers of the R219K may also be related to the fact that for the majority of their lifetime, carriers have had increased HDL-C compared to non-carriers.

The changes in HDL-C with age in the various R219K genotypes are mirrored by similar trends in the changes in cholesterol efflux with age (FIG. 25B) We have genotyped this variant in all individuals in whom we have measured efflux and who do not possess an ABC1 mutation. In AA individuals (n=30), cholesterol efflux increases with age, whereas in AB and BB individuals (n=24), efflux decreases with age (p-value comparing slopes=0.15). Thus, the differential age-related changes in HDL seen in the different R219K genotypes are consistent with similar functional changes in ABC1 activity.

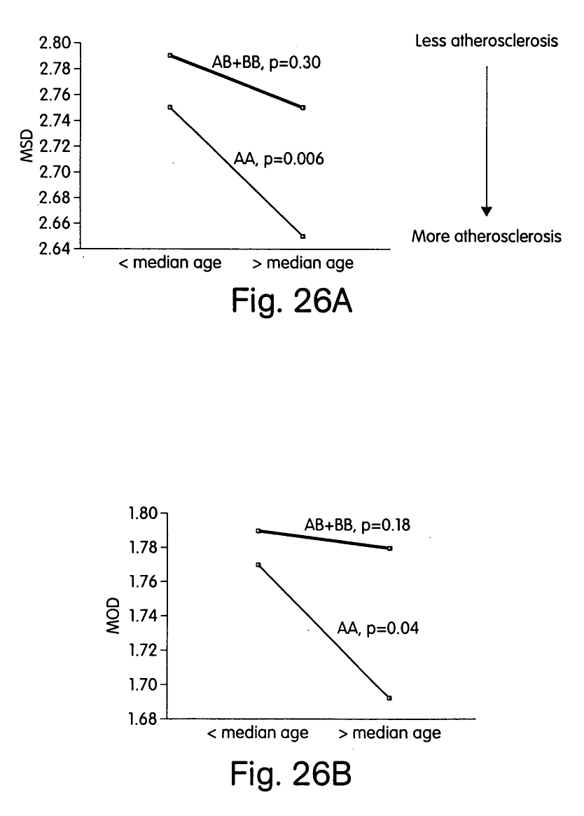

From FIG. 23 it can be seen that.triglyceride levels generally decrease with age, a finding seen in all R219K genotypes. The percent decrease in triglyceride levels was nearly half in carriers (9.3%) compared to non-carriers (17.3%, p=0.07). Differences between the genotypes are also observed in the MSD and MOD. In the non-carriers, MSD and MOD measurements decrease significantly with age, reflecting increased atherosclerosis in the older individuals (FIGS. 23 and 26). In contrast, in carriers of the R219K variant, these measurements do not significantly change with age. Thus, vascular disease progresses much more slowly with age in carriers of the R219K variant compared to non-carriers.

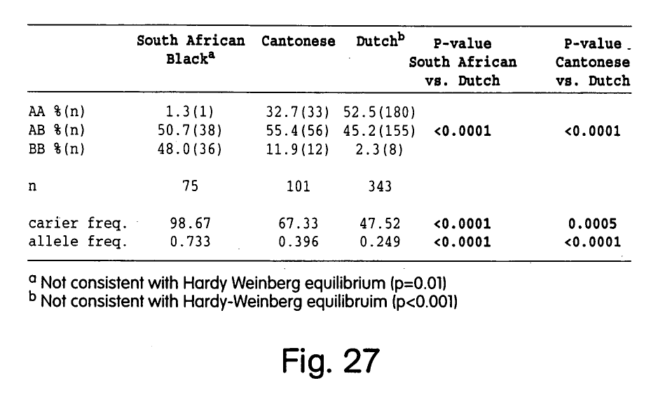

Populations of Asian and African origins have been shown to have increased HDL-C, decreased triglyceride levels, and decreased risk of CAD compared to Caucasian populations (Tyroler et al., Circulation 62 (Suppl. IV):IV-99-IV-107, 1980; Tao et al., International Journal of Epidemiology 21 (5):893-903, 1992; Brown et al., Arteriosclerosis and Thrombosis 13:1139-1158. 1993; Adedeji, Tropical and Geographical Medicine 46 (1):23-26, 1994; Simon et al., American Journal of Public Health 85 (12):1698-1702, 1995; Morrison et al., Metabolism 47 (5):514-521, 1998), a finding paralleling the phenotypic effects of this variant. Thus, as SNP frequencies can often differ within different ethnic groups, the frequency of this variant within these two population groups was examined (FIG. 27). This variant is seen much more commonly in individuals of either Cantonese or South African Black descent, in which it is the predominant allele. This suggests that the increased frequency of this variant may in part account for the increased HDL, decreased triglyceride levels, and decreased CAD observed in these populations compared to Caucasian populations.

Effect of Other cSNPs on Plasma Lipid Levels and Risk of CAD

No significant differences in lipid levels or CAD compared to respective non-carriers have been observed for carriers of the T774P (n=4), K776N (n=3) or E1172D variants (n=34). Carriers of the V825I (n=103 AB, 4 BB) had no obvious differences in lipid levels or baseline MSD or MOD. Carriers of the V825I variant did, however, have a significantly increased number of events during the trial (44% versus 33%, p=0.001).

No carriers of the S1731C variant were detected in the REGRESS population, but this variant was found in one of our FHA families (FHA2). In individuals heterozygous for ABC1 mutations, this variant was associated with significantly decreased HDL-C (0.16Å0.04, n=2 versus 0.64Å0.14, n=10; p=0.0009 in carriers versus non-carriers). In unaffected family members, however, while carriers of the S1731C had lower HDL-C compared to non-carriers (1.03Å0.22 versus 1.09Å0.23), this was not statistically significant. The control individual in whom this variant was also seen (FIG. 19) had low plasma HDL-C (0.72 mmol/L).

Similar to carriers of the R219K, carriers of the V771M variant (n=37), had no difference in HDL-C compared to non-carriers; however, a marginally significant interaction between age and genotype on HDL-C levels was noted (p=0.05). All but 2 carriers of the V771M variant are also carriers of the R219K variant. The interaction between age and genotype on HDL-C remains nearly significant when adjusted for R219K, genotype (p=0.11), thus this variant may have an age effect independent of that which can be attributed to the R219K. A trend to less CAD (increased MOD) was observed in carriers of this variant compared to non-carriers (1.89Å0.38 versus 1.76Å0.35, p=0.13).

For the 1883M cSNP, homozygous BB individuals (n=14) have increased progression in MOD (mean change of 0.53Å79 versus 0.1 IAO.25, p<0.001). BB individuals had an events rate double that of the AA individuals (21.4% versus 10.6%) although this was not statistically significant (p=0.19). No difference was observed in mead lipid levels between the 1883M genotypes. Furthermore, there were significantly more BB individuals than expected under Hardy-Weinberg equilibrium (14 BB, 86 AB, and 320 AA individuals observed compared to the expect 8 BB, 98 AB, and 314 AA individuals). As this cohort was selected for individuals with CAD, this might suggest a preferential inclusion of those with the BB genotype. The association of this variant with CAD is further supported by the significantly increased frequency of this variant in the premature CAD population (odds ratio for CAD in carriers of this variant=0.43, 95% confidence interval 0.22-0.85, p=0.01) (FIG. 19). These findings contrast with those of a very recent report which suggests that the homozygous carriers of this cSNP have increased HDL-C (Wang etal., Arteriosclerosis, Thrombosis and Vascular Biology 20:1983-1989, 2000).

Carriers of the V399A (AB, n=9) had a trend to higher HDL-C (1.03Å0.28 versus 0.92Å0.23, p=0.15) compared to individuals who were AA at this site (n=540). No events were observed in the AB group (compared to 14% in AA's, p=NS), and carriers had half the prevalence of a family history of CAD (22.2% versus 49.4%, p=0.18). Furthermore, consistent with this data, the carriers had a trend to increased baseline MOD (1.92Å0.32 versus 1.73Å0.35, p=0.13) and to less progression in MSD (−0.05Å0.10 versus 0.08Å0.19, p=0.16) during the trial. However, as the number of carriers of small, firm conclusions regarding the relationship of this variant to increased HDL-C and decreased CAD cannot be drawn.

Carriers of the R1587K variant (AB, BB) have decreased HDL-C compared to non-carriers in an allele-dose dependent trend (0.86Å0.16, 0.91Å0.23, and 0.94Å0.23 for BB, AB, and AA, respectively, p=0.03). No significant interaction with age was noted (p=0.32). Furthermore, on multiple regression including age, BMI, smoking, and triglyceride levels as covariates, the R1587K genotype is a significant predictor of HDL-C (p=0.027). However, no significant differences in CAD or events during the trial were evident in carriers compared to non-carriers.

### Summary of Association Studies of ABC1 Gene Variants and HDL Levels or Cardiovascular Disease

The following polymporphisms have been examined for their effect on cholesterol regulation and the predisposition for the development of cardiovascular disease. The polymporphisms are numbered from the nucleotide described as position 1 (Pullinger et al., supra), naming the first exon number 1.

Substitution of A for G at nucleotide 1051 (R219K). Carriers of this variant have reduced triglyceride levels, increased HDL cholesterol levels (particularly in younger individuals), and reduced CAD.

Substitution of C for T at nucleotide 1591 (V399A). This variant was associated with a trend towards increased HDL cholesterol in carriers.

Substitution of A for G at nucleotide 2706 (V771M). Carriers of this variant have been shown to have decreased CAD.

Substitution of C for A at nucleotide 2715 (T774P). This variant was seen less often in individuals with low HDL cholesterol levels or CAD than in controls.

Substitution of C for G at nucleotide 2723 (K776N). This variant has been found at a lower frequency (0.54% versus 1.89%) in a coronary artery disease population versus a control population of similar Dutch background.

Substitution of C for G at nucleotide 3911 (E1172D). This variant is seen at lower frequencies in individuals with low HDL and in some populations with premature coronary artery disease.

Substitution of A for G at nucleotide 5155 (R1587K). This variant is associated with decreased HDL cholesterol levels in carriers.

Substitution of G for C at nucleotide 5587 (S1731C). Two FHA individuals who have this variant on the other allele have much lower HDL cholesterol (0.155Å0.025) than the FHA individuals in the family who do not have this variant on the other allele (0.64Å0.14, p=0.0069). This variant has also been found in one general population French Canadian control with HDL at the 8^{th} percentile (0.92) and one French Canadian individual from a population selected for low HDL cholesterol levels and coronary disease (0.72).

Substitution of G for A at nucleotide 2723 (I883M). This variant has been seen at a much higher frequency in individuals of Dutch ancestry with premature coronary artery disease. Furthermore, homozygous carriers of this variant have significantly increased CAD progression compared to non-carriers.

Substitution of A for G at nucleotide 2868 (V825I). Carriers of this variant had significantly more CAD events than individuals who do not have this variant.

Substitution of C for G at nucleotide −191. Homozygous carriers of this variant have a three-fold increase in the frequency of coronary events (33.3% versus 11.2%, p=0.003) and a nearly double frequency of a positive family history of CAD (73.3% versus 47.7%, p=0.01).

Substitution of G for C at nucleotide −17. Carriers of this variant have significantly decreased coronary events (12.3% versus 18.2%, p=0.04) and a significantly decreased incidence of myocardial infarction (heart attack, 43.6% versus 52.8%, p=0.02).

Substitution of T for C at nucleotide 69. Carriers of this variant have increased CAD progression compared to non-carriers.

Substitution of G for C at nucleotide 127. Carriers of this variant have a trend towards decreased progression of CAD compared to non-carriers.

Insertion of CCCT at nucleotide −1163 in intron 1. Carriers of this variant have a trend to lower HDL cholesterol levels.

Substitution of G for A at nucleotide −1095 in intron 1. Homozygous carriers of this variant have a trend towards decreased HDL cholesterol and increased triglyceride levels compared to non-carriers.

Substitution of A for G at nucleotide −1027 in intron 1. Carriers of this variant are also carriers of the G(−720)A. Thus the effects attributed to that variant may also be attributed to carriers of this variant.

Substitution of A for G at nucleotide −720 in intron 1. Homozygous carriers of this variant had a trend towards an increased frequency of a positive family history of myocardial infarction.

Substitution of C for A at nucleotide −461 in intron 1. Carriers of this variant are also carriers of the A(−362)G. Thus the effects attributed to that variant may also be attributed to carriers of this variant.

Substitution of G for A at nucleotide −362 in intron 1. Carriers of this variant have decreased triglyceride levels compared to non-carriers.

Insertion of G at nucleotide 319. Carriers of this variant have increased CAD compared to non-carriers.

Substitution of G for C at nucleotide 378. Carriers of this variant are also carriers of the lnsG319. Thus the effects attributed to that variant may also be attributed to carriers of this variant.

### Functional Role of LXRE Binding Sites in ABC1 Genomic Sequence

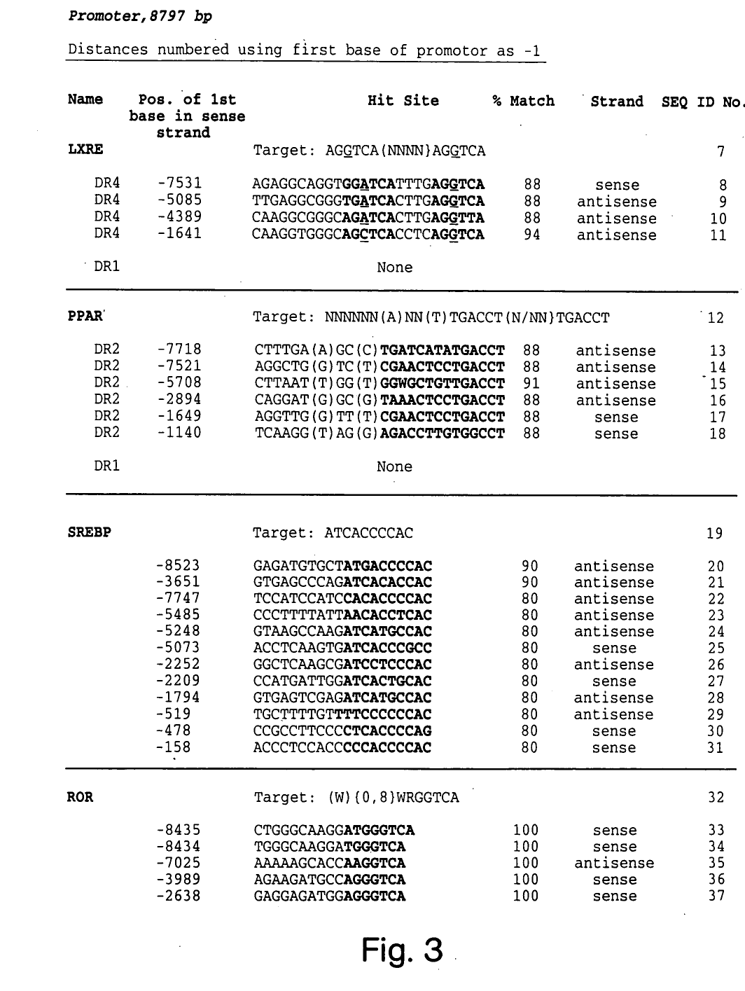

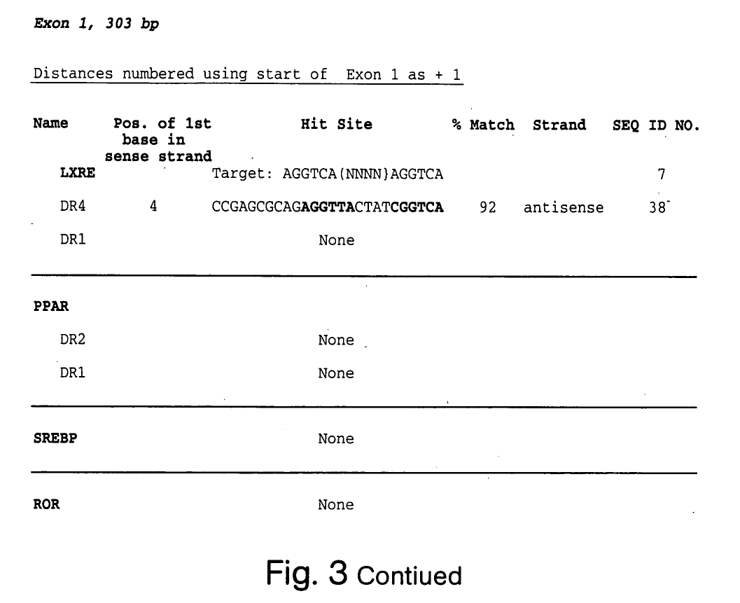

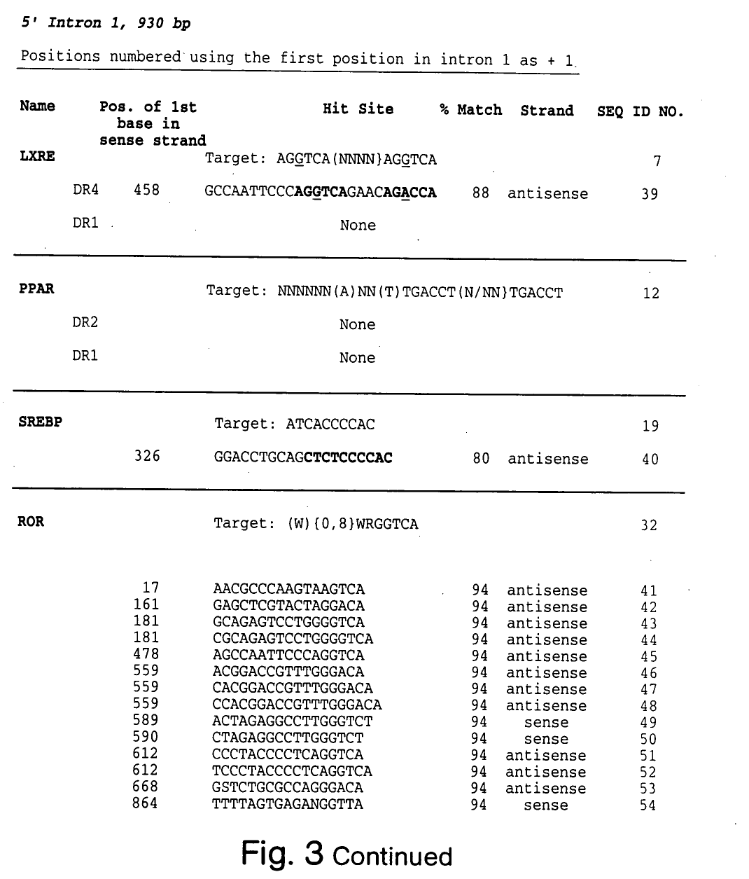

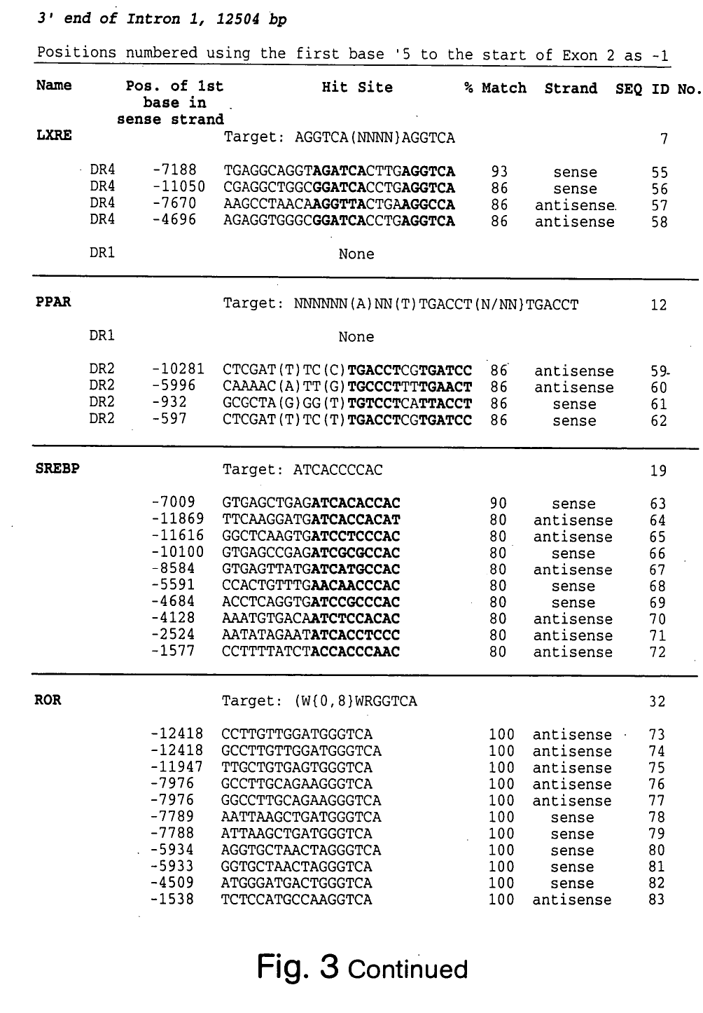

The functional role of three of the LXRE consensus binding sites identified in the ABC1 genomic sequence of SEQ ID NO: 1 was confirmed using standard gel shift assay experiments. Briefly, the sequences of the LXRE consensus binding sites located at −4389 and −1641 of the promoter region, +4 of exon 1, and −7670 and −7188 of 3′ intron 1 (FIG. 3) were tested in gel shift assays using the cyp7 LXRE described by Lehmann et al. as a positive control (Lehmann et al., J. Biol. Chem. 272:3137-3140, 1997).

In the first gel shift assay, binding of the labeled cyp7 LXRE probe was competed by a 400-fold excess of cold cyp7 probe, −4389 probe, −1641 probe, +4 probe, or −7670 probe. The signal disappeared completely in all cases.

For the next assay, the −4389, −1641, +4, −7670 and −7188 probes were labeled and binding to LXR-RXR complex was assayed. A signal similar to that of the positive control was observed for each of ABC1 LXRE probes, although the signal was slightly more intense for the +4 probe.

A competition assay was also performed using lesser quantities of each cold probe, i.e. 5-fold, 25-fold, and 50-fold more cold probe than labeled cyp7 probe. There was a dose dependent decrease in the signal for each of the probes. This decrease was mor significant for th +4 and −7670 probes. Moreover, the signal was not modified by competition with a cold Dr2 like probe, suggesting that th competition effect is indeed specific.

Thus, each of the tested potential LXRE binding sites seem to bind an in vitro LXR-RXR heterodimer. The LXRE binding site at +4 in exon 1 appears to have the highest affinity, closely followed by the LXRE binding site at −7670 in 3′ intron 1.

### Agonists and Antagonists

Useful therapeutic compounds include those which modulate the expression, activity, or stability of ABC1. To isolate such compounds, ABC1 expression, biological activity, or regulated catabolism is measured following the addition of candidate compounds to a culture medium of ABC1-expressing cells. Alternatively, the candidate compounds may be directly administered to animals (for example mice, pigs, or chickens) and used to screen for their effects on ABC1 expression.

In addition its role in the regulation of cholesterol, ABC1 also participates in other biological processes for which the development of ABC1 modulators would be useful. In one example, ABC1 transports interleukin-1β (IL-1β) across the cell membrane and out of cells. IL-1β is a precursor of the inflammatory response and, as such, inhibitors or antagonists of ABC1 expression or biological activity may be useful in the treatment of any inflammatory disorders, including but not limited to rheumatoid arthritis, systemic lupus erythematosis (SLE), hypo- or hyper- thyroidism, inflammatory bowel disease, and diabetes mellitus. In another example, ABC1 expressed in macrophages has been shown to be engaged in the engulfment and clearance of dead cells. The ability of macrophages to ingest these apoptotic bodies is impaired after antibody-mediated blockade of ABC1. Accordingly, compounds that modulate ABC1 expression, stability, or biological activity would be useful for the treatment of thes disorders.

ABC1 expression is measured, for example, by standard Northern blot analysis using an ABC1 nucleic acid sequence (or fragment thereof) as a hybridization probe, or by Western blot using an anti-ABC1 antibody and standard techniques. The level of ABC1 expression in the presence of the candidate molecule is compared to the level measured for the same cells, in the same culture medium, or in a parallel set of test animals, but in the absence of the candidate molecule. ABC1 activity can also be measured using the cholesterol efflux assay.

### Transcriptional Regulation of ABC1 Expression

ABC1 mRNA is increased approximately 8-fold upon cholesterol loading. This increase is likely controlled at the transcriptional level. Using the genomic sequence described herein, one can identify transcription factors that bind to the 5′ regulatory sequence by performing, for example, gel shift assays, DNAse protection assays, or in vitro or in vivo reporter gene-based assays. The identified transcription factors are themselves drug targets. In the case of ABC1, drug compounds that act through modulation of transcription of ABC1 could be used for HDL modulation, triglyceride modulation, atherosclerosis prevention, and the treatment of cardiovascular disease. For example, using a compound to inhibit a transcription factor that represses ABC1 would be expected to result in up-regulation of ABC1 and, therefore, up-regulation of HDL cholesterol levels and down-regulation of triglyceride levels. In another example, a compound that increases transcription factor expression or activity would also increase ABC1 expression, increase HDL levels, and decrease triglyceride levels.

Transcription factors known to regulate other genes in the regulation of apolipoprotein genes or other cholesterol- or lipid-regulating genes are of particular relevance. Such factors include, but are not limited to, the steroid response element binding proteins (SREBP-1 and SREBP-2), and the PPAR (peroxisomal proliferation-activated receptor), RXR, and LXR transcription factors. Several consensus sites for certain elements are present in the sequenced region 5′ to the ABC1 gene (FIG. 3) and thus are likely to modulate ABC1 expression. For example, LXRs may alter transcription of ABC1 by mechanisms including heterodimerization with retinoid X receptors (RXRs) and then binding to specific response elements (LXREs). Examples of such LXRs include LXRα and LXRβ. Compounds that modulate LXR-mediated transcriptional activation are likely to modulate ABC1 gene expression and thus are useful for modulating HDL cholesterol levels and triglyceride levels. Janowski et al. (Proc. Natl. Acad. Sci.. USA 96:266-271, 1999) described the role of naturally occurring oxysterols in LXR-dependent transactivation through the promoter for cholesterol 7a-hydoxylase (Cyp7a), which is the rate limiting enzyme in bile acid synthesis. Janowski further demonstrated that oxysterols bind directly to LXRs. The position specific mono-oxidation of the sterol side chain is required for LXR high affinity binding and activation. Enhanced binding could be achieved by use of 24-oxo ligands. Oxygens at more than one carbon on the side chain of cholesterol diminished LXR binding and activation as compared to monoxygenated analogs. LXR ligands were found to require a single stereoselective oxygen on the sterol side chain that functioned as a hydrogen acceptor. Introduction of dimethylamide exhibited the greatest binding and activation compared to an ester or carbonyl group.

Compounds known to modulate LXR activity include, without limitation, 24-(S),25-epoxycholesterol; 24(S)-hydroxycholesterol; 22-(R)-hydroxycholesterol; 24(R),25-epoxycholesterol; 22(R)-hydroxy-24(S),25-epoxycholesterol; 22(S)-hydroxy-24(R),25-epoxycholesterol; 24-(S),25-iminocholesterol; methyl-38-hydroxycholonat; N,N-dimethyl-3β-hydroxycholonamid; 24(R)-hydroxycholesterol; 22(S)-hydroxycholesterol; 22(R),24(S)-dihydroxycholesterol; 25-hydroxycholesterol; 22(R)-hydroxycholesterol; 22(S)-hydroxycholesterol; 24(S),25-dihydroxycholesterol; 24(R),25-dihydroxycholesterol; 24,25-dehydrocholesterol; 25-epoxy-22(R)-hydroxycholesterol; 20(S)-hydroxycholesterol; (20R,22R)-cholest-5-ene-3β,20,22-triol; 4,4-dimethyl-5-α-cholesta-8,14,24-trien-3-β-ol; 7α-hydroxy-24(S),25-epoxycholesterol; 7β-hydroxy-24(S),25-epoxycholesterol; 7-oxo-24(S),25-expoxycholesterol; 7α-hydroxycholesterol; 7-oxocholesterol; and desmosterol. Additional LXR-modulating compounds are described, for example, in Janowski et al., Nature 383:728-731, 1996; Lehman et al., J. Biol. Chem. 272:3137-3140, 1997; and Janowski et al., Proc. Natl. Acad. Sci. 96:266- 271, 1998, each of which is hereby incorporated by reference). In addition one in the art will recognize that synthetic sterols having LXR-modulating activity can be readily identified using screening methods known in the art (see, for example, Janowski et al., Proc. Natl. Acad. Sci. 96:266-271, 1998). Non-steroidal agonists such as RIP140 protein, antibodies (monoclonal or polyclonal) specific for LXRα or LXRβ; tetradecycloxy-furnacarboxylic acid (TOFA;); tetradecylthioacetic acid; as well as other fatty acids (see, for example, Tobin et al. Molec. Endocrin. 14: 741-752, 2000) are also useful LXR-modulating agents.

Additional transcription factors which may also have an effect in modulating ABC1 gene expression and thereby HDL levels, triglyceride levels, atherosclerosis, and CAD risk include REV-ERBA, SREBP-1 & 2, ADD-1, EBPα, CREB binding protein, P300, HNF 4, RAR, and RORα. Exemplary binding sites are depicted in FIG. 3. Additional binding sites for these factors can be found, for example, through examination of the sequence in SEQ ID NO: 1.

RXR heterodimerizes with many nuclear receptors, including LXR, and aids in transactivating the target gene. Thus, compounds that modulate RXR-mediated transcriptional activity will also modulate ABC1 expression. Numerous RXR-modulating compounds (rexinoid compounds) are known in the art, including, for example, hetero ethylene derivatives; tricyclic retinoids; trienoic retinoids; benzocycloalkenyl-alka:di- or trienoic acid derivatives; bicyclic-aromatic compounds and their derivatives; bicyclylmethyl-aryl acid derivatives; phenyl-methyl heterocyclic compounds; tetrahydro-napthyl compounds; arylthio-tetrahydro-naphthalene derivatives and heterocyclic analogues; 2,4-pentadienoic acid derivatives; tetralin-based compounds; nonatetraenoic acid derivatives; SR11237; dexamethasone; hydroxy, epoxy, and carboxy derivatives of methoprene; bicyclic benzyl, pyridinyl, thiophene, furanyl, and pyrrole derivatives; benzofuran-acrylic acid derivatives; aryl-substituted and aryl and (3-oxo-1-propenly)-substituted benzopyran, benzothiopyran, 1,2-dihydroquinoline, and 5,6-dihydronaphthalene derivatives; vitamin D3 (1,25-dihydroxyvitamin D3) and analogs; 24-hydroxylase inhibitor; mono-or polyenic carboxylic acid derivatives; tetrahdroquinolin-2-one-6 or 7-yl and related derivatives; tetrahydronaphthalene; oxyiminoalkanoic acid derivatives; LG 100268; and LGD 1069. Additional compounds include BRL 49653; troglitazone; pioglitazone; ciglitazone; WAY-120; englitazone; AD 5075; and darglitazone.

PPARs may alter transcription of ABC1 by mechanisms including heterodimerization with retinoid X receptors (RXRs) and then binding to specific proliferator response elements (PPREs). Examples of such PPARs include PPARα, β, γ and δ. These distinct PPARs have been shown to have transcriptional regulatory effects on different genes. PPARα is expressed mainly in liver, whereas PPARγ is expressed in predominantly in adipocytes. Both PPARα and PPARγ are found in coronary and carotid artery atherosclerotic plaques and in endothelial cells, smooth muscle cells, monocytes and monocyte-derived macrophages. Activation of PPARα results in altered lipoprotein metabolism through PPARα's effect on genes such as lipoprotein lipase (LPL), apolipoprotein CIII (apo CIII) and apolipoprotein AI (apo AI) and AII (apo AII). PPARα activation results in overexpression of LPL and apoA-I and apoA-II, but inhibits the expression of apo CIII. PPARα activation also inhibits inflammation, stimulates lipid oxidation and increases the hepatic uptake and esterification of free fatty acids (FFA's). PPARα and PPARγ activation may inhibit nitric oxide (NO) synthase in macrophages and prevent interleukin-1 (IL-1) induced expression of IL-6 and cyclo-oxygenase-2 (COX-2) and thrombin induced endothelin-1 expression secondary to negative transcriptional regulation of NF-KB and activation of protein-1 signaling pathway. It has also been shown that PPARα induces apoptosis in monocyte-derived macrophages through the inhibition of NF-KB activity.

Activation of PPARα can be achieved by compounds such as fibrates, β-estradiol, arachidonic acid derivatives, WY-14,643 and LTB4 or 8(s)HETE. PPARγ activation can be achieved through compounds such as thiozolidinedione antidiabetic drugs, 9-HODE and 13-HODE. Additional compounds such as nicotinic acid or HMG CoA reductase inhibitors may also alter the activity of PPARs.

Compounds which alter activity of any of the PPARs (e.g., PPARα or PPARγ) may have an effect on ABC1 expression and thereby could affect HDL levels, triglyceride levels, atherosclerosis, and risk of CAD. PPARs are also regulated by fatty acids (including modified fatty acids such as 3 thia fatty acids), leukotrienes such as leukotriene B4 and prostaglandin J2, which is a natural activator/ligand for PPARγ. Drugs that modulate PPARs may therefore have an important effect on modulating lipid levels (including HDL and triglyceride levels) and altering CAD risk. This effect could be achieved through the modulation of ABC1 gene expression. Drugs may also effect ABC1 gene expression and thereby HDL and triglyceride levels, by an indirect effect on PPARs via other transcriptional factors such as adipocyte differentiation and determination factor-1 (ADD-1) and sterol regulatory element binding protein-1 and 2 (SREBP-1 and 2). Drugs with combined PPARα and PPARγ agonist activity or PPARα and PPARγ agonists given in combination for example, may increase HDL levels or decrease triglyceride levels even more.

A PPAR binding site (PPRE element) is found 5′ to the ABC1 gene (FIG. 3). Like the PPRE elements found in the C-ACS, HD, CYP4Å6 and ApoA-I genes, this PPRE site is a trimer related to the PPRE consensus sequence. Partly because of its similarity in the number and arrangement of repeats in this PPAR binding site, this element in particular is very likely to be of physiological relevance to the regulation of the ABC1 gene.

### Additional Utility of ABC1 Polypeptides, Nucleic Acids, and Modulators

ABC1 may act as a transporter of toxic proteins or protein fragments (e.g., APP) out of cells. Thus, ABC1 agonists/upregulators may be useful in the treatment of other disease areas, including Alzheimer's disease, Niemann-Pick disease, and Huntington's disease.

ABC transporters have been shown to increase the uptake of long chain fatty acids from the cytosol to peroxisomes and, moreover, to play a role in β-oxidation of very long chain fatty acids. Importantly, in x-linked adrenoleukodystrophy (ALD), fatty acid metabolism is abnormal, due to defects in the peroxisomal ABC transporter. Any agent that upregulates ABC transporter expression or biological activity may therefore be useful for the treatment of ALD or any other lipid disorder.

ABC1 is expressed in macrophages and is required for engulfment of cells undergoing programmed cell death. The apoptotic process itself, and its regulation, have important implications for disorders such as cancer, one mechanism of which is failure of cells to undergo cell death appropriately. ABC1 may facilitate apoptosis, and as such may represent an intervention point for cancer treatment. Increasing ABC1 expression or activity or otherwise up-regulating ABC1 by any method may constitute a treatment for cancer by increasing apoptosis and thus potentially decreasing the aberrant cellular proliferation characterized by this disease. Conversely, down-regulation of ABC1 by any method may provide opportunity for decreasing apoptosis and allowing increased proliferation of cells in conditions where cell growth is limited. Such disorders include but are not limited to neurodeficiencies and neurodegeneration, and growth disorders. ABC1 could, therefore, be used as a method for identification of compounds for use in the treatment of cancer, or in the treatment of degenerative disorders.

Agents that have been shown to inhibit ABC1 include, for example, the anti-diabetic agents glibenclamide and glyburide, flufenamic acid, diphenylamine-2-carbonic acid, sulfobromophthalein, and DIDS.

Agents that upregulate ABC1 expression or biological activity include but are not limited to protein kinase A, protein kinase C, vanadate, okadaic acid, and IBMX1.

Those in the art will recognize that other compounds can also modulate ABC1 biological activity, and these compounds are also in the spirit of the invention.

### Drug Screens Based on the ABC1 Regulatory Regions, Gene, or Protein

The ABC1 protein and gene can. be used in screening assays for identification of compounds which modulate its activity and may be potential drugs to regulate cholesterol or triglyceride levels. The ABC1 5′ regulatory sequence and other regulatory regions (e.g., exon 1 and exon 2) can be used in screening assays for identification of compounds which modulate ABC1 expression and may be potential drugs to regulate lipid levels, including, for exampl, HDL-C, LDL-C, and triglycerides. Drug screens to identify compounds that modulate ABC1 expression may employ an ABC1 regulatory region operably linked to ABC1. Preferably, however, the regulatory region is operably linked to a reporter gene (e.g., a gene encoding GFP, chloramphenicol acetyltransferase, or beta-galactosidase).

Useful ABC1 proteins include wild-type and mutant ABC1 proteins or protein fragments, in a recombinant form or endogenously expressed. Drug screens to identify compounds acting on the ABC1 expression product may employ any functional feature of the protein. In one example, the phosphorylation state or other post-translational modification is monitored as a measure of ABC1 biological activity. ABC1 has ATP binding sites, and thus assays may wholly or in part test the ability of ABC1 to bind ATP or to exhibit ATPase activity. ABC1, by analogy to similar proteins, is thought to be able to form a channel-like structure; drug screening assays could be based upon assaying for the ability of the protein to form a channel, or upon the ability to transport cholesterol or another molecule, or based upon the ability of other proteins bound by or regulated by ABC1 to form a channel. Alternatively, phospholipid or lipid transport can also be used as measures of ABC1 biological activity.

There is evidence that, in addition to its role as a regulator of cholesterol levels, ABC1 also transports anions. Functional assays could be based upon this property, and could employ drug screening technology such as (but not limited to) the ability of various dyes to change color in response to changes in specific ion concentrations in such assays can be performed in vesicles such as liposomes, or adapted to use whole cells.

Drug screening assays can also be based upon the ability of ABC1 or other ABC transporters to interact with other proteins. Such interacting proteins can be identified by a variety of methods known in the art, including, for example, radioimmunoprecipitation, co-immunoprecipitation, co-purification, and yeast two-hybrid screening. Such interactions can be further assayed by means including but not limited to fluorescence polarization or scintillation proximity m thods. Drug screens can also be based upon functions of the ABC1 protein deduced upon X-ray crystallography of the protein and comparison of its 3-D structure to that of proteins with known functions. Such a crystal structure has been determined for the prokaryotic ABC family member HisP, histidine permease. Drug screens can be based upon a function or feature apparent upon creation of a transgenic or knockout mouse, or upon overexpression of the protein or protein fragment in mammalian cells in vitro. Moreover, expression of mammalian (e.g., human) ABC1 in yeast or C. elegans allows for screening of candidate compounds in wild-type and mutant backgrounds, as well as screens for mutations that enhance or suppress an ABC1-dependent phenotype. Modifier screens can also be performed in ABC1 transgenic or knock-out mice.

Additionally, drug screening assays can also be based upon ABC1 functions deduced upon antisense interference with the gene function. Intracellular localization of ABC1, or effects which occur upon a change in intracellular localization of the protein, can also be used as an assay for drug screening. Immunocytochemical methods will be used to determine the exact location of the ABC1 protein.

Human and rodent ABC1 protein can be used as an antigen to raise antibodies, including monoclonal antibo dies. Such antibodies will be useful for a wide variety of purposes, including but not limited to functional studies and the development of drug screening assays and diagnostics. Monitoring the influence of agents (e.g., drugs, compounds) on the expression or biological activity of ABC1 can be applied not only in basic drug screening, but also in clinical trials. For example, the effectiveness of an agent determined by a screening assay as described herein to increase ABC1 gene expression, protein levels, or biological activity can be monitored in clinical trails of subjects exhibiting altered ABC1 gene expression, protein levels, or biological activity. Alternatively, the effectiveness of an agent determined by a screening assay to modulate ABC1 gene expression, protein levels, or biological activity can be monitored in clinical trails of subjects exhibiting decreased altered gene expression, protein levels, or biological activity. In such clinical trials, the expression or activity of ABC1 and, preferably, other genes that have been implicated in, for example, cardiovascular disease can be used to ascertain the effectiveness of a particular drug.

For example, and not by way of limitation, genes, including ABC1, that are modulated in cells by treatment with an agent (e.g., compound, drug or small molecule) that modulates ABC1 biological activity (e.g., identified in a screening assay as described herein) can be identified. Thus, to study the effect of agents on cholesterol levels, triglyceride levels, or cardiovascular disease, for example, in a clinical trial, cells can be isolated and RNA prepared and analyzed for the levels of expression of ABC1 and other genes implicated in the disorder. The levels of gene expression can be quantified by Northern blot analysis or RT-PCR, or, alternatively, by measuring the amount of protein produced, by one of a number of methods known in the art, or by measuring the levels of biological activity of ABC1 or other genes. In this way, the gene expression can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) including the steps of (i) obtaining a pre-administration sample from a subject prior to administration of the agent; (ii) detecting the level of expression of an ABC1 protein, mRNA, or genomic DNA in the preadministration sample; (iii) obtaining one or more post-administration samples from the subject; (iv) detecting the level of expression or activity of the ABC1 protein, mRNA, or genomic DNA in the post-administration samples; (v) comparing the level of expression or activity of the ABC1 protein, mRNA, or genomic DNA in the pre-administration sample with the ABC1 protein, mRNA, or genomic DNA in the post administration sample or samples; and (vi) altering the administration of the agent to the subject accordingly. For example, increased administration of the agent may be desirable to increase the expression or activity of ABC1 to higher levels than detected, i.e., to increase the effectiveness of the agent. Alternatively, decreased administration of the agent may be desirable to decrease expression or activity of ABC1 to lower levels than detected.

The ABC1 gene or a fragment thereof can be used as a tool to express the protein in an appropriate cell in vitro or in vivo (gene therapy), or can be cloned into expression vectors which can be used to produce large enough amounts of ABC1 protein to use in in vitro assays for drug screening. Expression systems which may be employed include baculovirus, herpes virus, adenovirus, adeno-associated virus, bacterial systems, and eucaryotic systems such as CHO cells. Naked DNA and DNA-liposome complexes can also be used.

Assays of ABC1 activity includes binding to intracellular interacting proteins; interaction with a protein that up-regulates ABC1 activity; interaction with HDL particles or constituents; interaction with other proteins which facilitate interaction with HDL or its constituents; and measurement of cholesterol efflux. Furthermore, assays may be based upon the molecular dynamics of macromolecules, metabolites and ions by means of fluorescent-protein biosensors. Alternatively, the effect of candidate modulators on expression or activity may be measured at the level of ABC1 protein production using the same general approach in combination with standard immunological detection techniques, such as Western blotting or immunoprecipitation with an ABC1-specific antibody. Again, useful cholesterol- or triglyceride-regulating or anti-CVD therapeutic modulators are identified as those which produce an change in ABC1 polypeptide production. Agonists may also affect ABC1 activity without any effect on expression level.

Candidate modulators may be purified (or substantially purified) molecules or may be one component of a mixture of compounds (e.g., an extract or supernatant obtained from cells). In a mixed compound assay, ABC1 expression is tested against progressively smaller subsets of the candidate compound pool (e.g., produced by standard purification techniques, e.g., HPLC or FPLC; Ausubel et al.) until a single compound or minimal compound mixture is demonstrated to modulate ABC1 expression.

Agonists, antagonists, or mimetics found to be effective at modulating the level of cellular ABC1 expression or activity may be confirmed as useful in animal models (for example, mice, pigs, rabbits, or chickens). For example, the compound may ameliorate the low HDL levels of mouse or chicken hypoalphalipoproteinemias or may lower the triglyceride levels in animal models.

A compound that promotes an increase in ABC1 expression or activity is considered particularly useful in the invention; such a molecule may be used, for example, as a therapeutic to increase the level or activity of native, cellular ABC1 and thereby treat a low HDL or high triglyceride condition in an animal (for example, a human). If desired, treatment with an agonist of the invention may be combined with any other HDL-raising, triglyceride-lowering, or anti-CVD therapies.

One method for increasing ABC biological activity is to increase the stabilization of the ABC protein or to prevent its degradation. Thus, it would be useful to identify mutations in an ABC polypeptide (e.g., ABC1) that lead to increased protein stability. These mutations can be incorporated into any protein therapy or gene therapy undertaken for the treatment of low HDL-C or any other condition resulting from loss of ABC1 biological activity. Similarly, compounds that increase the stability of a wild-type ABC polypeptide or decrease its catabolism may also be useful for the treatment of low HDL-C or any other condition resulting from loss of ABC1 biological activity. Such mutations and compounds can be identified using the methods described herein.

In one example, cells expressing an ABC polypeptide having a mutation are transiently metabolically labeled during translation and the half-life of the ABC polypeptide is determined using standard techniques. Mutations that increase the half-life of an ABC polypeptide are ones that increase ABC protein stability. These mutations can then be assessed for ABC biological activity. They can also be used to identify proteins that affect the stability of ABC1 mRNA or protein. One can then assay for compounds that act on these factors or on the ability of these factors to bind ABC1.

In another example, cells expressing wild-type ABC polypeptide are transiently metabolically labeled during translation, contacted with a candidate compounds, and the half-life of the ABC polypeptide is determined using standard techniques. Compounds that increase the half-life of an ABC polypeptide are useful compounds in the present invention.

It is understood that, while ABC1 is the preferred ABC transporter for the drug screens described herein, other ABC transporters can also be used. The replacement of ABC1 with another ABC transporter is possible because it is likely that ABC transporter family members, such as ABC2, ABCR, or ABC8 will have a similar mechanism of regulation.

Exemplary assays are described in greater detail below.

### Protein-Based Assays

ABC1 polypeptide (purified or unpurified) can be used in an assay to determine its ability to bind another protein (including, but not limited to, proteins found to specifically interact with ABC1). The effect of a compound on that binding is then determined.

Protein Interaction Assays

ABC1 protein (or a polypeptide fragment thereof or an epitope-tagged form or fragment thereof) is harvested from a suitable source (e.g., from a prokaryotic expression system, eukaryotic cells, a cell-free system, or by immunoprecipitation from ABC1-expressing cells). The ABC1 polypeptide is then bound to a suitable support (e.g., nitrocellulose or an antibody or a metal agarose column in the case of, for example, a his-tagged form of ABC1). Binding to the support is preferably done under conditions that allow proteins associated with ABC1 polypeptide to remain associated with it. Such conditions may include use of buffers that minimize interference with protein-protein interactions. The binding step can be done in the presence and absence of compounds being tested for their ability to interfere with interactions between ABC1 and other molecules. If desired, other proteins (e.g., a cell lysate) are added, and allowed time to associate with the ABC polypeptide. The immobilized ABC1 polypeptide is then washed to remove proteins or other cell constituents that may be non-specifically associated with it the polypeptide or the support. The immobilized ABC1 polypeptide is then dissociated from its support, and so that proteins bound to it are released (for example, by heating), or, alternatively, associated proteins are released from ABC1 without releasing the ABC1 polypeptide from the support. The released proteins and other cell constituents can be analyzed, for example, by SDS-PAGE gel electrophoresis, Western blotting and detection with specific antibodies, phosphoamino acid analysis, protease digestion, protein sequencing, or isoelectric focusing. Normal and mutant forms of ABC1 can be employed in these assays to gain additional information about which part of ABC1 a given factor is binding to. In addition, when incompletely purified polypeptide is employed, comparison of the normal and mutant forms of the protein can be used to help distinguish true binding proteins.

The foregoing assay can be performed using a purified or semipurified protein or other molecule that is known to interact with ABC1 This assay may include the following steps.

1. Harvest ABC1 protein and couple a suitable fluorescent label to it;

2. Label an interacting protein (or other molecule) with a second, different fluorescent label. Use dyes that will produce different quenching patterns when they are in close proximity to each other versus when they are physically separate (i.e., dyes that quench each other when they are close together but fluoresce when they are not in close proximity);

3. Expose the interacting molecule to the immobilized ABC1 in the presence or absence of a compound being tested for its ability to interfere with an interaction between the two; and

4. Collect fluorescent readout data.

Another assay is includes Fluorescent Resonance Energy Transfer (FRET) assay. This assay can be performed as follows.

1. Provide ABC1 protein or a suitable polypeptide fragment thereof and couple a suitable FRET donor (e.g., nitro-benzoxadiazole (NBD)) to it;

2. Label an interacting protein (or other molecule) with a FRET acceptor (e.g., rhodamine);

3. Expose the acceptor-labeled interacting molecule to the donor-labeled ABC1 in the presence or absence of a compound being tested for its ability to interfere with an interaction between the two; and

4. Measure fluorescence resonance energy transfer.

Quenching and FRET assays are related. Either one can be applied in a given case, depending on which pair of fluorophores is used in the assay.

### Membrane Permeability Assay

The ABC1 protein can also be tested for its effects on membrane permeability. For exampl, beyond its putative ability to translocate lipids, ABC1 might affect the permeability of membranes to ions. Other related membrane proteins, most notably the cystic fibrosis transmembrane conductance regulator and the sulfonylurea receptor, are associated with and regulate ion channels.

ABC1 or a fragment of ABC1 is incorporated into a synthetic vesicle, or, alternatively, is expressed in a cell and vesicles or other cell sub-structures containing ABC1 are isolated. The ABC1-containing vesicles or cells are loaded with a reporter molecule (such as a fluorescent ion indicator whose fluorescent properties change when it binds a particular ion) that can detect ions (to observe outward movement), or alternatively, the external medium is loaded with such a molecule (to observe inward movement). A molecule which exhibits differential properties when it is inside the vesicle compared to when it is outside the vesicle is preferred. For example, a molecule that has quenching properties when it is at high concentration but not when it is at another low concentration would be suitable. The movement of the charged molecule (either its ability to move or the kinetics of its movement) in the presence or absence of a compound being tested for its ability to affect this process can be determined.

In another assay, membrane permeability is determined electro-physiologically by measuring ionic influx or efflux mediated by or modulated by ABC1 by standard electrophysiological techniques. A suitable control (e.g., TD cells or a cell line with very low endogenous ABC1 expression) can be used as a control in the assay to determine if the effect observed is specific to cells expressing ABC1.

In still another assay, uptake of radioactive isotopes into or out of a vesicle can be measured. The vesicles are separated from the extravesicular medium and the radioactivity in the vesicles and in the medium is quantitated and compared.

### Nucleic Acid-Based Assays

ABC1 nucleic acid may be used in an assay based on the binding of factors necessary for ABC1 gene transcription. The association between the ABC1 DNA and the binding factor may be assessed by means of any system that discriminates between protein-bound and non-protein-bound DNA (e.g., a gel retardation assay). The effect of a compound on the binding of a factor to ABC1 DNA is assessed by means of such an assay. In addition to in vitro binding assays, in vivo assays in which the regulatory regions of the ABC1 gene are linked to reporter genes can also be performed.

### Assays Measuring ABC1 Stability

A cell-based or cell-free system can be used to screen for compounds based on their effect on the half-life of ABC1 mRNA or ABC1 protein. The assay may employ labeled mRNA or protein. Alternatively, ABC1 mRNA may be detected by means of specifically hybridizing probes or a quantitative PCR assay. Protein can be quantitated, for example, by fluorescent antibody-based methods.

In Vitro mRNA Stability Assay

1. Isolate or produce, by in vitro transcription, a suitable quantity of ABC1 mRNA;

2. Label the ABC1 mRNA;

3. Expose aliquots of the mRNA to a cell lysate in the presence or absence of a compound being tested for its ability to modulate ABC1 mRNA stability; and

4. Assess intactness of the remaining mRNA at suitable time points.

In Vitro Protein Stability Assay

1. Express a suitable amount of ABC1 protein;

2. Label the protein;

3. Expose aliquots of the labeled protein to a cell lysate in the presence or absence of a compound being tested for its ability to modulate ABC1 protein stability; and

4. Assess intactness of the remaining protein at suitable time points

In Vivo mRNA or Protein Stability Assay

1. Incubate cells expressing ABC1 mRNA or protein with a tracer (radiolabeled ribonucleotide or radiolabeled amino acid, respectively) for a very brief time period (e.g., five minutes) in the presence or absence of a compound being tested for its effect on mRNA or protein stability;

2. Incubate with unlabeled ribonucleotide or amino acid; and

3. Quantitate the ABC1 mRNA or protein radioactivity at time intervals beginning with the start of step 2 and extending to the time when the radioactivity in ABC1 mRNA or protein has declined by approximately 80%. It is preferable to separate the intact or mostly intact mRNA or protein from its radioactive breakdown products by a means such as gel electrophoresis in order to quantitate the mRNA or protein.

### Assays Measuring Inhibition of Dominant Negative Activity

Mutant ABC1 polypeptides are likely to have dominant negative activity (i.e., activity that interferes with wild-type ABC1 function). An assay for a compound that can interfere with such a mutant may be based on any method of quantitating normal ABC1 activity in the presence of the mutant. For example, normal ABC1 facilitates cholesterol efflux, and a dominant negative mutant would interfere with this effect. The ability of a compound to counteract the effect of a dominant negative mutant may be based on cellular cholesterol efflux, or on any other normal activity of the wild-type ABC1 that was inhibitable by the mutant.

### Assays Measuring Phosphorylation

Glu89 in the wild-type chicken ABC1 polypeptide is likely to be part of a phosphorylation motif, and thus elimination of this phosphorylation motif by the E_K ABC1 mutation in the WHAM chicken (discussed further below) may be responsible for reduced biological activity of WHAM chicken ABC1. Thus, compounds that modulate the phosphorylation state of ABC1 are likely to be clinically relevant modulators of human ABC1 activity.

The effect of a compound on ABC1 phosphorylation can be assayed by methods that quantitate phosphates on proteins or that assess the phosphorylation state of a specific residue of a ABC1. Such methods include but are not limited to ³²P labeling and immunoprecipitation, detection with antiphosphoamino acid antibodies (e.g., antiphosphoserine antibodies), phosphoamino acid analysis on 2-dimensional TLC plates, and protease digestion fingerprinting of proteins followed by detection of ³²P-labeled fragments.

### Assays Measuring Other Post-Translational Modifications

The effect of a compound on the post-translational modification of ABC1 is based on any method capable of quantitating that particular modification. For example, effects of compounds on glycosylation may be assayed by treating ABC1 with glycosylase and quantitating the amount and nature of carbohydrate released.

### Assays Measuring ATP Binding

The ability of ABC1 to bind ATP provides another assay to screen for compounds that affect ABC1. ATP binding can be quantitated as follows.

1. Provide ABC1 protein at an appropriate level of purity and reconstitute it in a lipid vesicle;

2. Expose the vesicle to a labeled but non-hydrolyzable ATP analog (such as gamma ³⁵S-ATP) in the presence or absence of compounds being tested for their effect on ATP binding. Note that azido-ATP analogs can be used to allow covalent attachment of the azido-ATP to protein (by means of UV light), and permit easier quantitation of the amount of ATP bound to the protein; and

3. Quantitate the amount of ATP analog associated with ABC1

### Assays Measuring ATPase Activity

Quantitation of the ATPase activity of ABC1 can also be assayed for the effect of compounds on ABC1. This is preferably performed in a cell-free assay so as to separate ABC1 from the many other ATPases in the cell. An ATPase assay may be performed in the presence or absence of membranes, and with or without integration of ABC1 protein into a membrane. If performed in a vesicle-based assay, the ATP hydrolysis products produced or the ATP hydrolyzed may be measured within or outside of the vesicles, or both. Such an assay may be based on disappearance of ATP or appearance of ATP hydrolysis products.

For high-throughput screening, a coupled ATPase assay is preferable. For example, a reaction mixture containing pyruvate kinase and lactate dehydrogenase can be used. The mixture includes phosphoenolpyruvate (PEP), nicotinamide adenine dinucleotide (NAD⁺), and ATP. The ATPase activity of ABC1 generates ADP from ATP. The ADP is then converted back to ATP as part of the pyruvate kinase reaction. The product, pyruvate, is then converted to lactate. The latter reaction generates a colored quinone (NADH) from a colorless substrate (NAD+), and the entire reaction can be monitored by detection of the color change upon formation of NADH. Since ADP is limiting for the pyruvate kinase reaction, this coupled system precisely monitors the ATPase activity of ABC1.

### Assays Measuring Cholesterol Efflux

A transport-based assay can be performed in vivo or in vitro. For example, the assay may be based on any part of the reverse cholesterol transport process that is readily re-created in culture, such as cholesterol or phospholipid efflux. Alternatively, the assay may be based on net cholesterol transport in a whole organism, as assessed by means of a labeled substance (such as cholesterol).

For high throughput, fluorescent lipids can be used to measure ABC1-catalyzed lipid efflux. For phospholipids, a fluorescent precursor, C6-NBD-phosphatidic acid, can be used. This lipid is taken up by cells and dephosphorylated by phosphatidic acid phosphohydrolase. The product, NBD-diglyceride, is then a precursor for synthesis of glycerophospholipids like phosphatidylcholine. The efflux of NBD-phosphatidylcholine can be monitored by detecting fluorescence resonance energy transfer (FRET) of the NBD to a suitable acceptor in the cell culture medium. This acceptor can be rhodamine-labeled phosphatidylethanolamine, a phospholipid that is not readily taken up by cells. The use of short-chain precursors obviates the requirement for the phospholipid transfer protein in the media. For cholesterol, NBD-cholesterol ester can be reconstituted into LDL. The LDL can efficiently deliver this lipid to cells via the LDL receptor pathway. The NBD-cholesterol esters are hydrolyzed in the lysosomes, resulting in NBD-cholesterol that can now be transported back to the plasma membrane and efflux from the cell. The efflux can be monitored by the aforementioned FRET assay in which NBD transfers its fluorescence resonance energy to the, rhodamine-phosphatidylethanoline acceptor.

### Animal Model Systems

Compounds identified as having activity in any of the above-described assays are subsequently screened in any available animal model system, including, but not limited to, pigs, rabbits, and WHAM chickens. Test compounds are administered to these animals according to standard methods. Test compounds may also be tested in mice bearing mutations in the ABC1 gene. Additionally, compounds may be screened for their ability to enhance an interaction between ABC1 and any HDL particle constituent such as ApoAI, ApoAII, or ApoE.

### The Cholesterol Efflux Assay as a Drug Screen

The cholesterol efflux assay measures the ability of cells to transfer cholesterol to an extracellular acceptor molecule and is dependent on ABC1 function. In this procedure, cells are loaded with radiolabeled cholesterol by any of several biochemical pathways (Marcil et al., Arterioscler. Thromb. Vasc. Biol. 19:159-169, 1999). Cholesterol efflux is then measured after incubation for various times (typically 0 to 24 hours) in the presence of HDL3 or purified ApoAI. Cholesterol efflux is determined as the percentage of total cholesterol in the culture medium after various times of incubation. ABC1 expression levels and/or biological activity are associated with increased efflux while decreased levels of ABC1 are associated with decreased cholesterol efflux.

This assay can be readily adapted to the format used for drug screening, which may consist of a multi-Well (e.g., 96-well) format. Modification of the assay to optimize it for drug screening would include scaling down and streamlining the procedure, modifying the labeling method, using a different cholesterol acceptor, altering the incubation time, and changing the method of calculating cholesterol efflux. In all these cases, the cholesterol efflux assay remains conceptually the same, though experimental modifications may be made. A transgenic mouse overexpressing ABC1 would be expected to have higher than normal HDL levels.

### Knock-Out Mouse Model

An animal, such as a mouse, that has had one or both ABC1 alleles inactivated (e.g., by homologous recombination) is likely to have low HDL-C levels and higher than normal triglyceride levels, and thus is a preferred animal model for screening for compounds that raise HDL-C levels or lower triglyceride levels. Such an animal can be produced using standard techniques. In addition to the initial screening of test compounds, the animals having mutant ABC1 genes are useful for further testing of efficacy and safety of drugs or agents first identified using one of the other screening methods described herein. Cells taken from the animal and placed in culture can also be exposed to test compounds. HDL-C and triglyceride levels can be measured using standard techniques, such as those described herein.

### WHAM Chickens: an Animal Model for Low HDL Cholesterol

Wisconsin Hypo-Alpha Mutant (WHAM) chickens arose by spontaneous mutation in a closed flock. Mutant chickens came to attention through their a Z-linked white shank and white beak phenotype referred to as ‘recessive white skin’ (McGibbon, 1981) and were subsequently found to have a profound deficiency of HDL (Poernama et al., 1990).

This chicken low HDL locus (Y) is Z-linked, or sex-linked. (In birds, females are ZW and males are ZZ). Genetic mapping placed the Y locus on the long arm of the Z chromosome (Bitgood, 1985), proximal to the ID locus (Bitgood, 1988). Examination of current public mapping data for the chicken genome mapping project, ChickMap (maintained by the Roslin Institute; http://www.ri.bbsrc.ac.uk/chickmap/ChickMapHomePage.html) showed that a region of synteny with human chromosome 9 lies on the long arm of the chicken Z chromosome (Zq) proximal to the ID locus. Evidence for this region of synteny is the location of the chicken aldolase B locus (ALDOB) within this region. The human ALDOB locus maps to chromosome 9q22.3 (The Genome Database, http://gdbwww.gdb.orgI), not far from the location of human ABC1. This comparison of maps showed that the chicken Zq region near chicken ALDOB and the human 9q region near human ALDOB represent a region of synteny between human and chicken.

Since a low HDL locus maps to the 9q location in humans and to the Zq region in chickens, these low HDL loci are most probably located within the syntenic region. Thus we predicted that ABC1 is mutated in WHAM chickens. In support of this, we have previously identified an E_K mutation at a position that corresponds to amino acid 89 of human ABC1. This non-conservative substitution is at a position that is conserved among human, mouse, and chicken, indicating that it is in a region of the protein likely to be of functional importance.

Discovery of the WHAM mutation in the amino-terminal portion of the ABC1 protein also establishes the importance of the amino-terminal region. This region may be critical because of association with other proteins required to carry out cholesterol efflux or related tasks. It may be an important regulatory region (there is a phosphorylation site for casein kinase near the mutated residue), or it may help to dictate a precise topological relationship with cellular membranes (the N-terminal 60 amino acid region contains a putative membrane-spanning or membrane-associated segment).

The amino-terminal region of the protein (up to the first 6-TM region at approximately amino acid 639) is an ideal tool for screening factors that affect ABC1 activity. It can be expressed as a truncated protein in ABC1 wild-type cells in order to test for interference of the normal ABC1 function by the truncated protein. If the fragment acts in a dominant negative way, it could be used in immunoprecipitations to identify proteins that it may be competing away from the normal endogenous protein.

The C-terminus also lends itself to such experim nts, as do the intracellular portions of the molecule, expressed as fragments or tagged or fusion proteins, in the absence of transmembrane regions.

Since it is possible that there are several genes in the human genome which affect cholesterol efflux, it is important to establish that any animal model to be used for a human genetic disease represents the homologous locus in that animal, and not a different locus with a similar function. The evidence above establishes that the chicken Y locus and the human chromosome 9 low HDL locus are homologous. WHAM chickens are therefore an important animal model for the identification and testing of drugs that modulate cholesterol efflux.

The WHAM chickens' HDL deficiency syndrome is not, however, known to be associated with an increased susceptibility to atherosclerosis in chickens. This may reflect the shorter lifespan or, more likely, the impaired absorption of dietary cholesterol in these chickens. We propose the WHAM chicken as a model for human low HDL for the development and testing of drugs to raise HDL in humans. Such a model could be employed in several forms, through the use of cells or other derivatives of these chickens, or by the use of the chickens themselves in tests of drug effectiveness, toxicity, and other drug development purposes.

### Therapy

Compounds of the invention, including but not limited to, ABC1 polypeptides, ABC1 nucleic acids, other ABC transporters, LXR-modulating compounds, RXR-modulating compounds, and any therapeutic agent that modulates biological activity or expression of ABC1 identified using any of the methods disclosed herein, may be administered with a pharmaceutically-acceptable diluent, carrier, or excipient, in unit dosage form. Conventional pharmaceutical practice may be employed to provide suitable formulations or compositions to administer such compositions to patients. Any appropriate route of administration may be employed, for example, intravenous, perenteral, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspension; for oral administration, formulations may be in the form of tablets or capsules; and for intranasal formulations, in the form of powders, nasal drops, or aerosols.

Methods well known in the art for making formulations are found in, for example, Remington: The Science and Practice of Pharmacy, (19th ed.) ed. A. R. Gennaro AR., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

### Compounds

In general, novel drugs for the treatment of aberrant lipid levels and/or CVD are identified from large libraries of both natural product or synthetic (or semi-synthetic) extracts or chemical libraries according to methods known in the art. Those skilled in the field or drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and. synthetically produced libraries are produced, if desired, according to methods known in the art, e.g., by standard extraction and fractionation. methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their HDL-raising, triglyceride-lowing, or anti-CVD activities, or known for their ability to modulate ABC1 gene expression or ABC1 biological activity should be employed whenever possible.

When a crude extract is found to modulate ABC1 gene expression, ABC1 biological activity, or a combination thereof, further fractionation of the positive lead extract is necessary to isolate chemical constituent responsible for the observed effect. Thus, the goal of the extraction, fractionation, and purification process is the careful characterization and identification of a chemical entity within the crude extract having HDL-raising, triglyceride-lowering, or anti-CVD activities, ability to modulate ABC1 gene expression, or a combination thereof. The same in vivo and in vitro assays described herein for the detection of activities in mixtures of compounds can be used to purify the active component and to test derivatives thereof. Methods of fractionation and purification of such heterogeneous extracts are known in the art. If desired, compounds shown to be useful agents for the treatment of pathogenicity are chemically modified according to methods known in the art. Compounds identified as being of therapeutic value are subsequently analyzed using any standard animal model of diabetes or obesity known in the art.

It is understood that compounds that modulate activity of proteins that modulate ABC1 gene expression or activity are useful compounds for modulating HDL-C levels and triglyceride levels. Exemplary compounds are provided herein; others are known in the art.

Compounds that are structurally related to cholesterol, or that mimic ApoAI or a related apolipoprotein, and increase ABC1 biological activity are particularly useful compounds in the invention. Other compounds, known to act on the MDR protein, can also be used or derivatized and assayed for their ability to increase ABC1 biological activity. Exemplary MDR modulators are PSC833, bromocriptine, and cyclosporin A. Other examples of compounds that may be assayed for the ability to increase ABC1 biological activity include oxysterols and their derivatives.

### Screening Patients Having Low HDL-C or High Triglyceride Levels

ABC1 expression, biological activity, and mutational analysis can each serve as a diagnostic tool for low HDL or higher than normal triglyceride levels; thus determination of the genetic subtyping of the ABC1 gene sequence can be used to subtype low HDL or higher than normal triglyceride individuals or families to determine whether the low HDL or higher than normal triglyceride phenotype is related to ABC1 function. This diagnostic process can lead to the tailoring of drug treatments according to patient genotype, including prediction of side-effects upon administration of HDL increasing or triglyceride lowering drugs (referred to herein as pharmacogenomics). Pharmacogenomics allows for the selection of agents (e.g., drugs) for therapeutic or prophylactic treatment of an individual based on the genotype of the individual (e.g., the genotype of the individual is examined to determine the ability of the individual to respond to a particular agent).

Agents, or modulators which have a stimulatory or inhibitory effect on ABC1 biological activity or gene expression can be administered to individuals to treat disorders (e.g., cardiovascular disease, low HDL cholesterol, or a higher than normal triglyceride level) associated. with aberrant ABC1 activity. In conjunction with such treatment, the pharmacogenomics (i.e., the study of the relationship between an individual's genotype and that individual's response to a foreign compound or drug) of the individual may be considered. Differences in efficacy of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of ABC1 protein, expression of ABC1 nucleic acid, or mutation content of ABC1 genes in an individual can be determined to th reby select appropriate agent(s) for therapeutic or prophylactic treatment of the individual.

Pharmacogenomics deals with clinically significant hereditary variations in the response to drugs due to altered drug disposition and abnormal action in affected persons (Eichelbaum, M., Clin. Exp. Pharmacol. Physiol., 23:983-985, 1996; Linder, M. W., Clin. Chem., 43:254-266, 1997). In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). Altered drug action may occur in a patient having a polymorphism (e.g., an single nucleotide polymorphism or SNP) in promoter, intronic, or exonic sequences of ABC1. Thus by determining the presence and prevalence of polymorphisms allow for prediction of a patient's response to a particular therapeutic agent. In particular, polymorphisms in the promoter region may be critical in determining the risk of HDL deficiency, higher than normal triglyceride level, and CVD.

In addition to the mutations in the ABC1 gene described herein, we have detected polymorphisms in the human ABC1 gene (FIG. 4). These polymorphisms are located in promoter, intronic, and exonic sequence of ABC1. Using standard methods, such as direct sequencing, PCR, SSCP, or any other polymorphism-detection system, one could easily ascertain whether these polymorphisms are present in a patient prior to the establishment of a drug treatment regimen for a patient having low HDL, a higher than normal triglyceride level, cardiovascular disease, or any other ABC1-mediated condition. It is possible that some these polymorphisms are, in fact, weak mutations. Individuals harboring such mutations may have an increased risk for cardiovascular disease; thus, these polymorphisms may also be useful in diagnostic assays.

### Other Embodiments

All publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

What is claimed is:

1. A method for identifying a modulator of HDL-levels in an animal, comprising:
(a) contacting a cell with a test compound wherein said cell expresses an ABCA1-gene and comprises an LXR site, wherein said test compound is a modulator of LXR-activity and wherein said contacting occurs under conditions facilitating ABCA1-gene expression, and
(b) determining an increase in said ABCA1-gene expression as a result of said contacting,
wherein an increase in ABCA1-gene expression indicates ability to modulate HDL-levels in an animal thereby identifying said test compound as a modulator of HDL-levels.

2. The method of claim 1 wherein said test compound increases LXR-activity.

3. The method of claim 1 wherein said increase in ABCA1-gene expression is an increase in transcription of said ABCA1-gene.

4. The method of claim 1 wherein said increase in ABCA1-gene expression is an increase in translation of a protein encoded by said ABCA1-gene.

5. The method of claim 1 wherein said cell is a mammalian cell.

6. The method of claim 1 wherein said cell is a recombinant cell engineered to express said ABCA1-gene.

7. The method of claim 6 wherein expression of ABCA1-gen does not occur absent said engineering.

8. The method of claim 1 wherein said cell is a recombinant cell engineered to contain said LXR site.

9. The method of claim 8 wherein the cell does not contain said LXR site absent said engineering.

10. The method of claim 5 wherein said mammalian cell is a fibroblast.

11. The method of claim 5 wherein said mammalian cell is a macrophage.

12. The method of claim 5 wherein said mammalian cell is a mouse cell.

13. The method of claim 5 wherein said mammalian cell is a human cell.

14. The method of claim 1 wherein said ABCA1-gene encodes a mammalian ABCA1.

15. The method of claim 1 wherein said ABCA1-gene encodes a protein having an amino acid sequence with at least 50% identity to the amino acid sequence of SEQ ID NO: 5.

16. The method of claim 1 wherein said ABCA1-gene encodes a protein having an amino acid sequence with at least 85% identity to the amino acid sequence of SEQ ID NO: 5.

17. The method of claim 1 wherein said ABCA1-gene encodes a protein having an amino acid sequence with at least 90% identity to the amino acid sequence of SEQ ID NO: 5.

18. The method of claim 1 wherein said ABCA1-gene encodes a protein having an amino acid sequence with at least 95% identity to the amino acid sequence of SEQ ID NO: 5.

19. The method of claim 1 wherein said ABCA1-gene encodes a protein that comprises the amino acid sequence of SEQ ID NO: 5.

20. The method of claim 1 wherein said ABCA1-gene encodes a protein that comprises amino acid residues 1-60 of SEQ ID NO: 5.

21. The method of claim 1 wherein said LXR is LXRα.

22. The method of claim 1 wherein said test compound is an oxysterol.

23. The method of claim 1 wherein said increase in ABCA1-gene expression is the occurrence of ABCA1-gene expression where said expression does not occur absent said contacting.

24. A method for identifying an HDL-modulating agent, comprising administering to an animal an agent having ABCA1-modulating activity in the method of claim 1 and determining a change in HDL-levels in said animal following said administering.

25. The method of claim 24 wherein said agent was first identified as having ABCA1-modulating activity in the method of claim 1.

26. A method of treating a condition in an animal comprising administering to an animal afflicted therewith a therapeutically effective amount of an agent that has HDL-level modulating activity in the method of claim 1 and wherein said condition is a member selected from the group consisting of a lower than normal HDL-l vel, a higher than normal triglyceride level, inflammation, diabetes, obesity and cardiovascular disease.

27. The method of claim 26 wherein said agent was first identified as having HDL-level modulating activity using the method of claim 1.

28. The method of claim 26 wherein said condition is coronary artery disease.

29. The method of claim 26 wherein said inflammation is a member selected from the group consisting of rheumatoid arthritis, systemic lupus erythematosis (SLE), hypo-thyroidism, hyperthyroidism and inflammatory bowel disease.

30. The method of claim 26 wherein said condition is diabetes.

31. The method of claim 26 wherein said condition is obesity.

32. The method of claim 26 wherein said animal is a human being.

33. A method of increasing HDL-levels in an animal having lower than normal HDL-level comprising administering to said animal an effective amount of an agent that has HDL-level modulating activity in the method of claim 1.

34. The method of claim 33 wherein said agent was first identified as having HDL-level modulating activity using the method of claim 1.


SEQUENCE LISTING (256 sequences)

SEQ ID NO: 1 - DNA, 183999 bp, homo sapien
  misc_feature (1)...(183999): n = a, t, g, c or none; k = g or t; y = c or t; m = a or c; s = c or g; h = a, c or t; b = c, g or t; r = a or g
gtctataatggcatgccacagggctctaaaactttgcagttttatcattaactcaaatgaaatgtatacatgccgctgactcaacattttgagagacaacaaatacaatgaatatcaagatacatatatatataatatgtatttctttttgagatggagtttcactgttgttgtccaggctggagtacaatagcacgatcttggctcactgcaacctctgcctcccaggttcaagcaattgtcctgcctcagcctcccaagtagctaggattacaggcatgtgccaccacacctggctaattttgtattttttaagtagagatggggtttcaccatgttggtcaggctggtctcgaactcctgacctcaggtgatccacctgcctcagcatctcaaagtgctgggattacaggtgtgagccaccccacccggccatatatatatatttttgagatggagtcttactctgtcacccaggctggagtgcaatggcttgatctcggctcattgcaacctctgcctcccatgttcagatgattctcctgcctcagcctctcaagaagctgggattacaggtgcatgccaccatgcccaactaatttttatatttcatcatggggtttcaccatgttggccagggtggtgtcgaactcctgacctcaagtgatctgcctgccttcggcctcccaaagtgctgggattaccggcatgaaccaatacgcttggcaatatttttttaagaaaaaaaaatttcaggttgcaacagcatccaaaaagtaaccaatgattttaggtgaagggtgaagacaaatgtaaactcttttttttttttttgaaatggcgtcttgctctgtcgcctaggctggagtgcattggtgcaattgcgactcactgcaacctccacctcctggactcaaacgattctcctgcctcagcctcccgagtagctgggattataggctcgcgccgccacgccccgctaatttttgtgtttttagtagagacggggtttcaccatgttggccaggctggtctcaaactcttgacttcaagtgatccgcctgccttggcctcccaaagtgctgggattacaggcttgagccacccggtgaaatgtaaattgttaaacctgtgtttttgaaaatgcataagtataggataagggagaattgactttctgaagaccagaacattttagtcaatttcaaacacaatgtgagtcaattgtataaaacaggttccttatcctgatgaggataagaatagtatccttgtcagatggaaatgcccattcagctgtactttctagtggttacgcccatagtagcactgttgatggaaccaggtatctgactttaggaaagatgttccccaactggagctgacccagaggagcctgaccaacttggggaaagtttaaagatctcatcacgtggagaataggggaaggcaccaacacgtattgagtgtctactttgagcttaagggagaaggagaaaaggcagggaataaacggaggatggaataagaataggtaatcttccttaggtttaaataataagtgcttgccataggaaggagccccagaacacagttatcaataatagagactcacacagagcattctacactagagctgctgtcctcttgaccagaataagggtaaggtgtgtgtgcgtgtccaggaaagtaggcagctaggaggtgatcagagcatacactactgccgccacaattctaagtgtcttcccctaggggaatcctatttcttctcaggcacatttgtttattcattccatgttcactcttgttatttacttcttgccaggctttgtgttaagaattggggaaacaaggttgaataaacccagtctgtaaagaaaaggagctcacagtctggaggggcaaatgggcattgtgcctgcaagttggcccactgagagcctaagaagtgaagttatgaatccaggattactcagttatcaatgaagtgattaaacatcatccatacagaccttcagagctggagggaattttggatacctactcagcacatagttttcaaacagtgccttgtggaaccctagggcatttcttagggattgctgtgtgtgagagaggagattgaatcagaaggtgtctgggaccattctctactcacacttcaagcagagcagctccacttctatctgtattattatttttattatttatttattntatttattttgagacggagtctcgccctgtcgcccaggctggagtgcagtggcacgatctcagctcactgcaacctctgcctcccgggttcaagagattctcctgcctcagcctcctgagtagctggaattataggcctatgccaccacgcccagctaatttttgtatttttagtagagacggggtttcaccatgttggtcaggctggtctcgaactcctgaccttgtgatccaccggccttcgcctcccaaagtgctgggattacaggcattagccaccatgccccgcctatctgtattatttattcattattgctatgtgaatgaacctgaagaatgcttactgttactgctaagtatttaaccacaccccatgcccatgcaggatgatagtgaatagtggccaaaagatactataattagactcatgtaattaaggaatatttttgtcttgtacctattatgtgcctataaagactatgaaatctatttattcagtgatttattggaataccaaataagcaaagatcctatgtgctaaagattctaatattgtgctaagattttccttcagatgtttggctttctcaaattccctgagggctagaactttgccctactcatttgtgtttcccaagtgtctaacgcagtgcctgacacatacaggatctccaaacgcttgctgaatgtgtgaggaaggaattaaaataatgtaccgccgggcaaagtggctcatgcctataatcccagcactttgagagaccgaggtgggcagatcacttgaggtcaggagctcgagcccagcctggccaacatggtgaaaccccgtctctactaagaatacaaaaattagccgcgagttgtggcaggcgcctgtaatcccagctactcggaaggctgaagtgggagaatcacttgaacctgggaagcagaggtcgtagtgagccgagatcagaccgctgagctccagcctgggtgacagagcaagactccatctcgaaaaataaaagtaaaataaaataatgtactaactggacccagaacagattttccaattgattattgacaacaaaggaatctgaattatttaataaggtgaataagtacatattcatatatatatgtatatgtgtgtgtatttacatttttataaaagtgtaaaagtatatatactttttttcctttcttcaggtagaaacctctcctagattgtcactgaataaacattagcactaactatggcaatcaaatcacatattgattggtgtcagagaagaattgaacattcaactctgaagcagtgttatttcttccatctgcaaacactctgtcccccatcchttctttgtgtatcctggaatccaagtcataaataatgataggtatttctgtcaaaggtatttctcagaggagtgatatgtaactccctttcctctgatacactgactcactaagcagctacccttgtgaattccaattagcaatacttcttgctatgtctggtccaactttcagacaaacctagtgttcaggattcctatagccatttataggtgtagacaggaagcattcagatatccccagaggtacctgataaccagctgatccatgactgtctgtcttgggcttggccagcttgaaatcttgacattgtggttctcncccagagaaggtgccttttggatgtgagataaagacattatgactagatagtgcatggtgagggtgtttttctagttttaccgaagtgttgatctgtaaagctgctaccagcacacacnnanncacacacacacacacacacacacacacaattaaccacagatatcctcatgggaaattgtcttaggaaagaatggaagccaagattttatatatagaccacagaaggtgatgggtaatgttcttggaagggagttgacaggcaatagctataagttaactcaggaaagcaaagaaaatccccaaagagctaagggagaggttagagattctgctttttattagcaattcatagctctcaagtttcatacagtctttaaggctcccctcttcatatagaataaatgaaattattttataaattgttcctcagattcgtatctgtacattctgggaccacgagttgtagcaggatgtgattttcctcattctgggcatctaagttctacagttaaggacactgaaacaaacctttagtcgaataaagattggcacattgtttcttctcccataaacattgaatggtccaggaagggccaggtgtggtggctcacacctgtaatcccagcactttgggaggccaaggcaggtagatcacccgaggtcaggagttcaagaccagcctggccaagatggtgaaaccctgtctctactaaaaatacaaaaaggaggagtggtagtcccagcttttgcatgaatgtccacattgggactttatcttaaatgtgcaagacacaactcttggccaggcgtggtgaaccgagattgtgccactgcactccagccagagaatgcactccagcctgagcaacagagccagactccttctcagaaaaaaaaaaaaaaaaaaaaagacaactcttaaattggtgttctactgtccttgaaccaccatacctttgcagaagtcattctagatcatcaatctgaccataaaacacagtttgccaaactggcaactaactgcacctatttgtgattagtttggaagaaacttgaaagtgccccttttaactgtccacttattccctgcctgggcacggaattctctcccacgcttaataacggacacttttaaaaattatttttactccccacagagttggcagattgctgtttcagagagttaaatggaatgcctccagtggaagtatcccgttttctagaatggaaagtctatcttcacagtgtcataatccaggtgccctgggctgagacttcccctgcctaggcggtaccctgggtagcacagctgaactggctgtgaactaaacattcattttttattagcagccaggctggacagagatcacacaagaccaacctgacaacagcagcatgctgctctgcttcagaagtaattttttttttttttgagacaagagccttgctctatcacccaggcaaagtgcagtggcatgatctcggctcactgcaacctccaccttctgggatcaagcgattctcctgcctcagcctcccaagtagctgggattacaggcgcatgccaccatgcctggctaattttttgkacttttgtattttttttttttttgagatggagtcctcgctctgtcgcccgggctggagtggcctgagccaccgcacctggcccttcagaagtaatttttaaggaaggatcttgtcctgggtggggggttgggcaaggctgtgaaaagcaaacaaaaatcacatgtggcctaataggaggccagtggaaatacacatgatgaaaaagaaacttacaaaagcacattattaatttctgaacatgctaataccatccaataacaataagatctaatatttattgaatgctgattactcatcagctacattccaagtacttctacatgtacagttatgtgacacataagatgtttaggtcaccactgacagaatgtatgatggtgaccccataagattacaaaaattcctattgcctagtgacatatgtagcaagatgcattactcacgtgtaataagtaatgtgatgctgctgtaaacaaacctactgtgctgccatgcctatagaagtctaggacgggctgggcacagtggctcatgcctgtaatcccagtacggtgggaggccaaggcaggccgattgcttgagcccagaagttcaagaccagccggggcaacatagtgagagcctgtctctacaaaaaatacaagaacaaattagccaggcatggtggcgcatgcctttggtcccagctactcaggaggctgaggcaggaggatcacttaagcccaggaggttgagtctgcagtgagccatgatggtgccactgcactccaacctggagagagagtgagacccggttttaaaaaaaaaaaaaagcctaacaaataaaagtatgtatagtacagaatacttggtaatgataaacagtatgttactggtttatgtgtttactatactttttattttttagagtatactcctacttatttaaaaagaaaaaagaaagttaactgtaaaacaggcttagacaggtccttcaggaggtattccagaagaaaggcattgttatcacaggagatgaaagctccatgcatgttactgcccttgaagaccttccagtgggacaagatgtgaggtggaagtctatgatagtgatgatcctgaccctgtgtaggcctaggctaatatgtgtgtttttaacatattagtttttaacaacaaagtttaatcagttaaaacaatttttttaatagaaaaaagctcatagaataaggatatnaaagaaangacaatgttttgtttgtttgtttgtttgttttttgagacagagtctcgctnccatcacctgggctggagtgcagtggcacaatcttggctcagtgcaacctctgattcccaggttcaagtaattctcatgcctcaacctcccaagtagctgggattacaggcgcccatcaccacacccaactaacttttgtctttttagtagagatggggtttcatcatattggccaggcttgtcttgaactcctgacctcaggtgatccaccctcctcggcctcccaaagtgctgggattacggttgtaagccatcgtgcccggccagaaagaaaatgtttgtacagtatgtacgatgtatttgttttaagctaagtgttattatgaatgaatcatgtgtggtgtagtggctcatgcccgtaatcgcatcactttgggagaccgaggacggaggatagcttgagtccagagttctagaccagcctgaacaacgtggtgagacctcgtctctacgaaaatcatcaaaaattatccatgcgtgggggtacatacctatagtcctagctactcaggagactgaggtgaggggattgcttgaactcaagaagttgaagctgcagtgagctataattgtaccactgcatttcggcctaggtgaccccgcctcaaagaataataataaaagctgggcacggtggctcacgcctgtaatcccagcactttgggaggccaaggcgggcggatcacttgaggtcaggagtttgagaccagcctggctgacatggtgagaccccgtcgctactaaatacaaaaaattagccggtcgtggtggtgtgtgcctgtaatcccagctactcaggaagctgaggtgggagaattgcttgaacctgggaggcagaggttgcagtgagccgagatagcgccactgcactccagcctgagcaaaaaacaataataaataaataagtttgaaaatttaaaatgtttataaaattaaaaagttacagtgagctaagatttagtattaaagaatttttttataaacatgtagtgtaactctacagtgttaataaactctacagtagtgtacagtgacatcctaggccttcacattcactcatcactcactgactcacccagagcaacttccagtcctgaaagctccattcatggtaagtttcctatacaggcgtgccatttaaaaaatcttttaggccaggcgcggcggctcacgnctgtaatcccagcactttgggaggctgaggcaggcggatcacgagctcaggagatcgagaccatcctggctaacacggtgaaaccccgtctctactaaaaatacaaaagattagctgggtgtggtggtgggcgcctgtagtcccagctactcgggaggctgaggcaggagaatggcatgaacccaggaggcggagcttgacagtgagctgagatcacaccactgcgctccagcctgggccacagagcaagactccatctcaaaaaaaaaaaaaatcttttatactatattcttaatgtaccttttctatgtttagatacacaaataccactgtattacaattgcctactgtattcagtacagtaacatgctatatggttacgtagcctaggaacaataggctatattgttcctaggtatagggatgtggtatcctataccatctaggtttgtgtaagtttattttatgatgtttgcatgacgacagagtcacctaaggactcatttcttagaatatatttgtagttaagcaatgcatgactctattgactcatgaattcttaccacagacctatggggcagtactattgttaccctcattttataaatgataaaactgaggtacagagacagtaaataacttgaccacggtcattcagctactcaagagtcaaggctgggatttaaaaccagatcacatggtttcagagtgttcacacttacctactatactgtctcaagagcaaggatgttttggttcacttgacaaatgaagatagggacctctttcattataagcctattttaggctaaaatagaagggaaggggacacagtgaatccaggccttctggcatggctcctcagccctttctgagctggcctgggacagccttcctacctcactgatgccacttcctactgagcgactttcctgcctacctcactgatgccacttcctactgagcgactttcctgggctccagacccagtaagcgactttgcctgcacccaccttattttgctctactccctgtgcttttatgcctttacccatctgccctggaaagctcttctaacctttgaatgggtaaaggcataaatgtatgctggagaaatcctcagctcagggccaggcacgctggctcacgcctgtaatcccagcactttgggaggccgaggtgggtagatcacctgaggtcgggagttggagaccagcctgaccaacatggagaaacccctgtctctactaaaaatacaaaattagctgggcgtgatggcacatgcctgtaatcccacagctactcgggaggctgaggcaggagaattgcttgaacctgggaggcggaggttgcagtgagccaagattgcgcctctgcactccagcctgggcgacagagctagactctgcctcaaaaaaaaaaaacaaaaaaaagaaaaaaaagaaaaaaaagaaatcctcagctgagttgtcaactcctctttgaaactttctcagacctttcaggctgagtcgtcgctcatttgtgcttcctcagttcctggcttctaccttcttcatagcttgtttcatgtaatgttaattcttacttgctttctccctcttctaagctgagagctacttcaaagcatgggtaggacctagcacggtgtatgggacatgggtggtaccccgtaaatgtttactgaaaaaaaaatgcctaaagcaattgttaacatcatcagatagataattatgggcattcagagattctgtcttcaagcttatataaagaacttatttttggctctaattatcctgataattttctcattactttcacttattgtggcttgtggatcaattgttgacattttataaacatttcactatttgacaatgatgatactaaaatacgaattaagcaaccattctaaagatagtgatgatgataacatatacgctggtaacatctttattttcagccgtatcatggaatcctctgtttccattctgctaggtaggcaggtatgcaggtagaacttgtgagaggatatgatttttgtttccatcttagatatgacaggaacttggaatttttgacataaatgacgaacatccgggattcttaaacaatctttaaaaatggaatgccttaaaagctgggcgcagtggctcacgcttataatcccagcactttgggaggctgaggcaaatggatcacttgagttcaggagttcaagaccagcctggccaacatggtgaaaccccatctctactacaaatacaaatattagccgggcgtagtggcaggcgcctgtaatcccagctacttgggagcctgaggcaggggaattgcttgaacccaggaggccttggagattgcagtgagctgagactgcgccattgcactccagcctgggcaacaagagtgaaactccatctccggaaaaaaaaaaaaaaaaaaaaggaatgcctttgggaataatttatttataatttatgtataacatatagacaaaccattagtttgtcttatattttactaaatataaatttagtaaatataaatatttactaaatataaaaactcttagattttactaaagagttacaactaattggcctggcgtggtggctcacacctgtaatcccagcactttaggaggcagaggtgggccgatcacgaggtcaggagatcaagaccatcctggctaacacggtgaaactctgtctctactaaaaaaaaaaaaatacaaaaaattagccgggcgtggtggcaggcccctgtagtcccagctactcaggaggctgaggcaggagaatggcgtgaactaagcagaggcttccctaaaagtgatcttcaggataaaggcagaggaagaggctccatgactgggattggtgttgaggagagccagagaagcaagctacagaaaagagaaaaaattaatatgcaagagagtaaacaacacgaaggaaaagaacccagtgtggaaacactacacgtgagaaaggtgtctgtaaggatgttctacaaagcaaatgcttggatattaattcattgcagcaggagatggtaagcctcatgataaagaaggagaaaaaatcaagtcaagggctctgaggtactgacccaggtatacttgactatgccagcaactgtttagggggagatttgagctacacttgtagcaaaggcaaaatctgtaattagttgtaactctttttttttttgagatggtgtctcgctctgtcccccaggctggagtgcagtggtgtgatcttggctcactgcaagctccgcctcctgggttcaagtgattctccagcctcagcctcccaagtaattgggactacaggcatgcaccaccatgcccagctaatttttgtacttttattacagaccatgttttgccatgttcaccaagctggtctcaagctcctgacctcaagtgatccgtccgcctcggccntcccaaagcgctgagattataggcctgaaccaccgcgcctggcctaaagagatctaattcttagcaaagtttcaccagggagtctctcctcacccccaccccatccttcccacaaagaattagaacaatgtccctactacccctgctgtatctctgactttttactttaaatctcagcagaatattttactaaatgttttgatgtggttatataaaatcatccctgctgacaaggaaacactttttgaaaaaagttttcattatcaaacagtaagtacagctgactgccgtgacctttaacccatttctgagtctcccctcattggacttgggtggaggggactggtaccaataaagtcaaatgcttaataatttatgcaagtgcttgaagaaatttgaagttgaatatttctatcatcttgaaatggagaaagaatctgtaaacagcaaagccagacgccctaaaggaaaagatttacagattaaaataagattgcaatctggtaaaaatatttgcaacacatgtaacagtcagaaagttgaaacacttggtttaacawgagcttttawcagataaataaggaaagaataaacattggattttaaacaccgataaacatgaaaagatgtttaatctttatttttatttaatccatattatttttcagtttaatcaaagaaaatataaatcaaaacaataatacattttatatatatatatatatatatatatatatatatatatatatagtaggcataagttaaagactgataagactgttgaaaagggatgaaaaactaggcttactcataccaatatatatctattaggatggctaaagtaaaaaatactgaaaatatcaagtgctcaaaaggatatggagcaattggaaccctcagacatcgctgrtngagaaaacaaaatggtacagccaccctggagaacagtttagctgtttcttgtaaagttaaacatgcgcttaccatatgactcagcaatctcactcctgggtatttatgctaggaaaaggaaaatttatacttgcacacaaaaaacttgtaagtgaatctttatagcagctctattcataactgccaaaaactgagagaaaatgtcctttaatgtgtgaatggataaaccaactgtgcaacatccatgtaatgaaatactacttagcaataataataataatattaaaaaccccagaaccattgatgcatgcaacaaatatggataaatctcaaaagcattatgctgagtaaaagaagtcagtctgaaggatttcatactctaggattccatttatataacattattgaaacgacaaaattatggggacagagaatagatcagcggttgccaggggtttaggtgtgtggagagggtgtggctataaagaacatgcaagggaatttttttgggagatgaaatggatctgtatcctaattatggtcatggtaacacaaatctatacatgtgtttagattcatagaactgtataccaaaagaaaaaaagtcatttttactctcttaaaatgaaaaaagaaaaagcctgggcattctaacaccttgtttgtgagagtacacattgataccaagttttatggtgggcaattttgctataaatacggaaagtttgtctgttctattattcagcaatcccagttttgcaaaactatgctaaagaatctttgggggccgggcacggtggttcacgcctgtaatctcagcactttgggaggccgaggtggacgatcacctgaggtcaggagtttgagaccagcctggccaacattgggcaaccctgtctctactgaaaatacaaaaattagccgggcatggtggcgcatgcctgtagtccgagctactcgggaggctgaggcaggagaatcacttgaacctgggaggcagaggttgtagtgaactgagatcgtgccaccgcactccagcctgggcaacagagtgagaatccgtctcaaaaaaaaaaaaaaaaaaaaaaacaaaacaaaacaaaaaactttgtgtacgtgtgcaaagagaatacaaagatgatcatggctgcattttttaaatgactataaaaaagaggtacaaccagccaggtaaagtggtgtgcacctgtagtcccagctactcgggagggtgaggtgagaggaacacttgagtccaggagtttcaggccagcctgggcaacatagtgagacccctgtcccaaaaacaaaacaaaacaccaaaatgtctatctgtaggaatttgttttcaagttgtgatacataggtacagtgaaatattatacattcatttaaaatgatgataaaatctgtatttgtttacatgaaaaactgtccactataatgtagtgaaaataatagattacaaacaatatatatggaataaacttgtttagaaacaatttctagaagaaggtacaatggacagaattatctctgggaagtgggtttataatgattctcattttcttctttgtatctttttcatagtctttctacttttgttatgtctggacatttgattatgagcatgtattactgatctattttaaaaattgattttaatttttacaaaaactcatgtaaagcttgaaggttcgcattttagaccatgttaaaattttcctggatcaaaacagacttattcaaatatcttgtaccctgtcctccaaaattgcctgccaaaatacactacaaaagagagcatttagctgcatattttttggactgctgagatcaacaatattatttaccatggcttaaatttttaccctccagtatgtgtgggttacaaacactcttccacatttttgaggcattgcttttgatattttaaatgtaaattcagctgtgcgcggtggctaacgcctataatcccagcactttgggacgctgaggaaaggatcacttgaggtcaggagtttgagaccagtttagctaacgtggtgaaaccccgtctcttttaaaactacaaaaattaaccgggcatggtggcaggcacctgtaatcccagctactcaggaggctgaggcaggagaatcacttgaacttgggagacagaggttgcagtgagccgagatcatgccactgccctccagcctggccacagagcgacactccatctcaaaaaaaaaaaaaaaaaaaaaaaaaaaggccaggcgcagtggctcacgcctgtaatcccagcactttkggagcgccaaggtggssggatcaactgaggttgggagttcacgaccagcctgaccaacatgcagaaacccygtctctactaaaaatacaaaattagccgggtgtggtggtacatgcctgtaatcccagctactcgggaggctgaggcaggagaattgcttgaacccaggaggtgaaggttgtgttgagctgagatcccgccattgcactccagcctgggcaacgagcaaaactctgtctcaaaaaaaccgaaaaaattcccccaaaaaaccaaaaaaaaacagcaacaacaaaaaaatcaaataatgtaccttgtttagcataaagcataattatatgcatatggtgattgggaggatgaaatggaaaggttatttattactgacttcagaaattatgtcctgatagattgatggttgatttaaatataacttcttgtcaagcatctgtttttagaatcaaattactatgactctgcagtttccttgaaatctcatagtatcacatctctgtttgcctttgcatggttttaagaaaatgaggagtgtgaaacttcaaacttcgttttcattgtattacatttttgaatgacacactggtcatttcctagaaatataaggtgacaaatatttcacagaaacataaggtgctattatctcattcaatcttaggtcactcaaaactctttctctcccacacattgaagattcatttgggaatgcttttgtcttattgtgcacccccagtgaaggggtggtaagtgtttttcattttgcttcctttgtttatctacagggttccattcaataaacaaagggacttgggtcaaacttcaggctcttatgggtttggatgtaatctttggtctcatttttagttaccaacagagagtgttgcttctgacctctttgactcttccctgctgaatttactatgcctttgatacttgtgaagggtgagattttcgaggagtactgttgtttttgttagaggttgtaatgtctttcttcgctttgtgattcaagttgtgttcagttacaatcataagcatgtgcctaaaaaaatcagatgcaaactagcaaaagtagaaactcagggtgacagtctttaagaaaagatgcaattcttggggctgggtgcggtggcttatgcctataatcccagcatgttgggaggccaaggtgggcagatcgcccgaggtcaggagttcgagaccagcctggccaatgtggtgaaaccctgtctctactaaaaatacaaaaattagctgggcgtgctggtgggtgcctgtaatcccagctactcaggaggctgaggcaggagaattgctggaacctgggaggtggaggttgcagtgagcctagattgcgccattgcactccagcctgggcaacaagagcgaaactctgtctcaaaaaaaaaaaaaaaaaaaaagatgcaactcttattactgacacagaaatgaaaatttagttacatagtattgtaaaaggactatcagctaggtttagccttaccaagatttaggtaattcatttcctgctacactcatattctcagccacttccctcatcacattttcagggtgcagtatataatagcgtcaactcgtgtaattccccctactccccatgaacttctaggccaaggggccacacggggtggggcatatagtataaaggagtaaggcagactgttggagaaaaacagggattaggccaggtgcggtggctcacgtgtgtaatcccagcactttaggaggctgaggcgggcggatcacgaggtcaagagtttgagaccaggctgaccaacatggtgaaaccctgtctgtattaaaaatacaaaaattagctgggcacggtggcaggcgcctgtaatcccagctactcaggaggctgaggcaggagaattgcttgaacctaggaggcggcggttgcagtgagccaagatcacaccactgcactccagcctgggtgacagagtgagactctgtgtcaaaaacaaaaataaatacaaaaacaaaaacaaaaacaaaacaaaacaaaaaacaaggattagtgaggactttgcaaagtgcaaacagcatgctccatctaaagagagcttttcagtactagctgattgttgttgtgtggaaatacgtcttgtatggtgagatcttccaccttttcaacagaaatgagaaacacaaacatttctgtgtgacacttccaaacttgttggctatttctgtttttctttttttctttctttctttcttttttttttttgaggcagagttttgctctcgttgcccaggctggagtgcaatgacgtgatcttggctcgccgcaacctctgcctcctggattcaagcgattctcctgcctcagcctcccaagtagctgagacatgtgccgccatgcctgactcattttgtatttttagtagagacggggtttctccatgttggtcaggctggtctcgaactcctgacctcaggtgatccatctgcctaggcctctcaaagtgcagggattatgggtgtgagccactgcgcccggccctagttggttatttcaaacaaaatactaacactcttcagcccaaaccatgtgtcagagagctgggtgtggccagtgggcccctggtgtgcaactagtcttcagtgatgctactgtatgctagtgacagcacaactgaccactgaaaatatggtgcacgatggtgaacagattggatttcccgtatctctagaaccccagggctttganggcaattctgatagatggagggggagcgagangagagaaagaggagggagaggtatcacatgaaagagatactggactttctgctaataaatgggtgggtatttgggtgagtagataggaaattaaaaagaaatatgagagatgggcgtggtggcttgtgcctgtaatcccagtgacttgggaggctgaggtgggagtattgtttgagaccaggagtttgagatcagtctgggcagcatagtgagaccccgtccctaaacataaaattttaaaaaatcagnctaagcgtggtggtgcgtgcctgtaatcccagctacttaggaggctgaggcaggaggatcccttgagcccaggagttaganggctgcagtgagctatgattgtgccattttacactagcctgggtgagaaagcaagactcccacatctgagacagagagagaggagagaggcagaccatatttgaactccaggctggtgaataattcatactgattaatttaattgaaaatatttatttaaaatttgcagatcggagttttggaaaggttacatttattaaattattgttggactatgaggaacacattgttgcttggaataatatttaattatgatgatgatgatgatgataataaatgacaataataaacaactatcatttattgagcacataccatataccaggcccagtaccaagcagttcacatatattatacatctaatacccagagcaactctataaggcagctctatatgttgcttccatttgaaagatgaataaactgaggtacagagatgtgaagtaacatgytgatggacacccagatatkatytkcagataagaaggataagtcagaagatcagataagcagaaaggtttmmacacacaggactytcatttagtagtaaactyttatgattggtctaaaattctattttcttttacagtttacctactcatcaaatctgtcttaactatttttgtctctccaaattctcttctgttctttkktactatgtacacatgcttttaaaatatgttaggatttgctgtttccatgtgktaatttcttgtttgcctttcttcatttataaactctttcctcctactaattattgacaaactctatttttctttttttttttgaaaaactctatttttcatatcatttttctaaatttaaaaatagcatataaggctgggcatggtggctcacacctgtaatcccagcactttgggaggctgaggcgggtggatcacctgaggttgggagtttgagaccagcctggccaacatggtgaaaccctgtctcaactaaaattacaaaaaaaaaaagttagccgggcatggtgatgggcacctgtagtcccagctactcaggagattgaggcaggagaatcgcttgaacccgggaggtggaggttgcagtgagccgagatggggccactgcattccaacctgggcaacagagtgagaccctgtctcaaaaaataaagtatatatatatatatatatataaatatttacgtgtgtgtgtgtgtgtgtgtgtgtgtgtgtgtatacatatatatagcatgtgtttattttataaattttgtaaaatacagacatatatgaaaaaattacctataatcttaccatccacaggtgattgctgttagcacctatctgggattttttttttcttttttctttttctttttttttttttttgcagtcttttctaggcacatatattcaatgaaaaaggttatcaatatagtaacagttattttttattgaatgttaaccatacacataaatatagctttgctttttttttcaattcgcatttaccatgagcagtttctctatatcacaactctttgaaaacattattttcacctttcatggctatataacattctatcatgtgaatttatcaatgtgtattttagaattgttatcasatgattaagcatttttaaaatgttcactcatcaataaaatagtgacaacattcttgaacataagttttttttaccatactatcggatatttccttagtataaattcctccaagtaaaattagtgggccaaaggctttttttttttgaggcggagtttcgctctcgttgcccaggctggagtgcaatggtgtgatctcggttcaccacaacctcctcctcccaggttcaagcaattctcctgcctcagcctcctgagtagctgggattacaggcatgcactaccatgcctggctaattttgtagttttagtagagacagggtttctccatgttggtcaggctggtctcaaactcctgacctcaggtgatctgcatgcctcggcctcccaaagtgctgggattacaggcatgagccactggtcctggaccaaagcctcttgatacattttgctaaactatcagaatgttgggcctctttgcaccctactaacaccctcagtttttatgagtatgattacattattttccctcttttccccaacctcctattctattttccttagataccttaagactgcacccccaacttagctttctgttctcattcaggcagtgtgtccagggcttccaatcccagtggtgagtaggcagatgtgggggcttcctgttgaactctgttaggtaattgagccactcaaggggaaaccagctttggggtccgctctgtggcaagcccaggactctatngaatggagctcattcctcactaatttacacaccaggacaagtagccangctaagaaattgctctaatttcactggtcgtcagtcaacaaaagaatgaagatacagaaggaaaataaaaggcctgctcccacagtggggtcatagcacatctccacatttaacctttgacaaggcaaaagtatttcttctgtccagacaattccagaagtcataaggaatactgggcaaggatgggtcaaaacaacacagactattgagggcaacaaaggaaaaaccatgcactcctgaagaaatagatcccaaaatgctgaacgagaaagaggattgtctctctagaagtctaatagaaaacagtatctggaacattaaacgtaggattttttttttttctaatgacttactctcttattaatatgtcagagaaaagaatagctcctggctaagaaatacaacagtccctcatcccagaaatcacagccaggaatatggattcttaagtgtcaaaaaagtactctgaataaggaaagaaacgcagatgcactacttctattataataagcgcttgatttcttttaatcccctgagtccaattattttcttggcaaatttaagggtactgactgcttctgtgaatctattgttacacttgataatggatctgagttgggggtaatatttgcctatcaatttggatacttaaaaatctctctctctttcttcatataccctctatctcacaactttccatttaatgagggaagtgaatttcttttttctgcccctctttcctactgcttctagaataaaagcataacaggacgacaggagtggagatgagaggaagcatttccaagcaatgggaaagtatgatgagagtcgtgagttggtagaatgggggtgagtaaggggtggggtgtggatggatggaagaggatggaggaggaagcaggtcatatgatcaggcttcaaaggccttccaatatcttgttttcagaggctttgtaggtcttcttacagaactttgagggtatttttcctttgtgagcaaaggagaaccatagtaaattttgagggatgggctgggcgcggtggctcacgcctgtaatcccagcactgtggcaggcagaggcaggtggatcatttgaggtcaggagttcgagaccagcctggccaaaatggtgaaaccccgtctctactgaaaaaacaaaaagttagttggatgtggttgcatgtgcctataactccagctactcgggcagctgaggcagcagacttgctggaacccgggaggcagatgttgcaatgagttgagattgtgccactgcactccagtctgggagacagagtgagactccatctcaaaaaaaaagaaatttttttttttggagggagaaaatatatgattagattgtttttgtgttggtttgcttgtttgtttttttccccaagcaaaaaatcactttactgcaatagggaagacaaataggaaggggaagaaactagagacaggaacagcagtttggaggttctgcaatacagaagcccagatgtttggcttggacttagacactgggaatgaaaaataaatgatgaattaaaaaaataaaatatttgggaggtatacttgacctgaccttggtgctttttcaaatgagaagagaaagatagagacagatgaaaaagttaagcaaggatgactatgatttcccatagtgaatgtctggatgatgatatcattaaatgaaaatttttaaaaaggcagatggtagaaggagatttgaaggaaagacaagaaatttgtttgttttggatttacttgtagaatgacctgcaaaattttgtcctaataaatgttaacaagtggctttccatacaaaaaccaaaccaaacaaaaccctgatgtaaaccaaaaataaaattctgaggccccccttcaccatctgaatgaacttcctcctctgcaagggcactcttaaaatttaacatgaaagactggttcaggtcatgacgggaagtgggggtcggacaggcctcattatgcctctctggcattaacatcaacacagaccttaagtctgttaagaagcatttacaatctattctctctgaagcctgctacctgaaggcttcctctgcacactgagaactttggtctccacaatcctttatcttaagccagacatttcctttctattgatcccaggtctttagataaactcaaccaattgtcaaccagaaaaattttaaatctatctataacctagaagcccccacttcaagttgccctgcctttttgaactgaaccaatgtatttcttaaacttatttgattgaagtctcatatctccctaaaaaccaagctgcaccccaaccaccttgggtgcatgttcttaggatctcctgagggctgtctcctgagggccaagatcactcatatttggctcaccataaatctctaaatattttacagagttttactcttttcatcgacactgatttatattggatttccaatggtgtaatattcatgccgttggccgatttcaagctagctggtagagatatgcaaacaaacaaacaaacaaaaagagatatgcaaaaacaactcttgtgagcttgtgtgctggctccaacacaccatgctgctggatattctaccaggcagcatagggcgcacggtgggtgagcctggcaaggagacaagagctggaggtacagggcttaaatgaagccaagggcgtgaataatattgatgaggagagcaagtagaaatgaaaagagaaaaccagtattaggagatctataaagaaacaatagtcagaaaaagagtggttaaagaggcagaagaatcagaagagaaccagattcatgaaataaaagagaggaatcagtttcaggaaggaaagtgtggtcagtagtgtcaaataccasagactgaatagyctaaggatttaaaagaggtcaacagcwccaccaattaagcggtttttctgagcttaagatatttttctttatttttctatgttaaaaatattttataaagaaaaagaaaaaaagagatcacatttgcccccccaacccgtccctggcctctctctctctaaggaagtcgttaatgaatttgggcagaatagattcagagctgtgagagtgaaaatttgactgcagtgggtgaggtgttaataaaagggagggggtagagatgaaatctttccagcagtttggctcttaaggggatagcttgaaggaaagagggttggaagggtggtttttaaaaagaattatcactcactaatcaactagaaatccagtggaatatgcagtacttgtctgtaatccagcaggctaacttttttttttttttttttttgagacggagtctcgctctgtcacccaggctggagtgcagtggcatgatcttggcttaccgcaacctctgcctcccagttcaagcaattctcttgcctcagcctcccaagtagctggaactacaggcatgcaccaccatgcatggctaaattttttttgaatttttagtagagatgagtttcaccatgttggtcaggctggtctcaaattcctgacctcaagtgatcacccgcctcaacctcccaaagtgctgggattacaggccagagccacagtgcccagccaaggctaacctcttgatcccaatgacaaacagaacaaacatcttactcaagtccagaagcaataataattttgaatcttgcttgcatgtcaacaggagccacattaatacagaagaggatcacattggtccaattaaattgaattgattgagagcctctgcaatacacggtctactgcacaaataatgatggttcctggtacatttttatttgaccattgattgctcgatttgtttctgtgtctaactgtgtattggaattaagctgactcaatttgaactgcaggtccttttatccctcttattttttatttatttatttattttattttaaggcatttctcactctccagaaaatctctaagatttcagctattaggcatttgtctttttttttttttttttttttttttttgagactgagtcttgctgggttgcccaggctggaatgcagtgtcacaatctcggctcatcacaacctccacctccctgattcaagtgattctcctgcctcagactcccgagtagctgtgactataggtgcaaaccaccnacgcccagctaatttttgtatttttagtagagacaggttttcactatgttggccaagctggtctcaaactcctaacctcaagtgatctgcccgccttggcctcccaaagtgctgggattataggcatgagccaccgtgccccaccaggtcttcttaatcataacaaattcatcttaaaacatcattttaaaatatattattttttattcttcattgacttcctgcattactctattttttcagagtttctagcaaccagtatcattggtattttaaacaatgtgtacatgtacatttatgcagatgagttaacatatatcaaagcaacctccaaacaatgccatttaggtaatctccaatttaaaagcctcaatagaatgataagattgagcttttctgtagttccatgacctccagcagagtctgcaaggccacagctgcctgaaggttgattctgtaattagaagatgccagggtcatctcagaatagaacctcaagccacccaggctacatttacagaatcagcctctccagaaaaacagcaacaaaggagggccttcctatgtatttggaaggagtcacctagaggagggacttggggttttggtgttggtgtggggggcagggatgggatggggagggggaagcttattgaaatatactaaaagacaaaccaacctaagggctggagggaagaaaattcacacttgtaagcttcttttttaaggggcatctcttaggctctagcttttgagattcagtatatatatatttttgagtcttgctctgctggagtgcagtggtgtgatctgggctcactgcaagttctgcctctcaggttcacaccattcttctgcctcagcctcccgagcagctgggactacaggcgcctgccaccatgcccagctaattttttgtatttttagtagagacggggtttcaccctgttagccaggatggtctcgatctcctgatctcatgatccgcccgcctcggcctcccaaaatgctgggattacaggcgtgagccaccgcgcccggcccagtatttttgttttatgaagatattacatttgtaaagtatgagcttggtgtcagcaaacttatatccctgtgtacaaactggccgaatcacttagccactttgggccaaatcacttagctcttctaacagtaagaaatcaacaagaaaaataaacatttcaaacattacaatgtgttcatgtattcactgtgggggatgaccagattctcgaaaccacaggttgttcttagtgaaacaagtttgggttggggccatagacttgtgtatttagaatcaatggctgttctctctctgggactttgatttttttcttgggctcatcccctttttgtagtatcttatctttgtcttatttgtataggacttaactgttcccattcccttattagagcaatctaagtgattaccttcataccttttggaattatatgcttcaaaattccaaaaagaatgattttgggctgggcacagtggctcacacctataattccagcactttgagaggctgaggtggatcgcctgaggtcaggagtttgasrccawcctggccaacgatagtgaaaccccgtctctactaaaaaatataaaaaattagccgggcatggtggcaggtgcctgtaatctcagctactcgggaggtggaggttgcaatgagcccagatcgcaccattgcactccagcctgggcaacaaaaggtgaaactccatctcaattaaaaaaaaaataatgattttggtggtcgacttcaaataggtaggagaagaaggagagaggagatggagggtcasggagatctaattactctctaaaatcatgctaggaaagataacaccttttaataacactctctgcttttataacatcattctgccaaggagctcaaaggtttcaacamagttcactttcagaaaacccctttgaggaagacagaatatacatcttctctccmttttaaagatgaagaaacaggccgggcacaatggctaatgcctgtaatcccagcactttkggaggctgaggccasargatcgcttgagctccaragtttgagaccagcctggataacatggcaaaaccctgtctctacaaaaaaaatacgaaaattagatgggtgtggtggcatgcacctgtggtcccagctacttgggaggctaaggtgggaggatcgcttgagcccagggagtcaagtctacactgagccatgattggatcactgcactccagcctgggtagacagagcaagaccctgtctcaaacaaaatgaatgaaagagaaagaaagaaagagtgagaggagaggagatgaggggaggggagggtagcagggagggggggaggaaggaaggaaggaaggaaggaaaaaaagatgaaaaaagaaatacgcaacatgaaacagaggcagaaagactttacgtaaattgctcatcatgtggttgtcaagtttgaccccaaaacccaatttattgaccaaggttattctttgactgaggcaagggggtccgctctcctgggccttgggctttagaaagctcatctctggcctttctgagatccatccctttctttttatttttcttgacacggagtcttgctctgtcactcaggctggagtgcagtggcatgatctcgactcactgtaacctctgcctcccgggttcaagcgattctcctgcctcagcctcctgagataacaggcgctcgccaccacatctggctaatttttgtatttttagtaaagactgggtttcatcatgttggccaggttggtttcgaactcctgacctgaggtgagctgcccaccttggcctcccaaagtgctgggattacaggcatgagccactgcgcccagctcagatccatccctttctaagggcaaacagtccatggtgcaaaggggccatgccacccagagttatgagtacctgggactccagaattccttgcctggtggcctccacatgcacttccagggcctgcttgggcctcttctatgggtctgtcctgagtgttgatagaaccactgatgtgagtacctgggcttgagccgtggcctggagatcctgttgactgtagcatggagggggcttgtgcagctgaatgtctgyatgcaggtggtgggagttctggaatatgatggagctggaggtgggaagagaagtaggcttggggcagctctctcatgccacctcattctggccaaaactcaggtcaaactgtgaagagtctaaatgtgaatctgcccttcaaggtggctacaaaggtatctttgtcaaggtaggagaccttgtggcctccacgtgcacttccagggcctgcttgggcctcttctacgggtctgtcctgagtcttctatgnaatctgtccttcagggcagattcatatttagactcttcacagtttgacctgagttttggccagaataaggtgacatttagtttgttggcttgatggatgacttaaatatttagacatatggtgtgtaggcctgcattcctactcttgcctttttttttgcccctccagtgttttgggtagttttgctcccctacagccaaaggcaaacagakaagttggaggtctggagtggctacataattttacacgactgcaattctctggctgcacttcacaaatgtatacaaactaaatacaagtcctgtgtttttatcacagggaggctgatcaatataatgaaattaaaagggggctggtccatattgttctgtgtttttgtttgtttgtttcttttnnntnnntnnntgtttttgtggcctccttcctctcaatttatgaagagaagcagtaagatgttcctctcgggtcctctgagggacctggggagctcaggctgggaatctccaaggcagtaggtcgcctatcaaaaatcaaagtccaggtttgtggggggaaaacaaaagcagcccattacccagaggactgtccgccttcccctcaccccagcctaggcctttgaaaggaaacaaaagacaagacaaaatgattggcgtcctgagggagattcagcctagagctctctctcccccaatccctccctccggctgaggaaactaacaaaggaaaaaaaaattgcggaaagcaggatttagaggaagcaaattccactggtgcccttggctgccgggaacgtggactagagagtctgcggcgcagccccgagcccagcgcttcccgcgcgtcttaggccggcgggcccgggcgggggaaggggacgcagaccgcggaccctaagacacctgctgtaccctccannncnnccccaccccacccacctccccccaactccctagatgtgtcgtgggcggctgaacgtcgcccgtttaaggggcgggccccggctccacgtgctttctgctgagtgactgaactacataaacagaggccgggaacggggcggggaggagggagagcacaggctttgaccgatagtaacctctgcgctcggtgcagccgaatctataaaaggaactagtcccggcaaaaaccccgtaattgcgagcgagagtgagtggggccgggacccgcagagccgagccgacccttctctcccgggctgcggcagggcagggcggggagctccgcgcaccaacagagccggttctcagggcgctttgctccttgttttttccccggttctgttttctccccttctccggaaggcttgtcaaggggtaggagaaagagacgcamacacaaaagtggaaaacaggtaagaggctctccagtgacttacttgggcgttattgttttgtttcgaggccaaggaggcttcgggaagtgctcggtttcggggactttgatccggagccccacatccccaccacttgcaactcagatgggaccggaggcggtgttaaatggggagacgatgtcctagtacgagctctggtgaccccaggactctgcgctgctgcgcttgngggcttgcccgacggtggagaccggggagcatctctgggcgtggagacccgggcgcagtaccccgggctcagaggggtcgggggttcccggngcgtgctgagggcgctgctgccgggtggggagagctgcaggtccggcaccgagncgctgctttgttcggagggccctgagctggcntagnaaacccttctggttgcaggtcggccagtacctacggagacaaatgccagcactgagtcttcactcggttcttaagaagctggtctgttctgacctgggaattggctatatgctccccgggactggagcggcacagtcccggactgtgaatccgggaactcgagttggaggtgtcccaaacggtccgtggtgctattgctcactagaggccttgggtctttgntttgacctgaggggtagggaggtcctgcctacagtctccgtgcgctcagctgagctggtgtccctggcgcagagcgcggacgagttttgtttccttttctttttcttttttttctttcctttaagtctcggtctgtcgcccaggctggagtgcaatggaacgnatctccgctcactgcaacctccgcctcccgggttcaagcgattctcctgcctcagcctcctgagtagctgggattacaggcgcgtcaccacatccagctaatttttgtatttttagtagagacggggtttcaacttgttggccaggctggtctcgaaccctcgacctcaggtgatccaccgngcctcggcctccccaagtgctgggattataggcgtgagccaccgcgcgcggccgagttttgtttcttttaaaaacaagacttaggagagcctgcggagacccggaggtggggtgcccaatcctccctctcccacgttccctgcagccccatcttccagaccgttgctgctggtctctcggggcagcttctgcctgggcgcagatggggaagctgggccgaggtggtgccgtggaatgaccgggagtaaccccggcgggcggcgcagaactcggagctccgccgcggggctgggctgggctctgccgtgagggtgggggtgctgggcgcgcgggctgcggtggnccccggagactggcccgcagncgcctcctggccggaggacctaggaatcggccggctctactaggtgtctttgctcgcggttccgactgtgaatccggtgaagaccggtggttgcagacggggaggaactatgaggttgaggcgaaagcccgttttgtttttttttttttgtttttttggtttttttttttgttagcgtgtttgccaactcccaggccattggtaaagcaggaaggttcttggggcggcggacggtgccagggttatgtgtaggtgcctctttaggtatatcttttatcaaaaagaagcaaagaaataagattaaaaataaacaaagaaaaaagttgtctggcactggcagtaattggcctgcctttgcagcactgataccattagcttttaaaatccgacttttcattgacacttcaagaagagaatgggtagtatatacacattcatctcatagtggacaaatttcatatttaaaaaaaccttctgggtactgaaatcagcaagtcacttgccctccatggccgaatccctgcttcccacgaagagaacctcacaaaaatttcccccaagttaaagagtggaattttcttgatttttttnttcttttttttttaacggccgtagtttagaancccagacttaaattatgatcttcttttcaaacaaaacttaaagtccttaagttttcatctccccttttatttcaacctattcttctcatacctaccacaaaaataatggaggctttctgttgagaaactttccgtttctgttgagagtatcattctcttgagaaactttctcctaaatcagagaaagtatggaagcatggaaagtattcctgagtagaacctctacagatattacaatatttttcaaatacaaagtttccattgtcagcctgtttcccaagtgcttccacaaaccattaaataattccacaaaccattaaaataattaatgctagggaattttaggaaaacattggtttacaatcagaaggaccggggaagtgggtcttcagccttcacgatgactacaagccatttaagggactagaattgctactgttgtcagagcaatttaggagtctgtatttgagcacccgcatagtgttccagaatgacatatctgactgtaacctggacacgtgtgatatgttgtctcccctgcagatgagcatttgaaatctcaaccctcgtatttctacgagtgcaggcctataatggaccctgggcacattttttttttttttgagatgcagtctcgctctgtttcccaggctggagtgcagtggcacgatatggctcactgcaacctccacctcctgggttcaagagattctcctgcctcagcctcctgagtagctgggactataggcgcacgccaccatgcctggctagtttttgcattttcagtagagacagagtttcaccatgttggccaggatggtctcgatttcctgacctcttgatccacccgcctcggcctcccaaagtgctgggattacaggcgtgaggcaccgcgcccgacccctggacacattttgacttagaacatattttcggtttgtgtgagacagtgcattagtgcaggattggaaaagagtgatcaggaattgattgttttcaaggattggttccttctgctcaaggaagtcccattgtaaacataaaaaaatgaatgaaactgaagaagttcagtgacttagctttttattatcttctgtagtacttacctttttggagaggagttggttgggatatttttccatttaaattttttttttaaagggatcttctctcccgtaagccgggatacttaagctatatatgtagtggctacaaattaaggtcttcactgttttcatttttagctgctagaataagtgaacattaccttagatagactcttctaattatgaagatatctagatgtctagaaaatatcaaaatgcatgtggtttttgcatttctaaaatacttttaaaaccaaatactttttcttttttttttttttctgagatggagtcttgctcttttgcctaggctggagcgcagtagaatgatcttggctcactgcaacttccgcctctcaggttcaggtgattctcctgcctcaacctcctgagtagctgggattacaggtgcgtgccaccacacccggctagtttttgtgtttttagtagggacagggtttcaccatgttggccaggctggtctcaagctcctgacctcaagtgatctgccagcctcagcctcccaaagtgctgggattacaggcatgagccaccacacctggcctcaaatacatttttttaagtatccagatattaaataaataataccattatagtagttgttatggtcatttactctagcatcaaatgttaaaagatcattctgaacacttgttttgtttatgctgagagaaggcctactccaaaaaatgcaaccatcttcgtatctgcatgtggatacaaccatgaatggccaaagttattgcagtgttgaatagacacttatatagcactgtgtggcaagtactgtttgaaatgtttttcacgtgtttattcattgtatttattttgagatagggtcttgctctgttgcacagggtggagtgcagctgcacagacaaggctcactgcaacctcagcctcctgcgctcacgtgatcctcatacatcagcctcttaaggagctggggccacaggcacgcaccactgctcctggctaaatttttacaattttttgtaaagacaaggtctcactatcttgctcagactggtcttgaactcctgggctcaagtgatcctcccacatcagcctcccaaagtgctgggattacaggcataagccactgtgcaggtcatcaaatgctaattgaattttcacaacaaaaccatttattgtccctagtttacaagattaagtaaatgagaagctaatttttctctggctatataccttgcaagaggcagagctaagacttgaacccagccagagttctttaactccagcactaacatttcagctgctgcaaccagggagcttttcaaggatgatcaccacattctctacattcatctgctataatccttatcagaatctacagcctgtatcatattttccttgttgctgtgagtgggtcagccaaattctctttaacttgaaaccttggttgcgtagggattgcaacatcctggaaagaatagaataaaatttactcaactcaattttttacttggttcataatgaaaactatactattgcttcagtcagatgtttgcgaatagctgtgtgatctcaaaatgttttcctatgtgatctatagnaaaatggaatgatagagtattaggctgtaagggcctaaganaacaaaggaaaaagagaagtgaactgttagtttagttgtaaaacctaactttggtgaattgtaaaaatttgttataatacaatatgattcttgcttgtcctgtccttgatgaagttgtggaccttttgaaataagctatttctctgttactgctgttactgtttagaaatcaaatttagttttttcttaagatatacgtatttttggaagataaacacagtttcaaagtctgccttgttggctgggtgcactggctcattgttgtattcccagcactttgggaggccaaagcaggaggatcacttgaggntcaggnagttncaagacncagncctggcaaatatggtgaaaccccgtctctactaacaatacaaaaattagctgggcgtggtggtgggtgcttgtaatcccagctactgggattgggaggctgaagtagaagaattgcttgaacctgggaggcggaggttgcactgagtcgagatcgtgccactttactccaacctgggcgacagagtgagactccgtcttgaaaaaaaatgtctgccttgtaaaagtgaaataggatgagaaagtgcctttcttattaattggtgtaatgaattagaaataaactctttgaagacacctcttggtaaaaatagttacatttactgttgatttatggtatgttggatatgtttttaagttttccgtgtaataactcagttcattctcatgagtgaaataggtgcttttattgtctttatagatgggaaactgaggtataggcaggctaggtacattattatggagttcgtaagtagtggagctgaagtcgcatcccagacagtttggcttccgtgagtttaccaatctcatggtaaagactttgtcagactatcaaagttttgacaaatgaaatattagcaaaaggccaaaagggattctctattttcatttgagtatcttcacctgaaaatagttgcctgaataagtagcctgcatagaaaggtacattttagaaatacttgaggccagagaatgaaaagcttacataaaattgatttccggtggggccttcagttactctccattctacgaagaccacaaatagcattcaggcaaagagcatttattccaacaatggaggagcactggatttggttcctaaaacaaaataaagtttgaaatcctgtctttcccatgttgaaaacaaagttggtacaaaaccctttagcttttgcaaacctcctttaagacccgatttaaatgcytccctcctcatgaagctcttctggatccactcyttcccatcactaagttgaaagtaagatccccttctctttacttccattagacttggattacagcactctttgtatcatgtatttaattctgttttttaattacagttaacatttatttgtcttcctcttgagtgtatgcttctctagaggaaggtctttgattcattctcccctggccttaattcatcccacttaatatggaaaaaatttaataaatgctgacttgaataagtccaacaaggagaatgggaagctcatgtttgcttcctgtcttctaaaagactacttaagataacagggtaatcacagaaaagcattagaaatagagttatatgagaaacaactgtagttaaggctaggtttatgttagactgagaaattttagtgcatacttaagttatttaggccaggttactttttgtagaacaaacatttcagtttcgctcagtttcatttccgtttctgnaggcagctgtgatttaagaaaatgctctagtctgtggcattccatattcaagtactttgagttgtatattaatttatttttgttaataagagtgacatgactcactaagtaatttagagatttaaacacttttttaaaaaacagtaacttcatatgcattggatctattcttctataaagtcttttcttggggggtgtttgtttaaaattccccggtgttttctctgccaaatccaacttccaagaagcatttggaagtcaaaacattttatctggttagtcttaaagtccagatattttgtgatagctggtatttagtttatgatatttcccaggaagaactttttagtagttgaaccatttatgaaagacttccttgaagctaccttagagagttgatttagttcttcctaaataagtaaatagaatattagtattaggacatcttggagtatagatgcaaatattggtgaaaaagaacatggatatcagagtcaaattaatgtagattggaattctggttattactaatggatatctgacattaggcaagttgctgatcactctttgcctcagtttcatcatctgtaaaataggtatttgtgtttgtgtataatgtgaaccgtataatataatgcttggcctatagtgaaatttattcatacgagtgttttcagtgattttaaaagcttgctttaggccgggcgcgatggcttctgcctataattccagcactttgggaggccaaggtgggcggatcatgaggtcaggagttcgagaccagcctgaccagcatggtgaaaccccgtctctactaaaaatacaaaaattagctgggcgtggtggtgcaagcctgtaatcccagctactcaggaggcttaggcagaagaatcgcttgaacccaggaggcagagattgcagtgagccgagatggtgccactgcacagagcgagactccatctcaaaaaaacaaaacaaaacaaaacaaaaatcttgctttatagtttacttccacatcaaattgtctttatcccatgttacttgcattgatatcccagacatgaaaagaaaaaaagatgataacaatgacagttattaaattaggttccactcttattctagatcaccaattcatattactattcagacttggaacattaaattttagttaaactttttttcaaatatgcatataattgtcagtggttactatattttggggaagagattgttgacttctttgaagaaagatacggattttctcttcagaaraaatacacatgggctcatataatccaaattttatgtgtaattacagggtgttcatgaatgcccacaaatccattaagccatgtgaccttggacaagtcattttacttttctgttttttaggttgttggtctgtaaaatgatactacttgacttttaaagagcccttcaagctcttatgtcctctaaccccaggtctgtattcagaagaaggggtggtcctttaattagagccatctagagatctgaggaacatgctgggcattagtgtaacataccatgtggattttgagaggtaaagaaaaaataaccagggaatgcctcagagcattcctgatcagatcgatgacagaagaaaggaatgagagggaggagaggaagctgttgaaatttcctatttacctgctttgagtgaatgaagatttgaatcatagaaccagaaggggttctcatctgaaatgcaaaggaaggaggagttggtttaattcaataagtttcagttgagtaaacatgatttagtgagatactgttcttgcttctgactcaccatttggaaaatctctctaaaataaaattggactctccatctcggacatcattttgggtgtaggttttgcttttttttttgagatggagtctcgttatgttgcccaggctggagtgcagtggcgcaatctcggctcactgcaacttccgcctcccagattcaagcagttctcctgcctcaacctcctgagtagctgggactacaggcgtatgccacncatgtccggctaatttttgtatttttttaatagagacggggttttactatggtggctaggctggtctttaactcctgaccttgtgatctgcccaccttggcttcccagagtgctgggattacagatgtgagccacagtgcccggcctaagttttacttcttataatggactcctgttaagccaataggtgatgaaaggaaaccataaccaactcttcaggctcattcatccttcaagaatagcatgctagtacccatcctaggaggagaattggactatacctcatgaggatagtttgaagtatctcagaagaccctcactgggggtaggtcggtaagacacaaagctttctaaagcactgtaccaaatttgttgtttgagagatcataacaaattagaagtggaaagaagaaggagtaaaaggaagaagaggtttctggccaggagcagggagggggaaggagctgctaggaagatgtttggttgtcatatccctgttcacccttgctttgcaaaattcttgtaggatgccaggttgggagtaattgtttttcacaagagtcaaaccacgcttgttttcttgaagaagcaagtctttggagggtggtggcttgaaatctgttggatctggtatttaggtgatacactttgacataaaggncaacactgatgcaagcagcagctttccttggaaaggcagggagaaagtgaaggcccagactgatgagcttacactgacctgcagacccttctccattcccaggcatgtttggtggcagagtttaccttagttggggttaggctgttgtctggtactgtgagagagaaggaagaagaagatatgatattaacaacaacaatacatatttatatttgaaaattaaatgcactaatacacctatagtgctattaaacaaatttatttgttcagcaaatgtttgttaaacaccaatgtactgtggaaagtactaggtgctggacagaggtcaaaagactttttaagaatctgccaccattaatgatctctttctgcttggcattcaaggctctttgaaataagactgtgacccactttgatagttttgtcctggattataagacacatgctcgaaggaactatagctggttttctcaccagactgattaacatatagtatggtttggtacctgttaaatgagtctctctctacaggttttcatcttctactttaagaaccccttcctggcattgtttggctcctcattgttctggaatctcatgtccatctcatgtatttgccttggttcacacttgatcttctgctcatattcctcacctactgaaattttacccatcaaccagaccgtgggtgatggaacacagcatgggctagttcttcttatatatgatctcatttaattttcactggaactctgagataggtagcatttcagcccactcaagttgactcaaatttttgtaatcatcaatatattttaaaataactttatatttgacctgtaattggaaaaccaatatgagttatcataaatgaaaggtaattttaaaaataattaggatgaagacaaattatttttctcacagtctatgtataagataaactattggttcccaaaggccccagctgacaatgagacttctcttactttgttgaaaagggaattagcaagcattaaagaggtgtcaaaaagaagactaaacaaaagcttactcctttttttttttgagacgagtcttgctctgttgcccaggctggagtgcagtggcaccacctcggctcactgcaacctcttcctcctgggttcaagcgattctcctgcctcagcctccctagtagctgggattacaggtgcatgccaccacacccggctaatttttgtatttttagcagagatggggttcgtcatgttggccaggctggtctccaactcctgacctcaggtgatccacccacctcggcctcccaaagtgctagaatcacaggtgtgagccgccgcacccggccgcttactccttaatgtactaagaatgttatatataggctgaagaagtgctgaaaagaaccatattttctcatgatgtggttcaatgtttaatactgtgcttgttcatctcctaaaatcctctgaatatcacttaaattcatcctgtgtaactctcccacatttggggaaatactgagcttgcctattattatattagccccatatttcagatgatgcacctgagccgaggagaagttaaataacttgttcatggttccatatttggctaatggcagagccagggattcaaactcttgtctctctgactcccaggtttgtgcttttcccacttggctgaatttctcatgctacctcctccataacacctttcctagaacttttaaggaatgcttcccctgttctctcatagcattttaatgtaagttgccaaagtgtcccagtttgcacccctaccagcaatgtgggagaaaatagcaacatatttttgatgttggggtctagtgttacggtttcttctgctctttgggatgtatattccatggctactgaatacccaagtcccaaacagttttcttagactcagaaggtcatccacttcagcttcttcatcaaaaagacatattttctggctgggcacggtggctcacacctgtaatcacagcactttgggaggtggaggcaggagaattgcttgaacccaggaggctgaagttgcagtgagcccagatcgtgccactgccctccagcctgggtgacagagcgaaactctgtgtcaagaaacaaaaaaaaggacttgttttctgttccattacccacagtggtagaatggcgtgctaaatttattctccagctgccattaactgcaaattaaaatcttagtctcttgcctctttaatccaggcttcttcatactataccagaatttaggataactattacagtgccctttataggagagaaagaagaaattgtgtctgtagatgtctgttcctttcagcttaaaatggacactgaaatgttaaatattggactggcctcatttatttctcctgtctgttggtccaatttgaatcttaaggcgtctttcaactggaattttttgtttctctcaactaaaaattgttctttgtaagtttgaatcagaacaaaatcctgaatgttgagggtttcctaaaggctgtttctttatgcaaaagcctgaaacccgatgttgatgttggctgcttaaaattaactgtgaatcaaggcagggtttttatttttattttttttttactttaatgattgtgttaattatagtgaaaaccttgagttcacgagaaagaaagcctttggtcaagtattgtttattaagttgtcagtcttgttgcaggatttgcaaatttagtggaattagtgccatttttcagtttacaattccagtcacatttcacatgatcagagcatggctttcttctctgtggagcaaatagagggctgtctgacacttggttccagtggcttccattaagcagagtggatatgtccctggagtctgcagagaagggcatggcactctgaccccagatggcactccgttttgggacattgtccaattctagttcatagcatatgtgaccaacaccagctctcacctgatgtaaacacttagcgcgttgttgcttgggggattggattgtgtgaatttttcaaaactacagttgacagaaggaggctaccaaaaatgaaacccaataattccatttttgggaattattcccacttttgttccatttttcccactttgttctttggcacacagaatgtttgatttgtgaaaatcttaataacagtagttttttctataaggaacactcagaatcttgataatattggaataatacagatccttttgtaggatcctctcagacctcatataatagagttcatgtagtcaatatttaaagaaaaacacccttaagtttttgtttttcagaatcacaagtaagtggatttaaacttgtgatcttattcccctttcttctcttaatttagtgaggcagccagcgagagggtttgttttggttattctaaagaaggagtttgcttgtaagttttggagggcaagacttagactctgtgtctctgtgcttgccctggaactttgattaaattgtcactaaccgagttagctggccctcgccgggctgcagaaatagaagtgtcttgcacacatgacatatgactgtctcaagagctggctggtgaaaggacgttctggagaaggctgccgatactgtatgaactagaanctggacaagagcctggagattggataactcagtttggcgcaagtaaagggaataaaagtgttaaggtggcaaaattgtatccaggtgtttataggctccctgagttcctgacttgagcctatctatgggtttagagttcaaggctctttaccagtgctgacaatcttatactctaggttgaacctccggggaaggtgcccttgcttgatggcatgtttaccaggggttctagagcctcaatcacagattctctctagctcacatgaagttaatgaaaatgaatgtgcttccctacaaattagagaggctttgaggaaaaatcagattaaatgcactcctgcttgaacttatgtttcttagaacacagctggaaattttgtcacacaaacctttactttcagtgacatttcttgactggtttgttactgtagtgaatctgctttaactatcttttcttatcgctgaggttttacttccattctacatgtgattgtggagcgctgcgtcattgtgggttcagtgtagtggagagtaggaagatggtgagacacagtagcttgttgcacattgcttaatttatcagggatcactgatgagttagtacactagagaagattgtaggtagagctgaaaagatggaggaattataaggctcagatttctctctttttttttttttttaagatggagtttcactcttgttgtccaggctggaattcaatggcatgatctcggctcactgcaacctctgcctcccgggttcaagtgagacttgatggtctcacttgatggtttcctggctcagcctcctgagtagctgagattacaggcacccactaccatgcccagctacttttttgtatttttagtggagatggggttttatcatgttgaccaggctggtctcgaacttctgacctcaggtgatcacctgcctcagcctcccaaagtgcagggattacaggcatgagccactgcgtctnggccaaggctcagatttctaatagagatnttctaatggacatagaggctggaggaaatgggatnggacangganaaactgagtncnaggtgccaaannnaacttgtagggggccgggtgcggtggctcanncgnnccntgtaatcccagcactttgggaggctgaggcgggtggatcacgaggtcaggagatcgagaccatcctggctaacacggcgaaaccccgtctctactaaaaatacaaaaaattagccgggcgtgttggcaggcggctgtcggtagtcccagctactcgggaggctgaggcaggagaatggcgtgaagccgggaggcggagcttgcagtgagccgagatcgcgccactgcactccagcctgggcgacagagccagactccatctaaataaataaatagataaataaataaaataaaataaaaacttgtagggaggtggcagtgtgctatggaggataggtgcaacctctgtgagaatgtagagaaaatagtataagtgagtggtgaggacccccaagagggggttttatagtaaaacaatggtcagaagtggcaacaggataccgtataatgctttcacctctaccaatgcactgggtactggagagcgctccagtttgctctggaaaggccctttctgtggacaaagaatacagaaaagagattcctttaataaaccacccactctgtgtcccacccttgatgaatacttcactgtgaaattgccagaattaatcatggtaatagctactgtacacttactttgttccaggaactggataaatgttttacatacattatcagttctattttttggagaagatacaggggctcagagtccctagggttccagagctggtgagtagcagagtcaggattcaaacccagctttctctgactctaaaacctcctttcttcctgctgaaacaattaactcaagacaacaaaggagttaaggatttggggagttttctgcatggtagaatagacccaaaggaaaagaaagaaagacagtgactaagatttgggtttgtctgccccaccaaatgctttgagcactttcataaatataaatccttcaggttgggagaaggttgaacatctgaagacactgattcttcagagatgtaatccaaacaaagtgatctttggtgatatggtcactaaaccatttatcccaaaattctcttggaaaaccgtcctataacagcagggaacattatccagccaagtttttctgcaaataaagggttgctgatagaggcttgcctgcttgtgtttctngtnagcntncagggtgtttatgaattcactaatcccttcccttcagatcccttttattctggtgttatgattgtgactgnaaaaaaattgattttttttctatgacatagaatgttgaaaggttgatttcttttctagaggaaagattctttttttctatgtgctacataccccccgaccagggaaaaggcaaatagtggtattgtttgctgaagtcttcctttgaaggttgcttggtgtttgcttagtggaaatcagcaggggaagagaggctatctctnaacattttgttagangtttcttcgtnagttctatagtgatgaaacaaggacttggggtacaggacagatctgctttcagaaatcctggctcttgtgaggtttagaagccctgagaccatttagctggtggcaacgggaatgttgagggtgataaataggatctttggttgtccaagtatcagtgacatgatatagatggagttaaacctttaggatctccttatttatttgtttgtttatttttgagacagggtcttgcactgtagcccaggttggagtatggtggcatgatcataactcactgcagcctcaaactcctgggctcaagcgatcctgctgcctcagcctcccaaggtgttaggattacaggcatgagccgccacacccggtcaggatctcctgtaaaattatattgttgacaacatgaagaattatgcttctcaaaagctagttatagatttgtacaatattcatagatttcttgtttcagtttttacaaattcatagcccttattttgaaaattagctattagcaataattttgtctaggaaattggatgtgtattcaagtgaaagaaggaagtacagttacctattatcttattgtaactaacaatcaagtaagtgtgatgcatttggtactttaaaaactgcacccaagttacagattattggaattaataaaattcactggatctatatatttttaaacggacagtgtgatagcagaacctcttatagaatngatagaattcctctggaatgattggataacttcatttcatccttgacttttaccttggaggatttcttaccccttttggcttctcaaatttgactattaaaatgttgcctttaaaaataggaacacagtttcaggggggagtaccagcccatgacccttctgcaaggccccctaactcaaggtagtttccctggaactgtggtttatggaatgtttcaggagtgtgaggaggtataatttaaggctgtcctagcaaggatacccttaaggatagagggcccagtagcatctggaggccagaaaagttanaactgaggcagtcagattagcttcanggctcaattaagctgatgggtcagcctgggagaaattgcaggatgactctcaatatcccctcccacccccacagcagccacgatctgtctgtctttaatcatgggtgcagtgaacctgttctttccaggtgtcttggccttcagtaaccttgttaggcttgtccctgaacgtggctaccgatccaaagacacatgatcagagaggcaattagagaacagaccttttccaaagcaagcatgttctgttgggcttagaagtttcatgtcctaatattataggaccctgtgcatctctctggagatgaggcacatgagtcatatctgtgattcttgcttttgtgtcaacatctcatgaataggcaatcagagctttggcaccaatgtattttcagttcatatctgatgtagttaaatccacctcctgctttgtagtttactggcaagctgtttttgatataagacatctagaacactgtaaatatataacatttttatttgtctattatacctcaattacgaaaaagacatctagaagcaacctcatcaagagagatactgaggccgggcatggtagctcacacttgcaatcccattactttgggaggctgaggcaggtagatcacttgaggtcaagagtttgaaaccagcctggccaacatgttgaaaccctgtctctattaaaaatacaaaaaagttagctgggcttggtggtgggcacctgtaatcccagctactccggaggctgaggcaggagaatcacttgaacctgggaggcagaggttgcagtgagctgagatcacaccactgcactccaacctgggcaccagagtgagattacatctaaaaaataaaataaagtaataaaaaagagagatattgatagctgttgttggaaatttcaacttccatctcacttctggtaactttttggaagtttgttgaacaaagtggaatacacgcacatacacacacacacatactctcttgtttgtttaaggtttaatgaaatagctgtcatataatcactgtttttgaaagaggagaattagttgctatctgtacattttgggtatgtgaactatttggatagaactctgagaaatgcattcagaacaacaaacaaaatcataggagaaatagctaagtgggaaggggcatataagagttgttgaaaaagttatttcttgagaaaccagctctaatgctaggcaagtcacttgctttgggggaggcctcagcttctctgtctataagattgcagcaggggtgtagtgggaatgagtcttcaacattccaagagattttatctactaatacgacagtcaaatggagcatgactttgtggaagcctctcctcttccacccagaggggccaatttctctgtcccagtgagatgttgacacttgtatgatccctgcttggagacttccctcttctggaacctgccctggctcaggcatgagggctgactgtcacccttcgataggagcccagcactaaagctcatgtgttggcagtgttcttgcgggaaggaaaaagaccagccagcccatttgttactgcacaagcaaacagcttctggtagctgtacagatacatgcactttctttcctcactgtgtttccatagacagatttagtgctgtagaagagtagagggcagtcacgggaaggagttcctgtttttcttttggctatgccaaatggggaaaaatcctcctatcttgtctttttagtgtcatcctctctccccttttcttcttctttataattctcatctctcatctctcctggaaatgtgcatgtcaagttcaaaagggcacaatgttttggtgaggaagaggtgggagaacacgtgccaggtgctaactagggtcatcatttcccccttcacagccagcttcctgtgaatgtgtgtgtgtgtgtgtgtgtgtgtgtgtgtgtgtgtgtgtgtgtgtatttcttttgccagcatcactgaatctgtctgctgtctggtattccaggttttggtttagggaaaagtaaaagtaattttataatcccagctgtcatttaagccaccccntttgtgggtagcatatggtccactctctcagttcattgtcctaaagatgcttcatcagaaaggaataacttccaccccgttactctctgtccccttactctgctttatttttcttcgtcaatcctaccaccaccacccactgtttgaacaacccactattatttgtctgtttcccatccctggtagaataggagccccatgaatgaaggaactttgcttctgttgttcaccactgaatctctaaggtatggaacacacctggcatgtgataggcactcgataaatatttgttgtggctcatgggcaccttgcagagttaaggctgcagttgtttgtggaatttataagtggtaatgaatatttatctactattcctcttccaaggcgatcacacaataatcaggctttacactatccagttcttaggtcttccaagttatgacttgtgaggtatgttaattatgataatagaaggcagtttatttggttcagatttattgatgtgtaatttaccacagtaagacttcccctttacaaaagtatgatgagttttgacaaatggatacacatgtgtatctaccactgccatgctccttttcagtctgtcgtcccctccacccatgaccactggtcaccactgcagtgatttctgtccccttcatttcaccttttccagaatgtcatataaatggaatcatgcagtatgtagttttttgtgtctggcttatttttcttagcattaggcttttgggattcatccaggttgtcgcatgtaacagtagcttattcctttttatggctgagtaagtgtcccagttttatttatatatttatttatgaggaggtgtctcactctgtcacccaggctggagtgcggtagcgcgatctcagctcactgcaacctccgcctcccaggttcaagcaattctcctgcctcctgagtagctgggattacaggcacccaccgccacgcccaactaatttttatatttttagtagagatggggtttcaccatgttggccaggctgatctcaaactcttgacctcaggtgatccgcccacctctggctcccaaagtgctaggattacaggcatgagccactgtgcccagccccagttttatttattcaccagttgatggtcttttcgacaactaattgtttccagtttttggctattctgtataaggcttctataaatattcacaaatacctaggatgggatgactgggtcatataatagtactgtataaccttagcagaaactgtcaaactattttccaaagtggctcttccattttacaattccacagtgtattgagtcccagtgtctccatacacatgctagcacttttaatatttaatttagtgggtatgtaatgatatctcattgtggttttaatttgcatttctctgcagctaatgatgagtgtttctgcttatttgggaaggttttaatttagcagtctgttgtattctgtagatattaataacttcaaaatatcagtggcatttgcagttaaaatttccttaaaaaattggccaaaggtttccagcagtcacttctgccatgcccaaactgtatgaaacaaggctgaggtgtggagattgtcacattttggcaaggagtgatccacttgggtgactgatgagacccagagagcgtacgcctcgggcttgagggtgaggacgggcgggaagtcgactgcatggccctgctggccttgggaggctgcccagtccttagctaaagctggcagttatgggaaacagacttagattctattacgtttttcaggatgtcccaggagtcacctgggaagctcagcagtcctttgtgactttcaagcatatggtagaagctgctgaacacagagctccctctttggggataatttgcccaaatcatttaatcaggcttgagaaatgagttaccacaggtccaggagtgctgccacccttgaattctgacaccctatttctcctatccgtctcttaattaattaagcagacatccccaagtgcttacgacaagccaggacccttttgcatactaaggaaaacagggatgaaggaaacagaaatggtctctgctctgactcagaaggtagaaatcctctttcccagccaagtcttcctagggagcacgtaggaagggctctgaacccacgtgtcagttgcaggggaggatatcaggaaaggacattgaagaagtggagacctaagtttgagacctaggcattagccaggctagcagtgcttgaaaaagtgtcttaggacaagagaactcaccagtgaagtcccagtggtaggagagcgtgcagcatattctgagcctgtatacacatctccagggcattgcttagcaggtggggagtggcaagagagtaggctggagtcacagaagggaggccaggtagaccttggtgagcactggactctatgttcaggtgctgaggagctggcaaaaggttttaagtcggggagaggcatgttcagatatttggtctagctgagtaactttgggtgctctgtgacaaatggttgggagaccagtgaggtggcagttgcggtcatctaggagcaggatcagagtggcctattgactgggatgactgtgaagtgggatcctttccagccagtaactggaaatgtgtatgagggcagaagtgagtgtactgcatttgaaacattgagaaatctagtacatagtactgtctcttttatatcttttttttttttttttttgattttggtttgtttgttcactaacttggaaaactgatgtggaaatgtccctttggcttcagttacctgagcagaaggggccgggcattgccaaactctcctcttaggacagaattgctcccagtattgatcattgtgttctgagttgggggagcaaattgtgcaggaggccaggtcagtgccaaggtgggtgggaggaattggagcaggaagcttgcctaagtgtgcccagcaaagccacggtagaactttctactgtggctctatgctacttcttagcaaccttctccatgtgcttcctggagagtccttggagtcagaacctttttcttgaaacccagacactttacttccaagaaaatgctgtccaagaaaactcatccttcccttcttctcatgaacgttgtgtagaggtgtgtcttctcttcctttgagcttttccactcagggtttaggggaggtgatattctatatttgggtttggctctgggtactgcaacactaggctattaagatttcatccttactgctttgcccctcctatctttccagaaacccacaatggatttgctagaaataatggaacgtcctgtttggacaggatataaccatttctcagctagaggatattgttggaatgaagaaagataaatggggagaagggaactcacattgctttggcacttaaattaagccatgtactgtgttgggaaattatttatattatctcgttgaatccacagtagaacacagttgaacaccatacaaggtaagtattgtcatccttattttaccatgaggaaattgatgcttagagagcataaagccttggccaggggcacatagttgggaagccggggctaattcatgcctgggctctttctgatagttttccttttttaattgtcccctcctcattgttaccttggggatttcaagagattcatgtagcttctaaatcaacgaactgattcctggagagcagcttctgtatgagaaaaatctagctaattatttatttcagtgtctctggaatgcaagctctgtcctgagccacttagaaaacaatttgggatgacaagcatgtgtctcacaatgctgctctggttgccagtgctgtgctgccagttgtcatctttgaacaaactgatgcagtgctggtttaactcttcctctttttggagtaagaaactttggaggcctgtgtccttctagaagtttgctgagcaaatggtaaggaaaagaaataggtcctaaggcttgactatttcagagaatttcttgatttattggactgtcaatgaatgaattggaatacatagtggtaggctgtcttttcttctcagacactgcaatttcctccaatctcttgacttttctagaagttttaatccaagtccttgttgggtggtnagataaaagggtattgttctactagagactgaccttggcatggagatctcatttggactcacagatttctagtctagcgcttggttttgtatccatacctcgctactgcattcttagttccttctgctccttgttcctcatgcccagtgtcccaccctacccttgcccctactcctctagaggccacagtgattcactgagccatttcataagcacagctaggagagttcatggctaccaagtgccagcagggccgaattttcacctgtgtgtcctcccttccatttttcatcttctgccccctccccagctttaactttaatataactacttgggactattccagcattaaataagggtaactgctggatgggtggctgggatacacagaatgtagtatcccttgttcacgagaagaccttcttgccctagcatggcaaacagtcctccaaggaggcacctgtgacacccaacggagtaggggggcggtgtgttcaggtgcaggtggaacaaggccagaagtgtgcatatgtgctgaccatgggagcttgtttgtcggtttcacagttgatgccctgagcctgccatagcagacttgtttctccatgggatgctgttttctttccagagacacagcgctagggttgtcctcattacctgagagccaggtgtcggtagcattttcttggtgtttactcacactcatctaaggcacgttgtggttttccagattaggaaactgctttattgatggtgctttttttttttttttttgagacagagtctcgctctgtcgccatgctggagtgtagtggcacaatcttggctcactgcacctccgcctgccaggttcagcgattctcctgcctcagcctcccaagtagctgggactacaggtgcctgccaccatgcccagctaatttttgtatttttagtagagacggggtttcaccgtattggctaggatggtctcgatttcttgacctcgtgatccgcctgcctcggcctcccaaagtgctgggattataggcttgagccaccacgcctggccgatggtgctttttatcatttgaaggactcagttgtataacccactgaaaattagtatgtaaggaagttcagggaatagtataagtcactccaggcttgaggcaaaatttacaaatgctgctgactttgtatgtaaggggaggcattttcttagaanaagnagaggtaggtctctgggattccagtatgccatttccatcctcagtgtttttggccacctgagagaggtctattttcagaaatgcattcttcattcccagatgataacatctatagaactaaaatgattaggaccataacacgtagctcctagcctgctgtcggaacacctcccgagtccctctttgtgggtgaacccagaggctgggagctggtgactcatgatccattgagaagcagtcatgatgcagagctgtgtgttggaggtctcagctgagagggctggattagcagtcctcattggtgtatggctttgcagcaataactgatggctgtttcccctcctgctttatctttcagttaatgaccagccacnggcgtccctgctgtgagctctggccgctgccttccagggctcccgagccacacgctgggggtgctggctgagggaacatggcttgttggcctcagctgaggttgctgctgtggaagaacctcactttcagaagaagacaaacagtaagcttgggtttttcagcagcggggggttctctcattttttctttgtggttttgagttggggattggaggagggagggagggaaggaagctgtgttggttttcacacagggattgatggaatctggctcttatggacacagractgtgtggtccggatatggcatgtggcttatcatagagggcagatttgcagccaggtagaaatagtagctttggtttgtgctactgcccaggcatgagttctgatccctaggacctggctccgaatcgcccctgagcaccccactttttccttttgctgcagccctgggagccacctggctctccaaaagcccctaatgggcccctgtatttctggaagctgtgggtgaagtgagttagtggccccactcttagagatcaatactgggtatcttggtgtcaatctggattctttccttcaggcctggaggaatataataactgagacttgttttatttctgcagagggttctaagccattcacttcccagatgggccaataatgctttgagtaatctggagatcatctttaatgcgcaggtgaatggaactcttccacagagggatgtgagggctgtagagcagagtgaactncctgaaactcagacgtcagctctttgtctctctatctctgaacacccttccttagagatcccatctctaggatgcatttctctgtagttagtttctaagtctcttgttcctgttctgcctttatttttttttcctggattctaagccagtatccccacttggctgtcttaatgtagcttaacatgtctgtaatcaaaatgatcatctttctgagattcaaagggctataagggactttggagagaatttcattcagttttcctcaaactagaataatgcttgcactgtctgtaaaagaacaaaagtgtcaaagcatccttttgttcactaaatttccttttttattatagtgttacttaaatattaggaagtnaaaagtaggtataaacttcnttataggctgttattatacaactatatgacccatacatatttacaaattaagtgcagccaaaattgcaaaatcaataccattcaaattaataccttaaatgtggtgaggcagctgttgttcaactgaaaccaaattataagttgcatggcagtaaatgctatcatgctgatcattttgagtttggccagtctatatntatcatgtgctaatgattgaattctccacccatttttctacttgtatgaccttaatttgatggcacctgttccatcctcatgagtttgctacaattatactggtgccaacacaatcataaacacaaatataaacttgggctttgaaatcttgtgccagaacttggctttaaagtaagcatttaaaaaatccatatgtgtttattagactttgtttagatgactgttgaaatgaaaacaaagtgtttaaaatcctcttagagaacttaaatataatccctcagcaatatgtatacagatcttcctttgagaaaaactgattgtgttcagcctctcatgttacaaatggggaacctgaattctgaggtctctagtgagagaacagggactggaatctgtggantcctatctgttttaataataattgtaaagtataatagataatattatattaataaaataaaagcaaacacttagaatgagcttccatgtgtgaggcactaactgattaggcattattaactagatttattccttttaaggccccgcgatgtactgttatttccacatgttgtagctggggaacgtgctactcagagaggttaagtaacttgtctgaggtccacaccactaacaaggagcacaggtagggttcaaatccagataatctgactttggagctggcactctaactcaatgtgcctaatcgcttttcagtggtgtcattattttgcctattctccatctgagaatattgaagtttctgactccttccttgcctttctccctgcctcccgtggttatccccaggtcttggtgttccagtcctctatgtccgtccttactcttattcctttgctacagtgtgatccagggctcctgccccttcttatcctggtagagggggcccacttgctgggaaattgtctccgccatggtttatccatgttgtgtgtccattagtgagtagtgggaagaatcatatcatgttggcaatgaaaggggggctatggctctggggtagtctagtctgaacctcttattttacggatgagaaagctgaggtacaaagcagggaagggatttcttgaggtcacccagccagcaactgagctgcaaccagaagctgagatccccaggactagggccgagcctcattctgtcccatcacagtgacttttcttccctcctccaaactatttttattttttatttttttgcagctgcttagcagcttgaagttagaagaaagggcagggaaaaggttttccgtgcttagccagggaaggaatcctgcaacaggatgtggggttgggtcattcaaattgggccagactccactggtcttgttgcttcttgcttggtattgcagatgggtttaaaagtgttaggattagagagataggcaggtttagccaaaggcagtttgtagccttgtggcagagttctttttaaagaaggaagtgggatgcaacaccctgacacaaaggggcttaagttgttataccactgcctgctaacctgttttccttaactctcttcctgatttctaaaggaagtatattttgctgaatcagaaagaaaagtgatttatttcaggttgctgatgcttagattgttagagttggaaagatctggcttgcatcttgtacagctgacagaactggggctcaggggggcacaggtgcccagagttggtcagtcaggaaagtagcaccagaaccagtctcctggtggccctacagttgcagacccttttttgctttgctctctgtgtatactaaagcttctatgtctctgaatctcaagttctgactggtagctactttccaatccacctggcttagatttctagattatattgtttagacgtcagaacctcttaagggttttggggccacttgttagctcacatagtgagaaccagccctgcccattaggtaggggaagaagttagcagtccatgatagctgttgcctgcagcgtatggatgttcattgcacagttcctgtctcctgagatcctggagtgtatacgcttggcctcagagcccagcacagagcctggcccttgggacatgcttagtaagtatttactgaatgagtgggaaatgtcttaaggcccattagtttgcaggtcttgaggaggctcccttgcactaggaagaatagaaagcatacataaagcctgtgtgctgccgccaggaagactagaaacgctatgttcagcctggagctgaatggtataccccagagcaaccctgttgaaaggcagtgcttgccttttcattctgtgtcctggtttgctggtaactcctgggtcccctgcctctcctgtacccccattgtgcagactgaggggggaccatcagccagggttagttttccgctgtttctgttaggcaaagaataaattgaattgagttgtgaaagttgggtgcaaagctcagtttgggtccaaagtaacagttaacttgtgtgggtggcaggtattcagtacaaacagggctggggacaggaaggggaagagaacttcagagctttcacgatcctcatctggttttaggctgatccagaggccaaggtccccatggaacaaactggacaaagtgagggtggccacatggcctcttttcttttgcctttattattaattttctcaaatagatctgactagtcatgtggctgggaaaatagttaattgtgatttttttttttttaaactgagtctcactctattgcccaggctggagtgcagtggtatgatctcagctcgccgcaacctctgcctcccgggatcaagcaattgtcatgcctcagcctcccgggtagctgggattatgggcacacagcaccacgcctggctaatttttgtatttttagtagagacatggttttagcatgttggccaggctggtcttgaactcctgacctcaagtgatccacccacctcagccttccaatctgctgggattacaggcatgagccactgcacccagccagagtaccactatttgggcattctttaatgaaaaagaatgaactatccaaaaattaaaactcctcatttatgagcttttagagaattttacagagtagatggaaactctctgcatcctttccccacttctagtttcacctgacacatttcttccctgtccttactcctgggccggcagcagtggtcatgattccaatcccagcttggccaccatctgcctcagtggcctaggaaaactcctttctccagagctttagttttctcttctacggaatgaagaaagttaaaacaaatagacatttattgtttcatttggataaatatctattaagcatctattacttgtggtatggttagctgggtatatagtggtgaagcagctgggcatgagtactgctttcgtagagcttacagttcagtgaggccagcagatgtgaaacatatcatcacacaaataaaaatataactatcaactgtgatgaggattatgaaggaaaaaatccggcaaactatggtactggtgttagatactagcaggtgtgggtagggatttcatttagattgacaggttgtcacattaaagctgagagccctgaagttcaagcaatggttagccaggcaaagatcagaggcttagagatagggaaatccattccaggcagagagactgggggtgcctgtcccctaggtcagggaacagaagaaagccagtggcactggtggagtgaataagactggcgggggatgagttggtagtagacatgaccagatmatttagggcncaattctcnctggggnaaggagaattntaatttaatatttatttatttatttatttatttatttatttatttatttatttatttttcaagacggagtctagttctgtcgcccaggctggagtgcagtggagcaatctcggctcactgcaacttttgcctcctggtttcaagcgattctncctgcctcagncctcctagtagctgggattacagacgcccaccaccatgcccagctaantttttgtatttttatagagatgcggtttcaccatattggccaggctggtctgaaactcctgaccttgtgatcctcctacctcggcttcccaaagtgctgggattacaggcgtgacccacagtgccccctgagaatttaattttattttatgtgcaagaggattccctgaggtagtcaggccacattgtctggtgactcttgggatagagggaacttgaatgacaaaggcccaagaaagcaattgtaatcattacatatacatggaccattttatgctgttttcttctttcatttaacattatttagtgtgcgtgttcacatatttctaaatcatcttctgatttagaataatgatttctgatgtgtaggctgtgttttatagttttgaaagtaatactttgatatccattacttntcttgattctcacagcaattctgnaggtgtatgcgttgcaatttctgtttcacagatgaagagagtattgttaataagttaatggccgggcatggtggctcacacctataattccagcactttgggagaccaaggtgggcggatcacttgaggccaggaatttgagaccagcctggtcaatgtggtgacacccatctctactaaaaatacaaaaattagccaggcgtggtagcacttgcctgtaatcccagctatttgggagggtgaggcaggagaatttgcttgaacctggcaggtggaagttgcagtgagccaagattgcaccactgtactcctgcctgggtgacagagcgagactctgtctcaaaaaataaaaagttgctaagaggagggctgggatcttttggctccaaatctactgtgggatgatgcctttgacattcctgatagctgtgcagtaatccattaacacagtttttataagttcaaanccctgttgccaacatttagattgttccatgtgtgctgttacaaataaattactataaagattctatacatttaatcttttattatttttgtattatttctgtaggccaaaatctgaggaacaggattactaggttgaagggaaatggcccttgaagtgtctgatcagatgtctttccagaggatccaaccaatttaaatagccaccatcaatgcatgagactttgtagttcagggaaggcaggcctggttttaaaaatcatttcccctctctagcatttttctgatgtgatccttaagatttcactttagttttcccaggtctcattggcatgtatgctgttagggatgggtctaaaattaatttttcttcacattcatatcatgtcatcccagtgattatttaataaataatcacttgattaaatagtgattccttttctagttatttttgggacatttattaaaacctggatatggtggctcatgcctgtattcccagcactttgggaggctgaggtggggggattgcttgagactaggagttcaacaccagcctgggcagcatagcaagactccatctctataaaaataaggaaattagtcaggcatggtnggtacttgcctggagtcccagctacttggaaggctgaggtaggagaattgcttgagtccaggtggtcaaggctgcagtgagctatgaccatactactgtactccagcctgggcaacagagtgaaactctgtctgaaaaaaaaaaaaaaaaaaaaaaaaaannnnnnnaaaaaaaagatgtgtagggagcaattttggagttattcatttggtcatttgatatgtagttttagttttggtgctgatagagcccagaatgtaccctgaatttgatgaacattctgatatatgggggagctcattgtcccccacttacctttttgcctctcagaatatcttttgatatttttatctgttttttccccattgaatgttattaccttatcaagctcaaaaaagtaccctatcgctattttaagttcagttgtgttaaatctataaattagcttgggaaatttggatattaaatgaactcatgaagaagcagagtttagctctccttaattctcatcttcctttatttatcctactacagttctgtggttttcttttatgtaagaagcacatgntttggctaagttaatgcctaggtttttttgtttatgtgtccattctcactgtggatagtattctcttttccccacattatattaatttaactggttttcagagactaatagcaatgctattatttaggagaatttaccttggttctgattaacttacccatacttgcaaatcatttgcagctttttagttaactttgtgagttctcttagatttacgaccatgccagaaacagaaaggatattttcatctcttcctttctgatgtttattcttcttgtttcctttttttatcccccattatattctcaagaatctctcaatactaagaaatagcgacttcatttttcagcgncggagtgcattattttggctaccatgattcagaagcctcttgcctaaggcccaattttattctgctagttttctctgttctttgtacatggcccttgcgctgccctaaccttgaattaacgtggctaaatctcaagaatttaagagcaccgtgactgtgtcctcaggctagggagggaaatgggttcacagagtgactggattgtggtctatgaacttcggcagccagcagcaaaagtcaggcatgaataatcaagtggacagtgaacatctgtagtgtgggagatgttggcataactatgaatgatgattcaagagtggtttgatgcatattgaataacatgatgataagtactagactctgtgctaagccttctatgtgaaatacatttaattctcataataactctagagcagtggttctcgaccggggccggttatccccctaccccaccccaccctcacccttccaccagggacataacatctggagatatttttggttgtcacaatcctgggaatgtatgtgctgatatttagaggttgaggtcagggatgctgctgaacttcgtagaattcataggagagtctctcacaacacctatctggccccaaatgtcagtagggtcactatcaagaaaatctgctctagcagtgcctgctcatattatccccatgttgaaatagcaagatgggaagtgcaaagtggtgcttcggtactcttggagcagctttgactttggtgagaaacgccttttaaaaacaatgtttcttcccatcttcccaccccatggggaggtgtggggttgggtgggtaggcaccaaagcaagatttagaagagttttctgtaggaatttataatggtaaaggatcaacttcatttccaagctatttatgagggtttatgtttaggaaaagtgctaagcttagagaaggaggagaaatctgattttattaatgagtgtagccataatggcatatcctggcagaagtcaactttggtttctagagggaggctattatgaaaagaaatacctggaacattcccctgggtttggaaggtgagttctaggttcaatgatgggaagaattttagaggtccaagataaaagggcaaagattaaattttgtctctcatgagttctctggctcaggtggtgtgaactttgcagacagtctctttaattcactcatacatgctagtctcccagctcagcaagggctttgagagagcaggtgtctgtatgctctggtaagtgaaggcaaagtgcataaggaggttggggtccataatggcgaagagaaggagcccttcagtcagagtggctttgaatcttggctctgccatttgccaatcttggaccattgggcagtgtattaactctttgaatctcagcttcctcttctgtaaaatgtgtataacaagagtactaattggattgtttgatgattaaatgagttaatgtgtataaagcactcacaaccctggtacatagtaagacctttcattattattatcatcatcaattttttttaacctcttttcctgatctgcttacactcaccagcttcagctgctccaaatggcttgtaagattttttgtttgccctttgctgtcagttgccatggggaagatccattcatttttttcagtcaaccaacatattttgagcatctgctgccctacaggatcctagatatgggggctgcagagatatccaggaacataagccttgattaattgggtcagatcagtgctcagcagggctggcaagtgctaggtttcttttaagtggcatatcttaaaaggtatatgtcctnaaacatagctttgtgatggcagcatgatgggtacaaaagcacacacttaagtgtcagtagatctgggttcaaacattggtgcagtttcttatggctcgtaacttgttcaaacctcagtttcttcacttctaaaacggtaatgatacaacctacctcacagggttattatgaattaaatactggagatgagatacacaaaacgtcttgagntacacagtagctgcccaatattggctgtaagtattataaatctacaagctgtgaattaattttacctctctggatcctgnttgatatttctagaccattccacctagtggggccatttcctacctgagtcacccgtggtgtcaaatagaatgtcatgtggcctcctgagttgggtagaattggctgctcatctcaaccccgctactgactatctctgtgatttacccttcctccagccttagccttgctacatataaaatcaagacaataatgtttcctatctcacagggttgtcctgaggattaaattaagtaattaatataaaatgtgccttgtacatattgggccctaaataaacagtagctactatttatccttaaagtacaaatggtagtttcagagcttcaaggctgatggctatttatcttactcatactctttgtttagcttcatttttttcccctaatttcattagwattttcttttctcttttttttttttttttttttttttttttttgaggtgaagtctcactctgttgcccaggctggagtgcaatggagcgatcttggctcaccccaacctctgtctcctgggttcaaacagttctcctgcctcagcctcccgagtagctgggattacaggctcccgccaccatgcccagctatttttttgtattttcagtagagatggggtttcacccttttgaccaggctggtcttgaactcctgacctcatgatcaacccacctcagcctcccaaagtgctgggattacaggtgtgagccaccacgcccggcctcataagtattttctaaatttatttacagtcatgccatttaaaaggaaagttgtattcctgtctttgttaatatttataagtgattttattcagctacaagcttggaatggcatataattttgtattctgcttttttcacttaatattacatggctaatgatttctgtgtttcataaacattattctgatgatggcatgatatattgttgagtacatgtaccataattgaatcatttccctattgctatgcaattaagttgtttccaatattttgcaattataatgtttcaatgaatgaataactttatgcatatagctttttgatatcttaagttcagtttcctaggatgaatttccaggaatagtaattgggcaaatgggataaacatgactcttgaatacgtattgttaacattgctttcccaaagggctcaactgatttatatttccgtgttcattatcttttaaaccagctcatttactcaccaaacatttttaaagccattatcatgtggtaggcttagtaagaagaaagtgaccctaagggagaagcttatatataaatagggtccctggtgtaccaagtgctgatacagacacaaagtacctggggaaattgagatgagggagtcctggctcagctgggagaaaagttcattttcatagagtcatggttttgttctttggcagaaagaaaattgctttcttccccacccccacccccagctttattgaggtataattgacaaataaaaattgtatatctttaagatatgcaatgtgatatatatgtatatctcaacttaaaaaataagctacagaataaaaaggtgtttgctattaaaaaaaaagaaaaggctgaatgtcattcccaagcttggaaatttgagtatgttgcctctttgggattatttacagaaatattagcaagaccagccccatctttggtcttgagtactccactgtcagcatgctttcttccagagagggatccatttgcctttatttttcattctgttgtgccgtctatgcaaactattcttgatagttttatggtaacagtgtttttttgttccatgagatnaatttatacatgctcattgtggaaaatttagaaaagacaggaaagtattaaaanacatcacttttttttttttttttttttttttttttttaagnagacagagtcttgctctgtcgcccaggccggagtgcagtggcgtgatctcagctcacagcaacctccgcttcccaggtttaagtgattctcctgcctcagcctcccaagtagctgggagtacaggcatgcaccaccacgcccggctaattttgtatttttagtagagatggggtttcaccatgttggccaggctggtctcaaactcctgacctcaggtgatccgcctgccttggcctcgcaaagttctgggattataggcaggagccactgcgccagccacacctacgttcttatcatcctagtacatccactgtcattatcttgctgtatttccttctgcccagtctcactctgatcatgcagtggcgtgatcatgcagtgatctcggctcactgcaacctaggccttctgggttcgagtgattctcctgccttagcctcctgggttcaagtgattctcttgccttggcctcccaagtagctgggattacaggcatacacccccatgcccatctaatttttgtatttttagtagacacagcgtttcactaaaattttgtatttttagtagagatggggtttcaccatgttggccaggctggtctccaactcctgacctcaggtgatccgcctgccttggcctcacaaagtgattacaggcatgagccactgcatccatcgccaaaaagattttttaaaagagtttaatgtagaaccatatcaaaggtctttggaaataaaaaacagttttttaaaaatatcagaaataaaacaacaaataaataaataaataaaaacacccaaaacaatctgaagcacgagcacctagcagaaaggttcaattatgatctattcatagagtggaatatcaagtagacattacaggacatgttttaagattatattttatgtcatgggaaatgctctcccagtatgatgttaaatgaaaaaacagaatacaaaagtatatatgctgcatagtctcaatattgtagagaaaaaatattatttatgtatgcatgaaaaaagacaaaagatgttaacagagatccattgttacttcagtttactagggattgtctctgggaggtaggattaaggtgatttatatttacctttttaaacttttctgtatttttttattttcaaattttccataaaaatataaggacttgaagatcaagaaaaaatttctgctttggctcagtgcagtggctcacgcctgtaatcccagcagtttgggagccctaggggagaggatcacttgaacccaagagtttgacgttccagtgagctatgatctccggatcgtaccgcctggacgatggagcaagaccctgtctcaaaaaaaaaaatctttgctttttttttttgtttgtttttgagacggagtctctctctgttgccccagctggagtacagtggcacaatctcagctcaccgcaacctctgcctcctgggttcaagcgattctcttgcctcagcctcccaagtacctgggattccatgcacccaccactatgcccagctacttttttgtattttcagtagagacagggtttcaccatgttggccaggctggtctcgaattcctgacctcagctgatccaccggccttggcctcccaaagtgctgggattacaggcatgagccactgtgcccagcccaatcttttgctttttttaaaaaaagaagacaaaaagggattttataccagtattatcttggctgtgtgactctgaagccacagttgtaagttataattactctgaaacacaaggccctgtgactcttttgggctctttggtgtttatcttgattacaacgttggaatatagaaatgaaaggaatgggagaggtgatagacttcaggcagtgtaactagttgtctgaacactactggctcaattatattgtgtctagtgatttccatcttgtccgtctgctaatttatcgcctggtaactcactgaggcagggttttcctttggagaaacctcattgttttaaccagtgtatcatgcttgtttagaagttcaatgatctttttaactcatcggagaagatgatgaccagacctggacagatggggaaggactttgcactctctctttacagtcctgagtgcacacaggtcaatatggaactatgtgtgaattttcattgtctttgagagccctcttctctgccccatagggagcagctttgtgtgcaattagaggagcaagggttgtgtgtatttagcacagcaggttggcctggtcctctcctctcaacatagtcaccacatacctggcactatgctaaggctgggaatgcagacagatgggtgcctgctttcagagtgctcaatgtgctgaggaagccagcaacagaaacagatgatttcaggagctccaggaaaatgctacaggaggagtgtgcctgggttactggagtagcacaggaggagggcttctagctcaggctgagattttagtaaaggaaattatgccacgatgaatcctgaagaatgaatagaagtgaaccagataaagcacgataggaagcatcttcccttacctaagggaagacacagaggtatatggaatggtatgttaaaaggttgggactccaaacagttctgttaaagcttagagagtggtgggagagactggagaagttgattaattagtaaatgaagttgtctgtggatttcccagatcccagtggcattggatatccatattatttttaaatttacagtgttctatcttatttcccactcagtgtcagctgctgctggaagtggcctggcctctatttatcttcctgatcctgatctctgttcggctgagctacccaccctatgaacaacatgaatgtaagtaactgtggatgttgcctgagactcaccaatggcagggaaaatccaggcaattaacgtgggctaaattggacttttccaaagatgctgtctttgggaaacatcacacatgctttggatcagnaaaacctaggcttctaatttgttgataaggcatgaactcaggagactgttttcagtcctagtgaatggtgataattgtaattataacagtagacaacatctcttttacacattttaaatcatgaaaatagaataaccttactgataattttagaaagtggtgattaaaagcacatttaagataatgccttaacacctagtcttttccatatgcatgatgtcttaatcacacattgcaaatcatggaacacagaattttaagcagcatttgtgtagaacttctcagttttactaatattattttattttattctcataacaaccttgaatagaactcagatcatctgtcaatcatgtattttgataacagcctttacagtgagcatagaaaatacagtagtggctaacaacacaggctccagatgtcaggttatctgggtatgaattctggtgtcagcattcactaagcatatgaccttggacaagtgatttaagtttcttttaaacagagaatagtaatacctacctcatattattattgtcagtgtatcatcttacaatcacagtctttctcttagggctgggctcagtgggtggattgacactgcagaaatggccagatctaaaggatcaacatttacgtagctgggaaatgtagctgggacttcagtttcactgccctagtgatttttcctaccactaagcagctcagtccatacccctacgagacccacaagcttatgagatactgttcttccaggaaagcagtggggccagggccaccttttaattgtgtttcttggcctggtcccatctttctcacaatatatagcaacagttatttacttgctgattttctaatgcacatcacacatagtcatattaaacacacacacacacacacacacacacacacacccctcaagaaacattttctgagacgtgatttcctgatttcatcaaaaaagaaaagagcgggccaggcacagtgggaagtcaaggtgggtggatcacttgaggtcaggagtttgaaaccagcctggccaacacggtggaacctcgtctctactaaaaatacaaaaattagccaggcgtggtggcgcacacctgtaatcccagctactggggaggctgaggcaggagaattgcttcaacctgcgaggctgaggttgcagtgagccgagattgcgccattgcactccagcctgggcaacagagtgagactctgtctcaaaaaaaaaaaaaaaaaaaaaagcataaactgaaatttatatgcaatttatatgcctgtgagataattctgttttctcttttggaaccccaaagagatttttttgattgatgagcaaatacattttagattttatttaagcattatgccaagcaccactgaagtataagtttcaagggcaaactcagttttttcatctactagacgaatgattttctggaatgattacaagcaggcaangatggtgnntagtggaaatagcaaatgtcttcggcatcagacaagttggggtttgtttgtatcctgcctctgcccttcaccgaggttgtgatcttgggcagattgttgagttttaacctagattcctctgactccagatcataaattttcagaaaagttctgaaattcttgtatatactgatggtaaatgagacttttccttacatctatgcacttctttgtttgtttgttttgagatggtcttgctctgttgcccagactggagtgcagtagtgcaatctccgctcactacaatgtctgcctcccaggttccagtgagcctcctgcctcagcctcccaaatagctgagactacaggcatgtgccaccacgtccggctaatttttgtatttttagtagagacagggttttgccatgttgaccacactggtctcgaactcctggcctcaggtgattcgcccgcctcagcctcccaaagtgctgggattacaggcatgagccaccatgcccggccatatccatgcacttcttgcaaccttaccttcttttctcatcaccctccagggacctagttggaagagcagagttaaaagttaaggtgaaacttggagaggtgtcttgtccctaggaacaaaggactggtttgaaattctctgtaaatcttccccagttcaaaccagagttatcaaggtcttaaaaacttccctgggtcctgagagcccattatattatttacttgtcttcctgtacacccactgcctagtcctgatcctacttttgtttgcaaataggatggggcacaacgtacaaggaagggcctttgccacccctgctaagggataacctgaaataccttcaccatcactgccctgtgctgcttttcacctatgccagtctgtctacagtgccagtgtctcctggcattgaaaggggagaatcttttggtcctttgagtatttggttgggttacataaatctccctgaatgaagagcagctgacttaggcaaggggccttgtttggttttccttgaactattaacaggaagatagggagattaactgtgtaaatgttcaataggccagagtccctgcagagggtggccacagtgatcagatcttatcacatccttgctttgggtgttgcctctctggttggagtatggatagaaaagaaagaaagaccctatattgaaatgcaaagtgcagcaagtcctgactttggattaacttctcagcccatttgcatgaaaataaaaagatgaataaaacaaggttcccactttggagggaggtggtagctgtgagatggaaggagtgttcctgctgggcaacagcagagtaagtgctggggtagattcactcccacagtgcctggaaaatcctcataggctcatttgttgagtctttgtcctacaccaggcactctgcaaaaacgctttgcctgcaaggtctcatgcgatgctcaccacagctctgtgaagttaattgtacttttatcaccattttacagatgagaaaactgagggtatggggtcaatgacttggctaaagtcactgcttagcaagctgcagggactggatgtgaattccaattggtttgactccaaagcctgtgaagctacttgttcttcaccacctagagctgtggttcttgataactgtgaactcttttggggtcacaaatagccctgagaatatgatagaagcaggagctctggcctttctgtccatacctgaacaggtccttgggttaagagcccctcgtccagggcctattaatcttgatcctcataagcagcatccatgtattacggccgcaaaccaaactgtgccagaccgaatcctaggaccaagcccaaatatgtcccatcatccttttggtaagaagctcattgtaagaaagaaagaggagagcaagaggatgacctagtgcatggggcctcattgttttaattagtgacaaaacaacaataataacaacaaaacccccgaagcttcacagatgacatcagaccccaagcctgtgtgtttttcaggtgcccttgaggagctttgtagctggcagaggaggtgaaactgacaaatgtttggcagatggaggagagtaccagaggggtttgagatgagctaaattccaatctaaccgcaggtgtgaggaagaggcttggattgggaccatggagatgggggttctactcccagtcacgccagctgactttgcgagtgttctttgtcagtcactttatcttattttatttatttttatttttttgaaatggagtttcgctcttgtcgcccaggctggagtgaaatggcgcgatcttggctcactgcaacctccccctcctgagttcaagcgattctcctgcctcagcctccagagtacctgggattacaggcgcctgccaccaagcccatcgaatttttgtatgcttagtagagacagggtttcgccatgttggccagggtggtcttgaactcctgacctcaggtgatccgcccaccttggcctcccaaagtgctgggattacaggcgcgagccactgtgcccagcccacttcatcttaccgtagttacctccttagagtatgaaaaaataggcttagggcatccccaagtcccctctatgtctgagagctgaggctggctgtcaaagaggaactaaggatgccagggactttctgcttaggacccctctcatcacttctccaacgctggtatcatgaaccccattctacagatgatgtccactagattaagaatggcatgtgaggccaagtttccacctgagagtcagttttattcagaagagacaggtctctgggatgtggggaatgggacggacagacttggcatgaagcattgtataaatggagcctcaaaatcgcttcagggaattaatgtttctccctgtgtttttctactcctcgatttcaacaggccattttccaaataaagccatgccctctgcaggaacacttccttgggttcaggggattatctgtaatgccaacaacccctgtttccgttacccgactcctggggaggctcccggagttgttggaaactttaacaaatccatgtaagtatcagatcaggttttctttccaaacttgtcagttaatccttttccttcctttcttgtcctctggagaattttgaatggctggatttaagtgaagttgtttttgtaaatgcttgtgtgatagagtctgcagaatgagggaagggagaattttggagaatttggggtatttggggtatccatcacctcgagtatttatcatttctgtatgttgtgaacatttcaagtcctgtctgctagctattttggaatatactatatgttgttaatgatatcatgcagcagacgtgcatctgaatgggctggctctaggagctagagggtaggggctggcacaaagatgcatgctggaagggtccttgcccataagaagctgacagccaaggctaggggagttctgtcttctctgcatcaggtcacctctctcacctctgtcactgccccatcagactacaatgtctgcaggtctttctcccctgagtgtgagctccctgagcaaagcaggatgctgccccttccctttgtattcctggctcctggcttcagtgcctggacataagtatgggcataataagtgtcccccaaatgagacattgaggattcttcaaatgcacaggaccgtgatgtgagttaggacggagtaaggacgatgggatgtggctcaggacaatcctgaggaagctgcagctgcggcacgcagggccacactgtcatgttcatggaccctagactggctttgtagcctccatgggccccttccatacacaaatattaaaaattatatttcatgactgncattggtataaagatgaatataatccagaccagattcatgattattcatacatttttagtgtattaacttttaattctgcttttaaaataaattaaaacattctaatatgcccttaagagtatcccaggcccaggccactgagcctactgtggttcatggataagnttggccctgggggcatgtgtgtgcatgcatgtgtgtgcacatgcatgatgagccgggccttgaagggtggtaagatttgggtgtgtagaccaatggagaaaggcatttggggcagtgatgatgggtgggggagggaacatggtgatgaatggagctgggtgtggggagccatgggagtgggttagggccagcctgtggaggacctgggagccaggctgagttctatgcacttggcagtcacttctgtaaagcagcagaggcagttggcctagctaaagcctttcgccttttcttgcaccctttacagtgtggctcgcctgttctcagatgctcggaggcttcttttatacagccagaaagacaccagcatgaaggacatgcgcaaagttctgagaacattacagcagatcaagaaatccagctcaagtaagtaaaaaccttctctgcatccgtttataattggaaattgacctgcaccagggaaagagagtagcccaggtgtctggggcttgttcccattagatcttccccaaggggtttttctccttggtggctggcctgtggggcccctctccaggaggcattggtgaagaaactaggggagctggttgccacagacagtgatgtactaatcttctctgggaagacagaagaaaagtccccagggaagaatactacagacttggccttagggacagctaggggtgcagattgctgccaactgcattttttctgaagttggccatatggttgcagtgaatggatttatagacagagtatttctgtgcatataagagcaattacagttgtaagttgatatggataagtgaaagttaagcacttctttctaaaaagagaatgcaattcattttcccctaatcatttcaattagtctgantgggcatttgaacttgttgtctttaaaaagtgaaatctttacctctgatctggtaagtatccaggcaatttcttgtgtgccacccaggaggtatctggggagtgggcattttctgactgaggcattggctgccatagcatcagagcagccttccaggcagtggcctggcaaggggacagaggctggtgggagcagctggctgagtgcagccagtaatggcatgtgcatggtctgtagagaatgtagaagcaataatgaagccgataaaagctggtctgcattttattattatcatgcgccggtggttctaaacaatgtcagtgataaattactcctccccatcatggaccaatggctgccactgctccagggaagtgctttttattccgtttggtgtttagggagggatggagttggctggcctttgctgaaaggcctaccagtttgttttctatttggcaaaagaagaaatgataaagttytagagtttaaaccagactcagatttgagtttttttttttttttttttttttttttttaaggctacagaactgtgctttccttgggcagtaaaagaggcaatgggcaatgtgggacctgatngacaanagggaannngnnnncnnannnannnanngctgtcttaggggtggcatggaggaggtgctgcttcacagcagagagaggtatggctgtgcttggagtgtccacttagacaactcctggctgtgcagccaggccatcgagatgctgtttccttgacctgcaggtcctggtcttgcacatggatgtttcttctggtgcaggagacagaaaggtagcaacaacccctgatcaaagcctcagtccttccttatttactggagagcccctgctgattgaccagaggcacagctggggatatttcctttacctctgtagcaagagacagcgtggtgcagaggaaagtgctagcatacattaccctgtggctgcatgactttgtgaataggttagttagcaccctttcagccacttcttcttacctgttaatgagataaaacatgtaattgcttaaaaacagtatttggcacataggaagcacttagtgaatatgaattatgattttttttggagtggtgacatctcaaccaagccatttaacccctcagccttnactttcctcaactataaaatagcagctaacttgaaatgtaaactataaaacctaatgtagtatctggcacatagtagattcccaataaatgagagccagtattctttctaagacagtgatgcatttctgagcacctggccttgttcttctgccttgcaatttatgcagcagttgaaatagactggctgatgggggtaagttgtcaagcagactttctgatcttagtggaggagactgccttaaaacaacactaatttcctttttcttttcttttcttttcttttaagacagagcctcgctctgtcaccaggctggagtgcagtggcgcagtcttggctcactgcagcctctgcctcctgggttcaagcgattctcctgcctcagcctcctgagtagctgggactataggcatgctccaccatgcccaactaatttttgtatttttagtagagatgagatttcaccatgttggccaggatggtctcgatctcctaacctcgtgatctgcccgcctaggccttccaaagtgctgggattacaggcatgagccaccatgcctggccttctttgagaagctggagacatgagttaagtggtgaagaagccaaatctgtatctaaaaaccctacagtagtgtgcagagctctgaggagagaaggtcccttagattttgagtgtattattatgtcagtgcttgttttacatctctctgttcacgcagtatgtccccttttctgccttgcagctgtttcttaaattctttctttctttgcttgtcttgcagcacaaaacaggcttcagtatagggggaaatgcacagaaacactgccttttcctacaggaaatcagtaactttttactgattttgtttttatttacttattttatttgtttaaatttatttttagttttttttttttttagagacagggtctctttctgttacccaggctagagtgcagtggtgcccttagagctcactgagctcactgcagcctcgaattcctgggctcaagtgatcctcctgccttagcctcccgaagggctgggatgacaggcatgagccactgcacctggccaactttttgctgattgcgaatagcactcttgtcaatttcggagagaagctgagactggcatatgtcagtatggatccccacttagagacctgtgtttatctgcactgacaccccatcacagcatgatgagcttggccctcctgtgctgtctctcccagggctgggaggatccttgaagctgatctggtttggagctttgtcctcattcacctcctttaccacacacccaccttcccagggcggggatctaccactcactaagtagcccattctgggtgttgacagctctaattgttagaaaatattcaccaccctgttatgctttctagagaacaagtctaattctgttttccttgaaatagtcgaagacagctctcatgtttttctttccctgttttcccaaagtccatgattttttaggcaaaatggcctcctttcctcttatggaatgtttttcctccccatttctgcctctcctctggttgtgtttcagtatgtctgtgtgcttcttgaagtttactggaaattatgaaagtattctggcacagaggaggaagggatttttgcctcccttgttctgagtgctacatttccgttaatgcagtctgagattgtattaggcattttggcattcacgtcaccttgttgactcatattccatgtgcactcaaacaaaattgtgattattttaaataggcagaattgcaagttacgtgttctccatttctttgttgtattgttggctttttgaactaaagggaaaaatgtcttttttctgttttacatgtttagattccctatgctatcctatcctcccaaaaccattttagattctgattttgccatgtattatatctgatactcctttctcgtcatctagagatgtgataaacaactctctttggcctcattccagtcatcgataactgtgggacaaagacttgaagcttggatcagtccagtggagactaaccacccctgtagacccttttttcctcaactataaaatagcagctaacttgaaatgtaaactataaaacctaatgtagtatctggcacatagtagattcccaataaatgagagccagtattcttctaagacagtgatgcatttctgagcacctggccttgttcttctgccttgcaatttatgcagcagttgaaatagactggctgatgggggtaagttgtcaagcagactttctgatcttagtggaggagactgccttaaaacaccactaatttcctttttcttttcttttctttcttttttttttttaagacagagcctcgctctgtcnaccaggctggagtgcagtggcgcaatcttggctcactgcagcctctgcctcctgggttcaagtgattcttgattctgtagacactaccactcaggcctatattgtaatcagtgctgggccactgggctcctgcttctgtgatccagttgggaagtttatcttgttcttcccttcagcttgcatctgctaaattcgctggactatacacaggtgatttgtagatatggggatctctactcaaatactctcatgatttccttggctagagcatcattttatttccacttattggaagagaccttagagaccagttagttcatttatagataaattagttgattctgtcattcaacccttatatattgagcgtctcctatatgcgaatcactgttctaagtgccgagacacagaggtgtccaaaacaaatatggcccctctccatatggaatttctattctagagaagaatctgacccagaagggggaagtgactgtcccaagtctacacaaccacagaagggatattctgggaataaatcacggctaaacccccctgctgctccaggcagttctccttctacagtgctctattgtgctgttttaataatctttcaactgggaagaactcccatttcaggaattaagccgtggacaaatcttttaattatccttgaaatcatcctaataagaaatccaaggaggaagtcttacagggtgcctcacccacttttctcatcactggaactttttagacattttattattttcttcctaaaccagagtacaggcacacaagttgagtggtgtggtggctaaattaattaatgtttgcaaggcagtgtgagaagcattcattcatcttaaatacctatggtgactgcaactcagatgtaaaaattggataaatcctcagaaaccctagggaaagtgacatgtctgtattttgtctctgtgagatacagactggcagagataagtgtttctctgggtgagttttgtgtggtatctgggatgattttaggcagtacgtggatgagaacttttaattttaacccacatccaattgcaatttcatggaaattattgcttaggaggatgttcaacaggaaaaatataattaaagttaattcaaaagaaacattttctgtgaatatggtaaaacttgtgagagtagtttgtaaatgattgaagattggaaaacattggtataagagtgagtgtggggttttgtattaagattcattttgggaagaaatccatgctgcatccctcatgaagtgtgaactttgggcatgtgttgattctttctggcccagagtttacctgaagattagctgccctgagggtcactgagcattaaattagatgatgtctgtggatgactgatagtgaagctcatagcccacagttgacacataataaattcgagttgctttgcttccccttctgttcctggctgactgtttggcctttgccacttgttcggcctctctgggccttaagtttctttgccttgaaattggaactttctttggtgaaacaaccagaaaatgcttcagcccagaaacttggtcagtacttggatgggggatcacctgggactacccaaggatgtgggctgtctgctagactatagccccttgagggcaaggtgggcgccttgctcatggttccatcctaagccccagcacagtagtgggtgcatggtgagccattagtgaatctttgtggaatgaaggtgggagaaaataaaatacctgtacttcacagggtattgtgagggtcaagtaaaagtgctttaaaaaattgtattatatagtttattcccttgtgttagcccaggtcaacagagcctacgaataataatgatgacagaagttcttcaaaaagtcttggccttctttctttcacaaaattgccccccagagctttctggaagggcagccatgaacccagaggcctaaagtagatttactgggaagctaaaaatatttactttatttttcatagctcctttcaaggtcctctctgggggtcttagcaatatgtttacacagtggtatgtttttgtaaggtttgcaaaagtaagattttttaaaaatactatcttgttttaaaaagagagccccctaccaacttgtgtcagcctcaggcccccacctgcatctgctcctgccagggcatggtggggcaagaagcactgctccccttccaaagcttccttccttgccctggagtcatcctcactccccactccaagccacctgccatcgctgtgccccctctctggtgaatctggcattcttaggtgggctgagaagcagactggcccaagctaaggcctttctgatggggttgttgctgctgagaatcatgactgggtgggagaaggaggtgaccctttngctgtcttatttttactgtgtatttccttttcagctacttaaaatgtattgcttagtgatacctaatgggttcattagcctgcttcctactgaacatttccgctcaggcatccacttggtcccaaggcctgctcctctcccatattctgaaatctggactacagactctcattcaactccaggttgcactgtggacacagtcccctcttgagcaggtacctccttgcagtggttgggacgtcctacttggctcatagttgggaagtgcatatgctggagctgaagcctcttgccttcccgggatagggcgtcctcacatcccctctgagaagttccccagcttccctctgttccccgtttccacacttagcgaggctcttgtccactgctacatcccccatagccagtccctcagccttgccattgcttatgctggtctggaaacaattcctagacttgtggggcatctggggaagttctccatctttttttagctggcatgacccaagtggtgtgggcagggctgtggattctatggtgtggctggaagccaggtagcctctctctactgtacatggaactcagcaacttctgagtcaagcaagatcttagctctgcaggtgtcttgccctgtccaaagttatggccacaccagtaccttttaactctagaagcccagtaagtgttttgtgggaccgcaaagatcattttctagacctgctgaatatgcctagaacgggtagggatggctttcacgctgttcctagggctgacaagtcacacgtttctgggggtacatacacaccgcggtccctgtgaatggcactctccatgagaactgtgatgatttgagttgaatagtgcacagcctacatggttctctgccatggcctggagttccttatcttgccttctccagtgaggactagggctgcaactggcctactttggctcctgacttgggggattctgaaataccttttttttttaaggttgtggagctctctgaagcttataaggattttgccaggaaaagataagaaatatctttgggcattttgtcactgtgctggagatgaaccctttggaggacatatcaccttgttgaggtcaaggggcggaaagggacaggactggcagagagatccggggcagcagcctgccatcccgactgagtatggagtttctctctcccttcagctgcacttttgtgtggagtcagtggctcagctgccacttcccttatgttcatggcatgaatctggcttgttaggcctttcttttttttttttttttttgagatggagtctcactctgttgcccaggatggagtgcatggagtgtagtggtgtgatctcggctcactgcgaccaccgcctcctggattcaagcgattctcctgcctcagcctcctgagaagctgggattacaggcgcatgccacaacaccctgctaattttttaatttttatttatttattttttttcaaggcagaagaatttttcttagtacagaacaaaatggaatctcctatgtctacttctttctacacagacacagcaacaatctgatttctctatcttttccccacatttcccccttttctattcgacaaaactgccattgtcatcatggcccattctcaatgagctgctgggtacacctcccagatggggcggcggccgggtagaggggctcctcacttcccagaaggggcggccgggcagaggcgccccccacctcccggacggggcggctggccgggcgggggctgccccccacctccctcccggacggggtggctggccgggcgggggctggcccccacctccctcccggacggggcggctggctgccctgctaatnttttgtattttcagtagagatggggttttaccatgttggtcaggctggtctcgaactcnncnntngacctgttgatccaccntgnncctcangcnnctcccaaagtgttgagatntacaggcatgagccactgcgcccggctctgttntttttgtttgtttgtttgtttttgtttttgttttgagacggagtcttactctgttgcccaggctggagtgagtggagcggcacgatcttggttcactgcaacctctgcctcccgnggttcnaaacaattcttcctgcnctncagcctcccgaatagctgggattacanggcacttaccaccnaggcctggctaatttttgtattttttagtagnnangacggggttttgcncatgttggccatttgaactcctgaccatctttngaactcctgaccatgtttgcncatgttgaactcttgacttcagngtgcgttggccntcccaaagtgctgggattacaggtgtgagccaccatgcccagcctgtttggcctttctgatatgngncntcntgactaatctttttggaaattnagnnntccccagggttatactggattttacttagggaaaagggtcatgcnctctctggctgtcagnatttactgatagtactaaggnactcagntggggtgnnngnacctttgattcnntnggtttgatttttgaaaatncaaaaagacgtgagctccagggagcagggtggctttggtgancatggcaagatagttggctgtggcnagggagttgagggaagtgggtagaaaattaacatcttgtanaatattnncncctgggaaatataccttcgtgttaagagaacagacttggcagnccnnannngnnannntnnnggcctaggttcaaatcttggcttgatgcttatcagctgtgtaaccttggataattccatacatctctgtgcctcagtttcctcaaatggaataacaatagtacctccctcaggactattgtggcaaattaatggacgaataaggggaagcacttagtacagtgcctggcccagcataggtaccaggcttgttcttaagctcactgcatttttacaatcatcataaaatgcaggggatacacacatgaaggagccgaagttcagagaggccaagtaacttgcctaagaaagcacagctggcaaggggcagtaataggaccagaattccagtttctcgtgctctgttgttgttatatcctaaagagagcagctctgagtagccagaagcttccctaaagtcacaggacatggggcatgggctggctgggatgagaaaggagacaagagggcttctgaaagaaatgccagattcactccacttcctggcttcaggcaccgatggaatgtttcccaaggcccatctagaaagaacatcctgtgactcacagccacttctcattttctgtctgaaccccctcacccattcaggcagctgctaaagttgaggttacagcctcagactatattttctgtccttgtggaaccccagtgtgtcatcttgttgggagatctggtgatacatgtgtcaacattatgtcatcaaaatggaaattctttgaaatctttaggtgattgcaattcacgttctgtatgtatgcacttgtcaaaagttttgatttgaggccttagaattttatatttggaaacctttccactaccatgagttttcccagacctgtcaaagccaggctgcatctcagaaaccaggtctttgatttccatccagggcaagggcctgggcccagctgggctgtaagcaggtgggggtggggagcaacgctgcactgcaatgttgaaatattacttgaactaaatcaaatcaaagatcagctttactcagacaagaatagaaaacacaattgcattcgattacagaatagtgtgtatccccacaaatatcagactgcctttaaaaagttttgaattgttaacatcaagaacagtgttgcgtgtctcctgcttttccagcataaggtttatttattctgtgggtggcaaagagcaatttgggagtccagtttgtttctcattgaaagctttcccatttctggtcctcttgtcactgttgcattgaggcaccaaaaggcaatctcagtgcgacactattcaacagactaagttgcaccggataatgataccattttacatttttcatatattattacattgaaggcttcaaacagcactagcaggggcaaattggtattattatctccatttcattgatgaggaaaccgaggctcgaaagggtaatgtctgttgtccaagattaaagagtaaagttgtacgttgaatcggggtctgactcctaggcttagcattttctccccacactatgctgccatgttgcttattccaacattaggaagcataggtgccatccccagcttttgaggccaatatcacgatgaagcatttttaaaacatctcattaaattgctgatatagtggaaagaaccaaagctttgcagtcaaagctgtttgggttcaaatttaccacttgtttgttctatgaccctgagcaagatattctccagatctgtttcctcatttgaaaaatgggaataataatacgtttctttataggctgttcttaaagattctggaaaataatgctaatagtgtgcctaatgcttggtaaatatgagtcacttttctgtgcccacaaagcactactatgtcccttaataaattttgttaattttaaaagttagaaaaaaattaaactatttatacattgtgtatgttaattcttccctagaccagccttaggaagaatctcatccccaacttgtaaactcatctttttccgttctttttgtgcctggacttctcagggccctgcaggctgattctagtcccatgttgtgtggtgtttgaagtgtctggtcccttttttcagtgagagaccagctcatccttgggaactgaatgcctcaaactctcttttctttttctctcttccccttctgtttgatgtagtctttcctgtttctggactctgtttcttcatacttcctatctcttaccttcttttcactccttttgtcttccagctgtcctctctcatttttctgcctctctggtcttcaggtagagttttcatctcagctatcttcttgctttttctgatgttggttctttgtttcttcttctcattctgttcaggtccaaaattcatttgggtcaatgttatgtcttagtgttatcttccatttccttctgagcttcaaagccaggctgactgtgcccttccacgccctggccagtgtgaccaggacatcctttccctctggggctgcactggctctttgggggaattgttaccattcagggtcttcaaccctcattctagggacttccagtaacttctcccaccttccctcttctcagtaagacatgggtattgtcttattctgtttctgctgctagaagaaaattctcgagactgagtaatttatacacaatagaaatttacttctcacagttctggaggctgggaaatccaaaatcaaggtgttggcaggtttggtgtctggtgagggctgctctctgcttctaagatggtgccttgttgctgcatccttaggaggggacaaacgccatttcctcaactggcagaagggactgaagcctctccctcaagcccttttataggggtcctcattgtcaggcctctaagcccaagccaagccatcgcatcccctgtgacttgcacatatacgcccagatggcctgaagtaactgaagaatcacaaaagaagtgaaaaggccctgcctcgccttaactgatgacgttccaccattgtgatttgttcctgccccaccttaactgagtgattaaccctgtgaatttccttctcctggctcagaagctcccccactgagcaccttgtgaccccctgcccctgcccaccagagaacaaccccctttgactgtaattttccattaccttcccaaatcctataaaacggccccacccctatctccctttgctgactctcttttcggactcagcccacctgcagccaggtgaaaaaaacagctttattgctcacacaaagcctgtttggtggtctcttcacacagacgcacatgaaactcatggtatctgtgaggggccctaatgctattggtgagggcactgctcacataacttaatcaccccttaaaggccctgcctcttaatactgtcacattggtgatgaagtttcaatgtatgaactttgaggggggacacgttcaaatcatagcaggtgtgctattgccttactagcaaagtaatctgggggaagatgagtaatgtctcgttccacccctgattccagtgctgactgattctggagtggcagagagggttggaggctcaccgctctgctcacccgaccctctggccatctcctcttagaatgcaagggcagggattttgttacacagcgcctcttttgttataggctagctcctgccttcaaggagcactgagaaaaatattatccctttgaaccacaacactagtattttgggtactgtagccattcagaagtttgttgaactaatacaagtttatattatttgtaaaatactagaaggaatgtgtggttcctaaagttatggtattcccattggttgaaagagaaganctctggcttcctaatgccttggaaggttcagggagcataagcctaaatatcctggtttctcttgggaattcttcacagcttgtcatctatgactttatccacatttattttgaacatgtttaattttcagcttgtattttctcaaggagtatgtgtgagtcagcattcagagtacttaacacacttagtgtcattagtgataacttaacccacacgtataggaagctagacctccatttacagaaaaatggaggctcagagagcctaactgactttcccaaggccacataactggaaaattctggaactgagttttttcaggtatgtcttggtatccctcggtgagccttctctgtagagaaggaagtgtctcctgggttggaaagcccaggccgggatgaacagcatgggtgtccttaggctgtgtgtaccacacacccctggcctactctcccctgctctaaggagtcacctgaaggagcagctgcatcaccccgcctcaccccttctcctcagcacccaacaaccaggtcttcccagagagtgctcagctattggtgaagcaggcatcgtctattcttcaagaagacagcagagcatcaggaaaacaaaacaccaaaaaaataccataaagacactggagtgtatttgttaccagcccttgtataacagagaagtatttcaaggtcttatttgtaaggctggacaagctcaggctgattaaaaccacgagttagaatttgtctcccgctcccaggccctgctcagttgacagctcctagcaggctgggaggccccaccccacccccaccccaggggtccattgaagaagaccatctgggcctgggatgctgatgtgattttccctacttctccttttttttcatttgcctcctccacccacttctaaagcagctgattgctctggttccttttcatgttgagcaaaagagaagacagatcttgaaacctgagctggggactggagggcactgaccgagagtaaccccgtctggctcagctcagctcagcaaagggctttcctcctccgccttcctgagctgctctccctcctctcctttcctccttctttcccagcctcccccaagctccttcacccctctcttccctctcctccctcctcccaccttccttgtcctctatgctcagagaactgaaggaacactgaatggcctggaaagcaggacctcgcccccaccccgcgtcatacccctccgcctgcacgtgcaaatgtgttcctcagcttggtcccagaggcctggacctgggtcccagaggtcccagctggttccccggcttggtcccagaggcctggatgtgtggcaagaagctgagggttctgcttcgtttcttccagcccacaaacacacatgtcacctgttttttgttctttttcctttcctttgccccactttacagaagctcttggctaattggtaggtcactgtttgctcctccaggcaaaaccaaggaggctgagctcagcaggtcaagtattttggtctgtattgaagagactgtggtgggagagccctcttgaccttctgctggatagtgactttctcttctgctggcagcagtagcccagctccgcatcctgtgacagaggtcataccttccagttacccgagcgaacacttggagcaagtcagctttttcccctgaaacagcacgttcacgttcttgatctttggtttgcacaacaacctgtgaatgttattagccccgttttttttgtttgtttgttttttgagacagagtgtcgctcttgttgcccaggctggattgcaatggcgtgatctcggctcactgcaacctctgcctccaagcaattctcctgcctcagcttcctgagtagctgggattacaggcatgcgccaccacacccggctaattttgtatttttagtagagacggggtttcttcatgttggtcaggctggtctcgaacttccgacctcagttgatccacccgtcttggcctcccaaagtgctgtattagccccattttatatccaaggaaactgaggcttagggagatttaaataggggggaaagtatagcttcatgattacacagtcaggaaatggcagagctgactcttgaattcagaccctgtgatgctgtatccagtgctgattttgttttgccctgtgttcctttcagtgtcaggtaagaagacctcagagatcatcttctccaacccacacattttactgattataaaatccaggtccgcattaactcattggccaaagttaagtggctaaatgactctcctgttcaggaagtattccctgtcttgtgtggaatgattggtctttcgtccaggtcatcttccacctatatccatccttgcagtgactggtggatatgatcattcctggtgattccctgtaaaatactagatttatttattctttcataaagttatgagtagacaaatcagtcagcttcaacctttcctttaccttatagatagtatcagcagaaagaactcaataattctgtcttaggaactcagccaggctaatatgtattataatacacacatacatatgcacacacatacacacatgtgcataccttagatcacaaattcaacatctcaacttagccgccaaagccacnttctgtgtcttgtctattctgaatatgctttaaggttaaattagccatgaatctgttcagatcagtcattcctgaaaatgatgatgatattagccatcctttaccgggcaccttactcagtgcccggcattatactaggtctgcaggtacatttgctcatttaacacctagctcagtccaatgaggtggatgccattgtcatctccattttatcatttaatcaactcagccttcctctctcctgttccccactctgcttctagctgccactcacctgactctcctcttgatctgggctgacgtggctgagtgggtttagcagaactcttgtgtcctctttacttggtttgtttcaagttcatgtcctgcttcatcttttccctaactcactgcaaggtgaattttcttctctgcaatcggcaattccccaaatcacatcaggtcattaatttattcactcatttgtattcattcattcattagttcatccatccatctattcattcattcatccaatttactgagtgcctcctctgtgccaggcactgctggctttggaattaaaaaccctggccctcaaggaatttctattcttctgggagcaggatatttacatgggtgactgcnagcttgatctgtgtggtactagtggaaaacataggatgttgggcacaaaagagtcacaaaacgccgggcgcggtggctcatgcctctaatcctagcactttgggaggccaaggcgagtggatcacctgaggtcaggagttcaagaccagcctggccaacatgatgaaaccccaactctactaaaaatacaaaaaggaataatagctgggcgtgatggcgggtgcctgtaatcccaacaactcgggaggctgaggcaggagaattgcttgaacctggggctttggaggttgcaatgagccaagagcgggccacttcactccagcctgggtgaaagggtgaaactctgtattaaaaaaaaaaaaagtcacaaaaaagggctggccacctaacccagcttgaggatgggaaggccagggcaggcttcctggaggaggtgactcatgggccatgtcatgaggatggggtaagaggagaaggtatgtttcagacatctggctttcttaatattcttggctttccacccattttcctgccagcaacataggggagaagactgagaccagcagatacaaagccactgtactctgtcctcaccccctccttttttctccccttcctactacccagatctgaggttttgagaaatctcttctctaattatctgcttgttcattcatttgttgagaatctactatgtgcatagcattatgaccacaaaatgtcctggtcgttgtcatcatgagaggtcttaccccttcctctccagccacttctagggatttttgactctgtccttttccagaacttggctccagtctggttgctcgccatgaagcacttacagataaacctcatcttgggccagtgcttccatttactgtctccttttggcttgcttatccttccttctgccttcttgaattgatttgcctctttatctcccctttcagccttgaaagttcctcgaaggcagggactgtgtccccatcttttctataaatggcattgttgtacattggtaaagttgagtgaaaaaatctttttgataatcacaaattattaccatatattgagcacctaccatgatgagtatctctgatcttcatatctttgaaatgtggatactattaccttcacttaattggtaagaaaatttgtgctcagggaggtaaagtcactttctcacagtcacacagctatttcacaggagagtgcagtatcaaatttaggtttttctggtcccaaaccctgatgtttcccccacatcattgtttctcagacttggctgcaatcacctgaaaaattttattaaattcttttttttttttttttttgagacagagtctcgctctgtcacccaggctggagtgcaatggtgccatctctgctcagtgcaaccactgcctcccacgttcaagtgatcctcctgcttcagcctcccaagtagctgggattacaggcacccaccaccacactcagctaattttgatatgtttagtagagactgggtttcaccaggctgatctcaaactcctgacctcaagtgatctgcccacctcacccagccaggttttattaaattctgatgcctgggtcttaccctagcagttctcattagttgctccatggtgtggcctgggctttgggatttttgtgaagcccccagctgagtctaacgggcagtcatgtttgagaaccagtatactacaccatgttgccttcctgttttcacacaggttggtgctagtgtgtgagtctaagcctacatgggtggatatccacgtgatcaggctgcaagttctttgtagtggaggatctttggccctccctgctgcttcccaacccagcgaacctaccacaccatgcacgtgcacagccaagggttgttgactgtttaatcagtctctgggttcttatgtctcatccgggggattcactgataggaggctggagcttgatcacatccagatgttctcatcttcagagcctgttaacataacagaagtcttataaacatgctgagcacactcactggtctggagagtgttgcaggatgacctaggcccattgcaggggtggcctgggtcgccctcccacacttctgccaccctgcttgggaaggagggagctgttttgaagttccctggtgcaatatgatgtattgctctggctctgctcaggaggaagctatggcccacctagtcagtgagggttagctaatacgtgtattctgtttctgttgaggtttcacagaggccttttttgacccttcattttatagataaggcagtggaggcacaactacatgaaatgactcgacaaataaatggatttagaacccaggtttctgactcccagggtggtgctttttccatggttgtacagtgattaatgtctaccttttcacaccagtcctcaactgaagacaccagcttacaccttccttcctgtttctcccaagaacagaaagtgaccccggatgtcgctttctgttcctgggaaggcagttccagtggttagaagtcctgttcactcctggggtgtggcctggggatggtcctgacatccctgggctcttcctggacctggccagctaaaaggaaatctcctatgatggtactcagatacttttggaaccttgtcagccctaatcccatctcctagtgtttagtattcagctacccttcactgggcagtaattctgtgccaggcctgatctgggcattggtggtactaagaacgcatattccctatcctataggcatagtctggtagagacaacatgcaagtaaaacaatgttccttaaactgtggttccacacctccctcccccaacattaaaagtgtaagggatgcttattcaaatgtagatttgtaggctctgcactctagacccactatttcagaatctctggggactgggcccaagaaactgcattttcgcatgctccctaaatgaagcttaggtgctctgaggtttgacaactgcagtagagagcctaatgctaacagtgtagagtcacatgtgatgggaagcatcaggtaggtagcagtttgcaggagcactgattctgagggacactaactgggcctaagaacagctactggctgtcatgaggaataactaggagctagccatagaggggtagcagtgaatcatttctctagcgatgtaaatcttgctcaatttattctgtctatataactcaatattactgaagtttgcctaaagcagaatacacctggatcatacagcatttatgagagactggctgggctgtcaggccctcctgttactttatctctgcatgtgaccctcttasstccgcggattaactcctgtcctcattaagcctcacactgtagccccattttcagatcaaacctgtttctcttctggtaaatgatttcagtttgcaaagtttgccctctagaggttgcttagtgcctggccatgtgggctcagttcatgtggtcctgatgagctggttttatctttattacaaagaagttaggctgttaggagagtgggttggaaggagaagaggtagacagccaaatgagatgagtcagggaaaactatactgtttcggagtcatagggctcctaccaagcatctggtcagaaacctctcattttggagatcaagaaattgaggttcagaaagatgacatgaggtacgcagggacgccaccagacacagcctccaactctagaaactaaaattctggattcttagtgctgctttttctgttttgttgcactggattgaagccttttctaactgtactcagagggcctattatttagggagattccgtatgaaatcctttagcaatcaaatcatttaatagggctgatggtttaaatatatgttaatgtgttttcctaaagcctggcagacctgggttttggagctttgtatcacatgttttatgtttggaatgaaaatgagaccatgtctgtgaaggcactttgatatgcgtaatgcactctgccagtgtttgtcaaaacatggtccccaggtcagcagcatcagcatcacctgtaagtgattctccagtcgcatcccggcccaggmsmgggccmrrctcnyaggcyytgtgsmmggcsmggnnsggnnntgnccggcccggcccggcccggcccggcccggcagtctgcattttaacaagctctccaggtgattctgatgcatacttaagtttgagaaccattgcttgttttgcattaaacaggagattagtctctgcagcttgtgggaataaagctttaaatctctccaattttagctctgtgaaaaggcagtggggagacaggaatgaacggactagtgccacaaagctcaggtggggtgggtgagatcatttagaagagaaagaccgggcatggtggctcacgcctgtactgtcagcactttgggaggccaaggcaggttggatcacaaggtcaggagtttgagaccagcctgcctatcatggtgaaaccctgtctgtactaaagataaaaaaaaaaaaatttgccagtcatggtgatgcatacctgtaatcccagctactcgggaggctgaggcaggagaatctcttgaacccgggaggcgggggttgcagtgagctgagattccaccattgcactccaacctaggtgacagggtgagactccgtctcaaaataaaaaaaaaaaaagaaaaggaaaggctgtgtgtgtgtgtatgtgtgtgtgtgtgtgtgtgtgtgtgtgtaacagcaccatcacactgtttgagttgaggagcacatgctgagtgtggctcaacatgttaccagaaagcaatattttcatgcctctcctgatatggcgatgctcccctatctcattcctgtgtgtgtttagccaggcaactgttgatcatcaatattatgataacgtttctccactgtcccattgtgcccactttttttttttttttgagttacttactaaataaaaataaaacactatttctcaatagacttgaagcttcaagatttcctggtggacaatgaaaccttctctgggttcctgtatcacaacctctctctcccaaagtctactgtggacaagatgctgagggctgatgtcattctccacaaggtaagctgatgcctccagcttcctcagtagggctgatggcaattacgttgtgcagctactggaaagaaatgaataaacccttgtccttgtaatggtggtgaaggggagggaggtagtttgaatacaacttcacttaattttacttccctattcaggcaggaattgccaaaccatccaggagtggaatatgcaacctggcgtcatgggccagctggttaaaataaaattgatttctggcttatcacttggcatttgtgatgatttcctcctacaagggatacattttaagttgagttaaacttaaaaaatattcacagttctgaggcaataaccgtggttaagggttattgatctggaggagctctgtctaaaaaattgaggacaggagactttagacaagggtgtatttggagacttttaagaattttataaaataagggctggacgcagtggcactgagttgagaactgttgcttgctttgcattaaataggagatcagtccctgcagcttgtgggaataaggctttaaatctctccaattttagctctgtgagatggcactggggaaacagaaatgaacggactagtgtcacaaagctcaggtgggatggacgagatcacttcaaaggtctgtaatcccacgtctataatcccagcactttgggaggccaaggcgggaaaatcacttgaggtcaggagttcgagaccatcctggccaacatggcaaagcctgtctctactaaaaatatgaaaattagctcagcgtggtggcatgctcctgtagtcccagctactcgtgaggctgagacaggagaatcgtttgaacctgggaggcggaggttgcagtgagccaatatcacgccattgcactccagcctggctgacagagtgagactccatctcaaaaaaaaaaaaaaaaaaagaattttataaaatcaggaaataatattagtgtttatgttgaattttaactttagaatcatagaaaacttcctctggcatcattattagacagctcttgtgcagtgggtagcaccagacccagcttgcatggttattgatttttcagagacactttttgagcttattctctggcagaaaggggaactgcttcctcccctatctcgtgtctgcatactagcttgtctttacaagaagcagaagtagtggaaatgtttattcttgaaaataagctttttgcttcacatgatctagaatttttaaaattagaaaaatgtgcttactgcgtgcccttctgaaactaggaaaatatgccttgtgtttaccaattgtgtggttaggagatgggccaaaggcatcaggcttttgaaagtagttgcatttagcataatttccattgccccctgccaatttcatatctgtcacatctaatcagtttaaaataaggggcatcctaagcatggagatggtccttggatggtccttggagtttctgtattttcagtattcttttttttgagcatacaagacatttattgaaaaattcttgggatcaatacttgtgtaaggaaaggaaagggaacaaagcatgattgggcagaggcagaagatgacatcaacaaaggccctccgttgtcagtattctttttttttttttttttttgagttggagtctcgctctgtcacccaggctggagtgcagtggcgtgatctcagctcactacaacctctgcctcccaggttcaagtgattctctgcttcagcctcctgagtagctgggattacaggcatgcaccaccacacctggctaattgttgtatttttagtagagacggggtttcaccatgttggttggccagactggtcttgaactcctgacctcaggtgatccgtctgccttggcctcccaaagtgttggaattacagacatgacccactgcacccggcctgttgtcagtattcttaaacatagacactaactgtaggctgacagcctagcagcaaggaccagttaaagaaatgagtagaactgaagtgtgcttgagtatctctggcagtcagcaaaaacttaatgggaatcatggtaggccaaatgttctgcagtatttcaaaagctgcatgggttttgagaggcttttggtccatcactcaccttaggttgttctacaagcacatcagcctgccccaatttaaacagggcagttagtaatggtgtaataaagagtctgcattgttcattccttcaacaaatactggctataatgtttcagcactgtggatgcaaagtgagcaggataaacaggctcttctttcaaagcttgtggtccactggaccacgtatgaagtagaatagtttaggtccagaaaggcaattaagtaaaatatgaccaagaagaggctctctagtgggtttggtataaagaaaagataagaatgatttagaattggcctatcaatgagataagaggcctggctttctggcactctgctctagggcaagtaaaatggagaattccaaattctgaaattgttagaacatagttctgtgtcttagttaaatatctacacttacagataaatagcataaatgctttctccccatatttcagcccagtcctacttaaagacaacataaattgcaaaatagtgaggatgttgttcatctaataaaagtggttccaggaattcagactctggattcctgtttgccaaatcatgtgtcccactcttaagaaaacgagttggactctggatttttctttgcaagagggacaagagtgtgggagatactgagttaatgcaacttgcaggttttaagtgtcctgtcattgtgccttgtgctttgatacattctgagtttcagtaaagagacctgatgcattggactgttgcaatggaacctgttttaagatcttcaaagctgtattgatatgaagttctccaaaagacttcaaggacccagcttccaatcttcataatcctcttgtgcttgtctctctttgcatgaaatgcttccaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctggtgagtagacttgctcactggagaaacttcaagcactaatgctttcggaatgtgaggcttttccttggacagcatgactttgttttgtagaaaagtacggctggctgggagtttgtgatataatttagttcagtggtattctaagtgttcttagtgttctttcagacttttgggccatctcccaaagggtgaatgggaagaataagctgggtgtggctgagtttaagccaaaagttttttgtgcttgtttcaatcagagaagacctgctttttcatgtttttactattataatactaagcaagagctcatttgaaaacagagttcttcatatttaaaaaaaaaaagtcttgaaaccattgatgggaagatggatatctatttatgtttaaaaacccatcataaagatgacattgtgggctgtcacagttggaaggccctggaattagatgagaccacactatttagcttacttagtaataacattgcaaagaaaaattccgacgaagttttttcagcctaggaatcaatagttcagagaagcactctatgagaatacccattcattcttaaccaaaaaatactggtgagcctgagcagtttggtcatcagagtgttttatatagttccagaacaaatatgtctctaggtgttctgagagctctggtgaaattcctctcgctaccccaaacatcatcatttaatatccaggattctggttttctactcaccagatagattctcttaaaaccagggaaagattcctggaggaaggatgtatctggaaagagatgttccttattataataaaatgaaattgtaatactcttggattttgtgcagcacgaattctttatagagagttggtcctcccagagaattaagaatactcagtttctggaccctgttcccagatcataccctagaatgtgaccttagaaacacacttcaggattcatacctttgattgaccatcaaaaagtttttgtatcggccaggtgtggtggctcacgcctgtaatcccaccactttcggatgccacggcgggcagatcacgtgaggtcagcagtttgagaccagcctggccaacatggtgaaaccctgtctctacaaaaatacaaaaaatagccgggcgtgatggtgggcacctgtaattccagctactcgggaggctgaggcaggaggatcgcttgagcctaggaggtggaggctgcagtgagctgagatctgtctcactctgttgcccaggctggagtgcagtggcgcgatctncgactcactgcaacctccgtctctcatgcttaagtgattctcatgcctcagcctccgagtacctgggactacaggcacctgbcaccacgcccagctattttttgtattttntagtagacatagggtttcactatgttgcccagctggtctcgaactcctgagctcaagtgatctgcccacctcgggcctcccaaagtgttgggattaaaggcatgagtcaccgtgcctggtcccatgttataattttaaagtaaggtatatttctctacagggatctttgcaaccctaagtaanctggcctaaaaagttagagaagctgacttgtgcagacatttgcagcctgttggtcttttttgtgctgtgaatcatagagggtgaaaggttattatgaatggtacaaaactttgttacaaaaccattttcttggactgttttgggctgcttcactgcatgacaaatgctcaccctttcagctggaatgattgaaattttggaaaagatgggtgtttttagaagacattgtaatttgttccggtgctgtgcccattcattccatttcacttctgtttactcattaaacacctattgtgtacacaacccggtaaaatccctccactcacacaatgcctgaattatactcatagtagaatgactgtttagccctcatcatctgataattaacagctcaggtttcaacctgacagtatctctctgggaggattagcagcgtgacagagtgcagggaaatgcaccttcagaaccgtcagctacactgtgtcccatcctgctgtgttgtggttgtgccttgtggatgcgttggtttatgaccaggtattgattaaggtggctactaccaggtgctttctgcatatctcgggtttgtggagcactcaggttctgcttctgcccctctgctgttaccaagagacctctcttcaaaatggggctcttgagttagagtagaatgagtgatcaggattgttttgtgtaaagatgatttctgaggaaggctttaggatgaaatgacttccaaacattttgaaatgtgactcttacttattgaattaagcagggccttaattggaatgctgggactgatacttgatttgcattaagcagcctttttctattgctgcttggnttgaaatttcaacatttgtgatggtagatggatgtgacatgtgatgacattgcacatgggcagttaactgtgccaagaagtgcagcagtagcagcaaccggagatgcaaagcccaacatgatggggagagaaactcttctttcaatatgtgcttctgtaccaaaagtggaatttcacgagagacatattttgaaacatttctccttttgtgtgtgcgtgagtgtttccctgtttccagccaagggtattgtgagtttctcctgggcctccttcagaatctgggtgctctggaaagcagtgttttggcaacatggggaaagtatggcagtgtgggagggtcagctgggtctgggtttgaatattgcatttgaatattttaccagcattgatgtcggataaattatttagtccctgtaagcctcagttttctcttcttctacatacacataatatatttgactctttgttgtgattattggttacacatatgaagagcctggtgtggggcctggcacacaataggtgctcaataaatagaagttgataatttaattgacatgagtagtagaaattatgtccttgaaaacaattgcgtcaagatagaagttttcagccaggcacagtggctcacatctgttgtaatcccagcatattgtgggggccgaggcggatgaatcacttgaggccaggagttcaagaccagcctggccaacgtggtgaaatcccctctctactaaaaatacacatatttgccaggcaggcgtggtggcgcacacctgtaatcccagctactgaagaggctgaggcacaagaatcgcttgaacccaggaggtggaggttgcagtgagctgagatcactccactgcattccagccagcgtgacagagtgagactctgtctcagaaaaagaaaaaaaagatagaagttttcttctgtagatcagtgttagaactcataccaagcgaagtggtcctggtgagtatttcagtgaaaaactgcattcttgctcagatattgtcaagacttttcacccaaagattcttatttatgtctcagtccgtaccttgtgtgaaaattaatactggatgtcagaacgctgttgtgtttttaaagttccctggggttaagagcagtttccattaggtgttctctgctttttacttaaaaatcttactcatgcattgagcaatatttattcagttcttattatgtgtcaggtattttctaggagctggactcaactcaaaagatatccttttgatgagaacaaaggtgggtggatatatgaaatattatctgtgggataaatgcacttagtcatgagggagacttgttatggagtgcgctcattgtatttgtactgttgagttaacaacttctaggaggagctcagggccacctggcaggggcttcttttgtcttgctgctcagcaaggtgtattttgctgtagagtgtgctgggcaggtgaacttttcttaactttctcttgggtccttcctaaagcagcatgtacctttcccagagcgaggagagggccaccttcctgtctcacagaaacctccaatctgttttggactgcaggaaggagccatagtagtggaccagcaaattttggcccgagagattgatttgcttccgattgtactttttttttttattgctactataacaagtcaccaaatacttagcagtttataaccacatacatttattatttcataaggccagaagccaggatacagtagagctcacctaggtctacttcttagaggctcacagggctgatatcaaggtgtttgtggggctgtgtttttttgggggggctctgaagatgaatctgctttggagctcatccaggatatcagatgaattcagtcccgtgcagttgtaggactgaagtccatttccttgctggctgtcagcaggggctggtttttgctcctaaaggttgccatcattccttcttatgcttttcatgtgactcctctcccacaagtgggttgagtatctctcacactttaaacctctgacgcctcctgtcacatctgtctccagtcagagaaagttctttacttttaagggctcaggtgtttagtttgggcccattcacataatccaggataatctccttattttgaggttcataactttaattacatctacacagtgcatttgctatgaaatgtgcatattcacagactccaggaatagggtgtggacatctttgcaggggatattcagtctgctgtagctttcttgattggaaggaatagtttatcatatatttgaagtgttccctcagtgcctttggcttttgttgacccctctgcagctctctgctttttcttgcccatatttggaaagtgactctaaagcataataagcatcacctattagggtttttaatgtacaaaccaaacagaggtgactttgggaggagaacatctctgaactaggtatgagacattcatcgaaaaaaatccatcaagtgtttattgtatgtctgctttataccagcactgttctaggcactgaagttcgaccaccaacaaggcagttgtgatgcacttggagctttcattctgatgggcattgagatccaggctgaaggctgagtctgggaatttgaggaattctttgtaggtcctgggtctacagagtgagagctgtcctcgttccagtttcactgatgacctctcgaccagctccctcacagcagtctttgccaacacagacactgtgggctgtagtgggaggaatgtggggttgaatgagctaggtttgggctctgtccttgactcaccattgcctcagtgatgtgaaaatggtgtctgatccttaaggttggaagtccaggcatgcagatttatctccatctcaataacgtggggaaaaaaaagaagtggtttttgtggaaccagtggttccactgtccacgtgtcttccagtgtgcctagcacattcccaccaagtggatcttggttcatgagggaaagaagggaaagtgagggtgccccgaggctcccagaagacaagtaatgtcacagctgagctgtgtacagatcgaagaagcagatggataaggagtgaacaaagtcatccttgtcttgaggggtctttatttagcttcttcttgactcttagacaaggacccagaatacagatggggcttgttgttaccttcagcctcatggctttttagggctagatacttcagcttgttacatgcagtccttaaagcgtctgtgtgggtttttgcaggagagaacacttgctcgtgttctcctgtctggaacccggacattgttggaaagtatcagattttgtttggctttgtgtgatttgactgcccacctgtttacttgctttctcccagcaagcagccgcaatccccatgggttggtaatgggtggaatgacacactgtgtagatttactcttcagactctatgttcacctcattcttatgggaaaagaagagcactagctggtagatatagcagtgtgttaatatgactgctcaactccatttacacagacattttcaccttagttatactatttcttcattaaatattgttgccagatctaagatacaggtttaattttttcctctgaattatgtggtagtagatgtatttaactatgtttagaaacagcaaaaaatgaagcgtttgaatgcgttaaacacatctaatttgaaagttaatatttaggttgttgatttattttttaaaagattagaatattccttagaaatgtagtcttataatttcgtatttcaataaaaaatattaaaatgtttcccagaggaaatctttactgtcatagaaatcaccagaaagagatagcaattactcctgggtggtgatagtctttgatttgtggtttacttgttttcagtttgaactaaaacatactaatcccagcctgagttggattttgcatatggtcaaagtgaagtagagatttttgtctactttatgaagatatttaaaggacatttgaaatgtttcaatgaacacattgtaacatgcattcccaggaggaaaacaaaattgtgtattgtgttgaaaatactattccaatatatgtatacccacgtctcattttgtcatagaattcctgaaaaatttagatgtagatgagatttttataagttgaaaatattttcagttgcattttaattgcacagatgtgttttctacttctatttggctgtgactcctaatgcattgtaatattatcttccagtgttcatttcgtgtagtgataatggattaacaaatgcattcattcatttagtaaacatttactgagccctaatcagtgccatgctctggcccagggaggtagtcatgtgcaaggtccagccctctctgtttcattcctggttggggaatgggtgggacagaccaggaaaggagcagctgccgtatagagcaggagacgctctgctgggtgtgatacagtccctgatggggcctcctactgcagtctttccagtcaggaaagaaacatcctctaagtggggacctaaagaatggatgtcagccaggtaaagaacagggagtagaatgttccaggtagaggacatagtatatgcattggtccagagaggagagggaagagaaggtggcatttaggggaactgtacctgattgagatagttcagcatggagcagatatagctgtggggagtttgatgggagaaggaacgatgagacgtgatgcctgcagaggtgggcaaaggtcggctgaggcaggtctttcttcacgagccatgtgaggatatagggccttatcctcagagtaaagggagccattaaaggctccacaggctgagtgacttgatgaggtgaatacgaaacagagcatttctgtgaatgtgaatggcctttggagcagaactaagttcatgaggatggaaagaatataatgaagccatctcttagacccagaaagaatggggcacaacagcctttatctttctggggccaatgtcacagaatgccatgcttttaggaaacatggtatgttgtgattaacacattttgcagaagtgggtgggagcttttgaataataacagtaagcatttgtgcattcttcctgtaatgacattacagttatgatctgaaaatattgagtcatacatgaattcctgttatcttaactcagaaaatatagtccctcactaaaggttttattttccttctttttcccatttcctttacttcgtataagaaagtcacttgtctcctgggtgcaatggagacctatgtgagttcatagccaagagaatgtttttggttagaaaaataatagtaggaattccaagctgtgaattttttactgaagctctttggaaataggatttggcaagttttgtctgccttcgtcaagtaagcatgagcaggagagcacagttaatagcaggtgcagacacatgattctcagaccgtattttgtgttctagtttcaaggcatgaattctttcctggggttaattttattcaaggaagttatctgtctgttagatctgatatgtgctcaggccaacatagattctttacccttcctttcttcctgctcacctgtccttcctcttttatcttttcattgaattaaaaagaaaattatgaaatagtttcaacatgaaaaaaggtacagagaataacataaagaacactcctggctgggtgtggtggctcacgcctgcaatcccagcactttgggagtctgaggcagccagatcactggaggtcaggagttggagaccagcttggccaacatggtgaaacactgcctctactgaaaatacaagaattagccaggcatggtggcgtgcacttgtaatcccagctacgtgagagactgaggcaggagaattgcttgaacccaggagggggaggttgcagtgagctgagatcacaccactgcactctagcctgggtgacagagtgagactccgtcttcaaacaaacaaacaaacaaaaagaacactcctgtaccatcatccatcattttgccgtgctgactccaggttctatttaagaaataaaacattacaggtacagctgatgccacctctgtttccctagctcattcttcagagataactcttgtcttgcagttggatgttttaatcctctatatcantgtatacttacattctatgtataacaatatttggtactggcctaaatgtgttcacattgtataagtgtgcatattggcctgccacttcatttggaattatgttcttgagatttatcaatgttgatacatgtggaatctggttaatttttgccatagtattctatttttatactaaacttttaaaaatccatgcttctagtctttggcttattttttcaggttatggtatgttttggatgcacagaaaagtaaaattaagtcatgagcaaaatatctggataatccaagctttaaacttgatgtagaatttgaatcatgtgtgttttgttaaccctgtgatgtcaatccatgcctgattgtgtaactccaaccaatattcctttgaaaatggaaatttgtttatattgactacagattgccaatattattagtaaatgctgagcacttaatctcgaataaagaactagtttaaaaatgattctaacaatggcattgactgttctaccttattactcatgggtgggttcagccaatgtttctgttggagaccaaaaccaaaaacagtcaaattaaacaagcagtcaaacccaacatacagactactgataagaaggtcatatcataagatatggcattgaatttgtgtctgctaatgtaaaaatctgatgcccacagcaaacttaataaggacctatgtttacatccatgctcaattacattcctgggttaaacagtcatgctttaggccctgctgtgtgcctggagttttgctgaagtgtggggcttttaagagaaggagaataagcttgctccagagttaagaaatttaaactaaaagtcctaaagatgttggaaaaactattgcccttgaagatgtaaattcattaagttggagaagaccttttatacaaacaacagacccattcactgatttgtacccttcaggagacagatgaccggtaatggtgacaatgggtgaatgttgggtttggggtttttagaaacatctgcacttggtgactactgtatctaattggtgtgacaaacctggcaccccatgtgtttggcaccatcttgggtcctactcagggccaggtgaaccgagtggcctcttcactgcttcagagtctgaagcagattgtagtatgcggaccagacacagaatataccaccaagcacgttggcgcaaagcatactgggaagggaggctttgtgaacatggtgctggttctcaaacctcagtgctcaaaagagtctcctgaggattcctggatcacactcttaaacttctcattcagtaggtgttagctggttgagaatctgcatttttttttttttttctgagaccgagtctcactctgatgcccaggctggagtgtattggcgccatcttggctcactgcaacctctgcctcccaggttcaagcaattttcctgcctcagcctctctagtagctgggattacaagcacatgccaccatgcctggctaatttttgtacttttagtagagagggggtttcgccgtgttggccaggctcgtcttgaactcctgacctcaggtgatccacccactttggcttcccaaagtgttgagattacaggtgtgagccaccatgcccagcctgaatctgcatatttaacaagcaccacaggtgattctgatacagtagctccccaaacctcacagtgttagtgaatcccagtcatttacaattctgccatgattttggtcatattcaagtgcagctggtagcatttttagttaatatattttttaaattaagtcacttcttttggataattaaatttaattacaagggaagctataccactgctgtaaaaacatcacctgctttaaagagaaggtacataatgaatatacattaagataaagatgtatatgtgtgtgtgtgtgcacatataagtatacacatacctaccatagggattgagttttccttcaggtttttcaactgaaatgtcaactttgaggccagttaatatgtgtaagatatatgtgtgtatgtatgtctatacatatagacatatacgtaaaaacatacatggatgcatatagtatatctatacacaacctattatgcatatcatgtatatttcatccacttagtattatcttnttattttgccgtttggcaaatgctcagtaaaagaaaagggttagaaggggagaaaggcattttatcccaagccttcaggaatcaggatgaggatgtcttcaccttgtggtggggagtanattatacaattagagacagcacattggagntgtggctgatatgctgtgtgatgatagctctagctctctgcctagcagaggaaggacatttcaatagaagaaaaagtttaagaccttgccgagaaacagagaaaggatgtttgtctttttaagaagttgaaaaccctgtttgcagacaaaagccctccagttttggcagtaaactttcatgcaagggaagaaaaaggcaggggatgacattgttgacaattgtgaggaattaccatgtgccaggcactgtgcgaggggctttgtacatatcctctagttttagtgcttataaaaactctgtgatatgtgcacagcattttaaactttgctgcatagtcgagaaaatggaaggatggggaatttgagtcatttgcccagggttctatagctaccccaggttcccatgactggagaattggggcacagggtggcgggggagagtgagtgacaagaatcctaacaatcttatttccattgagtccttataaaagaagtggattaactaccacgtttttaagtttttcttaaatttaggttatgtggatctggcgtttcttgttttgtcctgggtttgttttgtttttgctatgctgtcttgaacatctgtcatcttgtaggcctaacggtaaacacaaaaacactttacctcctatagctttcaattaagatctctcagtttgtgtttgtaatagttttccaggcaagttctccctaggttcggcttctagtgtgttaacctttagttataaagtgaacccaaagagagaaagtagaaacaaaacacctcacctgtttttgctcatgaattactctctatggaaggaacaatcatgaacacctctgcgtatcacagaggcctatctgagtctgacgtttaagggagaccgcgtaggtccctttgaggactgtgaatgtgggagtcctgggactctggtgaagaacccgttccagaagagatgaatgagctggacaagttctttcatagaacctttaggcaggttttcttagaaatgcacattgaggattatgcttggatattgtgatgatcagaatgatactcaatcccttctgcatttggaattctctttgaaagaaaacatcccaggcagctatttctcagagatagtgagtcccagccacttctagacattttcttgtgtagtctacattataatttcacagcagtctctgatatgacaaatgtcaaaatagcccaaccttctctaaacttcagagatgtctgatatgatattgaataaaacaatgctcatagaaacatcaagaaaggtggattttccctggatacttttttcctgcttgacaaataacagtgaagaaactgatctcacgtctttttctctttggaagcctgaacactcagaacccaacttgaggctcctcagctatagcaattctgacttcacagtctgtaaattattgttcttttttttctttagcttatgctttctgccctaatttatcttttccctgttctaatgaattattgtcctatatctgctgtgcagttaggtgacatataacagcaattaaatatatgaattggtacatataaagatttgactaaaactcgatgtaaaaataagtgttctacattcaatttccagtgttagaaacagtgctgacttgaacagagtgacagaattccatctttccctatttttgacagctttaaactttatattttcttcctttcttgtgagccgtcattaactttgtttctcaaagnccattcccgtattacccatcttgcagacgcagacagatttgggaatttgcggtcagagttgtattggacacatccccccagcccacatgagatccttttaatctattgcatattaactagttttaagtacaatattcctacttcatttaaaaccattaatcaaagaatgagtttgaaaatgaacaaaatgcaaacttacagttagaaataattgtagtgtctttagttttggttaggagtcggtttcttgtttgttaaactcaagattgtgaacagttttaattcacttgtttatttccaatagagatttcaggtttacatttgaattcagaaacaaagttttctttctcattacagagaacactaaactctacatctcccttcccgagcaaggagctggctgaagccacaaaaacattgctgcatagtcttgggactctggcccaggaggtaagttgtgtctttccagtaccaggaagcggatcatccactgtatcagtattttcattcctgagtctggcaagaggtccttttgagttgaatatcacatgggatgtaatatcaattttcaaagtataagtgatgtaaacaataatgttttgatttccttattttagaaatgaagaaacctaaaactcatagatgtctcagagctaattggttagtggctaacagctggatatctagtttagaaccttctccattttttctttttgcccctaggtaatcatacatttgtaaagaggagaattatctctgccactgcccatgcactgcttttgtctgaccagcaatttctccatattgcttcttcagtagcaaggccaatcattttaccaacacacatgcttgctaactaacaggaataacgtggtacccctaattcagccctttcccttgaaagcatctggcttctgaggttcaactatgggaatatggtctcttaatgaacattaagttgagtttgccttttaggtccacatgttgacaaatgtatcagagtaatctctgtcctaggatcagagggcctgtaggcacttgcaaaagcagttagctctgactcccagccagtgcacactccacctttctgactcccagccttgtctcaaattaggcttggaagcgaggaactgtctggtgtcccccagcataggaagctgagccagggggcagtgctcacaaacaatacagactttaacgtgtaggatattggaaaataataatttgtggggaaattgtctcagacttggtccacccttatttttagctgcttctctaatccgtttttctttttttggtgcttgtatctaacctacccattttttggtgcttgcatcattttttcaaatatcaaaaacgaactttatgttttctaacaatgaaagtattgcatgttcattgtggaaaatgctgaagacttggaaaatacaaaaatgctgagatcaaacactattgatacgttagtgtatttcttcctgtcctgttctactttctttctttgaattctgctcacgtgtttctgactgatgaggtctgacttttgggttccttttccagaggagaagccttctttcagcttgccatttgttaccctggttatgaaggctggtaaccttttttactaggtagagaagctggaccaactggggttcttccagggggagaatgagaaagagaaactgttttgcaagtccgtagctatttctctagggccctgttagctgacattgacatgccttgcattgctctgcagatcccctcgcagccctctgtcccttgttcatttctggccttagagaaagcaaagcagggtctgtaacaggggaggctgcctctaaactcagggtttggttacagctgttttcacttacatcactggccctggttttttttttttttctggcattaaaaaaaaaaattggaagcaggtgatgttcccattgctgatgtggtggaaactctccaagtgaacaatatacgtttttcttggcagctgtttcttgtgccctgcttgctcctggtccaggacaagcaaggaccatctgcctctttcaatagaacacctccagatccctttgatcaaaagttactcattgtctgacttgctatttctgtgagataaatgggagaagatcaataaatgcacttgtttgtccagtcagcngtgtggaaagttgataattttgaccaaagcacaacccttgaaaggaaaagaaaaagggagtgaatgtcttctgagaagctgcctaggttcagacagtgtcacccatttccctgtatgctccacatgacaaacctgagtgggtctcatcatgtccattttgcagatggcaccaaggctcagaaaggttaggcaacttttccagtcacccaatgagttaattgacaaaactgggattcaaacccagaactgttggattccaaagcctgtgttgttgcctgcttcgtgaaaaactccagtagcgactggaatagaaaggagaaccttccaagaaagaaaatacgcactagcagaacctggaaattgggaggaaatgaggacttgaggaataagatgaatgaaagctgacctgagtttcacatctgggtgatgggaagggaggacagggaggcagcatctcagatgtccacccagcaccgaccagctgcctggcattgctaggtgttgaggactcagcagtgaacacgctaacttctctgctttcttggggcacgtatagggtgagagacagaaacaaacaggtcagtgtacaatgccacaggagggatatatgcagtgaagaaaaagcagggtaaggggcatagagcatgagaaggtgctttttttaaaggggktgattaggaaagctctctctaaggtgacagttggacctgaaggagatgatagcatgtctgtggtgagggaaggaaactccgaacaggaagaatggcagatacaaagacattgatgctagagcatgcctaaggaatgtgtttaaggaccagggaaagtgagcaagtggtggggggaggagaggagctcagagcaggaggaggtgagtgccatacaggcctggcaagactttggattcctgctgggtgagatgagaatccagcggagggcttgagggaggggacatgatgtgatctagagtttagactgtttacactctggttgttgggttgagaagagactgggatgggggaaagggaggacaaaggacattgtgctggattgagaaagcagtaagtcagtttcattcattcactcaaccgatgatgttcaaataccaccatcatccgtgggctaaaggatgaagagccatccctccctgagagtcaggaagcacttcccagataaagtttggagtgtgagctgaggtgtaggagaaagagtaagagtttacccctgaaacgggtgctgggaagagtcaatagtttggaataactcaataatttatggtgcttctttagaaagatttgctggctttatgtgggaagaaatttktttttttgattggggagtggtgggttggtggtgaggctgcctgtggaaagagaagtgagtgttttgactcactgttatttaaaaatctctagggctgttccaataagcaacaaaaggcaaaatggcctggttctctgtcccctttctgtctgtatgcctcgtacaggttatgaaaagaaaaagttgggaaaagctgtccacctcacctaattgtgttcttgtggagtgtgctagatgccccctctctggagaaaaaaaatccttgtggcctctgacccacctctggagagcctagttcccttctggaggcagaaggcaaagcttaggacctagagagtgctggaccacgccactcacaggaaccagcaggctgtgaggttgaaagctaggcatatggagctttccaggctgggtgcagggcctcgtggcccttcccctcccctctgtgctctatagctcagtcttcccaggcggtgtgaacacgcagtgacatttccaggaatacagggatttattaatgatttcttgtgaaatgtttggaaatacaaagtactctataaatatttcataatagcattggggctgagaactccacaaagtgccggaatacatttgcatgtaagacagaacgctgcctgggtcattgatgcctgttgagtggcagtcacagacactgcctagggtttctgactcacgctgttgggactgttctatgcagggcaccctcttgtgtggcataggatttgtgcctcaccacacactgttgtagctttgctgtcttgatgatgagtagagggcagtgtccaggccatggtataagcatctactgccccccagggttaccaaaaccaagccaagttgtgtctcagcgagctccgtgaagcatggagaagttgagtactcagagacatgacgtgacttttcaaaggctgtaagctgacgagggacatagctagggttcagacttgagtttttctttttctttttctttttcttttttttttaagactgagtcttgcttttgtcgcccaggctggattgcagtggtgcttggctcactgcaacctctgcctcccgggttcaagcaattctcctgcctcagcctccccagtagctgggattacaggcacctgccaccatgcctggccaacatttttgtatttttttagtagagatggggtttcaccatgttggccaggctggtcttgaactcctgacctcaggtgatccacccgcctcgacctcccaaagtactgggattacaggtgtgagccactgcacccggcccagactcgagtttttcatcttaatgctttttcattgcctgacactttactgagaccaagatagggaacttcacatacagtaccttttctcccaaggcggaagagggctgttcaatttctacactagagttcggggagttttagaaatgagtcagttatcgaggatgagagcagttcctgataggctcaaccacaatgagatgtagctgttcagagaaagcattcttttatctataaactggaagataatcccggtgaaacgaagcccagccccaggggcttcactaactccaggctgtgcttctcaaactttagtgagcataggaatcacctgggcatcttgtgaagctgtagatttgaattctgcaggtcggcagaggggtctcagaatccgcatttccaacaatgtctccagtaatgctgatgctgctcgtccctggaccacagattgggtagccaggttctggcaagctcatcccaaggctttgagatgacatcagacaaaatatgttctgggacatggcttttgagaggtcaagaaaataagatgtttctttctcttctcatccccaacccttgcactgcccttttctcccttcccctaccctcctttctgtccccatccctgacgccagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaagtgagtgtccatgcagaccccagccctgtccccaaccccatccctcccttagttctggccttggcctgtgtcatctcctccctctgtagcagcgttagatgtctacatgcccatttgcccaccagactgagctcttcctagaggagagaggcttctcttgaatagctacctgtccccagttctctgaatgcagcctggcacatctcaggtgcacagtagtgtttatcaatggaatgaatgattgacagccaaccttctggttttctgggggatgtggaagggtggcttccagggtgatcaagaatgagataatggcagaaggacaaatcctgcaagatctcacttatatatggaatatatgtaaggtagaaagtgtcagtttcacatgatgaataagttcctgggatcttgatgtacatcgtgatgactatagttagtaacactgtatagtatacttgaaatttgctaagagagtagatccgaagtgttcacactacacaaaaaaggcaactatgaggtgatggatttattaacagcttgattgtggtgatccttttacaaagtatacatatattaaaacatcacattgtataccttaaatatatacaatttttatttgtcagttgtaactcaaaaaagctagaaaagcatttttaaaaaggatgatgtactggtcttaatattaccattgagataagctttataataacataaaaagaaataacagtaatgataatagcaacaacaacaacaacaaagaactaacatttaagtagaatttcttgtgcactgtgcattctgtttaagttatctcattttaccctcatgataaccctgcagggaagattctttaaccccacatttcataggctcagagaggttaagtgccttggttagagccacatcagagttaatccacaagagccaggattcaagcccaaatctgcctggatctgtgctctctaagataactgttagtggtggcgtgtgtgttctcacactcagacatttgatctgccctttgtttcccattcttagctgcaaggcagtgttaaagaaccctgtgtctccatatccactccccacacttaagcacttttgtgggcccgtgtgccgtatgcctcgtggcagcagggatccaatgtcacagttttaggcagtggcatccttttccttgaaaacttgatgcaggggaacctttctccatttccaaccacaggtgtgtcnntttcagacactgagtgnaggcaggttttgtactttattgtaacacaagaaccttttcttctctggagtaaagcactccagacattcgcaagttgctttacaagccttaaaaggatggtattgtaggcaactttaattaaatcccatctcctcctctcccccagcttgcaagttgacccaaggaagccttcatttccatgacagacttaattgtgagggcatcctcattaaaaaaaaaaaaattctattatctttccagcatatagaagatacttggtatctaaaaatccctgaaaaacttagaatgaatttttaaaaatcagggatcctgctggataaccaaacccatttgtctgttacaacttttgtatttgggtttttgttaagtgtacatatactagtttgtgttaattaaagagaatttttttttttttttttgagagggagtctcgctccgttgcccaggctggagtgcagtggcgccatctcggctcactgcaagctccgcctcccgggttcacgccattctcctgcctcagcctcctgagtagctggaactgcaggtgcccgccaccatgcccagctaattttttttttttttgtattttgagtagagacggggtttcactgtgttagccaggatggtctcgatctcctgacctcgtgatccgcctgtctcggcctcccaaagtgctgggattacaggcgtgaaccactgcgccctgttgagaatttttttttttttttttgggagacagagtttcgctcttgttgcccgggctagagtgcagtgacacaatctcggctcactgcaacctctgcctcctgggttcaagcgattctcctgcctcagcctcatgcgtcaccacgcccagctaattttgtatttttagtagagacagggtttctccatgttggtcaggctggtctcgaactcccaacctcaggtggttcgcccgccttggcctcccaaagtgctgggattgcaggcatgagccactgcgcccagccccaaattttggtttttgcttgaaaactgaggtctgaattcagccttctggttgcccctcaagagtcagtttaaatgttggtcatgttagttgtcagtgaaaacaatggtgaggctggcatgagagtgtgaatctggatgggagggcttgtgcttcatgaaaacatttttccagatcagctcagtcgtgagttatccgtcattgacgttataataagctctgattatttatcaagcatcattctttatagatatctcagtttaatctgagataatcttctccacatctctccacatagatgttatgaattttacttttacagaggagccaactgaggctcagataagttacttattatatgactagtagtggtagagctggggtttcaactaagaactctctggctccaaagcccttgtaagtttctatcagtatatgaccatgcatatgagcatttgtctctcctcttcttcatagctccttactgcaatgatttgatgaagaatttggagtctagtcctctttcccgcattatctggaaagctctgaagccgctgctcgttgggaagatcctgtatacacctgacactccagccacaaggcaggtcatggctgaggtaagctgcccccagcccaagactccctccccagaatctccccagaactgggggcaaaaaactcaaggtagcttcagaggtgtgcgctaagtatactcacggctcttctggaattcccagagtgaaaacctcaagtctgatgcagaccagagctgggccagctccccagtcgtgggtatagaatcatagttacaagcaggcatttcttggggatggggaggactggcacagggctgctgtgatggggtatcttttcagggaggagccaaacgctcattgtctgtgcttctcctcctttttctgcggtccctggctccccacctgactccaggtgaacaagaccttccaggaactggctgtgttccatgatctggaaggcatgtgggaggaactcagccccaagatctggaccttcatggagaacagccaagaaatggaccttgtccgggtgagtgtccctcccattattaccatgtgcctgcttgatactggagaggtgagtttctggtcactttcccaggtgtgagtgaggtgagaattctttcagtttatctagctgggggaatgtagtgagcatagctaaagtcacagggcaccacctctccagaagtacaggccatggtgcagagataacgctgtgcatatcagcatccatgccactcacggtcaaatagcagttttctgcaaaacttagtgagggctggtgtttggaagtggagttgagtaattgcagtaccctattttcctttttngctgcagcctctcagccagccacagcatctccctgtgtcttggtaggttttggaaagaagtgtgggagcaaaagcatgatgttacatgtagactggcctgagatactcattctcagggcactgtgtgaatgatgagctgctgttactgtgtggaggggaaatgcacttagtgcttcagagccacttgaaagggataagtgctctagagacaattgggttcaaatgtggagcaggctgagcaagaacagaatgtctcctttgcctgagcctgagtgctgttaatcacatcttcctgccttgggctgagttagagaatcattagactatttcctgtttccatggtgagggaggcctcttccttttgtctctgctccccttaagaagcaggtgaggattttgccaggtttcttgttttgaaccttattgactttaagggcggctgggttttagagactgtacctacctagggggaacacttccgaagtttaggactattccctgatccgctgggaggcaggttactgaggaagtccctttaaaaacaaaggagtttatactgagaaaagcataaacagtgatttgtatggattcacactgactaatatagctcatgccattaaagtggggtctcttctctaaaggagggttatatgatctagccccgtagacctaagtgtggtttcagacctgttcttcctggtcctctccttggaatccatatttctactagttggactttttctgtttgtctggctctcagaggattataggaggccctgtgaagtgactcagtgaattttgatttgtgggcaagtagatggttccctagtctgaaattgactttgccttaggtgcttcaattcttcataagctcccagttcttaaaggacaagatccttgtaaacatggcaatggcattcattaggaatctagctgggaaaatccagtgtgtatgcttggaaatgagggatctggggctggagagaaaggcatgggcatgccttggagggacttgtgtgtcaagctgaggacctttactttaagctctaggggaccaggcaaggggagatgtagatacgttactctgatggggtggatgaattgaagaaggatgaggcaagaatgaaggcagagaccagggaggaggctctccaagtggccaaggcataaagcaagaaatgaggcctggtgactgcttagtggcagagcagtgaaagagagggaggcatcaaagtgagtctcgatttctagctgggtgggtggtagcgatgtccagtaggccagtggctactgaggtctgcagtggaggagggtggttgggctggagacagatgatgagggagtcatcagcctgtgggtggaagaaaagggaacctcttccaactgttttctttgcttcttccctctctttctctttttttttttttttggacagagtcttgctctgtcacccaggctgaaatgcagtggcatgatcttggctcaccacagcctccgcctcctgggttcaagcaattctcctgtctcagcctccagagtagctgggattacaggcacatatcactgtgcccggctaatttttgtattttcagtggagatgggatttcaccatgttggtcgggctggaatgaactcctgacctcaagtgatccacctgcctcagcctcccaaagtgttgggattacaggcatgagccaccgcgcccggcctttcttccctctcttaaagagtgtttatttaattccacaaacatgagcttgtcaccccctgtagcctggcatctcctacacgaggtgatggctgaggcttctgcttctgctggggtagctctgatctttctgctttctctggcactgtctacccatgttgcctcaccccacaggtcccagggcacctctctcgggcaagtcttggaaccctctgacactgatttgctctcttttctgagctgcttttagccacccatcctcgggacctgttttctctctgcctccacccctgcgggcagtcttaggtctcctgcccctcacgagcaccccagagaggccacgtgctcagtgatctcagtgggcgcatctttctagtcttgctattctttttggccatgttgttcagaaaccatactgggcagggccgacttcaccctaaaggctgcgtctcttcactctgcttttgtttgttccaaataaagtggcttcagaattgctaaccctagcctctgtgaacttgtgaggtacaattttgtgtctgttatgttaacaaaaatacatacataccttcctggtgatggtataaattgctattctctattggaaagcaatttggaatgaaaatttaaagaaccattttaaaatatgctatcctgcgtacctccattccacccacccccagggatgtagcctactgaaataattttaaagaagtcaccatatgagagaaaatgttattgctatattgttattgtgagaaattggaaatagactaaatgttcagcactataggaataattaatgaaattacatatactctatacaatcattatgctgccattgaaataataaatacaaaggcgcaaggggggaaaagcttataatgttagtgaaactaagactgatttttttataaagcagcagttttcagacccttggagactccaattcggtagaaccagagcttcatcttctctgtcgaagctgtgacaggagttgcaaatgcctctcctttttgctgagtttgcagctgctgtttttccggcagcacatctgtgcaggcctctgcctcggcccctctggnatctgctgattgagcagcggattgatctgtccttctctttcgtgttgacccatgtgaggaaccaactggcaaggganacaagaaatggaaataggcctcctttgcatcatgacctgtacatcctgcaattggaaaagattgtactttagttggtttaaccagcagcattatttttctaaactaagcagtaagaaggaattaggttttatgtgggatcaacagactgggtctcaaaagaggaaggtgatagaacacagtggggagggggaggtgcactagaaacagagggcctatgctttcattctggctttgctacttaatagctgtgtgacccaatcttagagacttaacctctctgaacttccattttctcatgtataaaatgggaaatattaaaggatactcactgggctggtggcttgtgcctgtaatcccagcacttggggaggttgaggtgggaggatcacttgagcccaggtgttcaagaccagcccaggcaacatggcaagactctgtctctatgaaaaaattaaaaattagccaggtgtggtggtgtgcacctgtagtcttagctacttggtaggctgagatgggaggatcacttgggcttgggaggtcaaggctgcggtgagctgtgattccatcactgcactccagcccgggcggcagagcgagacactgaatccaaacgacaacaacaacaaaaggcaaaaaaataaaagtgccctctttatggagttgtgtaaggtgaagcatatacactattcaacatagtaactatataaaggaagtattgttgttgttactgtagttaataccattaagtgagatgtttcgtatagtggaaagcacatggactctgaattcagactggtctgactttgagtctcagctccacatctagtaatactatgaccaagccctggttaaaatcatgtttttttttcttcagcctcagtcttctcacatataaaatagggacactgtcatttacctcagttttctgtgaggataaaacaacgacagtgtatatgcaagtattttgtaaattttgtagtgctcctcaagatttagttggtgtttactacttgtactttctcactggaatggcagatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggaggtgaatctgttgctgggatcatttagaaaagacttaacggcttctttctctgagacgttacaataaggttcaggcaggaggcaagtttagaaataatgtatagtctcatttacaaaactatccctcaagcctaacacaggatttgataacaaaaggcacttaataaatgttagttgagtggttgaatgagtaaataaactctagctttagtaaattaactctagcttattctatataggctcaagagaatatttctacccattttcttctaggttttcctatctcagtgactaatggtagcaaagcattcccttaaaaaggcattatttgtgaaacttatctaaaatcgaattcgggtccaattaaatttttgaaattttatattaaaaattatattagtagggatgggtaagaggtgttttggtctggttggttggttagttgctatgactcagaattgctaagaaaacagaaaagtaagataagatcattgttttaacctcttttcctccacaaaatcaataaataacatatccctaaattactcttagaatttctcttaaattgcagtgaaaaaccaaaatccttcattcttggttgaaggttggaaaactacgttagagaggattagagagagaggatgagcaatcgtgtagtcagcccttgcctcctagtgtaggatttgtctcagccactgcttgttgtcctggctgccaacgttctcatgaaggctgttcttctatcagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtaagtggaatcccatcacaccagcctggtcttggggaggtccagagcacctattatattaggacaagaggtactttattttaactaaaaatttggtagaaatttcaacaacaacaaaaaaactcaacttggtgtcatgattttggtgaaattggtacatgacttgctggaaggtttttcataggtcataaaataacagtatcttttgatttagcatttctactcaagggaattaattccaggaattttggtggcaggcacctgtaatcccagctactcgggaggctgaggcaggagaattgcttgaacccaggaggcagaggttgcagtgagctaagatcgcatcattgcactcccgcctgggcaataagagtgaaactccatctcaaaaaaaaaaaaagatacaaaaatagaaaaaggggcttggtaagggtagtagggttttgggcaatttttttttttttttttttttttattgtatggttctaaaggaatggttgattacctgtggtttggttttaggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatgtaagttacctgcaagccactgtttttaaccagtttatactgtgccagatgggggtgtatatatgtgtgtgcatgtgcatgcatgtgtgaatgatctggaaataagatgccagatgtaagttgtcaacagttgcagccacatgacagacatagatatatgtgcacacactagtaaacctctttccttctcatccatggttgccacttttatctttttatttttatttttttttttgagatggagtctcgctctgacgcccaggctggagtgcagtggctcgatctcggctcactgcaacctttgcctcccgggttcaagctattctcctgcctcagcctccacagtagctgggactacaggctcatgctgccacgcccggctgactttttgtattttagtagagacgaggtttcaccatgttacccaggctagacttcaactcctgagctcaggcaatccaccctccttggcctcccaaagtgctgggattacaggtgtgagccactgcacccagcccaccactttaattttttacactctacccttttggtcaaaatttgctcaatctgcaagcttaaaatgtgtcatgacaaacacatgcaagcacatactcacacatagatgcagaaacagcgtctaaacttataaaagcacagtttatgtaaatgtgtgcacttcttctccctaggtggtaaaccacatttcaaaacaacccaaataaaactgaacaaagcttcttcctcttagactttttagaaaatctttcagtgctgagtcactaagctgccaagttctcattgtgggaactatgcctttggatgtaatgatttcttctaagacaatgggcggaggtgtagttattgcagacatctgaaatatgtaatgtttcttccagattctggaaattctcttattctctgtggttggtggtggtggtgggatgtgtgtgtgtgtgtgtgtgtgtgtgtgtgtgtgtgtgtagggatcaggatgcgggaggagctgggttctgcttgtattggttctctgttttgcattgaatagtgtgtttccttgtatggctatctatagcttttcaaggtcaccagaaattatcctgtttttcaccttctaaacaattagctggaatttttcaaaggaagacttttacaaagacccctaagctaaggtttactctagaaaggatgtcttaagacagggcacaggagttcagaggcattaagagctggtgcctgttgtcatgtagtgagtatgtgcctacatggtaaagctttgacgtgaacctcaagttcagggtccaaaatctgtgtgcctttttactttgcacatctgcattttctattctagcttggaatctgaaacattgacaagagctgcctgaaatgtatgtctgtggtgtgattagagttacgataagcaagtcaatagtgagatgaccttggagatgttgaacttttgtgagagaatgagttgtttttttgttttggtttttagtactttaacataatctacctttagtttaagtatcgctcacagttacctagttactgaagcaagcccccaaagaaatttggtttggcaacactttgttagcctcgtttttctctctacattgcattgctcgtgaagcattggatcatacgtacatttcagagtctagagggcctgtccttctgtggcccagatgtggtgctccctctagcatgcaggctcagaggccttggcccatcaccctggctcacgtgtgtctttctttctccccttgtccttccttggggcctccagctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaaggtaaggcagcctcactcgctcttccctgccaggaaactccgaaatagctcaacacgggctaagggaggagaagaagaaaaaaaatccaagcctctggtagagaaggggtcatacctgtcatttcctgcaatttcatccatttatagttggggaaagtgaggcccagagaggggcagtgacttgcccaaggtcaacccagccgggtagcagctaagtaggatgagagtgcagggttcatgctttccagataaccacatgctcaactgtgccatgctgtctcattggtagtggttcatggcagcatctgaaagctatttattttcttagatatattgggtggcgattcttcctaagtttctaagaacaataatcagaaggatatatattgttgcaggttagactgtctggaagcagaggctgaaatagagtttgatgtatgggtatttatgagggctcaatacctatggaagagatatggaagatgcaggattgggcagagggaggagttgaactgtgatatagggccaaccccgtggggcactctagagaatatgcagcttgttggagttgttcttcatcgagctgaaacatccagccctttgtgctcccccaaggcctccctcctgacaccacctacctcagccctctcaatcaatcactggatgtgggctgccctgggaaggtcgtgccccagggcctacatggctctctgctgctgtgacaaacccagagttgctgatgcctgaggccgtctactgacagctgggcaacaaggcttccctgaatggggactctgggcagtgcagttttgtgtctgaaccatacattaatatatttatatccgaattttctttctctgcaagcatttcatataaagacacatcaggtaaaaataaatgtttttgaagcaaaaggagtacaaagagataagaactaactaatttaatactagttaccatctgttacaaatagttcctactgattgccaaggactgtttaaacacatcacatgggcttcttcttctatcctcactaacccttttaacagacaaggaaatgaggctcaggaaggtcaaggactttattgaggttccacagtaggatacagttcttgctaaaagcaacccctccctcatgctctgttatctaactgcaaggggaaggtcagtggcagaggtagtggtcccatggttggtgcataagagctgctctgagacaactgcatgctggtgggtcctgcagacatgtacccatcagccggagataggctcaaaatatccacaagagtttggatgattgtgggaatgcagaatccatggtgatcaagagggaaagtcaagttgcctggccattttccttggcttttagacagaaaagttacgtgggatattatctcccacagctcttctgtggtgccaccagtcatagtccttatataaggagaaaccagttgaaattacctattgaagaaacaaagagcaaactcgcccactgaaatgcgtagaaagccctggactctgttgtattcataactctgccattatttttctgcgtagttttgggtaagtcacttatcttctttaggatggtaatgatcagttgcctcatcagaaagatgaacagcattacgcctctgcattgtctctaacatgagtaggaataaaccctgtcttttttctgtagatcatacaagtgagtgcttgggattgttgaggcagcacatttgatgtgtctcttccttcccagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctgtgagtacctctggcctttcttcagtggctgtaggcatttgaccttcctttggagtccctgaataaaagcagcaagttgagaacagaagatgattgtcttttccaatgggacatgaaccttnagctctagattctaagctctttaagggtaagggcaagcattgtgttttattaaattgtttacctttagtcttctcagtgaatcctggttgaattgaattgaatggaatttttccgagagccagactgcatcttgaactgggctggggataaatggcattgaggaatggcttcaggcaacagatgccatctctgccctttatctcccagctctgttggctatgttaagctcatgacaaagccaaggccacaaatagaactgaaaactcttgatgtcagagatgacctctcttgtcttccttgtgtccagtatggtgttttgcttgagtaatgttttctgaactaagcacaactgaggagcaggtgcctcatcccacaaattcctgacttggacacttccttccctcgtacagagcagggggatatcttggagagtgtgtgagcccctacaagtgcaagttgtcagatgtccccaggtcacttatcaggaaagctaagagtgactcataggatgctcctgttgcctcagtctgggcttcataggcatcagcagccccaaacaggcacctctgatcctgagccatccttggctgagcagggagcctcagaagactgtgggtatgcgcatgtgtgtgggggaacaggattgctgagccttggggcatctttggaaacataaagttttaaaagttttatgcttcactgtatatgcatttctgaaatgtttgtatataatgagtggttacaaatggaatcattttatatgttacttggtagcccaccactnccctaaagggactctataggtaaatactacttctgcaccttatgattgatccattttgcaaattcaaatttctccaggtataatttacactagaagagatagaaaaatgagactgaccaggaaatggataggtgactttgcctgtttctcacagagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggtacactgctttgggcatctgtttggaaaatatgacttctagctgatgtcctttctttgtgctagaatctctgcagtgcatgggcttccctgggaagtggtttgggctatagatctatagtaaacagatagtccaaggacaggcagctgatgctgaaagtacaattgtcactacttgtacagcacttgtttcttgaaaactgtgtgccaggcagcatngcaaaatgttttatacacattgcttcatttaattctcacaaggcntactnctgaagtagttactataataaccagcaattttcaaatgagagaactgtgactcaaagacgttaagtaaccagctttggtcacacaactgttaaatgttggtacgtggaggtgaatccacttcggttacactgggtcaataagcccaggcgaatcctcccaatgctcacccaattctgtatttctgtgtcctcagagggggtacaactaggagaggttctgtttcctgagtacaggttgttaataattaaatatactagctctaaggcctgcctgtgatttaattagcattcaataaaaattcatgttgaatttttctttagtacttctttcttaatataatacatcttcttgaccaagtccaagaggaacctgcgttggacagttttcatatgagatcaaattctgagagagcaagatttaaccctttttggttcaccttctgatcctcccctaaggaggtatacatgaaatatttattactcctgcctgaacttctttcattgaatatgcaattttgcagcatgcagattctggatttaaattctgagtcttaacttactggctgagggaccttggataggctccttatccctcagtttcctcatctctaaaatggggatggcacctgccccgtgggttgttggaaggacttacagaggtgcagaatgtacgttgtacatagcaggtttcagcaaatgttagctccctctttccccacatccattcaaatctgttccttctccaaaggatgtgtcaaggaggaaatggacctggctgggaaaccctcagaatactgggatgatgctgagcttggctcatacctgtgctttgctttcaggccagtacggaattcccaggccctggtattttccttgcaccaagtcctactggtttggcgaggaaagtgatgagaagagccaccctggttccaaccagaagagaatatcagaaagtaagtgctgttgacctcctgctctttctttaacctagtgctgctgcctctgctaactgttgggggcaagcgatgtctcctgcctttctaaaagactgtgaaaccactccaggggcagagaaatcacatgcagtgtccctttccaaatcctcccatgccatttatgtccaatgctgttgacctattgggagttcacggtctcgatccctgagggacattttctttgttgtcttggcttctagaagagtatcttttacttgccccctcccaaacacacatttcatggtctcctaacaagctagaagaaagaggtaaagacaagcgtgattgtggaaccatagcctcgctgcctgcctgtgacatggtgacctgtgtatcagcctgtgtgggctgagaccaagtggctaccacagagctcagcctatgcttcataatgtaatcattacccagatccctaatcctctcttggctcttaactgcagacagagatgtccacagctcatcaaaggctctgccttctgggttctttgtgcttagagtggcttcctaaatatttaataggtcccttttctgccagtctcttctgtgcccatcccctgattgcccttggtaaaagtatgatgccccttagtgtagcacgcttgcctgctgttcctaatcatcttctcctacctcctctttacacctagctcctgtttcagtcacctagaaatgctcacagtcgctggaatatgtcatgttcttccacacctccatgcctttgtaggtactgtttgctctcacaggagaactttctctctaacttgcctatcttctcaactcctcctttctctccaagatctagttccggatcccctcccctgagcatccctccttggttctcaggtagtcagtcactctctgccctgaacttccatggcacgtgaaagaaaatctttttattttaaaacaattacagactcacaagaagtaatacaaattacatgagggggttcccttaaacctttcatccagtttccccaatggtagcagcatgtgtaactgtagaatagtatcaaaaccatgaaattgacataggtacaattcacaaaccttcttcagatttcactagctttatgtgcgctcatttgtgtgtgtgtgtgcgtatttagttctatgcaattttatcatgtgtgaattcatgtaattactagctcagtcaagctgcagaaatatctcattgtcacaaagctccttcatgctaccccttaantggccacnagccnacctcccttcttcctcagttcctgacacctgtcaaccactaatgcgttcctcgtttttacagttttattatttctagaatgttacataaatggaaccatacagtaggtatccttttgatactggcttttttttttttttcactcagcagtattcccttagatctatccaagttgtgtgtgtcaacagttcattcctcttcactgctgagtagtgttccctgggaggggtgtatcacagttccatggcatttttagatgtattttttaaacagctttcagcatcctctattttaattgttcatcaagtcctttttcccaatagactctgaatgctcctttatcatcgtattcccatcaccaacatcagtacccaaataggccctaaataaacatttatagcctcctgcctgcctgagaaaccagggtggacatggagagaaggcacttctgaaagttcaagcgcagtgcsctgtgtccttacactccactcctcagtgctttctgtgggttcatttctgtcttctctcctgtcacagtctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtaagaagagggtgtggttcccgcagaatcagccacaggagggttctgcagtagagttagaaatttataccttaggaaaccatgctgatccctgggccaagggaaggagcacatgaggagttgccgaatgtgaacatgttatctaatcatgagtgtctttccacgtgctagtttgctagatgttatttcttcagcctaaaacaagctggggcctcagatgacctttcccatgtagttcacagaattctgcagtggtcttggaacctgcagccacgaaaagatagattacatatgttggagggagttggtaattcccaggaactctgtctctaagcagatgtgagaagcacctgtgagacgcaatcaagctgggcagctggcttgattgccttccctgcgacctcaaggaccttacagtgggtagtatcaggaggggtcaggggctgtaaagcaccagcgttagcctcagtggcttccagcacgattcctcaaccattctaaccattccaaagggtatatctttggggggtgacattcttttcctgttttctttttaatctttttttaaaacatagaattaatatattatgagcttttcagaagatttttaaaaggcagtcagaaatcctactacctaacacaaaaattgtttttatctttgaataatatgttcttgtttgtccattttccatgcatgcgatgttaggcatacaaaatacattttttaaagaatactttcattgcaaattggaaacttcgtttaaaaaatgctcatactaaaattggcatttctaacccataggcccacttgtagttatttaccgaagcaaaaggacagctttgctttgtgtgggtctggtagggttcattagaaaggaatgggggcggtgggagggttggtgttctgttctctctgcagactgaatggagcatctagagttaagggtaggtcaaccctgacttctgtacttctaaatttttgtcctcaggtcaatcctgaccgggttgttccccccgacctcgggcaccgcctacatcctgggaaaagacattcgctctgagatgagcaccatccggcagaacctgggggtctgtccccagcataacgtgctgtttgacatgtgagtaccagcagcacgttaagaataggccttttctggatgtgtgtgtgtcatgccatcatgggaggagtgggacttaagcattttactttgctgtgtttttgttttttctttttttcttttttatttttttgagatggagtctcgctctgtagccaggctggactgtagtggcgcgatctcggctcactgcaaccttggcctcccaggttcaagcgattctcctgcctcagcctcccgagtagctgggactctaggcacacaccaccatgcccagctaatttttgtgtttttagtagagacggggtttcaccatgttggccaggatggtctcaatgtcttgacctcgtgatccgcccacctcggtctcccaaagtgctgggaacacaggcatgagccactgtgtctggccacattttactttctttgaatatggcaggctcacctccgtgaacaccttgagacctagttgttctttgattttaggagaagtgggaggtgaatggttgagctgtagaggtgacatcagcccagccagtggatgggggcttgggaaacattgcttcccattattgtcatgctggagggccctttagcccatcctctccccccgccaccctccttattgaggcctggagcagacttcccagacctggtagtgcttcagggccctggtatgatggacctatatttgctgcttaagacatttgctcccactcaggttgtcccatcagccataaggcccccagggagcccgtgtgatggagcagagagagacctgagctctgcaatcttgggcaaggcttttcccttatgtttcttcttatctaaagtgaacagctggggctcatgtgctccctcctcatctaaagtgaacacatggggctcatgtgcagggtcctccccgctttcagagcctgaggtcccctgaggctcaggaaggctgctccaggtgagtgccgagctgacttcttggtggacgtgctgtggggacagcccattaaagaccacatcttggggccctgaaattgaaagttgtaactgcctggtgcatggtggccaggcctgctggaaacaggttggaagcgatctgtcacctttcactttgatttcctgagcagctcatgtggttgctcactgttgttctaccttgaatcttgaagattatttttcagaaattgataaagttattttaaaaagcacggggagagaaaaatatgcccattctcatctgttctgggccaggggacactgtattctggggtatccagtagggcccagagctgacctgcctccctgtccccaggctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtgcggcccagagctaccttccctatccctctcccctcctcctccggctacacacatgcggaggaaaatcagcactgccccagggtcccaggctgggtgcggttggtaacagaaacttgtccctggctgtgcccctaggtcctctgccttcactcactgtctggggctggtcctggagtttgtcttgctctgtttttttgtaggtggaatgcagagaaagctatctgtggccttggcctttgtcgggggatctaaggttgtcattctggatgaacccacagctggtgtggacccttactcccgcaggggaatatgggagctgctgctgaaataccgacaaggtgcctgatgtgtatttattctgagtaaatggactgagagagagcggggggcttttgagaagtgtggctgtatctcatggctaggcttctgtgaagccatgggatactcttctgttakcacagaagagataaagggcattgagactgagattcctgagaggagatgctgtgtctttattcatctttttgtccccaacatggtgcactaaatttatggttagttgaaagggtggatgcttaaatgaatggaagcggagaggggcaggaagacgattgggctctctggttagagatctgatgtggtacagtatgaggagcacaggcaggcttggagccaactctggcntggccctgagacattgggaaagtcacaacttgcctcaccttctttgccgataataatagtggtgcnttacctcatagaggattaaattaaatgagaatgcacacaaaccacctagcacaatgcctggcatatagcaagttcccaaataaaatgcntactgttcttacctctgtgaggatgtggtacctatatatacaaagctttgccattctagggrtcatagccatacagggtgaaaggtggcttccaggtctcttccagtgcttacccctgctaatatctctctagtccctgtcactgtgacaaatcagaactgagaggcctcacctgtcccacatccttgtgtttgtgcctggcaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggtgagctgcagtcttggtgctgggctggtgttgggtctgggcagccaggacttgctggctgtgaatgatttctccatctccaccccttttgccatgttgaaaccaccatctccctgctctgttgcccctttgaaatcatatcatacttaaggcatggaaagctaaggggccctctgctcccattgtgctagttctgttgaatcccgttttccttttcctatgaggcacagagagtgatggagaaggtccttagaggacattattatgtcaaagaaaagagacttgtcaagaggtaagagccttggctancaaatgacctggtngttcctgctcattacttttcaatctcattgaccttaacttttaaactataaaacagccaatatttattaggcactgatttcatgccagagacactctgggcantgaaagaaagtaatgataatagttaattttatatagcgttgttaccatttacaaccttttttttttttttaacctctatcatctcaattaaagtgcagagagaccctgggaagaaggtaactatatttattatcccagatgagggaagtgaggcttgtagggaattggtagctgattcaaggtcacccagcaggtaaataacagtggtgggaccagacccaattaccaggtatgttttcctctgtaccgcagtacatgcctgagatttatttgtgtgttgaagccagtggtacctaatgtatttacatcccaacctgaaactcctatccacttatttaccttttaatgagcctcttaactcaagtgcagtctgaggaccagcagcatcaggatcacttgggaacttgttagaaattcagcaacctgggcccagctcagacctaccgaatcagaatctgtgcattttaacaaggttcttgagtggttgaacacacattaaagcatgagaagcattgaactagacatgtagccaggtaaaggccttgcctgagatggttggcaaaggcctcattgcagcattcattggcaggccacagttcttttggcagctctgcttcctgacctttcaccctcaggaagcgaggctgttcacacggcacacacatgccagacagggtcctctgaagccacggctgccagtgcatgtgtcccagggaaagctttttcctttagttctcacacaacagagcttcttggaagccctccccggcgaaggtgctggtggctctgccttgctccgtccctgacccgttctcacctccttctttgccatcaggaggacagtgtttctcagagcagttctgatgctggcctgggcagcgaccatgagagtgacacgctgaccatcggtaaggactctggggtttcttattcaggtggtgcctgagcttcccccagctgggcagagtggaggcagaggaggagaggtgcagaggctggtggcgctgactcaaggtttgctgctgggctggggctgggtggctgcgggkgtgggagcagcttggtggcgggttggcctaatgcttgctggggtgcctggggctcggtttgggagctagcagggcagtgtcccagagagctgagatgattggggtttggggaatcccttaggggagtggacactgaataccagggatgaggagctgagggccaagccaggagggtgggatttgagcttagtacataagaagagtgagagcccaggagatgaggaacagccttccagatttttcttgggtagcgtgtgtaggaggccagtgtcaccagtagcatatgtggaacagaagtcttgacccttgctatctctgcctagtcctaatggctggcttttcccaggaaggcttctgcttccatggactgttagattaaccctttatttaggtaaatgagggaacctactttataagcataggaaagggtgaagaatcttttaagattcctttactcaagttttcttttgaagaatcccagagcttaggcaatagacaccagactttgagcctcagttatccattcacccatccacccacccacccacccatccttccatcctcccatcctcccattcacccatccacccatccagctgtccacccattctacactgagtacctataatgtgcctggctttggtgatacaaaggtgaataagacatagtcctttcctttgcccccaaccctcagaccagagatgaacatgtggaatgacctaaacacctggaacaggtgtggtgtatgagcggcaggcctctgatgagagggtgggggatggccagccctcactccgaagcccctctgagttgattgagccatctttgcattctggtccctgcagatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaagtaagttaagtggctgactgtcggaatatatagcaaggccaaatgtcctaaggccagaccagtagcctgcattgggagcaggattatcatggagttagtcattgagtttttaggtcatcgacatctgattaatgttggccccagtgagccatttaagatggtagtgggagatagcaggaaagaagtgttttcctctgtaccacagtacatgcctgagatttgtgtgttgaaaccagtggtacctaacacatttacatcccaaccttaaactcctatgcacttatttaccctttaatgagcctctttacttaagtacagtgkgaggaacagcggcatcaggatcacttgggaacttgttagaaattcagcaacttgggcccagctcagacctactgaatcagaatcaggagcaattctctggtgtgactgtgtcacagccaggtatcaactggattctcatacataggaaatgacaaacgtttatggatggatagtctacttgtgccaggtgctgagatttgttttttgttttttgatttttttttaatcactgtgacctcatttaattctcaaaaaaagatgaaaaaatgaacactcaggaatgctgacatgagattcagaatcaggggtttggggcttcaaagtccatcctctctttatccatgtaatgcctccccttagagatacaacatcacagaccttgaaggctgaaggggatataaaagctgtctggccaagtggtctccaagcttgacagtgcagcagaatcacctggggatattattaaaaataaacatactaaggtttggcttcagggcctgtgaatcagaatttctggaggtgaggccttgaagtctgtatttctattgcatactttggacacagtggtctatagactagagtttggaaatgattgcgctcattcagattctcttctgatgtttgaattgctgccatcatatttctagtgctctatttcctcctgctcattctgtcttggataacttatcatagtactagcctactcaaagatttagagccacagtcctgaaagaagccacttgactcattccctgtaggttcagaataaatttcttctgcgcagtgtctgtcatagctttttttaaatttttttttatttttgatgagactggagttttgctcttattgcccaagctggagtgcagtggtgcgattttggctcactgcaacctccacctcccaggttcaagcgattctcctgcctcagcctcccaagtagctgagattacaagcatgtgctaccacgcccagctaattttgtatttttagtagagatgggttttatccatgttggtcaggctggtctcgagctccagacctcaggtgatctgcccgcctcggcctcccaaagtgctgggattataggcctgagccacagcgctcagccataactttaatttgaaaatgattgtctagcttgatagctctcaccactgaggaaatgttctctggcaaaaacggcttctctcccaggtaactctgagaaagtgttattaagaaatgtggcttctactttctctgtcttacggggctaacatgccactcagtaatataataatcgtggcagtggtgactactctcgtaatgttggtgcttataatgttctcatctctctcattttccagatattcctcaaggtggccgaagagagtggggtggatgctgagacctcaggtaactgccttgagggagaatggcacacttaagatagtgccttctgctggctttctcagtgcacgagtattgttcctttccctttgaattgttctattgcattctcatttgtagagtgtaggtttgttgcagatggggaaggtttgttttgttgtaaataaaataaagtatgggattctttccttgtgccttcagatggtaccttgccagcaagacgaaacaggcgggccttcggggacaagcagagctgtcttcgcccgttcactgaagatgatgctgctgatccaaatgattctgacatagacccaggtctgttagggcaagatcaaacagtgtcctactgtttgaatgtgaaattctctctcatgctctcacctgttttctttggatggcctttagccaaggtgatagatccctacagagtccaaagagaagtgaggaaatggtaaaagccacttgttctttgcagcatcgtgcatgtgatcaaacctgaaagagcctatccatatcacttcctttaaagacataaagatggtgcctcaatcctctgaacccatgtatttattatcttttctgcggggtcctagtttcttgtatacattaggtgtttaattgttgaacaaatattcattcgagtagatgagtgattttgaaagagtcagaaaggggaatttgctgttagagttaattgtaccctaagacttagatatttgaggctgggcatggtggctcatgccagtaatcccagcgctttgagaggctgaggtgggtagatcacctgaggtcaggagtttgagaccagtctgaccaacaaggtgaaaccccgtctctactaaatacaaaaaattagccgagtgtggtggcacatgcctgtcatcccagctacttgggaggctgaggcaggagaatcgcttgaacccaggaggcagaggttgcagtcagccacggttgcgccattgcactccagactgggcaacaagagtgaaaactccatctcaaaaaagaaaaaaaaagaattagatattttggatgagtgtgtctttgtgtgtttaactgagatggagaggagagctaagacatcaaacaaatattgttaagatgtaaaagcacatcagttaggtatcattagtttaggacaaggatttctagaaaatttttaggaacagaaaactttccagttctctcacccctgctcaaagagtgtatggctcttacattatatataactgcctgacttcatacagtatcagtacttagatcatttgaaatgtgtccacgttttaccaaaatataatagggtgagaagctgagatgctaattgccattgtgtattctcaaatatgtcaagctacgtacatggcctgtttcatagagtagtctataagaaattgatgacttgattcatccgaatggctggctgtaacacctggttacgcatgaacacctcttttcagttgtctcaagacacctttcttttctgtacttatcagacaaggactgaaaggcagagactgctactgttagacattttgagtcaagcttttccttggacatagctttgtcatgaaagccctttacttctgagaaacttctagcttcagacacatgccttcaagatagttgttgaagacaccagaagaaggagcatggcaatgccgaaaacacctaagataataggtgaccttcagtgttggcttcttgcagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcaggtgagacgtgctgttttcgccagagactctggcttcatgggtgggctgcaggctctgtgaccagtgaaggcaggatagcatcctggtcaagatatggatgccggagccagatttatctgtatttcaatcccagttctattccttgccagttgtgtatccgctggcaagttacttctctatgcctcaatctcctcatctgtaaaatggggataataatattacctgcaatacagggttgttacgaaaataaaaatgaataggtgcttagaatggggcctgacattagtaagtgcttagttttgtgtgtgtatatgttatttttattttggaggagaacataaaaaggacaaagtgtagaaaaactggttgggtgtattcagctgtcataacatgagagttgttatgcccagatgcacttgacatgtgaatttattagaaacatgatttttctctgagttgatgtttaactcaaactgatagaaaagataggtcagaatatagttggccaacagagaagacttgttagactattgtctgcatgtcagtgtttgcatgctaacttgcttagttagaaaggttaaattttttcactctataaaatcaagaaatatagagaaaaggtctgcagagagtctttcatttgatgatgtggatattgttaagagcgggagtttggagcatacagagctcaagttgaatcctgactttgctacttattggctatatgaccttgggcaagctgcttagtctctctgatcctcagttacctttgtttgttgatgatgaccattgataacacaaccataaataatgacaacatagagatagttctcattatagtagttgttatacagaattattcactcaatgttaattttctgcattgaaatcccagaacattagaattgggggcattatttgaatctttaaggttataaggaatacatttctcagcaataaatggaaggagttttgggttaacttataaagtatacccaagtcatttttttttcagagaagatatggtagaaagtcttaggaggttgaagaaggaattggatatttattctttctgagactatcatgggagataatgactatggttgtccatgattggagccgttgctgtagagttggttttattatagtgtaggatttgaatgggccatgtgttctcagacctcagattaaaawgagaaaactgaggccagtggggagcgtgacttcacatgggtacacttgtgctagagacagaaccaggattcaggacttctggctcctggtcctgggttcatggcccaatgtagtctttctcagtcttcaggaggaggaagggcaggacccagtgttctgagtcaccctgaatgtgagcactatttacttcgtgaacttcttggcttagtgcctctgccaggtggccataacctctggccttgtgttgccagagaaaaggtttagttttcaggctccattgcttcccagctgccaagaatgccttggtgcagcacagtcataggccctgcattcctcattgccgtgctggttggtcggggaggtgggctggactcgtagggatttgccccttggccttgtttctaacacttgccgtttcctgctgtccccctgccccctccactgcctgggtaaagattgtcttgccagctgtgtttgtctgcattgcccttgtgttcagcctgatcgtgccaccctttggcaagtaccccagcctggaacttcagccctggatgtacaacgaacagtacacatttgtcaggtatgtttgtcttctacatcccaggagggggtaagattcgagcagaccaaagatgtttacgagggccaagggaatggacttcagaattacacggtggaatgaattttactgctgcggctcaggtccctgtataagctaatactgcatgcatagaacagcagcgaactaaccctgaataataggccagtcttctgttgagcctttcagcctctctcctcttcatcctactgttgtcaggaacagccacatgtgttttaggtgaaataatccacccttgcaaaaatccatgattaagttataaaatatttggatttgtggagctgtgttttaattctgtaactgagtcacagggcacactgtcaaagcatagaacctccagagacttgttttctgcaaagtataattcatgtaattattatctattctgttatatttgggatgttaggtagtgtttgttctttagataaaaatatcccccactctgtaacaatacattaaatcaaagaaaaggacaaaggatttttctgggtcttgttagcaggagctttcttcagtcctgaaagatttgtagacctgtagatgggggaactgtgtcagtgatacaaaagggaagcatttaaaaaaaaaaaagtatatatatatatatatatatatatatgtaatgtgaattggcctctttttctctaagncccacattttncttcttacatagttcaggtttactttattttttcctttccggctgctgaccctgtattgcccgtagttgtggaacatagcatgtgtttgtgacctgtgcctgttatttttgtgctttctagttgtgcatgcaaagagtacaaagttttcttgccctttcttggaaaatcctgcttgtctgtgccaaagggataattgtgaaagcacttttgaaatacttaatgagttgattttcttcaaattaaaaaaaatatataaatgtatatgtgtatgtacatgtgtgtacacatacacacctttatacatacagcccatttaaaacaagctccactttggagtgctctacgtcaccctgatgccgaatacagggccagagtctgagatccttctgggtggtttctgtgttttgttcatttctgttttaagagcctgtcacagagaaatgcttcctaaaatgtttaatttataaaaacatttttatctctcgattactggttttaatgaattactaagctggctgcctctcatgtacccacagcaatgatgctcctgaggacacgggaaccctggaactcttaaacgccctcaccaaagaccctggcttcgggacccgctgtatggaaggaaacccaatcccgtgagtgccactttagccataagcagggcttcttgtgcttgttgcctggtttgatttctaatatgctgcatttatcaactgcatgccacattgtgaccgccagcatttgccctttgaattattattatgttttatttacaaaaagcgaaggtagtaaccgaactaaattatctaggaacaaacgtttggagagtcttctaacaccgtgcaaagcacgtcattacagacatttgtttactgatttagaaccttaatatttaatttaaatangcactttacacttactgatgaaatgcttttcctttctttctctcccagcccctgtacttaagtgcttcaataggctctcattatatatgatttttaggttttgcttatcagcttcttcgcttttataatctgaaaagatggcatatgaatttttataaaaagggacactttcttcttctcaaattgtatatttttattgtactttccttcaaaacccccttttaaaaagtaagcagtggataaataaattcagtgaagcatccatatgacccttaagtgagtgtaggggaagggaggtcaccagatcactgtgagtgaagatggtggagaggtgaggatcttatgaggccgtgctcaaggctggtagaggtgggttagtgtttccaggtttaggcagaatctcagctgaggtcatgaaacaacagtgatctctgaaaaattatggcaaggtgggaaggtgctggagaattggagagggggcaaacttgactttcaagtttcaatgggaagataggtgactctgcacaccacagaacagtgagcatgataacctgtttatacaaggttctagagcagatttctaaatggatagctactgtgtgcttgtttgttcttaattagtattggatagttactaaatacttgttagtacttagtacataatgggtggtaaatcctagcagctaatattggttcccaaataaccagatgacaaggatagagaaggacacagacacggcctatctggatttcatggtgcctttgattttccacatgaaggttgtgtagggaagatagaagcatgagatgagatgataatatagttatctggattcatcactggccagctgaaccatatgaactcatggattgatgctagcttaggaaggctctgtaggagccagaactgggctgagagccagcccatagagacaaaagaggcccggccctgacatcagagggttcaaacatgatgtctgagccccacctacagtctgccggaggtggttggaaggaagagcctttatccttacaattcttactgaaattcaaatttttaggttttgcaaaaaaatggtggacctgaaggaaatttgacaggagcatgtctcagctgtatttaaatttgtctcagccaatccccttttgaatgttcagagtgtaagcttcaggagggcagcgcgtcttagtgtgacttttctggtcagttcaggtgctttaaggagacaattagagatcaatctggaaaacttcatttgaatttttaatacataagaaaacaataagaaatagttaaaaatatatatttatataatatatatgtgtgtgtgtgtgtgtgtgtgtgtatatatatatatatntttatttnatttatttnnnnnnnnttttttgagatggagtctcgctctgttgcccaggctggagtgcagtggctcaatcttggctcactgccacctctgcctcccaggttcaagtgattctcctacctcagcctcctgagtagctgggattacaagcatgtgccaccacactggctaatttttctaattttagtagagatggagtttcaccatgttggacaggatggtcttgaactcctgacttagtgatccacccgccttcgcctcccaaagttctgggattacaggcatgagccatcgtgcctggcaattatatttaatatttaataataaggaaataattgctgtaactttactttaaattgtggaattctgaaactggaagggaactggaaatgacttgttgaatcaaatcattttaaacttttattttgccagtggaaaaaataagcccccaaaagagcaggggacctgctgnatgtcccacagtaattcagagctggagatgaggttgaaggctttgtgtcttatctccagggaaaatttgtagacagcgtagctctnttatgtgacgagcattctcaccccagtcatcccccaattctctactcatttgagaacataaattggatcttgccagtctctactcatttttcagcacatcgagcataagatccagactctttcccaggcctctctcatctggctcctctcctcctcctttatcattactcttcttcgtagcttatcctactccagccatgctgtcttcctattattcctaaaaartagaaatgcatttcttcctagggcctttgtacctgcacttgccatcgcttttgctcagaatgttctttttgccaagcttttgcccagcttgttctccatcattgttatgttttggctgaaatgtcttctcttagtaggttcattctccccagtcactgtctttttattttgctttattttgggccatctaaggttatcttattagtgtatttgttgttcgtctcctccatgggcatacacctccatgaaggcaggtattttcaccttaggccctcgaatatactggacagcatctggcacgtagtagatgctcaacgaatgtttgttgtgtgagcaaatggttggttgattggattgaactgagttcagtatgtaaatatttagggcctctttgcattctattttacttatgtataaaatgatacataatgatgatataaatgatgtcacagtgtacaaggctgttgtgggatcaagcaatcaaatgagatcatgcttgtcttttccaaatggtgagggaatagatgcatgtttgtggttgttacggaatgatcctgtgctcctgaggcaacagaaaggccaggccatctctggtaatcctactcttgctgtcttccctttgcagagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaagtgagtcactttcagggggtgattgggcagaaggggtgcaggatgggctggtagcttccgcttggaagcaggaatgagtgagatatcatgttgggagggtctgtttcagtcttttttgttttttgtttttttttctgaggcggagtcttgctctggtcgcccaggctggagtgctgtggcatgatcttgcctcactgcaacctccacctcccaggttcaagcgattctcctgcctcagcctcctgagtagctgggattacaggcacgcaccaccatgtctggctaatttttgtgtttttagtagagatagggtttcgccgtgttggctaggctggtctggaattcctgacctcaggtgatccacccgcctcggcctcccaaagtgctgggattacaggcgtgagccactacgcccagccctgtttcagtctttaactcgcttcttgtcataagaaaaagcatgtgagttttgaggggagaaggtttggaccacactgtgcccatgcctgtcccacagcagtaaagtcacaggacagactgtggcaggcctggcttccaatcttggctctgcaacaaatgagctggtagcctttgacaggcctgggcctgtttcttcacctctgaattagggaggctggaccagaaaactcctgtggatcttgtcaactctggtattcttagagactctgtttgggaaggagtcctgagccattttttttttcttgagaatttcaggaagaggagtgcttatgatagctctctgctgcttttatcagcaaccaaattgcaggatgaggacaagcaattctaaatgagtacaggaactaaaagaaggcttggttaccactcttgaaaataatagctagtccaggtgcggggtggctcacacctgtaatctcagtattttgggatgccgaggtggactgatcacctaaggtcaggagttcgaaaccagcttggccaatgtggcgaaaccctgtctctactaaaaattcaaaaattagccaggcatggtggcacatgcctgtaatcccagttacttgggaggctgaagcaggagaattgcttgaacctgggaggtggaggtcgcagggagccaaaattgcgccactgtactccagcctgagcaacacagcaaaactccatatcaaaaaataaaatgaataaaataacagctaatctagtcatcagtataactccagtgaacagaagatttattaggcatagtgaatgatggtgcttcctaaaaatctcttgactacaaagaatctcatttcaatgtttattgtttagatgttcagaataaattcttgggaaagaccttggcttggtgtaagtgaattaccagtgccgagggcagggtgaaccaagtctcagtgctggttgactgagggcagtgtctgggacctgtagtcaggtttccggtcacactgtggacatggtcactgttgtccttgatttgttttctgtttcaattcttgtctataaagacccgtatgcttggttttcatgtgatgacagagaaaacaaaacactgcagatatccttcaggacctgacaggaagaaacatttcggattatctggtgaagacgtatgtgcagatcatagccaaaaggtgactttttactaaacttggcccctgccktattattactaattagaggaattaaagacctacaaataacagactgaaacagtgggggaaatgccagattatggcctgattctgtctattggaagtttaggatattatcccaaactagaaaagatgacgagagggactgtgaacattcagttgtcagcttcaaggctgaggcagcctggtctagaatgaaaatagaaatggattcaacgtcaaattttgccacttagtagcaacttgaccaggtaactggttatccttttaaagccttagtttatctaaattgtgatattaatgttgctcttataagtttgtcatgaggactaaattaaatggtgtacatagagtgccttgggtactctctgatgggggactccatgataatttgtggtctcatggagggagctctgggaaggtttaggagcctgccttggctctgcagccttgggagagccttctagcttcccaggacatggcagcctagtgttgaatgcttggctcagcaaatgtttgttctcgtttccttcccatcaacttggtcagttggggtctttcagttaggagtatctcagtgactttaaatggcatgggcatgctggagtgatagtgaccatgagtttctaagaaagaagcataatttctccatatgtcatccacaattgaaatattattgttaattgaaaaagcttctaggccaggcacggtggctcatgcctgtaatcccagcactttaggaggccaaggcgggtggatcacttgaggtcaggagtttgagaccagcctggccaacatggggaaaccctgtctctactaaaaatacaaaataagctgggcgtggtggtgcgtgcctgtaatcccagctacttgggaggctgaggcaggagaattgcttgaatctgggaggcggaggttgcagtgagctgagttcatgccattgcattccagcctgggcaacaagagcgaaaccatctcccaaaagaaaaaaaaaagaaagaaaaagcttctagtttggttacatcttggtctataaggtggtttgtaaattggtttaacccaaggcctggttctcatataagtaatagggtatttatgatggagagaaggctggaagaggcctgaacacaggcttcttttctctagcacaaccctacaaggccagctgattctagggttatttctgtccgttccttatatcctcaggtggatatttactccttttgcatcattaggaataggctcagtgctttctttgaactgattttttgtttctttgtctctgcagcttaaagaacaagatctgggtgaatgagtttaggtaagttgctgtctttctggcacgtttagctcagggggaggatggtgttgtaggtgtncttggattgaagaaagccttggggattgtttgtcactcacacacttgtgggtgccatctcactgtgaggaggacagaagccctgtgaacatgtggagcacacaggggcacagacagatttagattaggcctgctttatagagtttctgcctagagcatcatggctcagtgcccagcagcccctccagaggcctctgaaatatttgatatactgatttccttgaggagaatcagaaatctcctgcaggtgtctagggatttcaagtaagtagtgttgtgaggggaatacctacttgtactttccccccaaaccagattcccgaggcttcttaaggactcaaggacaatttctaggcatttagcacgggactaaaaaggtcttagaggaaataagaagcgccaaaaccatctctttgcactgtatttcaacccatttgtccttctgggttttgaaggaacaggtgggactggggacagaagagttcttgaagccagtttgtccatcatggaaaatgagataggtgatgtggctacgtcagggggcccgaaggctccttgttactgatttccgtcttttctctctgccttttccccaagggccaggacccctggatctctgggcagagcagacgcaggcccctataatagccctcatgctagaarggagccggagcctgtgtataaggccagcgcagcctactctggacagtgcagggttcccactctcccaactccccatctgcttgcctccagacccacattcacacmcgagccactgggttggaggagcatctgtgagatgaaacaccattctttcctcaatgtctcagctatctaactgtgtgtgtaatcaggccaggtcctccctgctgggcagaaaccatgggagttaagagattgccaacatttattagaggaagctgacgtgtaacttctnngaggcaaaatttagccctcctttgaacaggaatttgactcagtgaaccttgtacacactcgcactgagtctgctgctgatgatactgtgcaccccactgtctgggttttaatgtcaggctgttcttttaggtatggcggcttttccctgggtgtcagtaatactcaagcacttcctccgagtcaagaagttaatgatgccatcaaacaaatgaagaaacacctaaagctggccaaggtaaaatatctatcgtaagatgtatcagaaaaatgggcatgtagctgctgggatataggagtagttggcaggttaaacggatcacctggcagctcattgttctgaatatgttggcatacagagccgtctttggcatttagcgatttgagccagacaaaactgaattacttagttgtacgtttaaaagtgtaggtcaaaaacaaatccagaggccaggagctgtggctcatgcctgtaatcctagcactttgggaggctgaagcgggtggatcacttgaggtcaggagttcgagaccagcctggcctacatgacaaaaccccgtatctactaaaaatacaaaaaaattagctgggcttggtggcacacacctgtaatcccagctacttgggaggctgaggcaggagaattgcttgaaccctgtaggaagaggttgtagtgagccaagatcgcaccgttgcactccagcctgggcaacaagagcaaaactccatctcaaaaaacaaattaaatccagagatttaaaagctctcagaggctgggcgcggtggcttacacctgttatcccagcattttgggatgccgaggcgggcaaagcacaaggtcaggagtttgagaccagcctggccaacatagtgaaaccctgtctctgctaaaaacatagaaaaattagccgggcatggtggcgtgcgcctgtaatcccagctactcgggaggctgaggtgagagaattrcttgaacccgggaggcggaggttgcagtgagcccagattgcaccactgcactccagcctgggcgacagagcaagactccatctcaaaaaaagctctcagaacaaccaggtttacaaatttggtcagttggtaaataaactgggtttcaaacatactttgctgaaayaatcactgactaaataggaaatgaatctttttttttttttttttaagctggcaagctggtctgtaggacctgataagtactcacttcatttctctgtgtctcaggtttcccatttttaggtgagaattaaggggctctgataaaacagaccctaggattgtggacagcagtgrtagtcctagagtccacaagtctgcttttgagtgatgggcccatgtatctggcacatctgcaggcagagcgtggttctggctcttcagatgatgccggtggagcactttgaggagtcctcaccccaccgtgataaccagacattaaaatcttggggctttgcatcccaggatttctctgtgattccttctagacttgtggcatcatggcagcatcactgctgtagatttctagtcacttggttctcaggagccgtttatttaatggcttcacatttaatttcagtgaacaaggtagtggcattgctcttcacagggccgtcctgttgtccacaggttccagattgactgttgccccttatctatgtgaacagtcacaactgaggcaggtttctgttgtttacaggacagttctgcagatcgatttctcaacagcttgggaagatttatgacaggactggacaccaraaataatgtcaaggtaaaccgctgtctttgttctagtagctttttgatgaacaataatccttatgtttcctggagtactttcaactcatggtaaagttggcaggggcattcacaacagaaaagagcaaactattaactttaccagtgaggcagtacggtgtagtgtagtgattcagagaatttgctttgccaccagacataccaggtaaccttgactaagttacttaacctatctaaacctcagttycctcatctgtgaaatggagacagtaatcatagctatttccaaactgttgtgagaattcaatgagttaaaggtataaggtcctcaccacagcgcctgcccacatagtcagtgatcactatgtcctgaacactgtaattacttcgccatattctctgatcatagtgttttgccttggtatgtgactagaatttctttctgaggtttatgggcatggttggtgggtatgcacctgcctgcaggagcccggtttgggggcattaccttgtacctggtatgttttctttcaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgtaagtgtggctgtgtctgtatagatggagtggggcaagggagagggttatggagaaggggagaaaaatgtgaatctcattgtaggggaacagctgcagagaccgttatattatgataaatctggattgatccaggctctgggcagaagtgataagtttacgaattggctggttgggcttcttgaactgcagaagagaaaatgacactgatatgtaaaaatcgtaacatttagtgaattcatataaagtgagttcaaaaattgttaattaaattataatttaattataagtgtttaatcagtttgatttgtttaaaaaccactgttttaaatttggtggaatatgtttttattagcttgtatctttaattcctaaattaagctgtgtgtgtgtgtgtgtgtgtgtgtgtgtgtgtgtgtgtgtgtgtgnntgtgaagtttaaagccaggatgagctagtttaaagtatgcagcctttggagtcatacagatctgggtttgaatctggtctctaaactttatagatgtatgatattaaatgaggcagttcatgtaaattgccaagcccagcactcagcacagagttgatatttcacacacattagatacctttcctgtatgtggagcatggcagttcctgtttctgctttactcctacaggatactaatataggacactaggatctttataccaagaccccatgtaatgggcttatgagaccattcttcttataaaaatctgacagaatttttgtatgtgttagatcaataggctgcatactgttattttcaagttgatttacagccagaaatattaatttatttgagtagttacagagtaatatttctgctctcatttagttttcaagccccactagtcctttgtgtgtgaaaatttacaacttactgctcttacaaggtcatgaacagtggaccaaagtgaatgccattaaccactctgacttccttcattagttttattgtgacagtggactcttttgacctcagtaataccagtttggcatttacattgtcatatttttagacttaaaaatgatcatcttaaccctgaataaaatgtgtctggtgaacagatgtttttccttgggctgtgcctcagatatctctgtgtgtgtgtacgtgtgtgtttgtctgtgtgtccatgtcctcactgattgagccctaactgcatcaaagacccctcagattttcacacgctttttctctccaggatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatggtaaggacacaggcctgctgtatctttctgatgtctgtcagggccatggattgatatggataagaaagaaagagctctggctatcatcaggaaatgttccagctactctaaagatgtatgaaaaagaaatagccagaggcaggtgatcactttcatgacaccaaacacagcattgggtaccagagttcatgtcacaccagagggaaaattctgtacacaatgatgaaaattaataccactaccacttaagttcctatgtgacaactttcccaagaatcagagagatacaagtcaaaactccaagtcaatgcctctaacttctctgatgggttttaacctccagagtcagaatgttctttgccttactaggaaagccatctgtcatttngaaaactctgtacattttatcagcagcttatccatccattgcaaatantgtttttgtgccagccacaatatattgcttctatttggaccaatatgggggatttgaaggaattctgaagttctaattatatttcaactctactttacaatatctccctgaaatatatctccctgtaacttctattaattataagctacacagagcaaatctaattcttctcccaccgaacaagtccctggatatttaaaaataactctcatactctcatttaacctgagtattacccagataagatgatatatgagaatacaccttgtaacctccgaagcactgtacaaatgtgagcaatgatggtggagatgatgatgagatctttgctgtttataccaagccccttagactgtgtcactcttctgatccggttgtccttgtatggccatgctgtatattgtgaatgtcccgttttcaaaagcaaagccaagaattaaccttgtgttcaggctgtggtctgaatggttatgggtccagagggagttgatctttagctcacacttctattactgcagcacaaagattttgcattttggaaggagcaccgtcttactggcaacttagtggtaaaccaaaacctccatttcacacaaatgattgtgaaattcgggtctccttcattctatacaaattcatttgatttttttgaaactaaactttatatttatccatattaaattacatgggttttatttttgttttatcttgattcagtaattactcctttcagtaaacacagactgagtgctgtgtgtctgacttatgccaggcataggtgattcagagatgaaaggtcaagtccctgaacccatctcttgtcttcctgggtattatctgtccctccctgctttagagctcctgaaatttgctagaagcatgtcttcatctaagttgttgataaacacatcaagtaggattggactgaggcagagccctgtagtctgaagctgcagttcttctagcggctgacaagccccactatcacttccctgctggtgctttgctctgccagctgtgaattctcataattgtcctatcgtcaagtctttatttctgcattttactgcttgatacactgtcaggacagactttaaaattattctcagtgcgatgaaacaattctgacattcatgttatgagcagttacctcataaatagattacatgtgagattgaacttgggcagactataatatagcattaatgatgaaacagacacagtcatcttcgggaagaagaatagaggcttatttgctgcctgtgaaattaaaattactctgactgggaatccatcgttcagtaagtttactgagtgtgacaccttggcttgactgttggaaagacagaaagggcatgtagtttataaaatcagccaaggggaaaatgcttgtcaaaatgtattgtcgggtattttgattaatagtttatgtggcttcattaattcagagttactctccaatatgtttatctgccctttcttgtctgataatggtgaaaacttgtgtgatgcattgtatatttgatttaggggtgaactggatgtctttgttttcacttttagtgcaattacgttgtccctgccacactggtcattatcatcttcatctgcttccagcagaagtcctatgtgtcctccaccaatctgcctgtgctagcccttctacttttgctgtatgggtaagtcacctctgagtgagggagctgcacagtggataaggcatttggtgcccagtgtcagaaggagggcagggactctcagtagacacttatctttttgtgtctcaacaggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaatgtgagtcatgcagagagaacactcctgctgggatgagcatctctgggagccagaggacagtgtttaattgtgatcttattccacttgtcagtggtattgacactgctgactgccttgtcctgtcttcagagtctgtcttccctgagaaggcaaagcacctttctttcttgctgtgccttacattttgctggtcaagcctttcagtttcttttgacagttttttttacttctttcttttttcaatgttgctcttaccaagagtagctcctctgccttccactttacacatgagagctgggcgacgncattcagtcctaaggcttttaccatcacctctcttggtgtttttattgtcatctctaagatcaatgcctttagccttgatcataaccttgaactctaatctcaaattctcacttgcctagtggattgctccatttagatagtatatagataccccaacctggatatgtcctagttttctttccccttggaacttaatgcttttcttgccatccctgtcacactcagtggcactaccatccactcggttgcccaagctggctcttagagttatcctagatgcttgctttgctgttgcagatttcccacattcaactggttatgttgtcagttcttccaggtatggacctctaaaataaggcttcctctccattccggttgtcattgcctttgtccaaacacagcacacaaggccttttacagttgcacaactcttcctgtccatacccaccacaccctttcccagctgtaagcttcagatgagttgcctccaaccaccatgctcctgtaggcctggcttgaaatgcccttcttctgtcacagggtctggtagtatatcccttgcccttcaagatttagctaaaatgtgaagctttccttacctgctgggaggtgttctctcttttctctgtgctctcagagtccttagtccatgcctccagtacaacgtacatccacttacatggtaatttcctgtttacatacttttcctactcggagtggagtctgtttcttaataattttgcctctcccatgccctagcacagtgcatccagcgtatagccccttattcagttggtagatatttggccactgttgccttgtgggatcataagttctgatgtatttgagaagaatttctaaaattctgacaaaatcctgaaactcaaatattgacccagacatgagcaatttgcttttcaaatgctaagggatttttaatggatttgctttaattaaatctagcctgtttctaagctttattcattatttctccatactcagagcatttctccagattttctaaagaatagaattttattgctacatatcatcagctatgcctgctgctatttaattggtatctgaattaaaaggtctggtttgtccctagagaatcaaattttttcttcactcccatatttcagaacttgatacatttttaggataaaccatgaatgacacccgtttcttctccctcaccctcccttccctcccatttttttttttttttttttttagaagctgaataatatcaatgatatcctgaagtccgtgttcttgatcttcccacatttttgcctgggacgagggctcatcgacatggtgaaaaaccaggcaatggctgatgccctggaaaggtttggtgagtgaagcagtggctgtaggatgctttaatggagatggcactctgcataggccttggtaccctgaactttgttttggaaagaagcaggtgactaagcacaggatgttcccccacccccatgcccagtgacagggctcatgccaacacagctggttgtggcatgggttttgtgacacaaccatttgtctgtgtctctgatagcattgagaaaagtgaaagggcagttttgaaggtaaggaaaatagtgttatttgcttggatccactggctcatgccactgtctgggttggttagaagcactggaaaagtcaaaccataactttgagaattaggtgatcagggaatcagaaggaaagatgcaaactttggctcttttaggcgaatcatgtgcctgcagatgaggtcatttattatcttttacacagtctataaaattataatgtattacatctttttctacctttagaatggttaaaaatatttctccggtagccatatgattattattcatccattagataatatagtcaaatgggccatgttatttactgttcatagaagaggggctttttgcaacttgggctacaaaggagatatgtaaggaatttaaggaatggttacatggaactagatttaattgaatctagtggtttaattgattcactaggatatatgctactgaaaggggaatctgcttaaagtgctttctgatatttattattactaaaacttagaatttattaaaaatactgactgtgaaaattacttgggtcgtttgcctttttaaaaggatttttggcatgtctcattaaaaaaagaaatactagatatcttcagtgaagttacaaatcgaatacacattggctctgaaattctgattgatactgggtcataaaaagttttcccaaatcagacttggaaagtgatcactctcttgttactcttttttccttgtcatgggtgatagccatttgtgtttattggaagatcggtgaattttaaggaacataggcccaaatttgaggaagggccatggtttttgatccctccattctgaccggatctctgcattgtgtctactaggggagaatcgctttgtgtcaccattatcttgggacttggtgggacgaaacctcttcgccatggccgtggaaggggtggtgttcttcctcattactgttctgatccagtacagattcttcatcaggcccaggtgagctttttcttagaacccgtggagcacctggttgagggtcacagaggaggcgcacagggaaacactcaccaatgggggttgcattgaactgaactcaaaatatgtgataaaactgattttcctgatgtgggcatcccgcagccccctccctgcccatcctggagactgtggcaagtaggttttataatactacgttagagactgaatctttgtcctgaaaaatagtttgaaaggttcatttttcttgttttttcccccaagacctgtaaatgcaaagctatctcctctgaatgatgaagatgaagatgtgaggcgggaaagacagagaattcttgatggtggaggccagaatgacatcttagaaatcaaggagttgacgaaggtgagagagtacaggttacaatagctcatcttcagtttttttcagctttatgtgctgtaacccagcagtttgctgacttgcttaataaaagggcatgtgttcccaaaatgtacatctataccaaggttctgtcaattttattttaaaaacaccatggagacttcttaaagaattcttactgagaattcttttgtgatatgaattcccattctcgaatacattggttttatatgcttacatttatgtgttagttattaaaacatactaatattgtatatctagtcaaaactgaggtagagagaataaatggttgattttgagtttgagtttcatagtccaaaaagctgatatattgcctgtgttcaagagggtctatatcagccctctagatgccagcatctccaaattttacttttttggaatctgtacagtatttgcaatatttttattacaaatttctactctgtggaatttaatttttaaaatacctgcaatacatatatatgttgaatagatgaaaaattatgtagatrataatgaatgatacggttctaaaaagacaggttaaaaagtaagttcacttttattttgagcttcagaatcattcagaagccagtcgccacaaacgcagaccaaggctcttggcacatcaaatatgcctatggcttagggttattgacaagtcttatgttgcagtgtatgtggtttatagtcctgccttccacagttgcttgggagagctgtgagtcactgaggcttatgaatgtttacattttgtttgttgcagatatatagaaggaagcggaagcctgctgttgacaggatttgcgtgggcattcctcctggtgaggtaaagacactttgtctatattgcgtttgtccctattagttcagactatctctacccaatcaagcaacgatgctcgttaagaggtaaaagtggattttaaaggcttctgtatttatgccaggatggagcaattagtcatcgagaagagagggaccctgtatgtcaagagaatgatttcagagaatccaatacaatttaagaaaaagcatggggctgggcgcagtgattcactcctgtaatcccagcactttgggaggccgaggtgggcggactcacgaggtcaggagattgagaccatcctggccaacatggtgaaaccccatctctactataaatacaaaaattagctgggcatagtagtgcattcctgtagtcccagctactcgggaggctgaggcaggagaattgcttgaacctaggagggggaggttgcccagattgcgctgctgcactccagcctggtgacagagtgagactcatgtcaacaacaaaaacagaaaaagcacgcacatctaaaacatgcttttgtgatccatttgggatggtgatgacattcaaatagttttttaaaaatagattttctcctttctggtttccgtttgtgttcttttatgcccttttgccagagtaggtggtgcnaatttggctangctggctttcattactgtttttcacnacnattaacntttggcctcaacttgacaactcaaataatatttataaatacagccacacttaaaatggtcccattatgaaatacatatttaaatatctatacgatgtgttaaaaccaagaaaatatttgattcttctctgatatttaagaattgaaggtttgaggtagttacgtgttaggggcatttatattcatgtttttagagtttgcttatacaacttaatctttccttttcagtgctttgggctcctgggagttaatggggctggaaaatcatcaactttcaagatgttaacaggagataccactgttaccagaggagatgctttccttaacaaaaataggtgagaaaagaagtggcttgtattttgctgcaaagactttgtttttaatttatttaaagaaataggttgttatttttgattacagtggtatttttagagttcataaaaatgttgaaatatagtaaagggtaaagaagcacataaaatcatccatgatttcaatatctagagataatcacaatttacatttcctttcagtctcattctcttcttttaacagctttattcaggtataatttacatacaatataatttgcttgttttttaagagtataatttagtgatttttggtaaattgagagttttgcaaccatcaccacaatccagttttagaacttttccatcaccccacatctgtcttatatacacatataaatgtgccatacaattgagatcatactgtatgtagaatttaaaattagtttttattgttaatgagtgtattatgaatatttcccagtgggttacatttcctaagatgtggaattttacattgctacataaaatccccctatgtacatgtacctataatttatttaataaattccttataaatgttggacacattagtttccatttttcactatgtaaatatgtccctgtatacatcttttattatttcctcaggaacaattcctacaaagtaaattgccctctctaaagagcatacaaattgactgagccaccgttaggccattttctgagactgcacaggtcacaaagcaatctgatctttgggaatacagctacattttataggcttcttagataatgttactctaagtactttaaatatgtggggcttctctgggcttttttttttttgagacggagtttcactcttactgcccaggctggagagcaatggcgcgaccttggctcactgcaacctccgcctcccaggttcaagcgattctcctgcctcagcctcctgagtagctgagattacaggtgcccgccacaatgcctgcctaatttttttgtattttcagtagagatggggtttcaccatgttggccagactggtctcgagctcctgacctcaggtgatccacctgcctcagcctcccaaagttctgggattacaggcatgagccactgcgcccggcttctctggacttattatgtggagagatagtacaaggcagtggctttcagagttttttgaccatgaccgttgtgggaaatacattttatatctcaacctagtatgtacacacagacatgtagacacatgtataacctaaagtttcataaagcagtacctactgttactaattgtagtgcactctgctatttcttattctaccttatactgcgtcattaaaaaagtgctggtcatgacccactaaatttatttcccaaaccactaatgaacaatgactcacaatttgaacacactggacagggggatagccaataaaattgaaaagagcaaggaaattaatgtattcatgatctcctctcctgtctcttacatttttgcagtagcaatgtaaaggaatcctaagagaacagacattctgggaatagcaggcctagcgctgcacaactgctttcctaggcttgctcctagtaccaagctcctgacgcatatagcagtggcagtaataaccagcccatagtaaggtttgtcacagggactggttgtaagaactgatttggttggtatagctgtgagggcctggcacggtgtccacgtgtgcctcaatcctaattctgaaaaaggctgaccctgggggtgctaattagatacacagagaggaatgaatgctgccagaaggccaagttcatggcaatgccgctgtggctgaggtgcagtcatcagtctggaacgtgaacactgaacttctctcacatgtgattcttcacttgactggcttcatagaaccccaaagccaccccaccaccacataaattgtgtctctaggttctgtgttgctcacactcaaaatttctgggccttctcatttggtgcatgtgaatggtgcatatgagtgaagtctaggatggggccttagcgttaaagccctggggtagtgtgactgagattgttggtaaagaatgtgcagtggttggcatgacctcagaaattctgaaatgggactgcacctgcagactgaagtgttcagagagccagggaggtgcaaggactggggagggtagaggcaggaaccctgcctgccaggaagagctagcatcctgggggcagaaaggctgtgctttcaagtagcagcagatgtattggtatctttgtaatggagaagcatactttacaggaacattaggccagattgtctaaccagagtatctctacctgcttaaaatctaagtagttttcttgtcctttgcagtatcttatcaaacatccatgaagtacatcagaacatgggctactgccctcagtttgatgccatcacagagctgttgactgggagagaacacgtggagttctttgcccttttgagaggagtcccagagaaagaagttggcaaggtactgtgggcacctgaaagccagcctgtctcctttggcatcctgacaatatataccttatggcttttccacacgcattgacttcaggctgtttttcctcatgaatgcagcagcacaaaatgctggttctttgtatctgctttcagggtggaaacctgtaacggtggtggggcagggctgggtgggcagagagggagtgctgctcccaccacacgagtcccttctccctgctttggctcctcaccagttgtcaggttatgattatagaatctagtcctactcagtgaaagaactttcatacatgtatgtgtaggacagcatgataaaattcccaagccagaccaaagtcaaggtgctttttatcactgtaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggtgagtataactgtggatggaaaactgttgttctggcctgagtggaaaacatgactgttcaaaagtcctatatgtccagggctgttgtatgattggcttgtcttcccccagggacagcagagcaaccttggaaaagcagagggaagcttctcccttggcacacactggggtggctgtaccatgcctgcagatgctcccaaatagaggcactccaagcactttgtttcttagcgtgattgaggctggatatgtgatttgatctttctctggaacattctttctaatcatctttgtgttcattccctgaaaatgaagagtgtggacacagctttaaaatccccaaggtagcaactaggtcatagttcctntacacacggatagatgaaaaacagatcagactgggaagtggcccttgaccttttttcttctgtagataagagcattgatgttattacgggaagaagcctttgaggcttttatgtattccacctcggtctggaatttgtttctgtaaggctaacagttgcaatatactagggtaatctgagtgagctggaattaaaaaaaaaaaggaatttcaccccaatcttatactgacttcaatagaggtttcagacaaaaagttgttttgtatatacttatcagtcatgaaaagataattacaactaaatggcctttttccttccctatttatttggagaaatttaattacataaaaaagtactcagaatatttgagtttcctgcatcaataagacatttataataatgaccttgtttacaaatgaatttgaaagttactctaattctttgattcatcaagaaataactagaatggcaagttaaaatttaagctgtttcaaagatgcttctgcatttaaaaacaaatttatctttgattttttttccccccagcaaataagacttattttattctaattacaggatgaacccaccacaggcatggatcccaaagcccggcggttcttgtggaattgtgccctaagtgttgtcaaggaggggagatcagtagtgcttacatctcataggtccgtagtaaagtcttgggttcctcactgtgggatgttttaactttccaagtagaatatgcgatcattttgtaaaaattagaaaatacagaaaagcaaagagtaaaacaattattacctgaaattatatatgcatattcttacaaaaatgcaagcccagtataaatactgctctttttcacttaatatattgtaaacattattccaagtcagtgcatttaggtgtcatttcttatagctggatagtattccattaggatatactcttatttaactattcccccttttgtagacatttggattatttccaacttgttcacaattgtaaacaccactacactgaacagcatcatccctatatccacatgtacttgtaacagaatacaattccctaggaagctggaatgctggaagtcatggtgatgttctcatggttacagagaatctctctaaaactaaaacctctttctgttttaccgcagtatggaagaatgtgaagctctttgcactaggatggcaatcatggtcaatggaaggttcaggtgccttggcagtgtccagcatctaaaaaataggtaataaagataatttctttgggatagtgcctagtgagaaggcttgatatttattcttttgtgagtatataaatggtgcctctaaaataaagggaaataaaactgagcaaaacagtatagtggaaagaatgagggctttgaagtccgaactgcattcaaattctgtctttaccatttactggttctgtgactcttgggcaagttacttaactactgtaagagttagtttccctggaagatctacctcctagctttgtgctatagatgaaatgaaaaaaatttacatgtgccagtactggtgagagcgcaagctttggagtcaaacacaaatgggtttgcatcctggccctaccaattatgagctctgagccatgggcaagtgactaactccctgggcctcagtttctctgtaacatctgtcagacttcatgggtccaggtgaggattaaaggagatcatgtatttacagcacatggcatggtgcttcacataaaataagtatttagtaaatgataactggttccttctctcagaaacttatttctgggcctgccaggggccgccctttttcatggcacaagttgggttcccagggttcagtattcttttaaatagttttctggagatcctccatttgggtattttttcctgctttcaggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtaagctttgagtgtcaaaacagatttacttctcagggtgtggattcctgccccgacactcccgcccataggtccaagagcagtttgtatcttgaattggtgcttgaattcctgatctactattcctagctatgctttttactaaacctctctgaacctgaaaagggagatgatgcctatgtactctataggattattgtgagaatttactgtaataataaccataaaaactaccatttagtgagcacctaccatgggccaggcattttacttggtgcctaatcctatttaaattagataaaaaagtaccaaataggtcctgacacttaagaagtactcagtaaatattttcttccctcttccctttaatcaagaccgtatgtgccaaagtaaatggatgactgagcagttggtgatgtaggggtggggggcgatatagaaagtcagtttttggccgggcgtggtggctcatgcctgtaatcccagcactttgggaggctgaggagcaggcagatcatgaggtcaggagatccagataatcctggccaacagggtgaaaccccgtctctactaaaaatacaaaaattagctgggcatggtggtgcgcacttgtagtcccagctacttgcgaggctgaggcaggagaattgctcgaacccaggaggtggaggttacagtgagccaaggtctcgccactgcactccagcctggggacagagcaagaccccatttcaaggggggaaaaaaagtctatttttaagttgttattgcttttttcaagtattcttccctccttcacacacagttttctagttaatccatttatgtaattctgtatgctcctacttgacctaatttcaacatctggaaaaatagaactagaataaagaatgagcaagttgagtggtatttataaaggtccatcttaatcttttaacaggtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacanttatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttttaattgcaacaatgcaaaagccaagaaagtataagggtcacaagtctaaacaatgaattcnttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccagaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgtctgtgttttcttcttgtggttgtatatatcaggtaaaatattttccaaagagccatgtgtcatgtaatactgaaccactttgatattgagacattaatttgtacccttgttattatctactagtaataatgtaatactgtagaaatattgctctaattcttttcaaaattgttgcatcccccttagaatgtttctatttccataaggatttaggtatgctattatcccttcttataccctaagatgaagctgtttktgtgctctttgttcatcattggccctcattccaagcactttacgctgtctgtaatgsgatctatttttgcactggaatatctgagaaattgmamaactagacaaaagtttcacaacagatttctaagttaaatcattttcatwaaarrraaaararaaaaaaaattttgtatgtcaataactttatatgaagtattaaaatgcatatttctatgttgtaatataatgagtcacaaaataaagctgtgacagttctgttggtctacagaaatttacttttgtgcatttgtggcaccacctactgttgaagggttataaagccattagaaaagtagaggggaagtgatttggatcaaaaggaaaaactttagaaaagattcaaatgttcccttaatcataaaagagaactgaggggactacttgaaaataaaaggttgttttgtattttcatgttggttaagatactgagntaacwggtattaagtgttagaggtttttagataaatattctgcttaatgattatgaagctgcactgagatttctgaaaatgctctgtagctgagcttatttaataaatgttcacttggtataggggaagctacaaaggcagccttcagtgtccttttgtttattcaaccaaaaatataaggacacaatgtagcagttatactgggaaggtgctgggggtggtggcaatggtgagcaggaaggcgaagtagatatggaaacagaaatgatactaatatcggtgattccttccttttttcctgtrataagtgctgtgcagacaacatatgagcagtgctgataaatgtaaatgtattkttcatagctcattaagaatcagtttcagaaagagatgtctgcttatttkgctrcttgaagaatccctgtcaaacagtccttttsaggaagtacaagaggctgtctctatttgtgacctcaggaatggctgtgacagtgtcgtgagcagtccttttcctgtggcacagatctgaactttgtgtgcagaaaaatcttggcttcaagtgagccaagatgccccctgagcatcagcatcacaacttcatcctcctatcttgaagttcatgttatagtgactttaatgaaatcatagaacactgtttcttcgtgnaacaatgacgagggagaggaaaaaactttattgaaaaataaaaaggcaggtaatttagatgaaaatatgttacccatgaggttttgtttttgctttttgtttttgtttttgagaaacagaatctcgctctgtcgtccaggctggagtgcagcggcatgatcttggctcactgcaacctccgcctcccgggttcaagcgattctcctcagcttcccaagtagctggtactacaggcatgcgccaccacaaccagctaatttttgtatttttagtagagatggggtttcactatacgttggccaggctggtctcaaactcctgacctaaggtgatccttctgccttgggctcccaaagtgctgggattacaggcatgagccaccttgcctggccctacccatgagccttgactaaaacattcttctatctgtagaaaagcccaaaagaacttttccagattcaaaaaacttggcactttgtaatggtaatgtttacattaagtaaaaaaaaaaaaaaaacccacttagcttcagttttcaagtgtttactgtgttgtcatgcacttcatttaattctcaacacctgccctatgaggtaaaaagtaccattttacatatgagtaaattacagctcagtggataagaaactcgtccaaaggtacaggttcagtcaagtggcagagggttctttttgttgaagttaggtatcagttaaaattgaccttgtaaaatcacatcagcatcaatatacattaatttaacaaatatttattgaactttactgtatgccagatacttctctaggtactagggggtacaatgtagaagaaaatagaattcctgctctaatgaatttatatttcagtggtgaaagatgatgtgtggacaaacacatctaatgtattttgacagcaatgggtgctaagaagaaaataagacatggtaattggataacaatggagagaagtcagagatggccttttctgaggagtgacatgctcagaatcgaataaaaagagcaggagcagcctttcaaggtgggtgcgaaggatattcccggaagaaagaataagtgttgcaaaagccccagtgggaaaaagcttggcaagctgaggtggtaaaagagctatctgtactaatgtctcttgtactgagttagcaaggacaagagtggcagggggtggaactggagagatagcagggaccacatctcacaggatctcgccagcctttttaaggtatttggattttattgttagtgcaacaaggagccactggagagttttaaacagtagtggtgtgacctactgtttcagaaagaacactggctactatgggaagaaaggacagtaggaagactaatagataatggtggattgaactaagatggtagcaacagatagggagctatggtaatgttcagtatccactttggagatagatccagcaggacttgctgacagactgcatgtaagggctgagggaaagagagctagcaaggtgacttctagtttgtgacctgaactaggtagatggttgtgttaaaaatgcagtgggtatttcagtctgaagctcaggggcaatgatggaaaataagataaatgtgtgcaagttggctgggcgtggtggtgcacgcctataatcacagcactttgggaggccgagacgggtggatcacttgaggtcaggcgtttgcgaccagcctaacatggtgaaaccccatctctaataaatacaaaaaaattagctgggcatggtggtgcatgcctgtaatccaagctacttgggaggctgagacaagagaatcgcttgcacctgggaggcggaggtttcagtgagctgagatcgcaccattgcactccagcctgggcaacaagagcgaaactccatctaaaaaaaaaagtgtgcaagttattggtacgtatatatttaagcctcgggactgaatgagattacctagagagaatgaagatagggaacattcttgagcctgagatcctataacatttagaagagaagctagccagggaaattgagagggactggccagtgagagagagtaaaatctgaagagtataatgccatatgttgaagctcagagaattattgtttcaggaaggcagaagttgtcaattatgtccaacattgctgagaagatgagtaaggaatgtaaatgagtaaaggcaggaatggctattgaatttgttaagacagaggtccttggtgaccttcctaagagtcatttcagcatgacaaggatgcagacctaactggaatagattttcagggaagactgagaggcaaggaaggggagactgcaaatatatgcaaatctatatttttgctttgaagggaaacagaaaacaggacaacagctggtttacagaacttttatttggattctttccttgtctgttaataatataggctgcacctgggcatcatcgtgcccttcctttttgattctgctgttttgcttttaatcctacaggcatttcttctggcaggagccttgtacacactctgggtggttcatgaccaccacggacttgccctttcaaggcagaagcctgtgattcaaaaacttccattccttccaatcgactattataaaactcattttgtctagtgcccatactcctttaccttaggatcctgggccaactgtcactaaagaaaacaaaccatttcctcctcctgggggaatgtgcttctttcaagaaggtgcttagagaatagacattctaggtgacagcagttatgaaagaggtgtctctaatgaacctaacacttcttccaacatactccaggttcaccctcctttgaagacagaaagtactacaacttcagttgccaccctagtcaggttcataggatttactcagtaggttaagagatggtgctgagaatgaagtccagagtttgagtctacaagtggcatttgcttctcctttgggacttctgtggcagggtgtggaaccaaatcagattggttgcatatctgtttctcttcctgaaaatacaattcaaataccttcatagtctgtgacagcaacttataaccaacagacaatggtattatttttatttattgatggcactcccccatagccagaacttctactgagtcattcatcgtgtattccaggaatactttccaaatggagtagtgaggcacttccaataacagttatgtaatctgttaacagtggctcaaaatgacatgtgaaggaatgatgtacttcttttttcagactcacagatctcatatgttaattcagtcaacaagtacttagggcttatcacatgccaggcacaggtaatatgacaataatgaaaacaaaaacccttgctgtcatagaattcggtttctagtgggaagagacatacattaacaaatcatatataagtaaaacattaattatggtgataacagaaaaagaaagcaggggagagggatagtgtcttagttcaggttgctgtaacaagagtaccatagacagggtggcttamacaacaaacatttctttctcacagatctagtggctgggaagtctaagaccagggtgccagcatgaagaagttctggtgaaggcttacattctggtttcctcacagtgagggctcttcacgcccttataaatgcactaattctattacccacatgacctcatctaaaactaactcacaaaggccaaacctcctatgttgagattaaggcttcaacataggaattttggggggacaaaaacattcagtctctagcagataggggccactgtgggaggggattacaatcttaaatagggttgtcagggaaggcttcatgagggcaaagacctgaggaggcatggaaatgatccacgcagatgcatgtctggggaagagcattccaggcagaggcagtgggaatggaccctcaggtaagagcatgtttagcaagttcagagaaaagcctcttaggtcaggtctggagcgagagtggaaaatgtaacaggacagatcatgtaaagacttgtagaccacagttagaggcttggacaataactctgaggatgacatgaaggtattgggagggggtgttgagcagaggagtgatatgatctggtttatattttagattattctgactgctatgttgaaaatagggttgagagcagaggggaagggcagcacaccagttaagaccacttcccaaaaatccagatgatgatgggcttgaaccaggagagttgctatagggatgtgaagcaatcaaattctagatatttgggacctgctgacaggtacataatggtgtatgagagaaaaggcagtgtgagaatgactctgaagtctgtggcctgagcaaaacagaaacctggagttgccaatagctgagataaagtttctggttaaagatcaagagctcagttttggacaagaggggtgtgagatttctaatagacattcaggtggaggtgtggggaacacacctggatgtgatgagcctggaattcaaattcacatgtgtgaatttgaaaaaagattcagaaaagaggtgtggactggaggagctatccacacacagatagcatttgaagccacaagactgggggtgatgacgcagggagtaagtataaatagaaaagagaagtggtccaaggactcagtccttacatcagaggtcaaagagacaagatggagactaggagatatgaaggcttatgcagtgagtgtttcggagaaacgaagaaacttcccagaagaaaggagaggtcaactgctaacaagtcaagtgaaatgagaactgggaaatgagttgacaagagcaatttggctggcgtgatcagagaatgtttaagagataatgtgagggaagaaaatggaggcaggaagtatgaactactcttttgagtcatattgtaaaggaaaaaaatggggtgattttactcttttttttttttaaagataggaaaaaaaagtatatttatatgctgaggagaagaatccagttgaaggggaatactgtggcttcaggagacaggaaaactgcctaagccacatccttcagcaagtgagtgttcaagtggagggtttgtctttgcttggggcacgggcggttcatccacagtgaaaggagagaaggcagggcatgtggggttggctgtagaaagcgggcagacggggtgggaacttatgaagactcttctgattgttattttctctgttaagtaaaaggaaaggtcattggctaagaatgaagatggacgcgcaggtgctgggagtcatcgaagagaatgaaagaatgaataaaatagagaaaaatacaatgctattatataaacctatacatttaaattactttcctcttcaaattagaccccctggaaggcaaatctctgtcatgttaagttttaggaaaagtcataatctacttggagctaagtatgaagaataaggtatatgatttaaaccacataattctatttttgcttaaaccggggaagcctttaaggtaacgtagagtgtacccattaaaaaagataaaaatgtcagcttccccttccctttaaatatagtaaggctcaaaggtaattgatgttcataaccctaaggtactataacagtctgaagcaaaatattaaatgtcttctgtagtattttagttaacttttaattttattggtaatttatgtgtagttaaccacatacttgtgcctcaactatttcacataaatgtgagatgtcttaggcctctttgaactctgcatggaaaagaatgctgaggaggtatgtctgtttaagctgccactgcctgcctctcataaataatatctcacattgcataatagcccagataaactgcagatcgcttagagcctgataaatgagaggaaactgtacagtttgttttccaaattgcccttggacagctaattcacaatttactattttatagctgtaagattttaaaaatataaaataacatagctgtaaatacagattatcagaagatagtctgtaaaaatgtaaatataatatagctgtaaatatacactatcggaagtaccaaatctagttttagtaggtagaggcatcaagtaaaatggaaatgaaagatgtttcaaggaaaactccaggacatctgtggcagtactaaagaaaccttccttctcaggttcccagcatgctattttattgatgtaacaacattttcaataaagttggtaaattatcactatattactgtcttatagcaacatagcaagagctttagagatcatataagtataaaatgtgaatttaaaaaaacaatgaatatgcaggatttttattagggcaagcgtttccataaccataaatatttctttaaaacaaataaatgtcccaagatctctgttagtgatccaaactaagtagaaattagtaaaattaattataaatgaacaatttcagcatataaaccaacaagtcttttctagatttttaacactgtgacccaattgcattattttccaagttagaatgactaataatcaatgaatgtaaaagcaataattaatacagatgacattgtacttttccacagtaaagaaataaacaatctaatatttttataaatcccattttatatcacaaaataacctttactaagcaaatttttttaaaatctcaggaactatagacatgatgaaaagatagatattttatataaataattcaaaaatactgtcaggcaaggaaatgtaaaatccttatttgagtaaaagaaaatgctataaagcaatgagttatcaaaatacagaagaggtattctaaaacaaatgaaaaaccaagatgatgaaatagtgacaactacttctaatgtgtaacagatactgaaatgccaaggtgaaagtgaactgaattatttcttaaagcagtggagaatatgtaactttcaaaaatgcaagaagcacagcaaattaactaattaacttaccacctccttcaaataaaagcgagaacctcttgggagaatttaagcaccattagcagacacatcttagagc

SEQ ID NO: 2 - DNA, 21 bp, homo sapien
ggaagtgttctaaaagagaaa

SEQ ID NO: 3 - DNA, 21 bp, homo sapien
agtaaagagggactagacttt

SEQ ID NO: 4 - DNA, 21 bp, homo sapien
agtaaagaggaactagacttt

SEQ ID NO: 5 - protein, 2261 aa, homo sapien [sequence omitted]

SEQ ID NO: 6 - DNA, 7860 bp, homo sapien
gtccctgctgtgagctctggccgctgccttccagggctcccgagccacacgctgggggtgctggctgagggaacatggcttgttggcctcagctgaggttgctgctgtggaagaacctcactttcagaagaagacaaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaactgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaa

SEQ ID NO: 7 - DNA, 16 bp, homo sapien
  misc_feature (7)...(10): n = a, t, g or c
aggtcannnnaggtca

SEQ ID NO: 8 - DNA, 26 bp, homo sapien
agaggcaggtggatcatttgaggtca

SEQ ID NO: 9 - DNA, 26 bp, homo sapien
ttgaggcgggtgatcacttgaggtca

SEQ ID NO: 10 - DNA, 26 bp, homo sapiens
caaggcgggcagatcacttgaggtta

SEQ ID NO: 11 - DNA, 26 bp, Homo sapien
caaggtgggcagctcacctcaggtca

SEQ ID NO: 12 - DNA, 24 bp, Homo sapiens
  misc_feature (1)...(18): n= a, t, g, c, other or none.
nnnnnnannttgacctnntgacct

SEQ ID NO: 13 - DNA, 24 bp, Homo sapiens
ctttgaagcctgatcatatgacct

SEQ ID NO: 14 - DNA, 24 bp, Homo sapiens
aggctggtctcgaactcctgacct

SEQ ID NO: 15 - DNA, 24 bp, Homo sapien
cttaattggtggwgctgttgacct

SEQ ID NO: 16 - DNA, 24 bp, Homo sapien
caggatggcgtaaactcctgacct

SEQ ID NO: 17 - DNA, 24 bp, homo sapien
aggttggtttcgaactcctgacct

SEQ ID NO: 18 - DNA, 24 bp, Homo sapein
tcaaggtaggagaccttgtggcct

SEQ ID NO: 19 - DNA, 10 bp, homo sapien
atcaccccac

SEQ ID NO: 20 - DNA, 20 bp, homo sapien
gagatgtgctatgaccccac

SEQ ID NO: 21 - DNA, 20 bp, homo sapien
gtgagcccagatcacaccac

SEQ ID NO: 22 - DNA, 20 bp, homo sapien
tccatccatccacaccccac

SEQ ID NO: 23 - DNA, 20 bp, homo sapien
cccttttattaacacctcac

SEQ ID NO: 24 - DNA, 20 bp, homo sapien
gtaagccaagatcatgccac

SEQ ID NO: 25 - DNA, 20 bp, homo sapien
acctcaagtgatcacccgcc

SEQ ID NO: 26 - DNA, 20 bp, homo sapien
ggctcaagcgatcctcccac

SEQ ID NO: 27 - DNA, 20 bp, homo sapien
ccatgattggatcactgcac

SEQ ID NO: 28 - DNA, 20 bp, homo sapien
gtgagtcgagatcatgccac

SEQ ID NO: 29 - DNA, 20 bp, homo sapien
tgcttttgttttccccccac

SEQ ID NO: 30 - DNA, 20 bp, homo sapien
ccgccttcccctcaccccag

SEQ ID NO: 31 - DNA, 20 bp, homo sapien
accctccacccccaccccac

SEQ ID NO: 32 - DNA, 16 bp, homo sapien
  variation (1)...(11): n at positions 2-9 is a, t, g or c; n at positions 1 and 10 is a or t; n at position 11 is a or g.
nnnnnnnnnnnggtca

SEQ ID NO: 33 - DNA, 18 bp, homo sapien
ctgggcaaggatgggtca

SEQ ID NO: 34 - DNA, 17 bp, homo sapiens
tgggcaaggatgggtca

SEQ ID NO: 35 - DNA, 17 bp, homo sapiens
aaaaagcaccaaggtca

SEQ ID NO: 36 - DNA, 17 bp, homo sapien
agaagatgccagggtca

SEQ ID NO: 37 - DNA, 17 bp, homo sapiens
gaggagatggagggtca

SEQ ID NO: 38 - DNA, 26 bp, homo sapiens
ccgagcgcagaggttactatcggtca

SEQ ID NO: 39 - DNA, 26 bp, homo sapien
gccaattcccaggtcagaacagacca

SEQ ID NO: 40 - DNA, 20 bp, homo sapien
ggacctgcagctctccccac

SEQ ID NO: 41 - DNA, 17 bp, homo sapien
aacgcccaagtaagtca

SEQ ID NO: 42 - DNA, 17 bp, homo sapien
gagctcgtactaggaca

SEQ ID NO: 43 - DNA, 17 bp, homo sapien
gcagagtcctggggtca

SEQ ID NO: 44 - DNA, 18 bp, homo sapien
cgcagagtcctggggtca

SEQ ID NO: 45 - DNA, 17 bp, homo sapien
agccaattcccaggtca

SEQ ID NO: 46 - DNA, 17 bp, homo sapien
acggaccgtttgggaca

SEQ ID NO: 47 - DNA, 18 bp, homo sapien
cacggaccgtttgggaca

SEQ ID NO: 48 - DNA, 19 bp, homo sapien
ccacggaccgtttgggaca

SEQ ID NO: 49 - DNA, 18 bp, homo sapien
actagaggccttgggtct

SEQ ID NO: 50 - DNA, 17 bp, homo sapien
ctagaggccttgggtct

SEQ ID NO: 51 - DNA, 17 bp, homo sapien
ccctacccctcaggtca

SEQ ID NO: 52 - DNA, 18 bp, homo sapien
tccctacccctcaggtca

SEQ ID NO: 53 - DNA, 17 bp, homo sapien
  misc_feature (2)...(2): n = c or g.
gntctgcgccagggaca

SEQ ID NO: 54 - DNA, 17 bp, homo sapien
  misc_feature (12)...(12): n = a, t, g or c.
ttttagtgaganggtta

SEQ ID NO: 55 - DNA, 26 bp, homo sapien
tgaggcaggtagatcacttgaggtca

SEQ ID NO: 56 - DNA, 26 bp, homo sapien
cgaggctggcggatcacctgaggtca

SEQ ID NO: 57 - DNA, 26 bp, homo sapien
aagcctaacaaggttactgaaggcca

SEQ ID NO: 58 - DNA, 26 bp, homo sapien
agaggtgggcggatcacctgaggtca

SEQ ID NO: 59 - DNA, 24 bp, homo sapien
ctcgatttcctgacctcgtgatcc

SEQ ID NO: 60 - DNA, 24 bp, homo sapien
caaaacattgtgcccttttgaact

SEQ ID NO: 61 - DNA, 24 bp, homo sapien
gcgctagggttgtcctcattacct

SEQ ID NO: 62 - DNA, 24 bp, homo sapien
ctcgatttcttgacctcgtgatcc

SEQ ID NO: 63 - DNA, 20 bp, homo sapien
gtgagctgagatcacaccac

SEQ ID NO: 64 - DNA, 20 bp, homo sapien
ttcaaggatgatcaccacat

SEQ ID NO: 65 - DNA, 20 bp, homo sapien
ggctcaagtgatcctcccac

SEQ ID NO: 66 - DNA, 20 bp, homo sapien
gtgagccgagatcgcgccac

SEQ ID NO: 67 - DNA, 20 bp, homo sapien
gtgagttatgatcatgccac

SEQ ID NO: 68 - DNA, 20 bp, homo sapien
ccactgtttgaacaacccac

SEQ ID NO: 69 - DNA, 20 bp, homo sapien
acctcaggtgatccgcccac

SEQ ID NO: 70 - DNA, 20 bp, homo sapien
aaatgtgacaatctccacac

SEQ ID NO: 71 - DNA, 20 bp, homo sapien
aatatagaatatcacctccc

SEQ ID NO: 72 - DNA, 20 bp, homo sapien
ccttttatctaccacccaac

SEQ ID NO: 73 - DNA, 17 bp, homo sapien
ccttgttggatgggtca

SEQ ID NO: 74 - DNA, 18 bp, homo sapien
gccttgttggatgggtca

SEQ ID NO: 75 - DNA, 17 bp, homo sapien
ttgctgtgagtgggtca

SEQ ID NO: 76 - DNA, 17 bp, homo sapien
gccttgcagaagggtca

SEQ ID NO: 77 - DNA, 18 bp, homo sapien
ggccttgcagaagggtca

SEQ ID NO: 78 - DNA, 18 bp, homo sapien
aattaagctgatgggtca

SEQ ID NO: 79 - DNA, 17 bp, homo sapien
attaagctgatgggtca

SEQ ID NO: 80 - DNA, 18 bp, homo sapien
aggtgctaactagggtca

SEQ ID NO: 81 - DNA, 17 bp, homo sapien
ggtgctaactagggtca

SEQ ID NO: 82 - DNA, 17 bp, homo sapien
atgggatgactgggtca

SEQ ID NO: 83 - DNA, 17 bp, homo sapien
tctccatgccaaggtca

SEQ ID NO: 84 - DNA, 23 bp, homo sapien
ttgaggacatgcggtacgtctgg

SEQ ID NO: 85 - DNA, 23 bp, homo sapien
ttgaggacatgtggtacgtctgg

SEQ ID NO: 86 - DNA, 21 bp, homo sapien
gcctacttgcaggatgtggtg

SEQ ID NO: 87 - DNA, 21 bp, homo sapien
gcctacttgcgggatgtggtg

SEQ ID NO: 88 - DNA, 23 bp, homo sapien
cctcattcctcttcttgtgagcg

SEQ ID NO: 89 - DNA, 20 bp, homo sapien
cctcattcctcttgtgagcg

SEQ ID NO: 90 - DNA, 21 bp, homo sapien
aaaagtctaccgagatgggat

SEQ ID NO: 91 - DNA, 21 bp, homo sapien
aaaagtctactgagatgggat

SEQ ID NO: 92 - DNA, 21 bp, homo sapien
ggccagatcacctccttcctg

SEQ ID NO: 93 - DNA, 21 bp, homo sapien
ggccagatcatctccttcctg

SEQ ID NO: 94 - DNA, 21 bp, homo sapien
acacaccacatggatgaagcg

SEQ ID NO: 95 - DNA, 21 bp, homo sapien
acacaccacacggatgaagcg

SEQ ID NO: 96 - DNA, 21 bp, homo sapien
cctggaagaagtaagttaagt

SEQ ID NO: 97 - DNA, 21 bp, homo sapien
cctggaagaactaagttaagt

SEQ ID NO: 98 - DNA, 21 bp, homo sapien
gctgcctgtgtgtcccccagg

SEQ ID NO: 99 - DNA, 21 bp, homo sapien
gctgcctgtgcgtcccccagg

SEQ ID NO: 100 - DNA, 22 bp, homo sapien
tagccattatggaattactgct

SEQ ID NO: 101 - DNA, 21 bp, homo sapien
tagccattatcaattactgct

SEQ ID NO: 102 - DNA, 26 bp, homo sapien
gatgaagatgaagatgtgaggcggga

SEQ ID NO: 103 - DNA, 20 bp, homo sapien
gatgaagatgtgaggcggga

SEQ ID NO: 104 - DNA, 21 bp, homo sapien
aatagttgtacgaatagcagg

SEQ ID NO: 105 - DNA, 21 bp, homo sapien
aatagttgtatgaatagcagg

SEQ ID NO: 106 - DNA, 20 bp, homo sapien
atagttgtacgaatagcagg

SEQ ID NO: 107 - DNA, 19 bp, homo sapien
atagttgtagaatagcagg

SEQ ID NO: 108 - DNA, 20 bp, homo sapien
gggtccaacccggacctgaa

SEQ ID NO: 109 - DNA, 20 bp, homo sapien
gggtccaacctggacctgaa

SEQ ID NO: 110 - DNA, 21 bp, homo sapien
cattatcttctctggccagga

SEQ ID NO: 111 - DNA, 20 bp, homo sapien
cattatctttttggccagga

SEQ ID NO: 112 - DNA, 20 bp, homo sapien
ggaactagtcccggcaaaaa

SEQ ID NO: 113 - DNA, 20 bp, homo sapien
ggaactagtctcggcaaaaa

SEQ ID NO: 114 - DNA, 17 bp, homo sapien
ccgggacccgcagagcc

SEQ ID NO: 115 - DNA, 17 bp, homo sapien
ccgggaccggcagagcc

SEQ ID NO: 116 - DNA, 20 bp, homo sapien
accagccacggcgtccctgc

SEQ ID NO: 117 - DNA, 21 bp, homo sapien
accagccacgggcgtccctgc

SEQ ID NO: 118 - DNA, 21 bp, homo sapien
acacgctgggggtgctggctg

SEQ ID NO: 119 - DNA, 21 bp, homo sapien
acacgctgggcgtgctggctg

SEQ ID NO: 120 - DNA, 21 bp, homo sapien
ctgggttcctgtatcacaacc

SEQ ID NO: 121 - DNA, 21 bp, homo sapien
ctgggttcctatatcacaacc

SEQ ID NO: 122 - DNA, 21 bp, homo sapien
ggcctaccaagggagaaactg

SEQ ID NO: 123 - DNA, 21 bp, homo sapien
ggcctaccaaaggagaaactg

SEQ ID NO: 124 - DNA, 21 bp, homo sapien
gcgggcatcccgagggagggg

SEQ ID NO: 125 - DNA, 21 bp, homo sapien
gcgggcatcctgagggagggg

SEQ ID NO: 126 - DNA, 21 bp, homo sapien
agggaggggggctgaagatca

SEQ ID NO: 127 - DNA, 21 bp, homo sapien
agggagggggactgaagatca

SEQ ID NO: 128 - DNA, 21 bp, homo sapien
tgactccaggtgaacaagacc

SEQ ID NO: 129 - DNA, 21 bp, homo sapien
tgactccaggcgaacaagacc

SEQ ID NO: 130 - DNA, 21 bp, homo sapien
gcaggactacgtgggcttcac

SEQ ID NO: 131 - DNA, 21 bp, homo sapien
gcaggactacatgggcttcac

SEQ ID NO: 132 - DNA, 21 bp, homo sapien
cgtgggcttcacactcaagat

SEQ ID NO: 133 - DNA, 21 bp, homo sapien
cgtgggcttcccactcaagat

SEQ ID NO: 134 - DNA, 21 bp, homo sapien
tcacactcaagatcttcgctg

SEQ ID NO: 135 - DNA, 21 bp, homo sapien
tcacactcaacatcttcgctg

SEQ ID NO: 136 - DNA, 17 bp, homo sapien
ccacttcggtctccatg

SEQ ID NO: 137 - DNA, 17 bp, homo sapien
ccacttcgatctccatg

SEQ ID NO: 138 - DNA, 18 bp, homo sapien
gaagagaatatcagaaag

SEQ ID NO: 139 - DNA, 18 bp, homo sapien
gaagagaatgtcagaaag

SEQ ID NO: 140 - DNA, 21 bp, homo sapien
gatctaaggttgtcattctgg

SEQ ID NO: 141 - DNA, 21 bp, homo sapien
gatctaaggtggtcattctgg

SEQ ID NO: 142 - DNA, 20 bp, homo sapien
gcgaccatgagagtgacacg

SEQ ID NO: 143 - DNA, 20 bp, homo sapien
gcgaccatgacagtgacacg

SEQ ID NO: 144 - DNA, 21 bp, homo sapien
ctggacaccagaaataatgtc

SEQ ID NO: 145 - DNA, 21 bp, homo sapien
ctggacaccaaaaataatgtc

SEQ ID NO: 146 - DNA, 21 bp, homo sapien
tcctatgtgtcctccaccaat

SEQ ID NO: 147 - DNA, 21 bp, homo sapien
tcctatgtgtgctccaccaat

SEQ ID NO: 148 - DNA, 21 bp, homo sapien
cagggtccaacccggacctga

SEQ ID NO: 149 - DNA, 21 bp, homo sapien
cagggtccaatccggacctga

SEQ ID NO: 150 - DNA, 22 bp, homo sapien
cgggggaagggcacgcagaccg

SEQ ID NO: 151 - DNA, 22 bp, homo sapien
cgggggaaggggacgcagaccg

SEQ ID NO: 152 - DNA, 17 bp, homo sapien
ggccgggaacggggcgg

SEQ ID NO: 153 - DNA, 17 bp, homo sapien
ggccgggaagggggcgg

SEQ ID NO: 154 - DNA, 21 bp, homo sapien
agtatcccttgttcacgagaa

SEQ ID NO: 155 - DNA, 25 bp, homo sapien
agtatccctcccttgttcacgagaa

SEQ ID NO: 156 - DNA, 21 bp, homo sapien
gtgacacccaacggagtaggg

SEQ ID NO: 157 - DNA, 21 bp, homo sapien
gtgacacccagcggagtaggg

SEQ ID NO: 158 - DNA, 25 bp, homo sapien
tatgtgctgaccatgggagcttgtt

SEQ ID NO: 159 - DNA, 25 bp, homo sapien
tatgtgctgaccgtgggagcttgtt

SEQ ID NO: 160 - DNA, 25 bp, homo sapien
cctccgcctgccaggttcagcgatt

SEQ ID NO: 161 - DNA, 25 bp, homo sapien
cctccgcctgccgggttcagcgatt

SEQ ID NO: 162 - DNA, 21 bp, homo sapien
gaaaattagtatgtaaggaag

SEQ ID NO: 163 - DNA, 21 bp, homo sapien
gaaaattagtctgtaaggaag

SEQ ID NO: 164 - DNA, 22 bp, homo sapien
cattttcttagaaaagagaggt

SEQ ID NO: 165 - DNA, 22 bp, homo sapien
cattttcttagagaagagaggt

SEQ ID NO: 166 - DNA, 20 bp, homo sapien
  misc_feature (11)...(11): n = g or t
tttaaaggggntgattagga

SEQ ID NO: 167 - DNA, 22 bp, homo sapien
  misc_feature (11)...(11): n = g or t
gaagaaatttntttttttgatt

SEQ ID NO: 168 - DNA, 19 bp, homo sapien
tctgtccccatccctgacg

SEQ ID NO: 169 - DNA, 20 bp, homo sapien
tctgtccccaatccctgacg

SEQ ID NO: 170 - DNA, 20 bp, homo sapien
aggagccaaacgctcattgt

SEQ ID NO: 171 - DNA, 21 bp, homo sapien
aggagccaaagcgctcattgt

SEQ ID NO: 172 - DNA, 21 bp, homo sapien
aagccactgtttttaaccagt

SEQ ID NO: 173 - DNA, 21 bp, homo sapien
aagccactgtatttaaccagt

SEQ ID NO: 174 - DNA, 22 bp, homo sapien
gctccctctagcatgcaggctc

SEQ ID NO: 175 - DNA, 22 bp, homo sapien
gctccctctagtatgcaggctc

SEQ ID NO: 176 - DNA, 21 bp, homo sapien
ttgcctgtttctcacagagcc

SEQ ID NO: 177 - DNA, 19 bp, homo sapien
ttgcctgtttctcagagcc

SEQ ID NO: 178 - DNA, 21 bp, homo sapien
  misc_feature (10)...(10): n = c or g
gcgcagtgcnctgtgtcctta

SEQ ID NO: 179 - DNA, 23 bp, homo sapien
  misc_feature (12)...(12): n = g or t
ctcttctgttancacagaagaga

SEQ ID NO: 180 - DNA, 21 bp, homo sapien
  misc_feature (11)...(11): n = a or g
cattctagggntcatagccat

SEQ ID NO: 181 - DNA, 22 bp, homo sapien
  misc_feature (11)...(11): n = g or t
aagtacagtgngaggaacagcg

SEQ ID NO: 182 - DNA, 22 bp, homo sapien
  misc_feature (12)...(12): n = a or g
attcctaaaaantagaaatgca

SEQ ID NO: 183 - DNA, 23 bp, homo sapien
tttctgtttcaattcttgtctat

SEQ ID NO: 184 - DNA, 23 bp, homo sapien
tttctgtttcagttcttgtctat

SEQ ID NO: 185 - DNA, 21 bp, homo sapien
ggcccctgccttattattact

SEQ ID NO: 186 - DNA, 21 bp, homo sapien
ggcccctgccgtattattact

SEQ ID NO: 187 - DNA, 21 bp, homo sapien
cactgtctgggttttaatgtc

SEQ ID NO: 188 - DNA, 21 bp, homo sapien
cactgtctggcttttaatgtc

SEQ ID NO: 189 - DNA, 22 bp, homo sapien
  misc_feature (11)...(11): n = a or g
tgagagaattncttgaacccgg

SEQ ID NO: 190 - DNA, 21 bp, homo sapien
  misc_feature (11)...(11): n = c or t
tttgctgaaanaatcactgac

SEQ ID NO: 191 - DNA, 22 bp, homo sapien
  misc_feature (11)...(11): n = c or t
aacctcagttncctcatctgtg

SEQ ID NO: 192 - DNA, 21 bp, homo sapien
aagaagtggcttgtattttgc

SEQ ID NO: 193 - DNA, 21 bp, homo sapien
aagaagtggcctgtattttgc

SEQ ID NO: 194 - DNA, 23 bp, homo sapien
  misc_feature (11)...(11): n = a or g
aactgatttgnttggtatagctg

SEQ ID NO: 195 - DNA, 20 bp, homo sapien
aataaagataatttctttgg

SEQ ID NO: 196 - DNA, 20 bp, homo sapien
aataaagatagtttctttgg

SEQ ID NO: 197 - DNA, 22 bp, homo sapien
ttcctgccccgacactcccgcc

SEQ ID NO: 198 - DNA, 22 bp, homo sapien
ttcctgcccccacactcccgcc

SEQ ID NO: 199 - DNA, 22 bp, homo sapien
  misc_feature (11)...(11): n = a or g
ttgggaggctnaggcaggagaa

SEQ ID NO: 200 - DNA, 22 bp, homo sapien
tgtcagctgttactggaagtgg

SEQ ID NO: 201 - DNA, 22 bp, homo sapien
tgtcagctgctgctggaagtgg

SEQ ID NO: 202 - DNA, 21 bp, homo sapien
aggagctggccgaagccacaa

SEQ ID NO: 203 - DNA, 21 bp, homo sapien
aggagctggctgaagccacaa

SEQ ID NO: 204 - DNA, 21 bp, homo sapien
aatgatgccaccaaacaaatg

SEQ ID NO: 205 - DNA, 21 bp, homo sapien
aatgatgccatcaaacaaatg

SEQ ID NO: 206 - DNA, 21 bp, homo sapien
gaggtggctccgatgaccaca

SEQ ID NO: 207 - DNA, 21 bp, homo sapien
gaggtggctctgatgaccaca

SEQ ID NO: 208 - DNA, 21 bp, homo sapien
ttccttaacagaaatagtatc

SEQ ID NO: 209 - DNA, 21 bp, homo sapien
ttccttaacaaaaatagtatc

SEQ ID NO: 210 - DNA, 21 bp, homo sapien
ggaagtgttccaaaagagaaa

SEQ ID NO: 211 - DNA, 34 bp, Artificial Sequence
  Synthetic primer
gtatttttgcaaggctaccagttacatttgacaa

SEQ ID NO: 212 - DNA, 28 bp, Artificial Sequence
  Synthetic primer
gattggcttcaggatgtccatgttggaa

SEQ ID NO: 213 - DNA, 20 bp, Artificial Sequence
  Synthetic primer
gctgctgtgatggggtatct

SEQ ID NO: 214 - DNA, 20 bp, Artificial Sequence
  Synthetic primer
acctcactcacacctgggaa

SEQ ID NO: 215 - DNA, 20 bp, Artificial Sequence
  Synthetic primer
caagtgagtgcttgggattg

SEQ ID NO: 216 - DNA, 21 bp, Artificial Sequence
  Synthetic primer
tgcttttattcagggactcca

SEQ ID NO: 217 - DNA, 25 bp, Artificial Sequence
  Synthetic primer
gtgatcccagcgtggtgtttgtctt

SEQ ID NO: 218 - DNA, 37 bp, Artificial Sequence
  Synthetic primer
gaaaggccagaggtactcacagcgaagatcttgaggg

SEQ ID NO: 219 - DNA, 21 bp, Artificial Sequence
  Synthetic primer
tcgttttattcagggactcca

SEQ ID NO: 220 - DNA, 20 bp, Artificial Sequence
  Synthetic primer
caagtgagtgcttgggattg

SEQ ID NO: 221 - DNA, 20 bp, Artificial Sequence
  Synthetic primer
cccatgcactgcagagattc

SEQ ID NO: 222 - DNA, 21 bp, Artificial Sequence
  Synthetic primer
gcaaattcaaatttctccagg

SEQ ID NO: 223 - DNA, 35 bp, Artificial Sequence
  Synthetic primer
gagaagagccaccctggttccaaccagaagaggat

SEQ ID NO: 224 - DNA, 19 bp, Artificial Sequence
  Synthetic primer
aaggcaggagacatcgctt

SEQ ID NO: 225 - DNA, 36 bp, Artificial Sequence
  Synthetic primer
gagcagttctgatgctggcctgggcagcgaccacga

SEQ ID NO: 226 - DNA, 20 bp, Artificial Sequence
  Synthetic primer
tctgcacctctcctcctctg

SEQ ID NO: 227 - DNA, 35 bp, Artificial Sequence
  Synthetic primer
cagcttgggaagatttatgacaggactggacacga

SEQ ID NO: 228 - DNA, 18 bp, Artificial Sequence
  Synthetic primer
atgcccctgccaacttac

SEQ ID NO: 229 - DNA, 27 bp, Artificial Sequence
  Synthetic primer
gtgcaattacgttgtccctgccacact

SEQ ID NO: 230 - DNA, 30 bp, Artificial Sequence
  Synthetic primer
ccatacagcaaaagtagaagggctagcaca

SEQ ID NO: 231 - DNA, 16 bp, homo sapien
  misc_feature (7)...(10): n = a, t, g or c
agatcannnnaggtca

SEQ ID NO: 232 - DNA, 16 bp, Homo sapien
agatcacttgaggtca

SEQ ID NO: 233 - DNA, 22 bp, Artificial Sequence
  Synthetic Primer
cagcgcttcccgcgcgtcttag

SEQ ID NO: 234 - DNA, 24 bp, Artificial Sequence
  Synthetic Primer
ccactcactctcgtccgcaattac

SEQ ID NO: 235 - DNA, 38 bp, Artificial Sequence
  Synthetic Primer
ctgctgagtgactgaactacataaacagaggccgggta

SEQ ID NO: 236 - DNA, 24 bp, Artificial Sequence
  Synthetic Primer
ccactcactctcgtccgcaattac

SEQ ID NO: 237 - DNA, 22 bp, Artificial Sequence
  Synthetic Primer
cagcgcttcccgcgcgtcttag

SEQ ID NO: 238 - DNA, 24 bp, Artificial Sequence
  Synthetic Primer
ccactcactctcgtccgcaattac

SEQ ID NO: 239 - DNA, 20 bp, Artificial Sequence
  Synthetic Primer
ctggctttctgctgagtgac

SEQ ID NO: 240 - DNA, 19 bp, Artificial Sequence
  Synthetic Primer
gatcaaagtccccgaaacc

SEQ ID NO: 241 - DNA, 30 bp, Artificial Sequence
  Synthetic Primer
actcagttgtataacccactgaaaatgagt

SEQ ID NO: 242 - DNA, 23 bp, Artificial Sequence
  Synthetic Primer
ttctatagatgttatcatctggg

SEQ ID NO: 243 - DNA, 30 bp, Artificial Sequence; identical to SEQ ID NO: 241
  Synthetic Primer

SEQ ID NO: 244 - DNA, 23 bp, Artificial Sequence
  Synthetic Primer
ttctatagatgttatcatctggg

SEQ ID NO: 245 - DNA, 20 bp, Artificial Sequence
  Synthetic Primer
tcatctaaggcacgttgtgg

SEQ ID NO: 246 - DNA, 20 bp, Artificial Sequence
  Synthetic Primer
cctcaagcctggagtgactt

SEQ ID NO: 247 - DNA, 20 bp, Artificial Sequence
  Synthetic Primer
atggcaaacagtcctccaag

SEQ ID NO: 248 - DNA, 20 bp, Artificial Sequence
  Synthetic Primer
accctagcgctgtgtctctg

SEQ ID NO: 249 - DNA, 20 bp, Artificial Sequence
  Synthetic Primer
atggcaaacagtcctccaag

SEQ ID NO: 250 - DNA, 20 bp, Artificial Sequence
  Synthetic Primer
accctagcgctgtgtctctg

SEQ ID NO: 251 - DNA, 20 bp, Artificial Sequence
  Synthetic Primer
tgtgtgtcctcccttccatt

SEQ ID NO: 252 - DNA, 20 bp, Artificial Sequence
  Synthetic Primer
cttggaggactgtttgccat

SEQ ID NO: 253 - DNA, 38 bp, Artificial Sequence
  Synthetic Primer
cccctcctgctttatctttcagttaatgaccagccccg

SEQ ID NO: 254 - DNA, 20 bp, Artificial Sequence
  Synthetic Primer
atccccaactcaaaaccaca

SEQ ID NO: 255 - DNA, 37 bp, Artificial Sequence
  Synthetic Primer
gccgctgccttccagggctcccgagccacacgctgcg

SEQ ID NO: 256 - DNA, 20 bp, Artificial Sequence
  Synthetic Primer
atccccaactcaaaaccaca